(19) United States
(12) Patent Application Publication
(10) Pub. No.: US 2011/0065645 A1
(43) Pub. Date: Mar. 17, 2011

(54) Compositions and Methods for Modulating Neuron Degeneration and Neuron Guidance

(75) Inventor: Yimin ZOU, San Diego, CA (US)

(73) Assignee: The Regents of the University of California, Oakland, CA (US)

(21) Appl. No.: 12/879,883

(22) Filed: Sep. 10, 2010

Related U.S. Application Data
(60) Provisional application No. 61/241,334, filed on Sep. 10, 2009.
(60) Provisional application No. 61/255,378, filed on Oct. 27, 2009.

(51) Int. Cl. A61K 38/17; A61K 31/713; A61P 25/00; A61P 25/28; A61P 25/16; C12N 5/02

(52) U.S. Cl. 514/17.8; 514/44 A; 514/17.7; 435/375

(57) ABSTRACT

Methods for inhibiting degeneration of a neuron, methods of treating a neurodegenerative disease, methods for promoting degeneration of a neuron are provided, methods for modulating neuron cell guidance of a neuron, as well as compounds useful in the methods of the invention, such as a Wnt compound, a Fzd3 dephosphorylating agent, a Fzd3 phosphorylating agent, or a SHH compound as disclosed herein.

### CROSS-REFERENCES TO RELATED APPLICATIONS

This application claims the benefit under 35 U.S.C. §1.119(e) of U.S. Application No. 61/241,334, filed Sep. 10, 2009, and U.S. Application No. 61/255,378, filed Oct. 27, 2009, the contents of which are incorporated by reference in the entirety.

### STATEMENT AS TO RIGHTS TO INVENTIONS MADE UNDER FEDERALLY SPONSORED RESEARCH OR DEVELOPMENT

This invention was sponsored by the U.S. government under grant RO1-NS047484 and RO1-NS046357. The U.S. government may have certain rights in the invention.

### BACKGROUND OF THE INVENTION

In recent years, increasing evidence suggests that Wnts, which have been better known as morphogens in early development, are conserved axon guidance molecules during nervous system wiring both in vertebrates and invertebrates (Zou, Y. 2004. Trends Neurosci 27:528-32; Fradkin, et al. 2005. J Neurosci 25:10376-8; Zou & Lyuksyutova. 2007. Curr Opin Neurobiol 17:22-8; Salinas, et al. 2008. Annu Rev Neurosci 31:339-358). Wnts are secreted glycoproteins, which bind to three classes of receptors, the Frizzleds, Ryk and ROR2 (Gordon & Nusse. 2006. J Biol Chem 281:22429-33; Logan & Nusse. 2004. Annu Rev Cell Dev Biol 20:781-810). It has also been shown that the Wnt family proteins are essential guidance cues along the A-P axis of the spinal cord and topographic map formation in the retinotectal projections in development and may play important roles in regulating adult CNS axon regeneration after spinal cord injury (Lyuksyutova et al. 2003. Science 302:1984-8; Liu, et al. 2005. Nat Neurosci 8:1151-9; Schmitt et al. 2006. Nature 439:31-7; Wolf et al. 2008. J Neurosci 28:3456-67; Liu et al. 2008. The Journal of Neuroscience 28:8376-8382).

Commissural axons, which originate from the dorsal spinal cord, first project along the dorsal-ventral axis to grow towards the ventral midline, the floor plate. The ventrally directed growth of commissural axons are guided by repulsive cues, the BMPs emanating from the dorsal midline, the roof plate, and attractive cues, Netrin-1 and Sonic Hedgehog, secreted from the floor plate (Zou et al. 2007. Curr Opin Neurobiol 17:22-8). Once these commissural axons cross the midline, they lose responsiveness to midline attractants and gain sensitivity to chemorepllents, the Slits and Semaphorins, emanating from the floor plate and the neighboring ventral gray matter, forcing them to make a 90° turn into their longitudinal trajectory (Zou et al. 2000. Cell 102:363-75). The dorsal populations of rodent commissural axons all turn anterior and project towards the brain. The anterior turning requires Wnt-Frizzled signaling. Several Wnts, including Wnt4, Wnt7b, Wnt7a and Wnt5a, are expressed in an anterior-posterior decreasing gradient along the spinal cord at the ventral midline and attract post-crossing commissural axons, which have crossed the midline to turn anteriorly. When the Wnt gradient was disrupted by adding Wnt inhibitors, secreted Frizzled-related proteins (sFRPs) or positioning Wnt4-secreting cell aggregates in “open-book” explant culture, commissural axons show specific A-P randomize growth after midline crossing. In Frizzled3 mutant embryos, spinal cord commissural axons lose A-P directionality in vivo (Lyuksyutova et al. 2003. Science 302:1984-8).

Studies in Drosophila midline axon pathfinding independently showed that DWnt5 is a chemorepellent and repels a subset of commissural axons via a receptor called, Derailed (Yoshikawa et al. 2003. Nature 422:583-8). The vertebrate homologue of Derailed, Ryk, is also a repulsive Wnt receptor and an anterior-high posterior-low Wnt gradient created by differential expression of Wnt1and Wnt5a, is required for the posterior growth of corticospinal tract axons in the spinal cord (Liu et al. 2005. Nat Neurosci 8:1151-9). Therefore, Wnts control A-P guidance of both ascending and descending axons in the spinal cord by attractive and repulsive guidance mechanisms. Wnt-Ryk signaling was also found to regulate the pathfinding of corpus callosum in the mammalian forebrain by a repulsive mechanism (Keeble et al. 2006. J Neurosci 26:5840-8). Studies in C. elegans showed that Wnt signaling control anterior-posterior directionality of the pathfinding of a number of axons and migration of neuroblasts (Pan et al. 2006. Dev Cell 10:367-77; Hilliard et al. 2006. Dev Cell 10:379-90; Prasad & Clark. 2006. Development 133:1757-66). Therefore, the A-P guidance mechanisms appear to be highly conserved in animal kingdom (Zou. 2006. Neuron 49:787-9.).

In addition to the role of Wnts in axon pathfinding, Wnt3 is also a positional cue for topographic mapping in the retinotectal system, acting as an laterally-directing mapping force for retinal ganglion cell axons, opposing the medially-directed force created by ephrinB1 gradient in the optic tectum (Schmitt et al. 2006. Nature 439:31-7). Wnt3 is expressed in a medial-high to lateral-low gradient in the optic tectum. Ryk is expressed in a dorsal-ventral (D-V) increasing gradient in the retinal ganglion cells. Therefore, the more ventral RGC axon branches are more strongly repelled by Wnt3 and Wnt3-Ryk signaling drives the interstitial branches to grow towards the lateral tectum. In the meantime, ephrinB1 is expressed in the same graded fashion and EphBs are expressed at higher levels in ventral RGCs. EphBs mediate attraction to ephrinB1. Therefore, the more ventral RGC axon branches are more attracted by ephinB1 towards the medial tectum. The balancing act between the medial (ephrinB1) and lateral (Wnt3) mapping forces ensures RGC axons to terminate at correct topographic positions. Remarkably, Wnt-Frizzled signaling is also required for proper dorsal-ventral retinotopic mapping in the Drosophila visual system (Sato et al. 2006. Nat Neurosci 9:67-75; Zou & Lyuksyutova. 2007. Curr Opin Neurobiol 17:22-8). Therefore, Wnts are conserved topographic mapping cues along the D-V axis.

Commissural axons of the developing spinal cord are guided to the ventral midline by a collaboration of chemoattractants (Netrin-1 and Sonic Hedgehog (Shh)) and chemorepellents (Bone Morphogenetic Proteins (BMPs)) secreted by midline floor plate and roof plate cells, respectively. Once these axons reach the floor plate they switch off their responsiveness to chemoattractants from the floor plate and become responsive to chemorepulsive cues also expressed by the floor plate cells and the surrounding ventral gray matter, including members of the Class 3 Semaphorins (Sema3B and Sema3F) and the Slit family proteins (Serafini, T., et al. Cell 78, 409-424, 1994; Kennedy, T. E., et al. Cell 78, 425-435, 1994; Serafini, T., et al. Cell 87, 1001-1014, 1996; Zou, Y., et al. Cell 102, 363-375, 2000; Charron, F., et al. Cell 113, 11-23, 2003; Long, H., et al. Neuron 42, 213-223, 2004). Neuropilin-2 mutant embryos showed severe guidance defects including stalling in the midline, overshooting to the contralateral side of the spinal cord and randomly projecting along the anterior-posterior axis (Zou, Y., et al. Cell 102, 363-375, 2000).

### BRIEF SUMMARY OF THE INVENTION

Provided herein, inter alia, are novel methods for inhibiting degeneration of a neuron, the method comprising contacting the neuron with a Wnt compound or a Fzd3 dephosphorylating agent thereby inhibiting degeneration of the neuron.

In one aspect, provided herein are novel methods of treating a neurodegenerative disease in a subject having or being at risk of developing the neurodegenerative disease by administering to the subject a Wnt compound or a Fzd3 dephosphorylating agent.

In another aspect, provided herein are novel methods of identifying an agent for use in inhibiting degeneration of a neuron. The methods comprise (a) contacting a neuron with a candidate agent; and (b) determining a level of degeneration of the neuron, wherein a lower level of degeneration of the neuron relative to a control, indicates the candidate agent inhibits degeneration of the neuron. Alternatively, the methods comprise (a) contacting a cell with a candidate agent; and (b) determining a level of Fzd3 phosphorylation or a level of Fzd3 internalization; wherein a reduced level of Fzd3 phosphorylation or an increased level of Fzd3 internalization relative to a control, indicates the candidate agent inhibits degeneration of the neuron.

In another aspect, provided herein are novel methods for promoting degeneration of a neuron. The methods comprise contacting the neuron with a Fzd3 phosphorylating agent thereby promoting degeneration of a neuron.

In another aspect, provided herein are novel methods for modulating neuron cell guidance of a neuron. The methods comprise contacting the neuron with a SHH compound thereby modulating neuron cell guidance.

### BRIEF DESCRIPTION OF THE DRAWINGS

FIG. 1. “Open-book” and “post-crossing” assay to test the function of signaling components in commissural axion guidance. FIG. 1A depicts a diagram showing the dorsal-ventral (pre-crossing) and anterior-posterior (post-crossing) trajectory of commissural axons. FIG. 1B depicts that in an “open-book” culture, commissural axons can turn anteriorly if the explant is long enough (e.g., 3 mm) to retain the Wnt gradient. FIG. 1C depicts that in the “post-crossing” assay, a short segment of spinal cord is dissected out with one side cut off but with the floor plate attached to the remaining half. Commissural axons will grow across the midline, exit the floor plate and grow into the collagen gel. When these “post-crossing” commissural axons are exposed to cell arrogates secreting Wnts, they will be attracted to grow towards the cell clump (2). See e.g., Balasubramanian, N.,et al. 2007, Nat Cell Biol 9:1381-91. This assay can be used to test whether a signaling component is required for Wnt attraction (by electroporating a function-blocking construct into the commissural neuron before the “post-crossing” explants are dissected and cultured. See e.g., Beeler, J. A., et al., 2006. Physiol Behav 87:870-80. FIG. 1D depicts that when Wnt signaling-blocking constructs, such as PKCζ⁻ or PI3Kγ⁻ dominant-negative constructs, are introduced into commissural neurons by electroporation, post-crossing axons will grow randomly along the A-P axis after crossing. The same assay can be used to test the function of other signaling-blocking constructs.

FIG. 2. Live imaging of vesicular movement in growth cone in “open-book” explants. FIG. 2A depicts an “open-book” explant electroporated with actin-GFP and VAMP7-mCherry fusion constructs. The white line with arrow indicates the border of the floor plate. Many axons are crossing the midline at this stage. FIGS. 2B-2D depict higher magnification at three time points (i.e., 0, 20 and 40 min, respectively), showing VAMP7 vesicles in different locations. With shorter time intervals, vesicles were seen moving towards the growth cone and entering the filapodia. Arrows indicate the vesicles that were not present in previous picture frames.

FIG. 3. Analogy between cell-cell interaction in Drosophila PCP signaling and potential axon-axon interaction among commissural axons during midline crossing and anterior turning. FIG. 3A depicts ildtype fly ommatidia with normal planar polarity (left panel) and cartoon showing planar polarity (right panel). FIG. 3B depicts wildtype fly wing epithelia with normal planar polarity. FIG. 3C depicts disrupted planar polarity of ommatidia in Vang Gogh mutant (left panel) and cartoon showing disrupted planar polarity (right panel). FIG. 3D depicts disrupted planar polarity of wing epithelial cells in Vang Gogh mutant;. FIG. 3E depicts that proper PCP signaling, asymmetric distribution of core components, requires communications among cells that are in contact. Flamingo, Vang Gogh and Prickle all have cell autonomous and non-autonomous functions. The cell non-autonomous functions are involved in cell-cell communication local signal amplification. FIG. 3F: Many commissural axons cross the midline and turn coordinately anteriorly after midline crossing along the entire length of the spinal cord. Their axon shaft and growth cone may interact with each during their pathfinding. The potential non-autonomous function may be involved in coordinated turning or amplification of graded signals as in PCP signaling in the fly wing epithelia or in axon tiling, preventing commissural axons to cross each other's path.

FIG. 4. Lgl1 overexpression changes membrane distribution in neurons and N2A cells visualized by confocal imaging. FIG. 4A depicts a cortical neuron expressing GFP. FIG. 4B depicts a cortical neuron expressing PKCz-GFP, showing similar morphology as in FIG. 4A. FIG. 4C depicts a cortical neuron expressing Lgl1-GFP. Axons are typically shortened and membranes appear to “collapse towards the cell body or dendrites (arrows indicate membrane “bulges” in or close to the soma). FIG. 4D depicts cortical neurons expressing both PKCz and Lgl1, resulting in intermediate phenotype between B and C (arrows indicate membrane “bulges” in or close to the soma and along axons). FIG. 4E depicts N2A cell expressing GFP. The rectangles on top and to the right side of the picture are a vertical “section” of the confocal image using the distribution of the GFP protein; FIG. 4F depicts N2A cell expressing PKCz-GFP. PKCz is concentrated more towards the cell membrane as shown in the “sections of the confocal images (arrows). FIG. 4G depicts N2A cell expressing Lgl1 shows spread out membrane morphology (arrows). Scale bars in FIGS. 4E-4G: 10 μm.

FIG. 5. Dopaminergic neurons from the substantia nigra fail to innervate the striatum in fz3(−/−) brains (from Wang et al, 2002. J Neurosci.). Tyrosine hydroxylase (FIGS. 5A-5B) and the dopamine transporter (FIGS. 5C-5D), markers for presynaptic nigrostriatal processes, are absent from the fz3(−/−) striatum at embryonic day 18. FIGS. 5E-5F:Tyrosine hydroxylase staining of cell bodies in the substantia nigra demonstrates normal numbers of dopaminergic neurons in the fz3(−/−) brain at embryonic day 18.

FIG. 6. Specific expression of DAT and cPet-1 in mdDA and HT neurons and design of conditional Frizzled3 and Ryk alleles. FIG. 6A depicts (Panels a-b) expression of DAT-Cre as revealed by Rosa26 reporter (LacZ) in SNc and VTA (b) (from Zhuang et al⁽⁶⁶⁾, and control with SERT-Cre (Panels c-d) FIG. 6B depicts expression of 5-HT,TPH, Sert (serotonin transporter), Lmx1b and cPet-1(Panel I and J) in Raphe nuclei (from Zhao et al⁽⁶³⁾). FIGS. 6C-6D) depict design of conditional alleles of Frizzled3 and Ryk. See Example 3 for references.

FIG. 7. Multiple Wnts attract TH axons in explant assays. FIG. 7 depicts histogram showing that embryonic mdDA explants dissected out from the ventral midbrain and cultured with cell clumps expressing various Wnts. TH axons are visualized by anti-TH staining Multiple Wnts showed significant attraction of TH axons with Wnt3a having the strongest effect.

FIG. 8. Costaining with Frizzled3, Vangl2 and α-tubulin antibodies in a dissociated spinal cord commissural axon growth cone. FIG. 8: Frizzled3 and Vangl2 are found localized in close vicinity and with some overlap. They may be present in the same “complex” or membrane “domain”, which is located asymmetrically in a growth cone. The specificity of the Frizzled 3 antibodies has been confirmed by staining in Frizzled3 knockout mice (no staining can be detected in Frizzled3 knockout mice). The Vangl2 antibody was also confirmed by staining in neurons transfected with Vangl2 RNAi. The Vangl2 antibody staining is identical as the pattern revealed by Vangl2-GFP fusion.

FIG. 9. β-catenin distribution can be regulated by Wnts, GSK3β and proteosome function. FIG. 9A: Immunostaining with a β-catenin antibody showing asymmetric localization in a subset of filapodia (arrowheads). FIG. 9B: In the presence of 2 mM LiCl, an inhibitor of GSK3β, β-catenin level is increased throughout the growth cone, canceling the asymmetry (arrowheads). FIG. 9C: A proteosome inhibitor, MG132, increases β-catenin level throughout the growth cone, eliminating asymmetry (arrowheads). FIG. 9D: Bath application of purified Wnt5a protein increases β-catenin level throughout the growth cone (arrowheads). FIG. 9E: Bath application of purified Wnt3a increases β-catenin level throughout the growth cone (arrowhead). FIG. 9F: Bath application of sFRP2 eliminates asymmetric localization of β-catenin.

FIG. 10. Immunostaining with rabbit polyclonal antibodies against tyrosine hydroxylase (TH), a dopaminergic neuron (DA) marker, in E13.5 wild type (left panel) or Frizzled3 knockout embryos (right panel). FIG. 10: Asterisks indicates the position of DA neuron cell bodies. Arrow indicates anteriorly projecting TH fibers. Arrowheads indicate abnormal posteriorly projecting TH fibers in KOs.

FIG. 11. Traces with phase diagrams for L and R L2 and L and R L5. FIG. 11: L and R L5 have synchronous bursts every minute or so whereas L2 is normal, which is very unusual. This result suggests that there is locomotor out put defects in Frizzled3 mutant mice, which may be due to the lack of 5-HT input. L2 is more anterior (rostral) and L5 is more posterior (caudal), suggesting that the defect is likely due to the lack of 5-HT input in the more caudal end (Frizzled3 KO mice has much reduced posterior projection of 5-HT axons) and not due to other axon defects because L2 is normal.

FIG. 12. PKC is required for cortical neuron survival. FIGS. 12A-12D: DAPI and TUNEL staining of culture cortical neurons in the presence of increasing concentration of a myristoylated pseudosubstrate peptide of PKCζ. Increasing percentage of neurons stained positively with TUNEL with increasing dose of the PKCζ inhibitor. FIG. 12E-12F: Axon disintegration induced by PKCζ inhibition as shown by Tuj1 staining Note the massive axon degeneration/fragmentation in F. FIGS. 12G-12I: Only atypical PKC but not the conventional PKC is required for neuronal survival. Note the massive axon degeneration/fragmentation in FIG. 12I. FIGS. 12J-12L: Knockdown of PKCζ or its substrate, Lgl1, with shRNA leads to axon degeneration. Note the axon fragmentation/beading as revealed by GFP staining in FIGS. 12K-12L.

FIG. 13. Axon degeneration procedes cell body death when PKCζ is inhibited. FIG. 13A:Tuj1 staining showed massive axon degeneration 6 hours after the addition of PKCζ inhibitor (almost 80% of the axons). FIG. 13B depicts histogram showing that TUNEL staining is only positive in 25% of the neurons at 6 hours. Order (left to right at each time point): 0 μM, 1 νM, 5 μM. FIG. 13C: Almost all of the neurons showed fragmented or shortened axons (arrow) but only one nucleus stains positive with TUNEL (arrow) at 6 hours. FIG. 13D: At 6 hours, no clear sign of axon degeneration is observed in cells treated with Aβ oligomers.

FIG. 14. Wnts are trophic factors for cortical neurons. Wnt5a, Wnt7a, Ryk antibodies and LiCl protect neurons from degeneration induced by PKCζ inhibition in culture. FIG. 14A: Tuj1 staining showing the loss of microtubule staining induced by pseudosubstrate peptide was reduced by Wnt5a, Wnt7a and Ryk antibodies. FIG. 14B: DAPI staining showing the nuclei size reduction caused by pseudosubstrate peptide was blocked by LiCl. FIG. 14C: Histogram of nuclei size with 0 um peptide, 5 um peptide and 10 um peptide. In no treatment, the peaks of 0 um peptide and those of the 5 um and 10 um are well separated (in bin 425 and bin 275, respectively). However, in Wnt5a, Ryk antibodies and LiCl treatment, the peaks are closer and nuclear size reduction is either partially or completely blocked. FIG. 14D: Evidence of Wnt-Frizzled3-aPKC signaling pathway being required for neuronal survival in vivo. Massive cortical axon degeneration occurs after the initial growth and morphogenesis of cortical neuron axons and dendrites.

FIG. 15. Hypothesis. FIG. 15 depicts a working hypothesis of how Wnt-Frizzled-PKCz signaling promotes neuronal survival and Ryk signaling may stimulate an active neuronal death.

FIG. 16. Reduction of activated PKCζ in the cell bodies and the axons of retinal ganglion cells after optic nerve crush. The levels of activated PKCζ (phosphorylated on Threonine 560) are similar in control and injured retina one day after injury. However, clear and progressive reduction of PKC activity from Day 3 to Day 8 was observed in injured retina. Panels A-C depict controls studies at 1,3 and 8 days, respectively. Panels D-F depicts injury studies at 1, 3 and 8 days, respectively.

FIG. 17. Vgl2 antagonizes Dvl1's feedback inhibition of Fzd3-mediated Wnt/PCP Signaling. FIG. 17A: Fzd3-mCherry (lanes1-4) or both Fzd3-mCherry and Dvl1-EGFP (lanes 5-7) were transfected into HEK293T cells and treated with Wnt5a as shown by immunoblot (IB) with anti-mCherry. Phospho-Jun level is increased when Wnt5a is added in HEK293T cells expressing Fzd3, but down regulated in HEK293T cells expressing both Dvl1 and Fzd3. 20 ug of input lysates were loaded and GAPDH IB was the loading control. Before lysis, the cells were surface biotinylated and immunoprecipitated (IP) with strepavidin beads and immunoblotted (IB) with anti-mCherry (lysates from Lane 1 were the non-biotinylated control). Avidin membrane IP shows that Fzd3 at the membrane appears in two bands (lane 2). However, Fzd3 only appeared in the upper band upon over-expression with Dvl1 (lane 5). FIG. 17B: Quantification of relative phospho-Jun level as measured by gel densitometry analysis with Image J. Fzd3 mediates Wnt5a activation of JNK, however, Dvl1 decreases p-Jun in response to Wnt5a. FIG. 17C: Vangl2 prolongs the PCP signaling. HEK293T cells were transfected with the indicated constructs and treated with Wnt5a. In the presence of Vangl2, the Dvl1-dependent decrease of Wnt-JNK activity (phospho-Jun levels) was attenuated. However, Lp mutant form of Vangl2 (S464N) was unable to prolong the JNK activity, rather promoted the decay in a Wnt5a dependent manner. FIG. 17D: Quantification and plotting of phospho Jun level after normalizing time zero to the value of 1. Fzd3 and Dvl1 co-transfection showed a Wnt5a dependent decrease in p-Jun levels (line). However, Vangl2 was able to attenuate the Dvl dependent decrease in p-Jun (Pink line). The mutant Vangl2, Lp, was unable to antagonize Dvl1 and resulted in Wnt dependent p-Jun level decrease (Black line). FIG. 17E: Fzd3-mCherry transfected 293T cells were subject to surface biotinylation and avidin IP. Protein phosphatase 1 treatment of the surface IP removed the upper Fzd3-mCherry band, which shows that the Fzd3 band shift is caused by phosphorylation. FIG. 17F: Serine 577 is required for Dvl1-induced Fzd3 hyperphosphorylation. A point mutation on amino acid 577 from Ser to Ala prevented the complete band shift to the upper band, suggesting it is one of the phosphorylation sites involved in Fzd3 hyperphosphorylation. GRK2 promotes Fzd3 hyperphosphorylation induced by Dvl1.

FIG. 18: Vgl2 antagonizes Dvl1 in regulating Fzd3 cell surface localization. FIG. 18A: Vgl2 and Lp proteins antagonize Dvl1-induced increase of Fzd3 on the plasma membrane. In the presence of Dvl1, more Fzd3-mCherry protein was present on the plasma membrane (lane 2), 3 fold increase as compared to Fzd3 alone (lane 1). Vgl2 and Lp counter the effect of Dvl1 and reduces Fzd3 levels at the membrane Fzd3 levels (lanes 3 and 4). FIG. 18B: Fzd3 surfaces levels quantified Image J analysis, the final units were normalized making Fzd3 alone (lane 1) equal to the value of 1. FIG. 18C: Fzd3 surface level oscillates in response to Wnt5a. HEK293T cells transfected with Fzd3-EGFP were subject to indicated times of Wnt5a stimulation, then were surface biotinylated and subject avidin IP and anti-EGFP IB for levels of Fzd3 at the membrane. Fzd3-EGFP level from the membrane and cytoplasm during the 2-hour Wnt treatment, first decreasing (lanes 3, 5 and 6) and increases (lane 4). This is a Frizzled3-specific effect because the receptor protein Insulin does not cycle from them membrane upon Wnt addition as shown by anti-Insulin IB. FIG. 18D depicts Wnt mediated surface expression levels as judged by normalized levels of Fzd3 at the membrane quantified with Image J analysis.

FIG. 19: Core PCP signaling components are found in the developing spinal cord and are enriched on pos-crossing commissural axons. FIG. 19A: Schematic view of an E11.5 spinal cord transverse section depicting the trajectory of the commissural axon (dotted line). The pre-crossing segment approaches the ventral midline. The crossing segment and the axon segment after mid-line crossing are also depicted. FIG. 19B: Schematic diagram of the PCP signaling pathway and the core PCP components. FIGS. 19C-19H: In situ hybridization of core PCP component genes, Celsr3, Fzd3, Vgl1, Vgl2, Prkl1 and Prk2 in E11.5 mouse spinal sections. FIG. 19I: In situ hybridization of the Netrin-1 receptor gene DCC at E11.5. Black arrow indicates commissural neuron cell body. FIG. 19J: Sense control for in situ hybridization using Celsr3 sense probe. Scale bar represents 100 um. FIGS. 19K-19L: Celsr3 and Fzd3 receptor expression in the E11.5 mouse spinal cord. Note the enrichment of signals in the post-crossing region of the spinal cord as denoted by white arrow. FIGS. 19M-19N: TAG-1 and L1 are expressed on pre- and post-crossing segments of commissural axons, respectively. White arrow denotes commissural axons. Scale bar: 100 um. FIG. 19O: Phosphorylated-JNK (pJNK) is enriched in post-crossing commissural axons (long arrow). FIGS. 19P-19S: Co-immunostaining in the mouse E11.5 spinal cord showing p-JNK positive axons (arrow) co-express TAG-1 (arrow). P-JNK localization in post-crossing segment is shown by long arrow. Short arrowheads highlight colocalization. Black and white scale bars are 100 um and grey scale bars are 20 um. FIGS. 19T-19W: Endogenous PCP proteins, Celsr3, Fzd3, Vangl2 and Dvl2, are present in commissural neuron growth cones. All components display punctate and plasma membrane localization in commissural growth cones. Scare bar: 10 um.

FIG. 20: Wnt5a activates JNK in dissociated commissural neurons and JNK activity is required for anterior-posterior guidance. FIG. 20A: Wnt5a increases JNK activity measured by p-Jun level after 48 hours of culture. Endogenous Fzd3 and Vgl2 proteins can be detected in these cultured commissural neurons. FIG. 20B: Quantification of p-Jun levels after addition of Wnt5a. FIG. 20C: Schematic of E13 rat spinal cord indicating regions used for western blot analysis (s.c., spinal cord). FIG. 20D: Western blot analysis of dorsal spinal cord (D.sc) enriched with pre-crossing axons, ventral spinal cord (V. sc) enriched with crossing and post-crossing axons and total spinal cord (T.sc) using anti-JNK (left) and anti-phosphorylated-JNK (right) antibodies. FIG. 20E: Inhibition of JNK activity caused A-P randomization of commissural axons. E13 rat open-book spinal cords were first cultured in vitro for 5-6 hours then treated with control diluents or a combination of two JNK inhibitors (JNKI-1 and SP600125) for 18 hours. JNK inhibitor-treated spinal cords resulted in 54.2% (SEM+/−6.39%) misguidance compared to only 9.00% (SEM+/−5.57%) with control.

FIG. 21: Frizzled3 mediates Wnt-stimulated commissural axon outgrowth. Commissural axon length were measured after 24 hrs of culture following electroporation with either EGFP, Fzd3-EGFP, Vang2-EGFP, Dvl1-EGFP or with both Fzd3-mCherry and Vangl2-EGFP in the absence (FIG. 21A) or in the presence of Wnt5a (FIG. 21B). FIG. 21C: The axon lengths are quantified. Bars show control growth and bars depict the effect of growth on Wnts. *Denotes P value of <0.005, **P value of <0.00005

FIG. 22: Both Fzd3 and Vgl2 can localize Dvl1 to plasma membrane of commissural axon growth cones. FIG. 22A: Dvl1-EGFP distribution in commissural neurons. FIG. 22B: Co-localization of Dvl1-EGFP Fzd3-mCherry to the plasma membrane when coexpressed. FIG. 22C: Flag-Vangl2 also targets Dvl1-EGFP to plasma membrane. FIGS. 22D-22D″: Dvl1-EGFP localization in a commissural axon growth cone. FIGS. 22E-22E″: Dvl1-EGFP and Fzd3-mcherry localization on growth cone plasma membrane when coexpressed. FIGS. 22F-22F″: Mutant Dvl1 (DEP domain), Dvl1(KM)-EGFP, does not co-localize with Fzd3-mCherry to the plasma membrane. FIGS. 22G-22G″: Flag-Vgl2 targets Dvl1-EGFP to the plasma membrane. Grey scale bars represent 20 um and white scale bars represent 5 um.

FIG. 23: The Looptail embryos display anterior-posterior defects in commissural axon guidance. FIG. 23A: Schematics of a mouse transverse section depicting the neuronal progenitor domains and neuronal cell types in the spinal cord at E11.5 (dl, dorsal interneurons; MN, motor neurons; dp, dorsal progenitor; p, progenitor). FIGS. 23B-23G: Pax7 and Lhx1/5 expression are unchanged in the Lp spinal cords. Transverse sections of E11.5 Vangl2+/+, Vanlg2+/Lp and the open neural tube Lp/Lp embryos were immunostained with transcription factors expressed by either spinal progenitors or neurons. Pax7 expression by the progenitor domains dp3, dp4, dp5, dp6, p0 and p3 did not change in the wildtype, heterozygous or homozygous embryos (FIGS. 23B-23D). Similarly, Lhx1/5 expression by the post mitotic neurons of dI2, dI4, dI6, dI3 and MN showed no gross differences (FIGS. 23E-23G). (Arrows, cell bodies expressing indicated transcription factors and location of the floor plate). FIG. 23H: Schematics of mouse E11.5 transverse section showing commissural axon trajectory. FIGS. 23I-23N): The dorso-ventral trajectory of commissural axons occurs in Lp spinal cords. TAG-1 immunostaining of E11.5 Vangl2+/+, Vanlg2+/Lp and Lp/Lp embryos sections show that commissural axons in all the embryos reach the midline (FIGS. 23I-23K). L1 immunostaining of E11.5 Vangl2+/+, Vanlg2+/Lp and Lp/Lp embryos sections (L-N). (White arrow, pre-crossing commissural axons; arrow, location of the floor plate; White arrow head, post-crossing commissural axons). FIG. 23O: Schematics of commissural axon trajectory in the open-book prep as revealed by DiI tracing. FIGS. 23P-23R: Vangl2+/Lp and Lp/Lp embryos display aberrant A-P guidance. In these embryos only 31.1% and 5.41% respectively of the DiI injection sites show correct A-P guidance. Arrows indicated posteriorly projecting axons. FIG. 23S: Quantification of DiI injection sites Vangl2+/+, Vanlg2+/Lp and Lp/Lp embryos. 89.2% (SEM+/−3.99%) of wildtype, 31.1% (SEM+/−5.16%) heterozygous and 5.14% (SEM+/−2.93%) of the Lp/Lp embryos showed correct DiI injection sites. Scale bars: 100 um.

FIG. 24: Fzd3 phosphorylation is modulated by PCP components. FIG. 24A: Schematic of all three characterized domains in Dvl1 including the location of Lys 438 to Met mutation that renders the protein PCP signaling defective. FIG. 24B: Dvl1 DEP domain mutant, Dvl1 (K438M)-EGFP cannot promote phosphorylation of Fzd3-mCherry at the membrane. Lysates from HEK 293T cells transfected with the indicated combinations were IB for anti-mCherry, anti-EGFP and anti-GAPDH. The surface biotinylated fractions were subject to IP, and anti-mCherry IB. Dvl1-EGFP and Fzd3-mCherry together results in the phosphorylated Fzd3 upper band (lane 5), however, the Fzd3-mCherry phospho shift disappears when we replace Dvl1, for Dvl1 (KM)-EGFP (lane 6). FIG. 24C: Vgl2 is phosphorylated. 3×Flag-Vgl2 transfected in 293T cells subject to anti-Flag IB showed two distinct migration patterns (lane 1). However, when subject to protein phosphatase 1 treatment the upper band (denoted by *) disappears, indicating that the upper band is a phosphatase sensitive moiety of Vgl2 (lane 2). 3×Flag-Lp, which harbors a S464N mutation is not phospho modified (lane 3). FIG. 24D: Vgl2 keeps Fzd3 dephosphorylated at the membrane. HEK293T cells were transfected with the indicated plasmids and subject to surface phosphorylation and strepavidin IP. Anti-mCherry IB reveals that Fzd3-Cherry in the presence of Vangl2 only is exists as a non-phospho form (lanes 1-3). However, when co transfected with only 3×Flag-Lp, both forms of Fzd3-mCherry is observed, similar to Fzd3-mCherry single transfection (lanes 4-6). In addition, 3×Flag-Vangl2 is phosphorylated and Lp mutation S464N in Vangl2 lacks this modification (anti-Flag IB). This also shows Vangl2 can be phosphorylated in the absence of Dvl1.

FIG. 25: Fzd3 cycling is dependent on concentration and Wnt batches. FIG. 25A. Fzd3 surface expression levels: Fzd3-EGFP cycles from and to the membrane in a Wnt dependent manner, however, the time when internalization and externalization occurs is dependent on the concentration and batch preparation of Wnt5a. High concentration (200 ng/ml) of Wnt5a from batch #1 (lines) results in internalization at 15 minute and two hour time points. Where as low concentration (100 ng/ml) of Wnt5a from batch #1 results in internalization to be delayed to 30 min of Wnt exposure (Light line). High concentration of Wnt5a batch #2 results in a different cycling response, internalization at 15 min and 60 mins. Therefore, the time of cycling is dependent on the source and concentration of Wnt5a, however, in all cases Fzd3 is cycle to and from the membrane. FIG. 25B: PCP components effect Fzd3 cycling to and from the membrane. The indicated constructs are transfected, Wnt5a treated, surface biotinylated followed by avidin-IP and visualized by IB. In the presence of Dvl1-, Fzd3 no longer oscillates from the membrane, Fzd3 is hyperphosphorylated and fails to internalize in a Wnt5a-dependent manner (lanes 5-7). The total Fzd3 surface amount is quantified in the graph, the line shows the behavior of Fzd3 at the membrane, and the line shows its behavior in the presence of Dvl1. However, in the presence of Vangl2, Fzd3 is dephosphorylated and internalization resumes (lanes 8-19 and line in graph), Lp fails to function like Vangl2 (lanes 11-13 and line in the graph.

FIG. 26: Schematic representation of a spinal open-book assay and DiI injection. FIG. 26A: Schematic spinal code diagram. FIG. 26B: Mouse E11.5 or Rat E13 spinal cords are dissected and open-book preparation of the spinal cords are prepared. FIG. 26C: Mouse open-books are fixed and DiI injected immediately. Rat open-books are cultured for 12-18 hrs then subsequently DiI injected. The day following DiI injection commissural axon tracks can be visualized.

FIG. 27: Commissural axon trajectory in the developing spinal cord and antibody specificity in the spinal cord. FIG. 27A: Three schematic of the murine spinal cord indicating the projections of commissural axons. On the left shows the bilaterally systematic trajectory of mouse E11.5 commissural axons in the dorsal (D)-ventral (V) plane. The middle schematic depicts the location of “pre” versus “post-crossing” transitions of commissural axon guidance in the D-V plane, where the midline (the floor-plate, FP) separates the two phases of axon guidance. Commissural axons are term “pre crossing” when axons are found before they cross the FP and are TAG-1 immmunoreactive. After midline crossing, the axons are termed “post crossing” and after crossing the FP, they down-regulate TAG-1 and turn on the protein expression of L1. The last schematic depicts the commissural trajectory in the anterior-posterior (A-P) plane. After crossing the spinal midline commissural axons turn anteriorly toward the brain following the Wnt protein gradient. FIG. 27B: Post-crossing enrichment of the Celrs3 immunoreactivity is diminished in the Celsr3−/− embryos. E11.5 spinal sections of Celsr3+/+, Celsr3+/− and Celsr3−/− embryos immunostained with the polyclonal antibody generated against the intracellular carboxyl terminal region of Celsr3 (amino acids 3099 to 3301). FIG. 27C: Fzd3 immunoreactivity is reduced in the post-crossing spinal section of E11.5 of Fzd3−/− embryos. FIG. 27D: Fzd3 immunoreactivity in spinal lysates from E11.5 embryos Fzd3 from heterozygous cross. The immunoreactivity is diminished in the heterozygous lysates and nearly absent in the homozygous lysates. FIG. 27E: Looptail embryos show a reduction in Vangl2 expression. E14.5 spinal lysates were IB with ant-Vangl2 and Lp/Lp embryo lysates show a reduction in Vangl2 signal. FIG. 27F: A schematic of C-terminal Lp tail mutation in the Vangl2 protein, where Ser 464 is mutated to an Asn. White Scale bars represent 100 um.

FIG. 28: Electroporated commissural neurons and growth cones. FIG. 28A: Schematic of how commissural neurons are electroporated ex-vivo, followed by dissection, dissociation and culturing. FIG. 28B depicts Fzd3-mCherry and FIG. 28C depicts EGFP-Vgl2 electroporated commissural neurons. FIGS. 28D-28D″: Fzd3-mCherry protein localization in an electroporated commissural growth cone. Fzd3-mCherry showed both punctate and cytoplasmic localization. FIGS. 28E-28E″: Flag-Vgl2 membraneous protein localization in the commissural growth cone. FIGS. 28F-28F″: Fzd3-mCherry and EGFP-Vgl2 localization in co-electroporated commissural growth cone showed more internalized Fzd3-mCherry localization when co-expressed with Vgl2. White line indicates 5 um. Alpha-Tubulin and 488-phalloidin depicts the shape of the growth cone.

FIG. 29: Celsr3 knock out embryos displays anterior-posterior commissural axon guidance defects. FIGS. 29A-29C: Pax7 expression by the progenitor domains did not change in the wildtype, heterozygous or homozygous Celsr3 embryo spinal cords at E11.5. FIGS. 29D-29F: Lhx1/5 expression remained unaltered in the Celsr3 wildtype, heterozygous and homozygous null embryos at E11.5 (arrows, cell bodies expressing indicated transcription factors, and location of the floor plate). FIGS. 29G-29L: The dorso-ventral trajectory of commissural axons were normal in Celsr3−/− embryos. TAG-1 immunostaining of E11.5 Celsr3+/+, Celsr3+/− and Celsr3−/− embryos sections show that commissural axons in all the embryos reach the midline (FIGS. 29G-29i). L1 immunostaining of E11.5 Celsr3+/+, Celsr3+/− and Celsr3−/− embryos sections (FIGS. 29L-29N). (Arrow, pre-crossing commissural axons, location of the floor plate, and post-crossing commissural axons). FIG. 29M: Celsr3+/+ and Celsr3+/− embryos show normal anterior posterior guidance of commissural axons, but Celsr3−/− embryos display randomized axons at E11.5. FIG. 29N: The injection sites for the Celsr3+/+, Celsr3+/− and Celsr3−/− embryos are quantified, 90.0% (SEM+/−10.0%), 94.6% (SEM+/−2.34%), and 6.00% (SEM+/−3.88%) respectively displayed correct anterior commissural axon DiI injection sites. Scale bars represent 100 um.

FIG. 30: Nkx2.2 and Islet1 staining in E11.5 spinal sections of Looptail and Celsr3 embryos. FIG. 30A: Vangl2+/+, Vanlg2+/Lp and the open neural tube Lp/Lp embryo spinal sections immunostained with Nkx2.2 and Islet1 show no gross defects in immunoreactivity. FIG. 30B: NKx2.2 and Islet1 immunoreactivity in Celsr3+/+, Celsr3+/− and Celsr3−/− embryos appear the same.

FIG. 31 Sonic Hedgehog induces Semaphorin responsiveness in pre-crossing commissural axons. FIG. 31A depicts a schematic diagram of rat E13 embryo microdissections of pre-crossing explants from the dorsal domains of the spinal cord. FP, floor plate. RP, root plate. FIG. 31b: Diagram of the modified pre-crossing collagen explant assay. Dorsal spinal cord pre-crossing explants were placed on a bottom collagen gel containing COS-7 cells secreting Netrin-1 or COS-7 cells secreting both Netrin-1 and Shh-N and cocultured for ˜16 h next to a COS-7 cell aggregate transfected either with Semaphorin-expressing constructs in top collagen gel (FIG. 31D-31E, 31G-31H right columns) or a control vector construct (FIG. 31C and FIG. 31F, left column) placed 200-400 μm from the explant. Repulsive function was assessed by the P/D ratio derived from the total length in the proximal (P) over the distal (D) quadrants of the explants. FIG. 31C-31H: In the presence of COS-7 cells secreting Shh-N from the bottom collagen, commissural axons were repelled by Sema3F and Sema3B (asterisks indicate reduced growth due to repulsion), whereas commissural axons are normally not repelled by Semaphorins in the absence of Shh (top row in c). FIG. 31i: Quantification of the total axon length P/D ratio. Graph shows means±s.e.m. error bars from three sets of experiments and n indicates the total number of explants quantified. **P<0.01 and ***P<0.0025. Scale bar represents 100 μm.

FIG. 32. Shh function is required for normal midline pathfinding of commissural axons. FIG. 32A-32i: Confocal images of DiI injections labeling commissural axons in open book explants as seen in the schematic diagram in FIG. 32J. FIGS. 32A-32F depict DiI injections showing commissural axon pathfinding during midline crossing. FIG. 32A: Untreated open book injection shows the majority of axons crossing the floor plate (FP) and turning anteriorly (single arrow). FIGS. 32B-32F: in the presence of the function-blocking Shh antibody, 5E1, some commissural axons fail to enter the FP (arrowheads in FIGS. 32B-32E), while others stall and form knots within the FP (double head arrow in FIGS. 32D-32D). Many axons fail to exit the FP (short double arrows FIGS. 32B-32F) and instead stall at the contralateral side of the FP forming distinct knots (asterisks in FIG. 32E). A few post-crossing axons loop back and recross the FP (single short arrow in FIG. 32D) and other postcrossing axons overshoot or wander into the contralateral side of the spinal cord instead of turning anteriorly (double long arrows in FIG. 32F) reminiscent of A-P axis misguidance. FIGS. 32G-32i: DiI images showing commissural axon pathfinding treated after midline crossing. FIG. 32G: DiI injection of an untreated spinal cord explant showing anterior turning of a stereotypical commissural axon trajectory after midline crossing projecting into the ventral and lateral funiculus alongside the FP (long single arrow). FIGS. 32H-32i: DiI injection of an open book explant treated with anti-Shh, 5E1 after midline crossing displaying defasciculation errors during their anterior trajectory. Post-crossing axons fan out from their fasciculated tracts and fall posteriorly (double long errors in FIG. 32H), while the majority of axons have already crossed the midline during the anti-Shh antibody treatment, some new axons are affected before crossing and fail to enter the FP (arrowheads in FIG. 32H) and a few others wandered within the FP (double head arrow in FIG. 32i). FIG. 32J: Schematic diagram of the open-book explant DiI assay showing a normal DiI injection crossing the midline and turning anteriorly. FIG. 32K: Summary of commissural axon pathfinding errors in anti-Shh treated spinal cord explants. FIG. 32L: Quantification of midline pathfinding behaviors in treated DiI injections as seen in FIGS. 32A-32F. n=number of DiI injections examined. Scale bar represents 50 μm (FIG. 32A and FIG. 32i) and 100 μm (FIG. 32G).

FIG. 33. The Shh receptor Patched-1 is required for normal midline crossing of commissural axons. FIG. 33A: Schematic diagram showing ex utero electroporation of rE13 embryos. FIG. 33B: Constructs expressing an EGFP vector control and a Shh insensitive form of Patched-1 which inhibits Smo signaling, Patched1^{Δloop2}. The Patched1^{Δloop2} construct is driven under a β-actin promoter conferring constitutive neuronal expression under IRES translational control. (c) Normal midline pathfinding of commissural axons electroporated with EGFP. Axons enter the midline, cross the floor plate (FP) and turn anteriorly soon after crossing (arrows). Some of the pre-crossing axon tracts are out of the image plane. FIGS. 33D-33G: Abnormal midline pathfinding of commissural axons expressing Patched1^{Δloop2}. The number of axons reaching the FP is significantly reduced; some axons appear misoriented and unable to enter the midline (arrowheads in FIGS. 33D-33G). Many axons loop back and recross the midline (double head arrows in FIGS. 33E-33G). A significant number of axons overshoot and wander into the contralateral ventral spinal cord (short arrows in FIG. 33D, FIG. 33E and FIG. 33G) and some even loop back (asterisk in FIG. 33D). Some of the overshot axons do not turn anteriorly immediately after midline crossing but rather turn gradually at more lateral positions (double arrowhead in FIG. 33E). Some of the pre-crossing axon tracts are out of image plane. FIG. 33H: Summary of misguidance behaviors observed in Patched1^{Δloop2} electroporated spinal cord explants. FIG. 33i: Quantification of commissural midline pathfinding behaviors in EGFP and Patched1^{Δloop2} expressing axons. The graph shows the percentage of axons displaying normal and misguided behaviors in electroporated spinal cords. The mean±SEM error bars are from three sets of experiments and n indicates the total number of explants quantified. *P<0.05 (Student's t-test). FIGS. 33J-33M: Patched-1 is highly enriched in commissural axons during midline crossing (arrows in FIG. 33J and FIG. 33L). Scale bars represent 50 μm (FIG. 33C-33E), 100 μm (FIGS. 33F-33G), and 100 μm (FIGS. 33J-33M).

FIG. 34. Smoothened is required for proper guidance of commissural axons during midline crossing. FIG. 34A: Schematic diagram depicting the pRS shRNA expression vector driving Smo shRNA under a U6 promoter (Origene). The shRNA expression cassette contains a 29 nt sequence targeting the Smoothened (Smo) protein (SEQ ID NO:44). FIGS. 34B-34E: COS-7 cells transiently transfected with a Smo-IRES-GFP construct (FIG. 34B) or co-transfected with Smo-IRES-GFP and Smo shRNA constructs (FIGS. 34C-34E). FIG. 34B: High-magnification image of GFP and Smo immunofluorescence (red) showing levels of the Smo-IRES-GFP expression. FIG. 34D: Reduced Smo staining (arrowheads) in Smo-IRES-GFP/Smo shRNA expressing cells. Few cells co-transfected with Smo shRNA still express Smo but at much reduced level (arrow in FIG. 34E). FIG. 34F: Western blot showing down regulation of Smo protein by Smo shRNA. Duplicate lanes of COS7 cell lysate, Smo expression construct plus a control of the empty shRNA vector and Smo expression construct plus Smo shRNA construct. α-tubulin was the loading control. FIGS. 34G-34J: Open book spinal cord cultures of rat E13 embryos co-electroporated with EGFP and a random shRNA vector (FIG. 34G) or the Smo shRNA expression vector (FIGS. 34H-34J). Open book explants are stained for GFP and the floor plate marker HNF-3β. FIG. 34G: Axons in open book explants of embryos electroporated with a random shRNA vector follow the stereotypic trajectory of commissural axons, enter the midline, cross and turn anteriorly on the contralateral side of the spinal cord in close contact with the floor plate (FP) border (arrows in FIG. 34G). FIGS. 34H-34J: In contrast, the majority of axons electroporated with the Smo shRNA construct wandered and failed to exit the FP and instead loop back into the midline (arrowheads in FIGS. 34H-34J). Post-crossing axons on the contralateral side overshoot away from the FP and do not turn anteriorly immediately after midline crossing but rather turn gradually at more lateral positions (asterisks in FIG. 34H and FIG. 34J). A few axons project normally and turn anteriorly close to the contralateral floor plate border (arrow in FIG. 34H). FIG. 34K: Schematic showing a summary of misguidance behaviors observed in Smo ShRNA electroporated embryos. FIG. 34L: Quantification of open book explants co-electroporated with EGFP and a random shRNA or Smo shRNA constructs. The graph shows the percentage of axons displaying normal and misguided behaviors in electroporated spinal cords during midline pathfinding. SEM error bars are from three sets of experiments and n indicates the total number of explants quantified. **P<0.01, *P<0.05 (Student's t-test). Scale bars represent 20 [m (FIG. 34B), 10 μm (c), 100 μtm (FIGS. 34D-34G), 50 μm (FIG. 34i), and 100 μm (FIGS. 34J-34L). Sequence of Smo target-loop

FIG. 35. Shh may induce Semaphorin responsiveness by modulating the activity of the cAMP/PKA pathway. FIGS. 35A-35E: Commissural axon projections during midline crossing revealed by DiI injections in open-book spinal cord treated explants. FIG. 35A: Normal trajectory of commissural axons in a vehicle (DMSO) treated spinal cord explant turning anteriorly after midline crossing. FIGS. 35B-35E: Abnormal midline pathfinding of commissural axons in the presence of the adenylyl cyclase activator, Forskolin. The majority of axons in Forskolin treated spinal cord explants stall at the contralateral side of the FP (double arrows in FIG. 35B and FIG. 35D) with prominent knotting (asterisks in FIG. 35B and FIG. 35C). In some cases, wandering axons reenter the FP (single short arrow in FIG. 35D) while others overshoot to the contralateral side of the FP without turning (long arrow in FIG. 35D). FIG. 35E: A number of injected axons projected randomly in the A-P direction (long arrow). FIG. 35F: Summary of commissural axon misguidance errors observed in Forskolin treated open book explants. FIG. 35G: Quantifications of midline pathfinding behaviors seen by DiI labeling in vehicle and Forskolin treated open book explants (FIGS. 35H-35K) Confocal images of spinal cord pre-crossing commissural explants in DMSO or Forskolin treated cultures. FIG. 35H and FIG. 35i: Symmetric outgrowth of pre-crossing commissural axons in the presence of Netrin-1/Shh expressing cells cultured in media containing either vehicle DMSO or Forskolin. FIG. 35J: Shh induced Semaphorin repulsion of pre-crossing commissural axons is maintained in DMSO treated explants (asterisk indicates repulsion), but repulsion significantly diminishes in pre-crossing explants cultured in Forskolin containing media (FIG. 35K). FIG. 35L: P/D ratios of the total axon bundle length in DMSO and Forskolin treated pre-crossing commissural explants. Graph shows mean average±SEM error bars from three sets of experiments and n indicates the total number of explants quantified. *P<0.05 (Student's t-test). FIG. 35M: Levels of activated phospho-PKA and of total PKA as seen by immunoblots of rE13 dorsal spinal cord primary cultures. The PKA inhibitor KT5720, Shh-N recombinant protein and the Smoothened agonist Purmorphamine reduce the level of activated PKA in dorsal spinal cord lysates. Scale bar represents 50 μm (FIG. 35A) and 100 μm (FIG. 35K).

FIG. 36. FIG. 36A depicts pre-crossing commissural axons grow radially and straight towards a COS cell aggregate transfected with Vector control. FIGS. 36B-36C: Pre-crossed axons are repelled away (arrow indicating bending) from a Sema3F-secreting cell aggregate when Shh is present. FIG. 36D depicts design of collagen explant assay in collagen gel. The bottom layer contains COS cells secreting Netrin-1 only or Netrin-1 plus Shh. The top layer contains cell aggregate and pre-crossing explants.

FIG. 37. Patched1 protein (red immunofluorescence) is found expressed in dissociated commissural neurons from E13 rat dorsal spinal cord (TAG-1-positive, cyan immunofluorescence). DAP1 stains for cell nuclei.

FIG. 38. FIGS. 38A-38C depict Dil tracing of commissural axons in “open-book” explaints cultured in contro DMSO or in the presence of a PKA inhibitor KT5720. Many axons failed to enter the midline (upward arrows in FIG. 38B and FIG. 38C) and very few axons stall at the contralateral border of the floor plate (double downward arrows in FIG. 38C. FIG. 38D: Schematics of definition of phenotypes. FIG. 38E: Quantification of percentage of injection sites with various phenotypes. Abbreviation: FP, floor plate.

FIG. 39. A model for the role of Shh in modulating the sensitivity of axons to guidance cues. In commissural axons, Shh may act as a second messenger regulator through the Smo Gai transduction pathway, inhibiting adenylyl cyclase (AC) activity, which in turn downregulates cAMP intracellular levels and subsequently PKA activity. The axonal ratio of cyclic nucleotides cAMP and cGMP has been shown to be important in switching axonal responses to guidance molecules 15 and a decrease in cAMP levels could favor Semaphorin/cGMP signaling and allow axons to respond to repulsion. Concomitantly, as in the Drosophila counterpart, downregulation of cAMP/PKA signaling could affect the function of effector molecules involved in silencing Semaphorin signaling, such as the A-kinase anchor proteins, AKAPs 16, which remains to be studied in vertebrates.

### DEFINITIONS

As used herein, “agent” or “biologically active agent” refers to a biological, pharmaceutical, or chemical compound or other moiety. Non-limiting examples include simple or complex organic or inorganic molecule, a peptide, a protein, an oligonucleotide, an antibody, an antibody derivative, antibody fragment, a vitamin derivative, a carbohydrate, a toxin, or a chemotherapeutic compound. Various compounds can be synthesized, for example, small molecules and oligomers (e.g., oligopeptides and oligonucleotides), and synthetic organic compounds based on various core structures. In addition, various natural sources can provide compounds for screening, such as plant or animal extracts, and the like. A skilled artisan can readily recognize that there is no limit as to the structural nature of the agents of the present invention.

The term “candidate agent” refers to any molecule of any composition, including proteins, peptides, nucleic acids, lipids, carbohydrates, organic molecules, inorganic molecules, and/or combinations of molecules which are suspected to be capable of modulating (e.g., inhibiting or promoting) a measured parameter (e.g., activity, expression, signal transduction, neuron degeneration) in a treated cell (tissue or subject) in comparison to an untreated cell (tissue or subject).

The term “antagonist” as used herein refers to a molecule having the ability to inhibit a biological function of a target polypeptide. Accordingly, the term “antagonist” is defined in the context of the biological role of the target polypeptide. While preferred antagonists herein specifically interact with (e.g. bind to) the target, molecules that inhibit a biological activity of the target polypeptide by interacting with other members of the signal transduction pathway of which the target polypeptide is a member are also specifically included within this definition. Antagonists, as defined herein, without limitation, include antibodies and immunoglobulin variants, peptides, peptidomimetics, non-peptide small molecules, antisense molecules, and oligonucleotide decoys.

The term “agonist” as used herein refers to a molecule having the ability to initiate or enhance a biological function of a target polypeptide. Accordingly, the term “agonist” is defined in the context of the biological role of the target polypeptide. While preferred agonists herein specifically interact with (e.g. bind to) the target, molecules that initiate or enhance a biological activity of the target polypeptide by interacting with other members of the signal transduction pathway of which the target polypeptide is a member are also specifically included within this definition. Agonists, as defined herein, without limitation, include antibodies and immunoglobulin variants, peptides, peptidomimetics, non-peptide small molecules, antisense molecules, and oligonucleotide decoys.

The term “modulator” means any compound that interacts with the target protein. The interaction is not limited to a compound acting as an antagonist, agonist, partial agonist, or inverse agonist of the target protein. In some embodiments, the compounds of the present invention act as an antagonist of the target protein. In some embodiments, the compounds of the present invention act as an agonist of the target protein. In some embodiments, the compounds of the present invention act as a partial agonist of the target protein. In some embodiments, the compounds of the present invention as an inverse agonist of the target protein.

The term “effective amount” or “therapeutically effective amount” refers to the amount of an active agent sufficient to induce a desired biological result. That result may be alleviation of the signs, symptoms, or causes of a disease, or any other desired alteration of a biological system. The term “therapeutically effective amount” is used herein to denote any amount of the formulation which causes a substantial improvement in a disease condition when applied to the affected areas repeatedly over a period of time. The amount will vary with the condition being treated, the stage of advancement of the condition, and the type and concentration of formulation applied. Appropriate amounts in any given instance will be readily apparent to those skilled in the art or capable of determination by routine experimentation.

As used herein, “treatment” or “treating,” or “palliating” or “ameliorating” are used interchangeably herein. These terms refer to an approach for obtaining beneficial or desired results including but not limited to therapeutic benefit and/or a prophylactic benefit. By therapeutic benefit is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient may still be afflicted with the underlying disorder. For prophylactic benefit, the compositions may be administered to a patient at risk of developing a particular disease, or to a patient reporting one or more of the physiological symptoms of a disease, even though a diagnosis of this disease may not have been made. Treatment includes preventing the disease, that is, causing the clinical symptoms of the disease not to develop by administration of a protective composition prior to the induction of the disease; suppressing the disease, that is, causing the clinical symptoms of the disease not to develop by administration of a protective composition after the inductive event but prior to the clinical appearance or reappearance of the disease; inhibiting the disease, that is, arresting the development of clinical symptoms by administration of a protective composition after their initial appearance; preventing re-occurring of the disease and/or relieving the disease, that is, causing the regression of clinical symptoms by administration of a protective composition after their initial appearance.

The term “pharmaceutically acceptable salt” refers to salts derived from a variety of organic and inorganic counter ions well known in the art and include, by way of example only, sodium, potassium, calcium, magnesium, ammonium, tetraalkylammonium, and the like; and when the molecule contains a basic functionality, salts of organic or inorganic acids, such as hydrochloride, hydrobromide, tartrate, mesylate, acetate, maleate, oxalate and the like.

A “subject,” “individual,” or “patient,” is used interchangeably herein, which refers to a vertebrate, preferably a mammal, more preferably a human. Mammals include, but are not limited to, murines, simians, humans, farm animals, sport animals, and pets. Tissues, cells and their progeny of a biological entity obtained in vitro or cultured in vitro are also encompassed.

As used herein, “promote” or “increase,” or “promoting” or “increasing” are used interchangeably herein. These terms refer to the increase in a measured parameter (e.g., activity, expression, signal transduction, neuron degeneration) in a treated cell (tissue or subject) in comparison to an untreated cell (tissue or subject). A comparison can also be made of the same cell or tissue or subject between before and after treatment. The increase is sufficient to be detectable. In some embodiments, the increase in the treated cell is at least about 10%, 20%, 30%, 40%, 50%, 60%, 70%, 80%, 90%, 1-fold, 2-fold, 3-fold, 4-fold or more in comparison to an untreated cell.

As used herein, “inhibit,” “prevent” or “reduce,” or “inhibiting,” “preventing” or “reducing” are used interchangeably herein. These terms refer to the decrease in a measured parameter (e.g., activity, expression, signal transduction, neuron degeneration) in a treated cell (tissue or subject) in comparison to an untreated cell (tissue or subject). A comparison can also be made of the same cell or tissue or subject between before and after treatment. The decrease is sufficient to be detectable. In some embodiments, the decrease in the treated cell is at least about 10%, 20%, 30%, 40%, 50%, 60%, 70%, 80%, 90%, or completely inhibited in comparison to an untreated cell. In some embodiments the measured parameter is undetectable (i.e., completely inhibited) in the treated cell in comparison to the untreated cell.

The term “selective inhibition” or “selectively inhibit” as referred to a biologically active agent refers to the agent's ability to preferentially reduce the target signaling activity as compared to off-target signaling activity, via direct or indirect interaction with the target.

A “therapeutic effect,” as that term is used herein, encompasses a therapeutic benefit and/or a prophylactic benefit as described above. A prophylactic effect includes delaying or eliminating the appearance of a disease or condition, delaying or eliminating the onset of symptoms of a disease or condition, slowing, halting, or reversing the progression of a disease or condition, or any combination thereof

The term “co-administration,” “administered in combination with,” and their grammatical equivalents, as used herein, encompasses administration of two or more agents to an animal so that both agents and/or their metabolites are present in the animal at the same time. Co-administration includes simultaneous administration in separate compositions, administration at different times in separate compositions, or administration in a composition in which both agents are present.

The term “in vivo” refers to an event that takes place in a subject's body.

The term “in vitro” refers to an event that takes places outside of a subject's body. For example, an in vitro assay encompasses any assay run outside of a subject assay. In vitro assays encompass cell-based assays in which cells alive or dead are employed. In vitro assays also encompass a cell-free assay in which no intact cells are employed.

The term “ex vivo” pertains to a biological process or reaction on a target tissue, a body structure taking place outside of the human or animal body, in order to reimplant said tissue or structure on the candidate subject. By extension, according to the present invention, the term ex vivo refers also to taking out from the body a small fragment from the human or animal body, and taking in vitro a sample of said fragment, or using it as a sample. According to the present invention, the term “ex vivo” also refers to tissues or body structures, or small fragments of them, removed from humans or animals after death, “post mortem”, for subsequent analysis.

The term “small molecule mimetic” as used herein refers to a small molecule (e.g., having a molecular weight of less than about 700 daltons) that mimics the biological activity of a reference gene product (e.g., a Wnt polypeptide or a functional fragment thereof), by substantially duplicating the targeting activity of the reference gene product, but it is not a polypeptide or a peptoid per se. In some embodiments, a small molecule mimetic is a peptide mimetic has a molecular weight of less than about 700 daltons.

The terms “peptidomimetic” and “peptide mimetic” refer to a synthetic chemical compound that has substantially the same structural and functional characteristics of a naturally or non-naturally occurring polypeptide (e.g., Wnt). Peptide analogs are commonly used in the pharmaceutical industry as non-peptide drugs with properties analogous to those of the template peptide. These types of non-peptide compound are termed “peptide mimetics” or “peptidomimetics” (Fauchere, J. Adv. Drug Res. 15:29 (1986); Veber and Freidinger TINS p. 392 (1985); and Evans et al. J. Med. Chem. 30:1229 (1987), which are incorporated herein by reference). Peptide mimetics that are structurally similar to therapeutically useful peptides may be used to produce an equivalent or enhanced therapeutic or prophylactic effect. Generally, peptidomimetics are structurally similar to a paradigm polypeptide (i.e., a polypeptide that has a biological or pharmacological activity), such as found in a polypeptide of interest, but have one or more peptide linkages optionally replaced by a linkage selected from the group consisting of, e.g., —CH2NH—, —CH2S—, —CH2—CH2—, —CH═CH— (cis and trans), —COCH2—, —CH(OH)CH2—, and —CH2SO—. The mimetic can be either entirely composed of synthetic, non-natural analogues of amino acids, or, is a chimeric molecule of partly natural peptide amino acids and partly non-natural analogs of amino acids. The mimetic can also incorporate any amount of natural amino acid conservative substitutions as long as such substitutions also do not substantially alter the mimetic's structure and/or activity. For example, a mimetic composition is within the scope of the invention if it is capable of carrying out at least one activity of a polypeptide of interest. In some embodiments, a peptidomimetic or peptide mimetic is a small molecule mimetic (e.g., when the peptide mimetic has a molecular weight of less than about 700 daltons). In some embodiments, a peptidomimetic or peptide mimetic is not a small molecule mimetic (e.g., when the peptide mimetic has a molecular weight of greater than about 700 daltons).

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymers.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an α carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Naturally encoded amino acids are the 20 common amino acids (alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine) and pyrrolysine and selenocysteine.

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid that encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention.

The following eight groups each contain amino acids that are conservative substitutions for one another:
1) Alanine (A), Glycine (G);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V);
6) Phenylalanine (F), Tyrosine (Y), Tryptophan (W);
7) Serine (S), Threonine (T); and
8) Cysteine (C), Methionine (M) (see, e.g., Creighton, Proteins (1984)).

“Percentage of sequence identity” is determined by comparing two optimally aligned sequences over a comparison window, wherein the portion of the polynucleotide sequence in the comparison window may comprise additions or deletions (i.e., gaps) as compared to the reference sequence (e.g., a polypeptide of the invention), which does not comprise additions or deletions, for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The terms “identical” or percent “identity,” in the context of two or more nucleic acids or polypeptide sequences, refer to two or more sequences or subsequences that are the same sequences. Two sequences are “substantially identical” if two sequences have a specified percentage of amino acid residues or nucleotides that are the same (i.e., 60% identity, optionally 65%, 70%, 75%, 80%, 85%, 90%, or 95% identity over a specified region, or, when not specified, over the entire sequence), when compared and aligned for maximum correspondence over a comparison window, or designated region as measured using one of the following sequence comparison algorithms or by manual alignment and visual inspection. The invention provides polypeptides that are substantially identical to the polypeptides, respectively, exemplified herein (e.g., any of SEQ ID NOs: 1-28), as well as uses thereof including but no limited to use for treating or preventing arthritis or joint injury. Optionally, the identity exists over a region that is at least about 50 nucleotides in length, or more preferably over a region that is 100 to 500 or 1000 or more nucleotides in length, or the entire length of the reference sequence.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well known in the art. Optimal alignment of sequences for comparison can be conducted, e.g., by the local homology algorithm of Smith and Waterman (1970) Adv. Appl. Math. 2:482c, by the homology alignment algorithm of Needleman and Wunsch (1970) J. Mol. Biol. 48:443, by the search for similarity method of Pearson and Lipman (1988) Proc. Nat'l. Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Ausubel et al., Current Protocols in Molecular Biology (1995 supplement)).

Two examples of algorithms that are suitable for determining percent sequence identity and sequence similarity are the BLAST and BLAST 2.0 algorithms, which are described in Altschul et al. (1977) Nuc. Acids Res. 25:3389-3402, and Altschul et al. (1990) J. Mol. Biol. 215:403-410, respectively. Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) or 10, M=5, N=−4 and a comparison of both strands. For amino acid sequences, the BLASTP program uses as defaults a wordlength of 3, and expectation (E) of 10, and the BLOSUM62 scoring matrix (see Henikoff and Henikoff (1989) Proc. Natl. Acad. Sci. USA 89:10915) alignments (B) of 50, expectation (E) of 10, M=5, N=−4, and a comparison of both strands.

The BLAST algorithm also performs a statistical analysis of the similarity between two sequences (see, e.g., Karlin and Altschul (1993) Proc. Natl. Acad. Sci. USA 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

“Nucleic acid” refers to deoxyribonucleotides or ribonucleotides and polymers thereof in either single- or double-stranded form, and complements thereof. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs).

The term “inhibitory nucleic acid” means an RNA, DNA, or combination thereof that interferes or interrupts the translation of mRNA. Inhibitory nucleic acids can be single or double stranded. The nucleotides of the inhibitory nucleic acid can be chemically modified, natural or artificial. The terms “short-inhibitory RNA” and “siRNA” interchangeably refer to short double-stranded RNA oligonucleotides that mediate RNA interference (also referred to as “RNA-mediated interference,” or RNAi). RNAi is a highly conserved gene silencing event functioning through targeted destruction of individual mRNA by a homologous double-stranded small interfering RNA (siRNA) (Fire, A. et al., Nature 391:806-811 (1998)). Mechanisms for RNAi are reviewed, for example, in Bayne and Allshire, Trends in Genetics (2005) 21:370-73; Morris, Cell Mol Life Sci (2005) 62:3057-66; Filipowicz, et al., Current Opinion in Structural Biology (2005) 15:331-41.

Inhibitory nucleic acids, such as siRNA, shRNA, ribozymes, or antisense molecules, can be synthesized and introduced into cells using methods known in the art. Molecules can be synthesized chemically or enzymatically in vitro (Micura, Agnes Chem. Int. Ed. Emgl. 41: 2265-9 (2002); Paddison et al., Proc. Natl. Acad. Sci. USA, 99: 1443-8 2002) or endogenously expressed inside the cells in the form of shRNAs (Yu et al., Proc. Natl. Acad. Sci. USA, 99: 6047-52 (2002); McManus et al., RNA 8, 842-50 (2002)). Plasmid-based expression systems using RNA polymerase III U6 or H1, or RNA polymerase II U1, small nuclear RNA promoters, have been used for endogenous expression of shRNAs (Brummelkamp et al., Science, 296: 550-3 (2002); Sui et al., Proc. Natl. Acad. Sci. USA, 99: 5515-20 (2002); Novarino et al., J. Neurosci., 24: 5322-30 (2004)). Synthetic siRNAs can be delivered by electroporation or by using lipophilic agents (McManus et al., RNA 8, 842-50 (2002); Kishida et al., J. Gene Med., 6: 105-10 (2004)). Alternatively, plasmid systems can be used to stably express small hairpin RNAs for the suppression of target genes (Dykxhoorn et al., Nat. Rev. Mol. Biol., 4: 457-67 (2003)). Various viral delivery systems have been developed to deliver shRNA-expressing cassettes into cells that are difficult to transfect (Brummelkamp et al., Cancer Cell, 2: 243-7 (2002); Rubinson et al., Nat. Genet., 33: 401-6 2003). Furthermore, siRNAs can also be delivered into live animals. (Hasuwa et al., FEBS Lett., 532, 227-30 (2002); Carmell et al., Nat. Struct. Biol., 10: 91-2 (2003); Kobayashi et al., J. Pharmacol. Exp. Ther., 308: 688-93 (2004)).

Methods for the design of siRNA or shRNA target sequences have been described in the art. Among the factors to be considered include: siRNA target sequences should be specific to the gene of interest and have ˜20-50% GC content (Henshel et al., Nucl. Acids Res., 32: 113-20 (2004); G/C at the 5′ end of the sense strand; A/U at the 5′ end of the antisense strand; at least 5 A/U residues in the first 7 bases of the 5′ terminal of the antisense strand; and no runs of more than 9 G/C residues (Ui-Tei et al., Nucl. Acids Res., 3: 936-48 (2004)). Additionally, primer design rules specific to the RNA polymerase will apply. For example, for RNA polymerase III, the polymerase that transcribes from the U6 promoter, the preferred target sequence is 5′-GN18-3′. Runs of 4 or more Ts (or As on the other strand) will serve as terminator sequences for RNA polymerase III and should be avoided. In addition, regions with a run of any single base should be avoided (Czauderna et al., Nucl. Acids Res., 31: 2705-16 (2003)). It has also been generally recommended that the mRNA target site be at least 50-200 bases downstream of the start codon (Sui et al., Proc. Natl. Acad. Sci. USA, 99: 5515-20 (2002); Elbashir et al., Methods, 26: 199-213 (2002); Duxbury and Whang, J. Surg. Res., 117: 339-44 (2004) to avoid regions in which regulatory proteins might bind. Additionally, a number of computer programs are available to aid in the design of suitable siRNA and shRNAs for use in the practice of this invention.

Ribozymes that cleave mRNA at site-specific recognition sequences can be used to destroy target mRNAs, particularly through the use of hammerhead ribozymes. Hammerhead ribozymes cleave mRNAs at locations dictated by flanking regions that form complementary base pairs with the target mRNA. Preferably, the target mRNA has the following sequence of two bases: 5′-UG-3′. The construction and production of hammerhead ribozymes is well known in the art.

Gene targeting ribozymes necessarily contain a hybridizing region complementary to two regions, each of at least 5 and preferably each 6, 7, 8, 9, 10, 11, 12, 13, 14, 15, 16, 17, 18, 19 or 20 contiguous nucleotides in length of a target mRNA. In addition, ribozymes possess highly specific endoribonuclease activity, which autocatalytically cleaves the target sense mRNA.

With regard to antisense, siRNA or ribozyme oligonucleotides, phosphorothioate oligonucleotides can be used. Modifications of the phosphodiester linkage as well as of the heterocycle or the sugar may provide an increase in efficiency. Phophorothioate is used to modify the phosphodiester linkage. An N3′-P5′ phosphoramidate linkage has been described as stabilizing oligonucleotides to nucleases and increasing the binding to RNA. Peptide nucleic acid (PNA) linkage is a complete replacement of the ribose and phosphodiester backbone and is stable to nucleases, increases the binding affinity to RNA, and does not allow cleavage by RNAse H. Its basic structure is also amenable to modifications that may allow its optimization as an antisense component. With respect to modifications of the heterocycle, certain heterocycle modifications have proven to augment antisense effects without interfering with RNAse H activity. An example of such modification is C-5 thiazole modification. Finally, modification of the sugar may also be considered. 2′-O-propyl and 2′-methoxyethoxy ribose modifications stabilize oligonucleotides to nucleases in cell culture and in vivo.

Inhibitory oligonucleotides can be delivered to a cell by direct transfection or transfection and expression via an expression vector. Appropriate expression vectors include mammalian expression vectors and viral vectors, into which has been cloned an inhibitory oligonucleotide with the appropriate regulatory sequences including a promoter to result in expression of the antisense RNA in a host cell. Suitable promoters can be constitutive or development-specific promoters. Transfection delivery can be achieved by liposomal transfection reagents, known in the art (e.g., Xtreme transfection reagent, Roche, Alameda, Calif.; Lipofectamine formulations, Invitrogen, Carlsbad, Calif.). Delivery mediated by cationic liposomes, by retroviral vectors and direct delivery are efficient. Another possible delivery mode is targeting using antibody to cell surface markers for the target cells.

For transfection, a composition comprising one or more nucleic acid molecules (within or without vectors) can comprise a delivery vehicle, including liposomes, for administration to a subject, carriers and diluents and their salts, and/or can be present in pharmaceutically acceptable formulations. Methods for the delivery of nucleic acid molecules are described, for example, in Gilmore, et al., Curr Drug Delivery (2006) 3:147-5 and Patil, et al., AAPS Journal (2005) 7:E61-E77, each of which are incorporated herein by reference. Delivery of siRNA molecules is also described in several U.S. Patent Publications, including for example, 2006/0019912; 2006/0014289; 2005/0239687; 2005/0222064; and 2004/0204377, the disclosures of each of which are hereby incorporated herein by reference. Nucleic acid molecules can be administered to cells by a variety of methods known to those of skill in the art, including, but not restricted to, encapsulation in liposomes, by iontophoresis, by electroporation, or by incorporation into other vehicles, including biodegradable polymers, hydrogels, cyclodextrins (see, for example Gonzalez et al., 1999, Bioconjugate Chem., 10, 1068-1074; Wang et al., International PCT publication Nos. WO 03/47518 and WO 03/46185), poly(lactic-co-glycolic)acid (PLGA) and PLCA microspheres (see for example U.S. Pat. No. 6,447,796 and U.S. Patent Publication No. 2002/130430), biodegradable nanocapsules, and bioadhesive microspheres, or by proteinaceous vectors (O'Hare and Normand, International PCT Publication No. WO 00/53722). In another embodiments, the nucleic acid molecules can also be formulated or complexed with polyethyleneimine and derivatives thereof, such as polyethyleneimine-polyethyleneglycol-N-acetylgalactosamine (PEI-PEG-GAL) or polyethyleneimine-polyethyleneglycol-tri-N-acetylgalactosamine (PEI-PEG-triGAL) derivatives.

Examples of liposomal transfection reagents of use with this invention include, for example: CellFectin, 1:1.5 (M/M) liposome formulation of the cationic lipid N,NI,NII,NIII-tetramethyl-N,NI,NII,NIII-tetrapalmit-y-spermine and dioleoyl phosphatidylethanolamine (DOPE) (GIBCO BRL); Cytofectin GSV, 2:1 (M/M) liposome formulation of a cationic lipid and DOPE (Glen Research); DOTAP (N-[1-(2,3-dioleoyloxy)-N,N,N-tri-methyl-ammoniummethylsulfate) (Boehringer Manheim); Lipofectamine, 3:1 (M/M) liposome formulation of the polycationic lipid DOSPA and the neutral lipid DOPE (GIBCO BRL); and (5) siPORT (Ambion); HiPerfect (Qiagen); X-treme GENE (Roche); RNAicarrier (Epoch Biolabs) and TransPass (New England Biolabs).

In some embodiments, antisense, siRNA, or ribozyme sequences are delivered into the cell via a mammalian expression vector. For example, mammalian expression vectors suitable for siRNA expression are commercially available, for example, from Ambion (e.g., pSilencer vectors), Austin, Tex.; Promega (e.g., GeneClip, siSTRIKE, SiLentGene), Madison, Wis.; Invitrogen, Carlsbad, Calif.; InvivoGen, San Diego, Calif.; and Imgenex, San Diego, Calif. Typically, expression vectors for transcribing siRNA molecules will have a U6 promoter.

In some embodiments, antisense, siRNA, or ribozyme sequences are delivered into cells via a viral expression vector. Viral vectors suitable for delivering such molecules to cells include adenoviral vectors, adeno-associated vectors, and retroviral vectors (including lentiviral vectors). For example, viral vectors developed for delivering and expressing siRNA oligonucleotides are commercially available from, for example, GeneDetect, Bradenton, Fla.; Ambion, Austin, Tex.; Invitrogen, Carlsbad, Calif.; Open BioSystems, Huntsville, Ala.; and Imgenex, San Diego, Calif.

As used herein, the term “dominant negative mutant” of a protein refers to a mutant polypeptide or nucleic acid, which lacks wild-type activity and which, once expressed in a cell wherein a wild-type of the same protein is also expressed, dominates the wild-type protein and effectively competes with wild type proteins for substrates, ligands, etc., and thereby inhibits the activity of the wild type molecule.

The dominant negative mutant can be a polypeptide having an amino acid sequence substantially similar (i.e., at least about 75%, about 80%, about 85%, about 90%, about 95% similar) to the wild type protein. The dominant negative mutant can also be a polypeptide comprising a fragment of the wild type protein, e.g., the C-domain of the wild-type protein. The dominant negative mutant can be a truncated form of the wild type protein.

### DETAILED DESCRIPTION OF THE INVENTION

### I. Introduction

Provided herein, inter alia, are novel methods for modulating neuron degeneration and neuron guidance using modulators of Wnt pathaway and/or modulators of sonic hedgedog (SHH) pathway. In one aspect, neuron degeneration can be modulated, e.g., inhibited or promoted, by modulators of Wnt pathaway. For example, provided herein are novel methods for inhibiting degeneration of a neuron by contacting the neuron with a Wnt compound or a Fzd3 dephosphorylating agent. Such Wnt compounds or Fzd3 dephosphorylating agents can therefore be used to treat a neurodegenerative disease in a subject having or being at risk of developing the neurodegenerative disease. Provided also are novel methods for promoting degeneration of a neuron by contacting the neuron with a Fzd3 phosphorylating agent.

In another aspect, neuron growth and guidance can be modulated by modulators of SHH pathway. For example, provided herein are novel methods for modulating neuron cell growth and guidance by contacting the neuron with a SHH compound. Such SHH compounds can therefore be used for regenerating an neuron.

### II. Method for Modulating Neuron Degeneration

### 1. Method for Inhibiting Neuron Degeneration

Provided herein are novel methods and compositions for inhibiting degeneration of a neuron by contacting the neuron an agent thereby inhibiting degeneration of a neuron. In some embodiments, the agent is a Wnt compound or a Fzd3 dephosphorylating agent. In some embodiments, the Wnt compound is a Wnt peptide, a small molecule Wnt mimetic, or a Wnt agonist. For example, the Wnt peptide can be a polypeptide comprising an amino acid sequence having at least 90% identity to SEQ ID NO: 1, 2, 3, 4, 5, 6, 7, 8, 9, 10, 11, 12, 13, 14, 15, 16, 17, 18, or 19. In some embodiments, the Fzd3 dephosphorylating agent is a Vgl2 peptide or a Vgl2 mimetic, or a Dvl1 antagonist (e.g., a siRNA targeting Dvl1 or a Dvl1 antibody). Accordingly, it was discovered in the present invention that a Wnt compound or a Fzd3 dephosphorylating agent can be used for treating a neurodegenerative disease (e.g., amyotrophic lateral sclerosis, Alzheimer's disease or Parkinson's disease) in a subject having or being at risk of developing the neurodegenerative disease.

In some embodiments, the methods as described herein result in at least a 10% decrease (e.g., at least 15%, 20%, 25%, 30%, 35%, 40%, 45%, 50%, 55%, 60%, 65%, 70%, 75%, 80%, 85%, 90%, or even 100% decrease) in the degeneration of a population of neurons or in the degeneration of axons or cell bodies or dendrites of a neuron in a population of neurons as compared to a control population of neurons. In some embodiments, the methods as described herein result at least a 10% decrease (e.g., at least 15%, 20%, 25%, 30%, 35%, 40%, 45%, 50%, 55%, 60%, 65%, 70%, 75%, 80%, 85%, 90%, 95%, or even 100% decrease) in the number of neurons (or neuron bodies, axons, or dendrites thereof) that degenerate in a subject compared to the number of neurons (or neuron bodies, axons, or dendrites thereof) that degenerate in a subject that is not administered the one or more of the agents described herein. In some embodiments, the methods as described herein result in at least a 10% decrease (e.g., at least 15%, 20%, 25%, 30%, 35%, 40%, 45%, 50%, 55%, 60%, 65%, 70%, 75%, 80%, 85%, 90%, or even 100% decrease) in one or more (e.g., 1, 2, 3, 4, 5, 6, 7, 8, or 9) symptoms of a neurodegenerative disease and/or condition. In some embodiments, the methods as described herein result in at least a 10% decrease (e.g., at least 15%, 20%, 25%, 30%, 35%, 40%, 45%, 50%, 55%, 60%, 65%, 70%, 75%, 80%, 85%, 90%, 95%, or even 100% decrease) in the likelihood of developing a neurodegenerative disease and/or condition.

The methods of inhibiting neuron degeneration include in vitro, in vivo, and/or ex vivo methods. In some embodiments, the methods are practiced in vivo, i.e., the agent inhibiting neuron degeneration is administered to a subject. In some embodiments, the methods are practiced ex vivo, i.e., neurons to be treated form part of a nerve graft or a nerve transplant in a subject. In some embodiments, the methods are practiced in vitro.

The methods of inhibiting neuron degeneration can be used to inhibit or prevent neuron degeneration in patients newly diagnosed as having a neurodegenerative disease or at risk of developing a new neurodegenerative disease. On the other hand, the methods of inhibiting neuron degeneration can also be used to inhibit or prevent further neuron degeneration in patients who are already suffering from, or have symptoms of, a neurodegenerative disease. Preventing neuron degeneration includes decreasing or inhibiting neuron degeneration, which may be characterized by complete or partial inhibition of neuron degeneration. This can be assessed, for example, by analysis of neurological function.

### 2. Method for Promoting Neuron Degeneration

In addition, provided herein are novel methods and compositions for promoting degeneration of a neuron by contacting the neuron with an agent thereby promoting degeneration of a neuron. In some embodiments, the agent is a Fzd3 phosphorylating agent such as a Dvl1 peptide or a Dvl1 mimetic, or a Vgl2 antagonist (e.g., a siRNA targeting Vgl2 or a Vgl2 antibody).

### III. Wnt Peptides

Wnts are secreted cysteine-rich glycosylated proteins that play a role in the development of a wide range of organisms. Wnts are thought to function in a variety of developmental and physiological processes since many diverse species have multiple conserved Wnt genes (McMahon, Cell, 62:1073-1085, 1992; Nusse and Varmus, Cell, 69:1073-1087, 1992). Wnt can be a human Wnt protein, or a Wnt protein from any other species, such as mouse or chick. The Wnt growth factor family includes at least 19 genes identified in mammals, including Wnt1, Wnt2, Wnt2b, Wnt3, Wnt3a, Wnt4, Wnt5a, Wnt5b, Wnt 6, Wnt7a, Wnt7b, Wnt8Wnt8b, Wnt9a, Wnt9b, Wnt10a, Wnt10b, Wnt11, and Wnt16. Similar numbers of Wnt genes are present in other vertebrate species. Further Wnts may be discovered and/or characterized in the future, and those of skill will be able to employ any such Wnts in the context of the invention. Further, those of skill will be able to use the teachings herein to obtain and use Wnts of any species in the context of the invention.

As used herein, “Wnt peptide” or “Wnt polypeptide,” or “Wnt protein” are used interchangeably herein. In some embodiments, a Wnt peptide comprises a full-length amino acid sequence that is encoded by a Wnt gene. The human and murine full-length native amino acid sequences are described by GenBank accession number in the Table 1. Further, summary of human and murine Wnts is provided in Miller, Genome Biology, 3:3001.1-3001.15, 2001. In some embodiments, a Wnt peptide comprises an amino acid sequence that is longer than a full-length Wnt protein if additional non-Wnt amino acids are included in the sequence. In some embodiments, a Wnt peptide comprises a truncated sequence of a full-length Wnt protein, a mutated Wnt protein, or a Wnt amino acid sequence that is less than the full-length amino acid sequence of a Wnt, as long as the amino acid sequence retains an acceptable level of the equivalent biological activity of a full-length Wnt protein.

In some embodiments, a Wnt peptide is a polypeptide comprising an amino acid sequence having at least 60% identity, optionally 65%, 70%, 75%, 80%, 85%, 90%, 95%, 99% or 100% identity to the entire or a portion (as discussed below) of SEQ ID NOs:1, 2, 3, 4, 5, 6, 7, 8, 9, 10, 11, 12, 13, 14, 15, 16, 17, 18, or 19. In some embodiments, a Wnt peptide is a polypeptide comprising an amino acid sequence having at least 60% identity, optionally 65%, 70%, 75%, 80%, 85%, 90%, 95%, 99% or 100% identity to the entire or a portion (as discussed below) of SEQ ID NOs:20, 21, 22, 23, 24, 25, 26, 27, 28, 29, 30, 31, 32, 33, 34, 35, 36, 37, or 38.

TABLE 1
human and murine Wnts

| HUMAN | | MOUSE | |
|---|---|---|---|
| | Amino Acid | | Amino Acid |
| Wnt1 | NP005421 (SEQ ID NO: 1) | Wnt1 | NP067254 (SEQ ID NO: 20) |
| Wnt2 | AAH29854 (SEQ ID NO: 2) | Wnt2 | AAH26373 (SEQ ID NO: 21) |
| Wnt2B | NP078613 (SEQ ID NO: 3) | Wnt2B | NP033546 (SEQ ID NO: 22) |
| Wnt3 | NP110380 (SEQ ID NO: 4) | Wnt3 | P17553 (SEQ ID NO: 23) |
| Wnt3A | NP149122 (SEQ ID NO: 5) | Wnt3A | NP033548 (SEQ ID NO: 24) |
| Wnt4 | NP110388 (SEQ ID NO: 6) | Wnt4 | NP033549 (SEQ ID NO: 25) |
| Wnt5A | NP003383 (SEQ ID NO: 7) | Wnt5A | NP033550 (SEQ ID NO: 26) |
| Wnt5B | AAH01749 (SEQ ID NO: 8) | Wnt5B | AAH10775 (SEQ ID NO: 27) |
| Wnt6 | NP006513 (SEQ ID NO: 9) | Wnt6 | NP033552 (SEQ ID NO: 28) |
| Wnt7A | AAH08811 (SEQ ID NO: 10) | Wnt7A | AAH49093 (SEQ ID NO: 29) |
| Wnt7B | NP478679 (SEQ ID NO: 11) | Wnt7B | NP033554 (SEQ ID NO: 30) |
| Wnt8A | NP490645 (SEQ ID NO: 12) | Wnt8A | NP033316 (SEQ ID NO: 31) |
| Wnt8B | NP003384 (SEQ ID NO: 13) | Wnt8B | NP035850 (SEQ ID NO: 32) |
| Wnt9A | NP003386 (SEQ ID NO: 14) | Wnt9A | NP647459 (SEQ ID NO: 33) |
| Wnt9B | NP003387 (SEQ ID NO: 15) | Wnt9B | NP035849 (SEQ ID NO: 34) |
| Wnt10A | AAH52234 (SEQ ID NO: 16) | Wnt10A | AAH14737 (SEQ ID NO: 35) |
| Wnt10B | NP003385 (SEQ ID NO: 17) | Wnt10B | NP035848 (SEQ ID NO: 36) |
| Wnt11 | NP004617 (SEQ ID NO: 18) | Wnt11 | NP033545 (SEQ ID NO: 37) |
| Wnt16 | NP476509 (SEQ ID NO: 19) | Wnt16 | NP444346 (SEQ ID NO: 38) |

The term “Wnt peptide” includes any amino acid sequence that includes fewer consecutive amino acids of a Wnt than the full-length amino acid sequence of a Wnt. “Wnt peptide” includes not only consecutive amino acid sequences from a human Wnt, but also homologs thereof such as sequences from any other species, such as mouse. Thus, for example, a Wnt peptide can include, but is not limited to, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, about 100, about 110, about 120, about 130, about 140, about 150, about 160, about 170, about 180, about 190, about 200, about 210, about 220, about 230, about 240, about 250, about 275, about 300, about 325, about 350, about 375, about 400, about 425, about 450, about 475, about 500, about 525, about 550, about 575, about 600, about 625, about 650, about 675, about 700, about 725, about 750, about 775, about 800, about 825, about 850, about 875, about 900, about 925, about 950, about 975, about 1000, about 1100, about 1200, about 1300, about 1400, about 1500, about 1750, about 2000, about 2250, about 2500 or greater amino molecule residues of a Wnt, and any range derivable therein, as long as the amino acid sequence includes less than the full-length consecutive amino acid sequence of a Wnt. Included within the definition of “Wnt peptide” are potential amino acid sequences that include additional amino acids, other than Wnt amino acid sequences. A Wnt peptide as used herein typically refers to a Wnt peptide having the known functions of the Wnt protein.

The term “Wnt peptide” includes any amino acid sequence that includes ten or more consecutive amino acid sequence of a Wnt amino acid sequence. “Wnt peptide” includes not only consecutive amino acid sequences from a human Wnt, but from any other species, such as mouse. Thus, for example, a Wnt peptide may include 10 or more consecutive amino acids of a Wnt. Additional amino acids can also be included, which may be other than Wnt amino acid sequences. In some embodiments, a Wnt peptide has a molecular weight of greater than 700 daltons.

Amino acid sequence mutants of a Wnt peptide also are encompassed by the present invention, and are included within the definition of “Wnt peptide.” Amino acid sequence mutants of a Wnt of any species, such as human and mouse Wnt, is contemplated by the present invention. Amino acid sequence mutants of a Wnt can be substitutional mutants or insertional mutants. Insertional mutants typically involve the addition of material at a non-terminal point in the peptide. This may include the insertion of a few residues; an immunoreactive epitope; or simply a single residue. The added material may be modified, such as by methylation, acetylation, and the like. Alternatively, additional residues may be added to the N-terminal or C-terminal ends of the peptide. In some embodiments, a Wnt peptide is a conservatively modified variants of native Wnt proteins or a conservatively modified variants of active fragments of native Wnt proteins. In some embodiments, a Wnt peptide is a peptidomimetic or peptide mimetic.

The Wnt peptide encompasses variants and truncations of native Wnt proteins as well as variants and truncations of active fragments of native Wnt proteins. Active variants can be identified in any number of ways known to those of skill in the art. In some embodiments, amino acid alignments of active proteins can be established to identify those positions that are invariant or that are include conserved amino acid changes. In some embodiments, the Wnt peptide comprises consensus sequences comprising the invariant amino acids between certain areas (e.g., native full-length or a domain) of one particular native Wnt proteins (e.g., Wnt1) of different species (e.g., human, mouse, or other mammals). In some embodiments, the Wnt peptide comprises consensus sequences comprising the invariant amino acids between certain areas (e.g., native full-length or a domain) of two or more native Wnt proteins of a same species (e.g., human, mouse or mammalian Wnt1, Wnt2, Wnt2b, Wnt3, Wnt3a, Wnt4, Wnt5a, Wnt5b, Wnt 6, Wnt7a, Wnt7b, Wnt8Wnt8b, Wnt9a, Wnt9b, Wnt10a, Wnt10b, Wnt11, and Wnt16).

In some embodiments, a Wnt peptide may be fused to a particular protein, polypeptide, or peptide sequence that promotes facilitated intracellular delivery of the fusion protein into the targeted cell. Specific examples include fusion proteins utilizing the HIV TAT sequence (Nagahara et al., Nature Medicine, 4:1449-1452, 1998), the third helix of the Antennapedia homeodomain (Antp) (Derossi et al., J. Biol. Chem. 269:10444-10450, 1994), and the HSV-1 structural protein VP22 (Elliott and O'Hare, Cell, 88:223-233, 1997). Fusion partner sequences can further include, but are not limited to, amino acid tags, non-L (e.g., D-) amino acids or other amino acid mimetics to extend in vivo half-life and/or protease resistance, targeting sequences or other sequences.

PEGylation or incorporation of long-chain polyethylene glycol polymers (PEG) can optionally be introduced into the Wnt peptides of the invention (e.g. within the polypeptide chain or at either the N- or C-terminal), e.g., to extend in vivo half-life. Introduction of PEG or long chain polymers of PEG increases the effective molecular weight of the peptide, for example, to prevent rapid filtration into the urine. In some embodiments, a Lysine residue in the Wnt peptide is conjugated to PEG directly or through a linker. Such linker can be, for example, a Glu residue or an acyl residue containing a thiol functional group for linkage to the appropriately modified PEG chain. An alternative method for introducing a PEG chain is to first introduce a Cys residue at the C-terminus or at solvent exposed residues such as replacements for Arg or Lys residues. This Cys residue is then site-specifically attached to a PEG chain containing, for example, a maleimide function. Methods for incorporating PEG or long chain polymers of PEG are well known in the art (described, for example, in Veronese, F. M., et al., Drug Disc. Today 10: 1451-8 (2005); Greenwald, R. B., et al., Adv. Drug Deliv. Rev. 55: 217-50 (2003); Roberts, M. J., et al., Adv. Drug Deliv. Rev., 54: 459-76 (2002)), the contents of which is incorporated herein by reference. Other methods of polymer conjugations known in the art can also be used in the present invention. In some embodiments, poly(2-methacryloyloxyethyl phosphorylcholine) (PMPC) is introduced as a polymer conjugate with the ANGPTL3 proteins of the invention (see, e.g., WO2008/098930; Lewis, et al., Bioconjug Chem., 19: 2144-55 (2008)). In some embodiments, a phosphorylcholine-containing polymer conjugate with the ANGPTL3 proteins can be used in the present invention. A person of skill would readily recognize that other biocompatible polymer conjugates can be utilized. Alternatively, incorporation of PEG or PEG polymers through incorporation of non-natural amino acids (as described above) can be performed with the Wnt peptides. This approach utilizes an evolved tRNA/tRNA synthetase pair and is coded in the expression plasmid by the amber suppressor codon (Deiters, A, et al. (2004). Bio-org. Med. Chem. Lett. 14, 5743-5). For example, p-azidophenylalanine can be incorporated into the present polypeptides and then reacted with a PEG polymer having an acetylene moiety in the presence of a reducing agent and copper ions to facilitate an organic reaction known as “Huisgen [3+2]cycloaddition.”

### IV. Small Molecule Wnt Mimetics

The Wnt compound provided herein can also be a small molecule Wnt mimetic, e.g., a small molecule having a molecular weight of less than about 700 daltons. Accordingly, in some embodiments, a small molecule Wnt mimetic is a Wnt peptide (e.g., a peptidomimetic or a peptide mimetic) having a molecular weight of less than about 700 daltons.

In addition, a small molecule Wnt mimetic can be designed by: (a) identifying the pharmacophoric groups responsible for the biological activity of a reference gene product (e.g., a Wnt polypeptide or a functional fragment thereof); (b) determining the spatial arrangements of the pharmacophoric groups in the active conformation of the reference gene product; and (c) selecting a pharmaceutically acceptable template upon which to mount the pharmacophoric groups in a manner that allows them to retain their spatial arrangement in the active conformation of the reference gene product. For identification of pharmacophoric groups responsible for biological activity (e.g., neuron degeneration inhibiting activity or neuron degeneration promoting activity), mutant variants of the reference gene product can be prepared and assayed for relevant biological activity. Alternatively or in addition, the three-dimensional structure of a complex of the reference gene product and its target molecule (e.g., a receptor) can be examined for evidence of interactions, for example the fit of a peptide side chain into a cleft of the target molecule, potential sites for hydrogen bonding, etc. The spatial arrangements of the pharmacophoric groups can be determined by NMR spectroscopy or X-ray diffraction studies. An initial three-dimensional model can be refined by energy minimization and molecular dynamics simulation. A template for modeling can be selected by reference to a template database and will typically allow the mounting of 2-8 pharmacophores. A small molecule mimetic is identified wherein addition of the pharmacophoric groups to the template maintains their spatial arrangement as in the Wnt11 gene product.

A small molecule Wnt mimetic can also be identified by assigning a hashed bitmap structural fingerprint to the reference gene product based on its chemical structure, and determining the similarity of that fingerprint to that of each compound in a broad chemical database. The fingerprints can be determined using fingerprinting software commercially distributed for that purpose by Daylight Chemical Information Systems, Inc. (Mission Viejo, Calif., United States of America) according to the vendor's instructions. Representative databases include but are not limited to SPREI'95 (InfoChem GmbH of Munich, Germany), Index Chemicus (ISI of Philadelphia, Pa., United States of America), World Drug Index (Derwent of London, United Kingdom), TSCA93 (United States Envrionmental Protection Agency), MedChem (Biobyte of Claremont, Calif., United States of America), Maybridge Organic Chemical Catalog (Maybridge of Cornwall, England), Available Chemicals Directory (MDL Information Systems of San Leandro, Calif., United States of America), NCI96 (United States National Cancer Institute), Asinex Catalog of Organic Compounds (Asinex Ltd. of Moscow, Russia), and NP (InterBioScreen Ltd. of Moscow, Russia). A small molecule mimetic of a reference gene product is selected as comprising a fingerprint with a similarity (Tanamoto coefficient) of in some embodiments at least 0.85 relative to the fingerprint of the reference gene product.

Additional techniques for the design and preparation of small molecule Wnt mimetics can be found in U.S. Pat. Nos. 5,811,392; 5,811,512; 5,578,629; 5,817,879; 5,817,757; and 5,811,515. Any small molecule or peptide mimetic of the presently disclosed subject matter can be used in the form of a pharmaceutically acceptable salt.

### V. Fzd3 Dephosphorylating Agent

A Fzd3 dephosphorylating agent refers to an agent that reduces Frizzled-3 (Fzd3) phosphorylation or hyperphosphorylation, and/or promotes Fzd3 internalization or endocytosis thereby reduces Fzd3 level on cell surface. Accordingly, in some embodiments, a Fzd3 dephosphorylating agent can be a Vang Gogh Like protein 2 (Vgl2) peptide, a Vgl2 mimetic, or a dishevelled 1 (Dvl1) antagonist.

### 1. Vgl2 Peptides

Vgl2 is a protein involved in the control of early morphogenesis and patterning of both axial midline structures and the development of neural plate. Vgl2 plays a role in the regulation of planar cell polarity, particularly in the orientation of stereociliary bundles in the cochlea. Vgl2 interacts through its C-terminal region with the N-terminal half of DVL1, DVL2 and DVL3. The PDZ domain of DVL1, DVL2 and DVL3 is required for the interaction. Vgl2 also interacts with the PDZ domains of MAGI3, SCRIB/SCRB1 and FZD3. Vgl2 peptides can be a human Vgl2 protein, or a Vgl2 protein from any other species, such as mammal, primate, rat, mouse, rabbit, bovine, dog, cat, pig, etc. The human Vgl2 full-length native amino acid sequences are described by GenBank Accession No.:NP_{—}065068 (SEQ ID NO:46). Vgl2 is a four-transmembrane protein having an intracellular C-terminal domain (residue 238-521; SEQ ID NO:47). The murine Vgl2 full-length native amino acid sequences are described by GenBank Accession No.:NP_{—}277044 (SEQ ID NO:48).

In some embodiments, a Vgl2 peptide is a polypeptide comprising an amino acid sequence having at least 60% identity, optionally 65%, 70%, 75%, 80%, 85%, 90%, 95%, 99% or 100% identity to the entire or a portion (as discussed below) of SEQ ID NO:46, 47, or 48.

The term “Vgl2 peptide” includes any amino acid sequence that includes fewer consecutive amino acids of a Vgl2 than the full-length amino acid sequence of a Vgl2. “Vgl2 peptide” includes not only consecutive amino acid sequences from a human Vgl2, but also homologs thereof such as sequences from any other species, such as mouse. Thus, for example, a Vgl2 peptide can include, but is not limited to, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, about 100, about 110, about 120, about 130, about 140, about 150, about 160, about 170, about 180, about 190, about 200, about 210, about 220, about 230, about 240, about 250, about 275, about 300, about 325, about 350, about 375, about 400, about 425, about 450, about 475, about 500, about 525, about 550, about 575, about 600, about 625, about 650, about 675, about 700, about 725, about 750, about 775, about 800, about 825, about 850, about 875, about 900, about 925, about 950, about 975, about 1000, about 1100, about 1200, about 1300, about 1400, about 1500, about 1750, about 2000, about 2250, about 2500 or greater amino molecule residues of a Vgl2, and any range derivable therein, as long as the amino acid sequence includes less than the full-length consecutive amino acid sequence of a Vgl2. Included within the definition of “Vgl2 peptide” are potential amino acid sequences that include additional amino acids, other than Vgl2 amino acid sequences. A Vgl2 peptide as used herein typically refers to a Vgl2 peptide having the known functions of the Vgl2 protein.

The term “Vgl2 peptide” includes any amino acid sequence that includes ten or more consecutive amino acid sequence of a Vgl2 amino acid sequence. “Vgl2 peptide” includes not only consecutive amino acid sequences from a human Vgl2, but from any other species, such as mouse. Thus, for example, a Vgl2 peptide may include 10 or more consecutive amino acids of a Vgl2. Additional amino acids can also be included, which may be other than Vgl2 amino acid sequences. In some embodiments, a Vgl2 peptide has a molecular weight of greater than 700 daltons.

Amino acid sequence mutants of a Vgl2 peptide also are encompassed by the present invention, and are included within the definition of “Vgl2 peptide.” Amino acid sequence mutants of a Vgl2 of any species, such as human and mouse Vgl2, is contemplated by the present invention. Amino acid sequence mutants of a Vgl2 can be substitutional mutants or insertional mutants. Insertional mutants typically involve the addition of material at a non-terminal point in the peptide. This may include the insertion of a few residues; an immunoreactive epitope; or simply a single residue. The added material may be modified, such as by methylation, acetylation, and the like. Alternatively, additional residues may be added to the N-terminal or C-terminal ends of the peptide. In some embodiments, a Vgl2 peptide is a conservatively modified variants of native Vgl2 proteins or a conservatively modified variants of active fragments of native Vgl2 proteins. In some embodiments, a Vgl2 peptide is a peptidomimetic or peptide mimetic.

The Vgl2 peptide encompasses variants and truncations of native Vgl2 proteins as well as variants and truncations of active fragments of native Vgl2 proteins. Active variants can be identified in any number of ways known to those of skill in the art, as described in section III, supra. In some embodiments, the Vgl2 peptide is a C-terminal region of the full-length Vgl2 protein (e.g., residue 238-521). In some embodiments, the Vgl2 peptide is a variant and/or truncation of the C-terminal region of the full-length Vgl2 protein.

In some embodiments, the Vgl2 peptide comprises a full-length amino acid sequence that is encoded by a Vgl2 gene. In some embodiments, the Vgl2 peptide is an amino acid sequence mutant of a Vgl2 peptide. In some embodiments, a Vgl2 peptide is a conservatively modified variants of native Vgl2 proteins or a conservatively modified variants of active fragments of native Vgl2 proteins. In some embodiments, a Vgl2 peptide is a peptidomimetic or peptide mimetic. Fusion proteins, PEGylation or incorporation of long-chain polyethylene glycol polymers (PEG) of the Vgl2 peptides are also included and can be designed as described in section III, supra.

### 2. Vgl2 Mimetics

The Fzd3 dephosphorylating agent provided herein can also be a small molecule Vgl2 mimetic, e.g., a small molecule having a molecular weight of less than about 700 daltons. Accordingly, in some embodiments, a small molecule Vgl2 mimetic is a Vgl2 peptide (e.g., a peptidomimetic or a peptide mimetic) having a molecular weight of less than about 700 daltons. Additional small molecule Vgl2 mimetics can be designed and identified using methods disclose in section IV, supra.

### 3. Dvl1 Antagonists

The Fzd3 dephosphorylating agent provided herein can also be a Dvl1 antagonist. In some embodiments, the Vgl2 antagonist is an antibody to Dvl1. In some embodiments, the Dvl1 antagonist is a dominant negative mutant of native Dvl1. In some embodiments, the Vgl2 antagonist is an inhibitory nucleic acid. In some embodiments, the Vgl2 antagonist is a siRNA targeting Vgl2.

Exemplary Dvl1 antagonists include NSC668036 (9,15-Diisopropyl-2,2,6,12-tetramethyl-4,7,10,13-tetraoxo-3,8,14-trioxa-5,11-diazahexadecan-16-oic acid; Shan et al., Biochemistry, 29:15495-15503, 2005), FJ9 (Fujii et al., Cancer Res 67:573-579, 2007). Exemplary Dvl1 antagonists also include, but are not limited to, those described in You et al., Mol Cancer Ther, 7:1633-1638, 2008, WO04092346, WO06007542.

### VI. Fzd3 Phosphorylating Agent

A Fzd3 phosphorylating agent refers to an agent that promotes Frizzled-3 (Fzd3) phosphorylation or hyperphosphorylation, and/or decreases Fzd3 internalization or endocytosis thereby increases Fzd3 level on cell surface. Accordingly, in some embodiments, a Fzd3 dephosphorylating agent can be a Dvl1 peptide, a Dvl1 mimetic, or a Vgl2 antagonist.

### 1. Dvl1 Peptides

Dvl1 protein is a cytoplasmic phosphoprotein that regulates cell proliferation, acting as a transducer molecule for developmental processes, including segmentation and neuroblast specification. Dvl1 peptides can be a human Dvl1 protein, or a Dvl1 protein from any other species, such as mammal, primate, rat, mouse, rabbit, bovine, dog, cat, pig, etc. The human Dvl1 full-length native amino acid sequences are described by GenBank Accession No.: NP_{—}004412 (SEQ ID NO:49). The murine Dvl1 full-length native amino acid sequences are described by GenBank Accession No.: NP_{—}034221 (SEQ ID NO:50). Dvl1 possesses three conserved domains, a DIX domain, a PDZ domain, and a DEP domain (Wong et al., Nat Struct Biol, 7:1178-1184, 2000). In some embodiments, the Dvl1 peptide comprises the DPZ domain of Dvl1.

In some embodiments, a Dvl1 peptide is a polypeptide comprising an amino acid sequence having at least 60% identity, optionally 65%, 70%, 75%, 80%, 85%, 90%, 95%, 99% or 100% identity to the entire or a portion (as discussed below) of identity to SEQ ID NO: 49-50.

The term “DVl1 peptide” includes any amino acid sequence that includes fewer consecutive amino acids of a DVl1 than the full-length amino acid sequence of a DVl1. “DVl1 peptide” includes not only consecutive amino acid sequences from a human Dvl1, but also homologs thereof such as sequences from any other species, such as mouse. Thus, for example, a DVl1 peptide can include, but is not limited to, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, about 100, about 110, about 120, about 130, about 140, about 150, about 160, about 170, about 180, about 190, about 200, about 210, about 220, about 230, about 240, about 250, about 275, about 300, about 325, about 350, about 375, about 400, about 425, about 450, about 475, about 500, about 525, about 550, about 575, about 600, about 625, about 650, about 675, about 700, about 725, about 750, about 775, about 800, about 825, about 850, about 875, about 900, about 925, about 950, about 975, about 1000, about 1100, about 1200, about 1300, about 1400, about 1500, about 1750, about 2000, about 2250, about 2500 or greater amino molecule residues of a Dvl1, and any range derivable therein, as long as the amino acid sequence includes less than the full-length consecutive amino acid sequence of a Dvl1. Included within the definition of “Dvl1 peptide” are potential amino acid sequences that include additional amino acids, other than Dvl1 amino acid sequences. A Dvl1 peptide as used herein typically refers to a Dvl1 peptide having the known functions of the Dvl1 protein.

The term “Dvl1 peptide” includes any amino acid sequence that includes ten or more consecutive amino acid sequence of a Dvl1 amino acid sequence. “Dvl1 peptide” includes not only consecutive amino acid sequences from a human Dvl1, but from any other species, such as mouse. Thus, for example, a Dvl1 peptide may include 10 or more consecutive amino acids of a Dvl1. Additional amino acids can also be included, which may be other than Dvl1 amino acid sequences. In some embodiments, a Dvl1 peptide has a molecular weight of greater than 700 daltons.

Amino acid sequence mutants of a Dvl1 peptide also are encompassed by the present invention, and are included within the definition of “Dvl1 peptide.” Amino acid sequence mutants of a Dvl1 of any species, such as human and mouse Dvl1, is contemplated by the present invention. Amino acid sequence mutants of a Dvl1 can be substitutional mutants or insertional mutants. Insertional mutants typically involve the addition of material at a non-terminal point in the peptide. This may include the insertion of a few residues; an immunoreactive epitope; or simply a single residue. The added material may be modified, such as by methylation, acetylation, and the like. Alternatively, additional residues may be added to the N-terminal or C-terminal ends of the peptide. In some embodiments, a Dvl1 peptide is a conservatively modified variants of native Dvl1 proteins or a conservatively modified variants of active fragments of native Dvl1 proteins. In some embodiments, a Dvl1 peptide is a peptidomimetic or peptide mimetic.

In some embodiments, the Dvl1 peptide comprises a full-length amino acid sequence that is encoded by a Dvl1 gene. In some embodiments, the Dvl1 peptide is a variant or a truncation of native Dvl1 proteins as well as variants and truncations of active fragments of native Dvl1 proteins. In some embodiments, the Dvl1 peptide is an amino acid sequence mutant of a Dvl1 peptide. In some embodiments, a Dvl1 peptide is a conservatively modified variants of native Dvl1 proteins or a conservatively modified variants of active fragments of native Dvl1 proteins. In some embodiments, a Dvl1 peptide is a peptidomimetic or peptide mimetic. Fusion proteins, PEGylation or incorporation of long-chain polyethylene glycol polymers (PEG) of the Dvl1 peptides are also included and can be designed as described in section III, supra.

### 2. Dvl1 Mimetics

The Fzd3 phosphorylating agent provided herein can also be a small molecule Dvl1 mimetic, e.g., a small molecule having a molecular weight of less than about 700 daltons. Accordingly, in some embodiments, a small molecule Dvl1 mimetic is a Dvl1 peptide (e.g., a peptidomimetic or a peptide mimetic) having a molecular weight of less than about 700 daltons. Additional small molecule Dvl1 mimetics can be designed and identified using methods disclose in section IV, supra.

### 3. Vgl2 Antagonists

The Fzd3 phosphorylating agent provided herein can also be a Vgl2 antagonist. In some embodiments, the Vgl2 antagonist is an antibody to Vgl2. In some embodiments, the Vgl2 antagonist is a dominant negative mutant of native Vgl2.

In some embodiments, the dominant negative Vgl2 can have an amino acid sequence of wild type Vgl2 or fragments thereof except Ser residue at positions 464 in the wild type Vgl2 is deleted or substituted with amino acid that results in Fzd3 dephosphrylation. In one example, the dominant negative Vgl2 is an S464N mutant. The S464N mutant, as defined herein, can be a full-length Vgl2 or fragments thereof. In another example, the dominant negative Vgl2 is a C-terminal region of Vgl2 having a S464N mutation.

In some embodiments, the Vgl2 antagonist is an inhibitory nucleic acid. In some embodiments, the Vgl2 antagonist is a siRNA targeting Vgl2.

### VII. Methods for Modulating Neuron Cell Guidance

Provided herein are novel methods and compositions for modulating neuron cell guidance of a neuron by contacting the neuron with a SHH compound. In some embodiments, the SHH compound is a SHH compound or a small molecule SHH mimetic. For example, the SHH peptide can be a polypeptide comprising an amino acid sequence having at 90% to SEQ ID NOs:51-52. Accordingly, it was discovered in the present invention that a SHH compound can be used for regenerating a neuron (e.g., an axon). In some embodiments, the neuron cell guidance facilitates regeneration of a neuron.

The methods for neuron regeneration and/or modulating neuron cell guidance include in vitro, in vivo, and/or ex vivo methods. In some embodiments, the methods are practiced in vivo, i.e., the neuron regenerating agent or the agent modulating neuron cell guidance is administered to a subject. In some embodiments, the methods are practiced ex vivo, i.e., neurons to be treated form part of a nerve graft or a nerve transplant in a subject. In some embodiments, the methods are practiced in vitro.

Provided herein also are novel methods and compositions for causing guidance defect of a neuron by contacting the neuron with a SHH antagonist thereby causing guidance defect of the neuron. In some embodiments, the SHH antagonist is an antibody to SHH (e.g., a monoclonal antibody 5E1 as detailed in the Examples). In some embodiments, the SHH antagonist is a dominant negative mutant of native SHH. In some embodiments, the SHH antagonist is an inhibitory nucleic acid. In some embodiments, the SHH antagonist is a siRNA targeting SHH. Exemplary SHH antagonists include GDC-0449 (2-chloro-N-(4-chloro-3-(pyridin-2-yl)phenyl)-4-(methylsulfonyl)benzamide), SANT-1 ((4-Benzyl-piperazin-1-yl)-(3,5-dimethyl-1-phenyl-1H-pyrazol-4-ylmethylene)-amine). Exemplary SHH antagonists also include, but are not limited to, those described in WO2006/050351. Exemplary SHH antibodies include, but are not limited to, those described in WO2009/086324.

In addition, a SHH antagonist further includes an antigonist to SHH pathway. The SHH pathway antagonist can be any agent (e.g., small molecule, antibody, small interfering RNA, etc) that exerts its inhibitory affect on the pathway through an interaction with one or more components of the pathway, e.g., the hedgehog ligand, smoothened, patched, or GIi. In some embodiments, the SHH antagonist is an antagonist to Patched-1, e.g., an antibody to Patched-1 or an inhibitory nucleic acid targeting (e.g., a siRNA or shRNA) Patched-1. In some embodiments, the SHH antagonist is an antagonist to Smoothened, e.g., an antibody to Smoothened or an inhibitory nucleic acid targeting (e.g., a siRNA or shRNA) Smoothened.

Exemplary SHH pathway antagonists include, but are not limited to, those described in U.S. Pat. No. 7,230,004, U.S. Patent Application Publication No. 2008/0293754, U.S. Patent Application Publication No. 2008/0287420, and U.S. Patent Application Publication No. 2008/0293755, the entire disclosures of which are incorporated by reference herein. Examples of other suitable hedgehog inhibitors include those described in U.S. Patent Application Publication Nos. US 2002/0006931, US 2007/0021493 and US 2007/0060546, and International Application Publication Nos. WO 2001/19800, WO 2001/26644, WO 2001/27135, WO 2001/49279, WO 2001/74344, WO 2003/011219, WO 2003/088970, WO 2004/020599, WO 2005/013800, WO 2005/033288, WO 2005/032343, WO 2005/042700, WO 2006/028958, WO 2006/050351, WO 2006/078283, WO 2007/054623, WO 2007/059157, WO 2007/120827, WO 2007/131201, WO 2008/070357, WO 2008/110611, WO 2008/112913, WO 2008/131354, and WO2010/085654. Exemplary SHH antagonists include MK-4101 (Merck), GDC-0449 (Genentech), XL-139 (BMS-833923) (Bristol Myers Squibb), LDE 225 (Novartis), PF-04449913 (Pfizer), robotnikinin, and Cur-61414 (G-024856).

### VIII. SHH Compounds

Provided herein are sonic hedgehog (SHH) compounds for modulating neuron cell guidance of a neuron comprising contacting the neuron with a SHH compound thereby modulating neuron cell guidance. As used herein, a SHH compound can be a SHH peptide or a small molecule SHH mimetic.

Sonic hedgehogs (SHHs) are secreted proteins that regulate patterning of the developing central nervous system. SHH can be a human SHH protein, or a SHH protein from any other species, such as mammal, primate, rat, mouse, rabbit, bovine, dog, cat, pig, etc. The human SHH full-length native amino acid sequences are described by GenBank Accession No.:AAA62179 (SEQ ID NO:51). The SHH precursor protein is composed of the amino acids 1-462 of the sequence described in the EMBL database under No.:L38518. The amino acids 1-23 represent the signal peptide, the amino acids 24-197 represent the mature signal domain, the amino acids 32-197 represent the signal domain shortened by 8 amino acids and the amino acids 198-462 represent the auto-processing domain after autoproteolytic cleavage. The murine SHH full-length native amino acid sequences are described by GenBank Accession No.:NP_{—}033196 (SEQ ID NO:52).

In some embodiments, a SHH peptide is a polypeptide comprising an amino acid sequence having at least 60% identity, optionally 65%, 70%, 75%, 80%, 85%, 90%, 95%, or 99% identity to SEQ ID NOs:51-52.

The term “SHH peptide” includes any amino acid sequence that includes fewer consecutive amino acids of a SHH than the full-length amino acid sequence of a SHH. “SHH peptide” includes not only consecutive amino acid sequences from a human SHH, but also homologs thereof such as sequences from any other species, such as mouse. Thus, for example, a SHH peptide can include, but is not limited to, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, about 100, about 110, about 120, about 130, about 140, about 150, about 160, about 170, about 180, about 190, about 200, about 210, about 220, about 230, about 240, about 250, about 275, about 300, about 325, about 350, about 375, about 400, about 425, about 450, about 475, about 500, about 525, about 550, about 575, about 600, about 625, about 650, about 675, about 700, about 725, about 750, about 775, about 800, about 825, about 850, about 875, about 900, about 925, about 950, about 975, about 1000, about 1100, about 1200, about 1300, about 1400, about 1500, about 1750, about 2000, about 2250, about 2500 or greater amino molecule residues of a SHH, and any range derivable therein, as long as the amino acid sequence includes less than the full-length consecutive amino acid sequence of a SHH. Included within the definition of “SHH peptide” are potential amino acid sequences that include additional amino acids, other than SHH amino acid sequences. A SHH peptide as used herein typically refers to a SHH peptide having the known functions of the SHH protein.

The term “SHH peptide” includes any amino acid sequence that includes ten or more consecutive amino acid sequence of a SHH amino acid sequence. “SHH peptide” includes not only consecutive amino acid sequences from a human SHH, but from any other species, such as mouse. Thus, for example, a SHH peptide may include 10 or more consecutive amino acids of a SHH. Additional amino acids can also be included, which may be other than SHH amino acid sequences. In some embodiments, a SHH peptide has a molecular weight of greater than 700 daltons.

Amino acid sequence mutants of a SHH peptide also are encompassed by the present invention, and are included within the definition of “SHH peptide.” Amino acid sequence mutants of a SHH of any species, such as human and mouse SHH, is contemplated by the present invention. Amino acid sequence mutants of a SHH can be substitutional mutants or insertional mutants. Insertional mutants typically involve the addition of material at a non-terminal point in the peptide. This may include the insertion of a few residues; an immunoreactive epitope; or simply a single residue. The added material may be modified, such as by methylation, acetylation, and the like. Alternatively, additional residues may be added to the N-terminal or C-terminal ends of the peptide. In some embodiments, a SHH peptide is a conservatively modified variants of native SHH proteins or a conservatively modified variants of active fragments of native SHH proteins. In some embodiments, a SHH peptide is a peptidomimetic or peptide mimetic.

In some embodiments, the SHH peptide comprises a full-length amino acid sequence that is encoded by a SHH gene. In some embodiments, the SHH peptide is a variant or a truncation of native SHH proteins as well as variants and truncations of active fragments of native SHH proteins. In some embodiments, the SHH peptide is an amino acid sequence mutant of a SHH peptide. In some embodiments, a SHH peptide is a conservatively modified variants of native SHH proteins or a conservatively modified variants of active fragments of native SHH proteins. In some embodiments, a SHH peptide is a peptidomimetic or peptide mimetic. Fusion proteins, PEGylation or incorporation of long-chain polyethylene glycol polymers (PEG) of the SHH peptides are also included and can be designed as described in section III, supra.

The SHH compound provided herein can also be a small molecule SHH mimetic, e.g., a small molecule having a molecular weight of less than about 700 daltons. Accordingly, in some embodiments, a small molecule SHH mimetic is a SHH peptide (e.g., a peptidomimetic or a peptide mimetic) having a molecular weight of less than about 700 daltons. Additional small molecule SHH mimetics can be designed and identified using methods disclose in section IV, supra.

### IX. Neurons

As used herein, the term “neuron” include a neuron and a portion or portions thereof (e.g., the neuron cell body, an axon, or a dendrite). The term “neuron” as used herein denotes nervous system cells that include a central cell body or soma, and two types of extensions or projections: dendrites, by which, in general, the majority of neuronal signals are conveyed to the cell body, and axons, by which, in general, the majority of neuronal signals are conveyed from the cell body to effector cells, such as target neurons or muscle. Neurons can convey information from tissues and organs into the central nervous system (afferent or sensory neurons) and transmit signals from the central nervous systems to effector cells (efferent or motor neurons). Other neurons, designated interneurons, connect neurons within the central nervous system (the brain and spinal column). Certain specific examples of neuron types that may be subject to treatment or methods according to the invention include cerebellar granule neurons, dorsal root ganglion neurons, and cortical neurons.

The term “neuronal degeneration” is used broadly and refers to any pathological changes in neuronal cells, including, without limitation, death or loss of neuronal cells and any changes that precede cell death. The pathological changes may be spontaneous or may be induced by any event and include, for example, pathological changes associated with apoptosis. The neurons may be any neurons, including without limitation sensory, sympathetic, parasympathetic, or enteric, e.g. dorsal root ganglia neurons, motor neurons, and central neurons, e.g. neurons from the spinal cord. Neuronal degeneration or cell loss is a characteristic of a variety of neurodegenerative disorders. In some embodiments, the neuron is a sensory neuron. In some embodiments, the neuron is a motor neuron. In some embodiments, the neuron is a damaged spinal cord neuron.

In some embodiments, degeneration occurs in a portion of the neuron such as the neuron cell body, an axon, or a dendrite. Accordingly, the degeneration can be inhibited in the degenerated portion or portions of the neuron. In some embodiments, the degeneration of an axon of the neuron is inhibited. In some embodiments, the degeneration of a cell body of the neuron is inhibited. The axon can be an axon of any neuron. For example, in some embodiments, the axon is a spinal cord commissural axon, or an upper motor neuron axon, or a central nervous system axon.

As described herein, the methods provided herein can be carried out in vivo, such as in the treatment of neurodegenerative diseases, neurological disorders or injuries to the nervous system. The methods can also be carried out in vitro or ex vivo, such as in laboratory studies of neuron function and in the treatment of nerve grafts or transplants. Accordingly, in some embodiments, the neuron forms part of a nerve graft or a nerve transplant. In some embodiments, the neuron is ex vivo or in vitro. In some embodiments, the nerve graft or the nerve transplant forms part of an organism, human or non-human (e.g., mammal, primate, rat, mouse, rabbit, bovine, dog, cat, pig, etc.).

### X. Diseases

Wnt compounds or Fzd3 dephosphorylating agents can be used in methods for inhibiting neuron (e.g., axon) degeneration. These compounds are, therefore, useful in the therapy of, for example, (i) disorders of the nervous system (e.g., neurodegenerative diseases), (ii) conditions of the nervous system that are secondary to a disease, condition, or therapy having a primary effect outside of the nervous system, (iii) injuries to the nervous system caused by physical, mechanical, or chemical trauma, (iv) pain, (v) ocular-related neurodegeneration, (vi) memory loss, and (vii) psychiatric disorders. Non-limiting examples of some of these diseases, conditions, and injuries are provided below.

Examples of neurodegenerative diseases and conditions that can be prevented or treated according to the invention include amyotrophic lateral sclerosis (ALS), trigeminal neuralgia, glossopharyngeal neuralgia, Bell's Palsy, myasthenia gravis, muscular dystrophy, progressive muscular atrophy, primary lateral sclerosis (PLS), pseudobulbar palsy, progressive bulbar palsy, spinal muscular atrophy, progressive bulbar palsy, inherited muscular atrophy, invertebrate disk syndromes (e.g., herniated, ruptured, and prolapsed disk syndromes), cervical spondylosis, plexus disorders, thoracic outlet destruction syndromes, peripheral neuropathies, prophyria, mild cognitive impairment, Alzheimer's disease, Huntington's disease, Parkinson's disease, Parkinson' s-plus diseases (e.g., multiple system atrophy, progressive supranuclear palsy, and corticobasal degeneration), dementia with Lewy bodies, frontotemporal dementia, demyelinating diseases (e.g., Guillain-Barre syndrome and multiple sclerosis), Charcot-Marie-Tooth disease (CMT; also known as Hereditary Motor and Sensory Neuropathy (HMSN), Hereditary Sensorimotor Neuropathy (HSMN), and Peroneal Muscular Atrophy), prion disease (e.g., Creutzfeldt-Jakob disease, Gerstmann-Straussler-Scheinker syndrome (GSS), fatal familial insomnia (FFI), and bovine spongiform encephalopathy (BSE, commonly known as mad cow disease)), Pick's disease, epilepsy, and AIDS demential complex (also known as HIV dementia, HIV encephalopathy, and HIV-associated dementia).

The methods of the invention can also be used in the prevention and treatment of ocular-related neurodegeneration and related diseases and conditions, such as glaucoma, lattice dystrophy, retinitis pigmentosa, age-related macular degeneration (AMD), photoreceptor degeneration associated with wet or dry AMD, other retinal degeneration, optic nerve drusen, optic neuropathy, and optic neuritis. Non-limiting examples of different types of glaucoma that can be prevented or treated according to the invention include primary glaucoma (also known as primary open-angle glaucoma, chronic open-angle glaucoma, chronic simple glaucoma, and glaucoma simplex), low-tension glaucoma, primary angle-closure glaucoma (also known as primary closed- angle glaucoma, narrow-angle glaucoma, pupil-block glaucoma, and acute congestive glaucoma), acute angle-closure glaucoma, chronic angle-closure glaucoma, intermittent angle-closure glaucoma, chronic open-angle closure glaucoma, pigmentary glaucoma, exfoliation glaucoma (also known as pseudoexfoliative glaucoma or glaucoma capsulare), developmental glaucoma (e.g., primary congenital glaucoma and infantile glaucoma), secondary glaucoma (e.g., inflammatory glaucoma (e.g., uveitis and Fuchs heterochromic iridocyclitis)), phacogenic glaucoma (e.g., angle-closure glaucoma with mature cataract, phacoanaphylactic glaucoma secondary to rupture of lens capsule, phacolytic glaucoma due to phacotoxic meshwork blockage, and subluxation of lens), glaucoma secondary to intraocular hemorrhage (e.g., hyphema and hemolytic glaucoma, also known as erythroclastic glaucoma), traumatic glaucoma (e.g., angle recession glaucoma, traumatic recession on anterior chamber angle, postsurgical glaucoma, aphakic pupillary block, and ciliary block glaucoma), neovascular glaucoma, drug-induced glaucoma (e.g., corticosteroid induced glaucoma and alpha-chymotrypsin glaucoma), toxic glaucoma, and glaucoma associated with intraocular tumors, retinal deatchments, severe chemical burns of the eye, and iris atrophy.

Examples of types of pain that can be treated according to the methods of the invention include those associated with the following conditions: chronic pain, fibromyalgia, spinal pain, carpel tunnel syndrome, pain from cancer, arthritis, sciatica, headaches, pain from surgery, muscle spasms, back pain, visceral pain, pain from injury, dental pain, neuralgia, such as neuogenic or neuropathic pain, nerve inflammation or damage, shingles, herniated disc, torn ligament, and diabetes.

Certain diseases and conditions having primary effects outside of the nervous system can lead to damage to the nervous system, which can be treated according to the methods of the present invention. Examples of such conditions include peripheral neuropathy and neuralgia caused by, for example, diabetes, cancer, AIDS, hepatitis, kidney dysfunction, Colorado tick fever, diphtheria, HIV infection, leprosy, lyme disease, polyarteritis nodosa, rheumatoid arthritis, sarcoidosis, Sjogren syndrome, syphilis, systemic lupus erythematosus, and amyloidosis.

In addition, the methods of the invention can be used in the treatment of nerve damage, such as peripheral neuropathy, which is caused by exposure to toxic compounds, including heavy metals (e.g., lead, arsenic, and mercury) and industrial solvents, as well as drugs including chemotherapeutic agents (e.g., vincristine and cisplatin), dapsone, HIV medications (e.g., Zidovudine, Didanosine, Stavudine, Zalcitabine, Ritonavir, and Amprenavir), cholesterol lowering drugs (e.g., Lovastatin, Indapamid, and Gemfibrozil), heart or blood pressure medications (e.g., Amiodarone, Hydralazine, Perhexiline), and Metronidazole.

The methods of the invention can also be used to treat injury to the nervous system caused by physical, mechanical, or chemical trauma. Thus, the methods can be used in the treatment of peripheral nerve damage caused by physical injury (associated with, e.g., burns, wounds, surgery, and accidents), ischemia, prolonged exposure to cold temperature (e.g., frost-bite), as well as damage to the central nervous system due to, e.g., stroke or intracranial hemorrhage (such as cerebral hemorrhage).

Further, the methods of the invention can be used in the prevention or treatment of memory loss such as, for example, age-related memory loss. Types of memory that can be affected by loss, and thus treated according to the invention, include episodic memory, semantic memory, short-term memory, and long-term memory. Examples of diseases and conditions associated with memory loss, which can be treated according to the present invention, include mild cognitive impairment, Alzheimer's disease, Parkinson's disease, Huntington's disease, chemotherapy, stress, stroke, and traumatic brain injury (e.g., concussion).

The methods of the invention can also be used in the treatment of psychiatric disorders including, for example, schizophrenia, delusional disorder, schizoaffective disorder, schizopheniform, shared psychotic disorder, psychosis, paranoid personality disorder, schizoid personality disorder, borderline personality disorder, anti-social personality disorder, narcissistic personality disorder, obsessive-compulsive disorder, delirium, dementia, mood disorders, bipolar disorder, depression, stress disorder, panic disorder, agoraphobia, social phobia, post-traumatic stress disorder, anxiety disorder, and impulse control disorders (e.g., kleptomania, pathological gambling, pyromania, and trichotillomania).

In addition to the in vivo methods described above, the methods of the invention can be used to treat nerves ex vivo, which may be helpful in the context of nerve grafts or nerve transplants. Thus, the compounds provided herein can be useful as components of culture media for use in culturing nerve cells in vitro.

The compounds can be optionally combined with or administered in concert with each other or other agents known to be useful in the treatment of the relevant disease or condition. Thus, in the treatment of ALS, for example, the compounds can be administered in combination with Riluzole (Rilutek), minocycline, insulin-like growth factor 1 (IGF-I), and/or methylcobalamin. In another example, in the treatment of Parkinson's disease, inhibitors can be administered with L-dopa, dopamine agonists (e.g., bromocriptine, pergolide, pramipexole, ropinirole, cabergoline, apomorphine, and lisuride), dopa decarboxylase inhibitors (e.g., levodopa, benserazide, and carbidopa), and/or MAO-B inhibitors (e.g., selegiline and rasagiline). In a further example, in the treatment of Alzheimer's disease, inhibitors can be administered with acetylcholinesterase inhibitors (e.g., donepezil, galantamine, and rivastigmine) and/or NMDA receptor antagonists (e.g., memantine). The combination therapies can involve concurrent or sequential administration, by the same or different routes, as determined to be appropriate by those of skill in the art. The invention also includes pharmaceutical compositions and kits including combinations as described herein.

### XI. Assays

### 1. Assays for Inhibiting Neuron Degeneration

Additional agents useful for practicing the methods provided herein can be identified using standard screening methods known in the art. These assays can also be used to confirm the activities of agents found to have a desired activity, which are designed according to standard medicinal chemistry approaches. After an agent is confirmed as being active with respect to a particular target, the agent can be tested in models of neuron or axon degeneration, as well as in appropriate animal model systems.

Cell-Based and In Vitro Assays for inhibiting Neuron Degeneration include, for example, (i) anti-Nerve Growth Factor (anti-NGF) antibody assays, (ii) serum deprivation/potassium chloride (KCl) reduction assays, (iii) rotenone degeneration assays, and (iv) vincristine degeneration assays. Additional assays for assessing neuron or axon degeneration that are known in the art can also be used in the invention.

NGF is a small, secreted protein involved in differentiation and survival of target neurons. Treatment of cultured neurons with NGF results in proliferation of axons, while treating such neurons with anti-NGF antibodies results in axon degeneration. Treatment of neurons with anti-NGF antibodies also leads to several different morphological changes that are detectable by microscopy, and which can be monitored to observe the effects of candidate inhibitors. These changes include varicosity formation, loss of elongated mitochondria, accumulation of mitochondria in varicosities, cytoskeletal disassembly, and axon fragmentation. Agents that are found to counter any of the morphological changes induced by anti-NGF antibodies can be considered as candidate inhibitors of neuron or axon degeneration, which may, if desired, be tested in additional systems, such as those described herein.

The serum deprivation/KCl reduction assay is based on the use of cultures of cerebellar granule neurons (CGN) isolated from mouse (e.g., P7 mouse) brains. In this assay, the neurons are cultured in a medium including KCl and then are switched to medium containing less KCl (Basal Medium Eagles including 5 mM KCl), which induces neuron degeneration. Agents that are found to block or reduce neuron degeneration upon KCl withdrawal, which can be detected by, for example, analysis of images of fixed neurons stained with a neuronal marker (e.g., anti-class III beta-tubulin) can be considered as candidate inhibitors of neuron or axon degeneration, which may, if desired, be tested in additional systems, such as those described herein.

Another model of neuron or axon degeneration involves contact of cultured neurons with rotenone (2R,6aS,12aS)-1,2,6,6a,12,12a-hexahydro-2-isopropenyl-8,9-dimethoxychromeno[3,4-b]furo(2,3-h)chromen-6-one), which is a pesticide and insecticide that naturally occurs in the roots and stems of several plants, interferes with mitochondrial electron transport, and causes Parkinson's disease-like symptoms when injected into rats. Agents that are found to block or reduce degeneration of neurons cultured in the presence of rotenone, which can be detected by, for example, analysis of images of fixed neurons stained with, e.g., an antibody against neuron specific beta III tubulin, can be considered as candidate inhibitors of neuron or axon degeneration, which may, if desired, be tested in additional systems, such as those described herein.

An additional model of neuron or axon degeneration involves contact of cultured neurons with vincristine, an alkaloid that binds to tubulin dimers and prevents assemble of microtubule structures. Agents that are found to block or reduce degeneration of neurons cultured in the presence of vincristine, which can be detected by, for example, analysis of images of fixed neurons stained with, e.g., an antibody against neuron specific beta III tubulin, can be considered as candidate inhibitors of neuron or axon degeneration, which may, if desired, be tested in additional systems, such as those described herein.

The results obtained in the primary binding/interaction assays herein can be confirmed in in vitro and/or in vivo assays of axon degeneration. Alternatively, in vitro and/or in vivo assays of axon degeneration may be used as primary assays to identify inhibitors and antagonists as described herein.

In vivo assays for use in the invention include animal models of various neurodegenerative diseases, such as animal models of amyotrophic lateral sclerosis (ALS), Alzheimer's disease, Parkinson's disease, and multiple sclerosis (e.g., experimental autoimmune encephalitis (EAE) in mice). In addition, spinal cord and traumatic brain injury models can be used. Non-limiting examples of in vivo assays that can be used in characterizing inhibitors for use in the invention are described as follows.

In the case of amyotrophic lateral sclerosis (ALS), a transgenic mouse that expresses a mutant form of superoxide dismutase 1 (SOD1) recapitulates the phenotype and pathology of ALS (Rosen et al., Nature 362(6415):59-62, 1993). In addition to the SOD1 mouse, several mouse models of amyotrophic lateral sclerosis (ALS) have been developed and can be used in the invention. These include motor neuron degeneration (Mnd), progressive motor neuropathy (pmn), wobbler (Bird et al., Acta Neuropathologica 19(1):39-50, 1971), and TDP-43 mutant transgenic mice (Wegorzewska et al., Proc. Natl. Acad. ScL U.S.A., e-published on Oct. 15, 2009). In addition, a canine model has been developed and can be used in the invention (hereditary canine spinal muscular atrophy (HCSMA)).

Animal models that simulate the pathogenic, histological, biochemical, and clinical features of Parkinson's disease, which can be used in characterizing inhibitors for use in the methods of the present invention, include the reserpine (rabbit; Carlsson et al., Nature 180:1200, 1957); methamphetamine (rodent and non-human primates; Seiden et al., Drug Alcohol Depend 1:215-219, 1975); 6-OHDA (rat; Perese et al., Brain Res. 494:285-293, 1989); MPTP (mouse and non-human primates; Langston et al., Ann. Neurol. 46:598-605, 1999); paraquat/maneb (mouse; Brooks et al., Brain Res. 823:1-10, 1999 and Takahashi et al., Res. Commun. Chem. Pathol. Pharmacol. 66:167-170, 1989); rotenone (rat; Betarbet et al., Nat Neurosci. 3:1301-1306, 2000); 3-nitrotyrosine (mouse; Mihm et al., J Neurosci. 21:RC149, 2001); and mutated α-synuclein (mouse and Drosophila; Polymeropoulos et al., Science 276:2045-2047, 1997) models.

Genetically-modified animals, including mice, flies, fish, and worms, have been used to study the pathogenic mechanisms behind Alzheimer's disease. For example, mice transgenic for β-amyloid develop memory impairment consistent with Alzheimer's disease (Götz et al., MoI. Psychiatry 9:664-683, 2004). Models such as these may be used in characterizing the inhibitors.

Several animal models are used in the art to study stroke, including mice, rats, gerbils, rabbits, cats, dogs, sheep, pigs, and monkeys. Most focal cerebral ischemia models involve occlusion of one major cerebral blood vessel such as the middle cerebral artery (see, e.g., Garcia, Stroke 15:5-14, 1984 and Bose et al., Brain Res. 311 :385-391, 1984). Any of these models may also be used in the invention.

### 2. Assay for Modulating Neuron Cell Guidance of a Neuron

Suitable assays that detect changes in neuron growth patterns include, for example, those disclosed in Hastings, WIPO Publication WO 97/29189 and Walter et al., Development 101:685-96, 1987. Assays to measure the effects on neuron growth are well known in the art. For example, the C assay (e.g., Raper and Kapfhammer, Neuron 4:21-9, 1990 and Luo et al., Cell 75:217-27, 1993) can be used to determine collapsing activity of an agent of interest on growing neurons. Other methods that can assess inhibition of neurite extension or divert such extension are also known. See, Goodman, Annu Rev. Neurosci. 19:341-77, 1996. Conditioned media from cells expressing a protein of interest, or aggregates of such cells, can by placed in a gel matrix near suitable neural cells, such as dorsal root ganglia (DRG) or sympathetic ganglia explants, which have been co-cultured with nerve growth factor. Compared to control cells, protein-induced changes in neuron growth can be measured (as disclosed by, for example, Messersmith et al., Neuron 14:949-59, 1995 and Puschel et al., Neuron 14:941-8, 1995). Neurite outgrowth can be measured using neuronal cell suspensions grown in the presence of molecules of the present invention. See, for example, O′Shea et al., Neuron 7:231-7, 1991 and DeFreitas et al., Neuron 15:333-43, 1995.

The screening assays for inhibiting neuron degeneration and for modulating neuron cell guidance of a neuron specifically discussed herein are for the purpose of illustration only. A variety of other assays are well known to those skilled in the art and may also be used in the present invention. The assays described herein may be used to screen libraries of compounds including, without limitation, chemical libraries, natural product libraries (e.g., collections of microorganisms, animals, plants, etc.), and combinatorial libraries comprised of random peptides, oligonucleotides, or small organic molecules. In a particular embodiment, the assays herein are used to screen antibody libraries including, without limitation, human, recombinant, synthetic, and semisynthetic antibody libraries. The antibody library can, for example, be a phage display library, including monovalent libraries, displaying on average one single-chain antibody or antibody fragment per phage particle, and multi-valent libraries, displaying, on average, two or more antibodies or antibody fragments per viral particle. However, the antibody libraries to be screened in accordance with the present invention are not limited to phage display libraries. Other display techniques include, for example, ribosome or mRNA display (Mattheakis et al., Proc. Natl. Acad. Sci. U.S.A. 91:9022-9026, 1994; Hanes et al., Proc. Natl. Acad. Sci. U.S.A. 94:4937-4942, 1997), microbial cell display, such as bacterial display (Georgiou et al., Nature Biotech. 15:29-34, 1997), or yeast cell display (Kieke et al., Protein Eng. 10:1303-1310, 1997), display on mammalian cells, spore display, viral display, such as retroviral display (Urban et al., Nucleic Acids Res. 33:e35, 2005), display based on protein-DNA linkage (Odegrip et al., Proc. Acad. Natl. Sci. U.S.A. 101:2806-2810, 2004; Reiersen et al., Nucleic Acids Res. 33:e1O, 2005), and microbead display (Sepp et al., FEBS Lett. 532:455-458, 2002).

### XII. Screening Methods

Provided herein are methods for identifying an agent for use in inhibiting degeneration of a neuron. In some embodiments, the method comprises (a) contacting a neuron with a candidate agent; and (b) determining a level of degeneration of the neuron, wherein a lower level of degeneration of the neuron relative to a control, indicates the candidate agent inhibits degeneration of the neuron. In some embodiments, the control (e.g., the control neuron) is in the absence of the candidate agent.

Provided also are methods for identifying an agent for use in inhibiting degeneration of a neuron. In some embodiments, the method comprises (a) contacting a cell with a candidate agent; and (b) determining a level of Fzd3 phosphorylation or a level of Fzd3 internalization. A reduced level of Fzd3 phosphorylation and/or an increased level of Fzd3 internalization indicates the candidate agent inhibits degeneration of the neuron. In some embodiments, the control (e.g., the control neuron) is in the absence of the candidate agent.

Provided further are methods for identifying an agent for use in promoting degeneration of a neuron. In some embodiments, the method comprises (a) contacting a cell with a candidate agent; and (b) determining a level of Fzd3 phosphorylation or a level of Fzd3 internalization. A increased level of Fzd3 phosphorylation and/or an decreased level of Fzd3 internalization indicates the candidate agent promotes degeneration of the neuron. In some embodiments, the control (e.g., the control neuron) is in the absence of the candidate agent.

Provided further are methods for identifying an agent for use in modulating neuron cell guidance of a neuron. In some embodiments, the method comprises (a) contacting a cell with a candidate agent; and (b) measuring modulation of neuron cell growth or guidance. In some embodiments, the control (e.g., the control neuron) is in the absence of the candidate agent.

### XII. Kits

The present invention provides a kit comprising a Wnt compound. In some embodiments, the kit further comprises a Fzd3 dephosphorylating agent. The kit can also comprise a mammalian cell, e.g., a neuron cell.

Exemplary Wnt compounds that can be used in the kit of the present invention include a Wnt peptide, a small molecule Wnt mimetic, or a Wnt agonist. In some embodiments, a polypeptide comprising an amino acid sequence having at least 90% identity to SEQ ID NOs:1, 2, 3, 4, 5, 6, 7, 8, 9, 10, 11, 12, 13, 14, 15, 16, 17, 18, or 19 is used. Exemplary Fzd3 dephosphorylating agents that can be used in the kit of the present invention include a Vgl2 peptide or a Vgl2 mimetic, or a Dvl1 antagonist. In some embodiments, a siRNA targeting Dvl1 or a Dvl1 antibody is used.

### XIII. Pharmaceutical Compositions

In some embodiments, the treatment compound (e.g., Wnt compounds or Fzd3 dephosphorylating agents) may from part of a pharmaceutical composition. The pharmaceutical composition may include a treatment compound, as disclosed herein, and a pharmaceutically acceptable excipient. A “pharmaceutically acceptable excipient” includes pharmaceutically and physiologically acceptable, organic or inorganic carrier substances suitable for enteral or parenteral administration that do not deleteriously react with the active agent. Suitable pharmaceutically acceptable carriers include water, salt solutions (such as Ringer's solution), alcohols, oils, gelatins, and carbohydrates such as lactose, amylose or starch, fatty acid esters, hydroxymethylcellulose, and polyvinyl pyrrolidone. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the active agent.

In one embodiment, the treatment compound (e.g., Wnt compounds or Fzd3 dephosphorylating agents) forms part of a pharmaceutical composition, wherein said pharmaceutical composition comprises said treatment compound and a pharmaceutical acceptable excipient. In one embodiment, the pharmaceutical composition includes a permeabilizer (e.g., a salicylate, a fatty acid, or a metal chelator).

The pharmaceutical composition can be formulated for any route of administration, including enteral, oral, sublingual, buccal, parenteral, ocular, intranasal, pulmonary, rectal, intravaginal, transdermal, and topical routes. Parenteral administration includes, but is not limited to, intravenous, intramuscular, subcutaneous, intradermal, intraperitoneal, intrastemal, intraarterial injection and infusion.

The pharmaceutical composition can be formulated for immediate release or modified release, e.g., modified, sustained, extended, delayed, or pulsatile release, using known methods and excipients.

In one embodiment, the pharmaceutical composition is formulated as a topical composition, an injectable composition, an inhalant, a sustained release composition, or an oral composition. The treatment compound is preferably formulated for parenteral administration, e.g., by subcutaneous injection. If subcutaneous or an alternative type of administration is used, the compounds may be derivatized or formulated such that they have a protracted profile of action.

In another embodiment, the pharmaceutical composition is formulated as a peptide micelle, a targeted micelle, a degradable polymeric dosage form, a porous microsphere, a polymer scaffold, a liposome, or a hydrogel.

The treatment compound may be formulated according to known methods to prepare pharmaceutically useful compositions. An exemplary formulation would be one that is a stable lyophilized product that is reconstituted with an appropriate diluent or an aqueous solution of high purity with optional pharmaceutically acceptable carriers, preservatives, excipients or stabilizers (Remington's Pharmaceutical Sciences 16th edition (1980)). The pharmaceutical composition may include a pharmaceutically acceptable buffer to achieve a suitable pH for stability and for administration.

For parenteral administration, the treatment compound is formulated in a unit dosage injectable form (solution, suspension, or emulsion) with a pharmaceutically acceptable carrier. Preferably, one or more pharmaceutically acceptable anti-microbial agents may be added, such as phenol, m-cresol, and benzyl alcohol.

In one embodiment, one or more pharmaceutically acceptable salts (e.g., sodium chloride), sugars (e.g., mannitol), or other excipients (e.g., glycerin) may be added to adjust the ionic strength or tonicity.

The dosage of the composition of the invention to be administered can be determined without undue experimentation and will be dependent upon various factors including the nature of the active agent (including whether metal bound or metal free), the route of administration, the patient, and the result sought to be achieved. A suitable dosage of mimetic to be administered IV or topically can be expected to be in the range of about 0.01 to 50 mg/kg/day, preferably, 0.1 to 10 mg/kg/day, more preferably 0.1 to 6 mg/kg/day. For aerosol administration, it is expected that doses will be in the range of 0.001 to 5.0 mg/kg/day, preferably, 0.01 to 1 mg/kg/day. Suitable doses will vary, for example, with the compound and with the result sought.

The concentration of mimetic presentation in a solution used to treat cells/tissues/organs in accordance with the methods of the invention can also be readily determined and will vary with the active agent, the cell/tissue/organ and the effect sought.

Antibodies of the invention (and adjunct therapeutic agent) can be administered by any suitable means, including parenteral, subcutaneous, intraperitoneal, intrapulmonary, intranasal, and, if desired for local treatment, intralesional administration. Parenteral infusions include intramuscular, intravenous, intraarterial, intraperitoneal, and subcutaneous administration. In addition, antibodies can be administered by pulse infusion, particularly with declining doses of the antibody. Dosing can be by any suitable route, e.g., by injections, such as intravenous or subcutaneous injections, depending in part on whether the administration is brief or chronic.

The location of the binding target of an antibody used in the invention can be taken into consideration in preparation and administration of the antibody. When the binding target is an intracellular molecule, certain embodiments of the invention provide for the antibody or antigen-binding fragment thereof to be introduced into the cell where the binding target is located. In one embodiment, an antibody of the invention can be expressed intracellularly as an intrabody. The term “intrabody,” as used herein, refers to an antibody or antigen-binding portion thereof that is expressed intracellularly and that is capable of selectively binding to a target molecule, as described in Marasco, Gene Therapy 4:11-15, 1997; Kontermann, Methods 34:163-170, 2004; U.S. Pat. Nos. 6,004,940 and 6,329,173; U.S. Patent Application Publication No. 2003/0104402, and PCT Publication No. WO 03/077945. Intracellular expression of an intrabody is effected by introducing a nucleic acid encoding the desired antibody or antigen-binding portion thereof (lacking the wild- type leader sequence and secretory signals normally associated with the gene encoding that antibody or antigen-binding fragment) into a target cell. Any standard method of introducing nucleic acids into a cell may be used, including, but not limited to, microinjection, ballistic injection, electroporation, calcium phosphate precipitation, liposomes, and transfection with retroviral, adenoviral, adeno-associated viral and vaccinia vectors carrying the nucleic acid of interest.

In another embodiment, internalizing antibodies are provided. Antibodies can possess certain characteristics that enhance delivery of antibodies into cells, or can be modified to possess such characteristics. Techniques for achieving this are known in the art. For example, cationization of an antibody is known to facilitate its uptake into cells (see, e.g., U.S. Pat. No. 6,703,019). Lipofections or liposomes can also be used to deliver the antibody into cells. Where antibody fragments are used, the smallest inhibitory fragment that specifically binds to the binding domain of the target protein is generally advantageous. For example, based upon the variable-region sequences of an antibody, peptide molecules can be designed that retain the ability to bind the target protein sequence. Such peptides can be synthesized chemically and/or produced by recombinant DNA technology (see, e.g., Marasco et al., Proc. Natl. Acad. Sci. U.S.A. 90:7889-7893, 1993).

Entry of modulator polypeptides into target cells can be enhanced by methods known in the art. For example, certain sequences, such as those derived from HIV Tat or the Antennapedia homeodomain protein are able to direct efficient uptake of heterologous proteins across cell membranes (see, e.g., Chen et al., Proc. Natl. Acad. Sci. U.S.A. 96:4325-4329, 1999).

When the binding target is located in the brain, certain embodiments of the invention provide for the antibody or antigen-binding fragment thereof to traverse the blood-brain barrier. Certain neurodegenerative diseases are associated with an increase in permeability of the blood-brain barrier, such that the antibody or antigen-binding fragment can be readily introduced to the brain. When the blood-brain barrier remains intact, several art-known approaches exist for transporting molecules across it, including, but not limited to, physical methods, lipid-based methods, and receptor and channel-based methods.

Physical methods of transporting the antibody or antigen-binding fragment across the blood-brain barrier include, but are not limited to, circumventing the blood-brain barrier entirely, or by creating openings in the blood-brain barrier. Circumvention methods include, but are not limited to, direct injection into the brain (see, e.g., Papanastassiou et al., Gene Therapy 9:398-406, 2002), interstitial infusion/convection-enhanced delivery (see, e.g., Bobo et al., Proc. Natl. Acad. Sci. U.S.A. 91:2076-2080, 1994), and implanting a delivery device in the brain (see, e.g., Gill et al., Nature Med. 9:589-595, 2003; and Gliadel Wafers™, Guildford Pharmaceutical). Methods of creating openings in the barrier include, but are not limited to, ultrasound (see, e.g., U.S. Patent Publication No. 2002/0038086), osmotic pressure (e.g., by administration of hypertonic mannitol (Neuwelt, E. A., Implication of the Blood-Brain Barrier and its Manipulation, Volumes 1 and 2, Plenum Press, N.Y., 1989)), permeabilization by, e.g., bradykinin or permeabilizer A-7 (see, e.g., U.S. Pat. Nos. 5,112,596, 5,268,164, 5,506,206, and 5,686,416), and transfection of neurons that straddle the blood-brain barrier with vectors containing genes encoding the antibody or antigen-binding fragment (see, e.g., U.S. Patent Publication No. 2003/0083299).

Lipid-based methods of transporting the antibody or antigen-binding fragment across the blood-brain barrier include, but are not limited to, encapsulating the antibody or antigen-binding fragment in liposomes that are coupled to antibody binding fragments that bind to receptors on the vascular endothelium of the blood- brain barrier (see, e.g., U.S. Patent Application Publication No. 2002/0025313), and coating the antibody or antigen-binding fragment in low-density lipoprotein particles (see, e.g., U.S. Patent Application Publication No. 2004/0204354) or apolipoprotein E (see, e.g., U.S. Patent Application Publication No. 2004/0131692).

Receptor and channel-based methods of transporting the antibody or antigen-binding fragment across the blood-brain barrier include, but are not limited to, using glucocorticoid blockers to increase permeability of the blood-brain barrier (see, e.g., U.S. Patent Application Publication Nos. 2002/0065259, 2003/0162695, and 2005/0124533); activating potassium channels (see, e.g., U.S. Patent Application Publication No. 2005/0089473), inhibiting ABC drug transporters (see, e.g., U.S. Patent Application Publication No. 2003/0073713); coating antibodies with a transferrin and modulating activity of the one or more transferrin receptors (see, e.g., U.S. Patent Application Publication No. 2003/0129186), and cationizing the antibodies (see, e.g., U.S. Pat. No. 5,004,697).

Antibody compositions used in the methods of the invention are formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The antibody need not be, but is optionally formulated with one or more agent currently used to prevent or treat the disorder in question. The effective amount of such other agents depends on the amount of antibodies of the invention present in the formulation, the type of disorder or treatment, and other factors discussed above. These are generally used in the same dosages and with administration routes as described herein, or about from 1 to 99% of the dosages described herein, or in any dosage and by any route that is empirically/clinically determined to be appropriate.

For the prevention or treatment of disease, the appropriate dosage of an antibody (when used alone or in combination with other agents) will depend on the type of disease to be treated, the type of antibody, the severity and course of the disease, whether the antibody is administered for preventive or therapeutic purposes, previous therapy, the patient's clinical history and response to the antibody, and the discretion of the attending physician. The antibody is suitably administered to the patient at one time or over a series of treatments. Depending on the type and severity of the disease, about 1 μg/kg to 15 mg/kg (e.g., 0.1 mg/kg-10 mg/kg) of antibody can be an initial candidate dosage for administration to the patient, whether, for example, by one or more separate administrations, or by continuous infusion. One typical daily dosage might range from about 1 μg/kg to 100 mg/kg or more, depending on the factors mentioned above. For repeated administrations over several days or longer, depending on the condition, the treatment would generally be sustained until a desired suppression of disease symptoms occurs. One exemplary dosage of the antibody would be in the range from about 0.05 mg/kg to about 10 mg/kg. Thus, one or more doses of about 0.5 mg/kg, 2.0 mg/kg, 4.0 mg/kg, or 10 mg/kg (or any combination thereof) may be administered to the patient. Such doses may be administered intermittently, e.g., every week or every three weeks (e.g., such that the patient receives from about two to about twenty, or, e.g., about six doses of the antibody). An initial higher loading dose, followed by one or more lower doses may be administered. An exemplary dosing regimen comprises administering an initial loading dose of about 4 mg/kg, followed by a weekly maintenance dose of about 2 mg/kg of the antibody. However, other dosage regimens may be useful. The progress of this therapy is easily monitored by conventional techniques and assays.

Certain aspects of the invention can be described in greater detail in the non-limiting Example that follows.

### Examples

### Example 1

### Characterization of PCP and aPKC/A-B Polarity Components in Wnt Signaling and A-P Axon Guidance

Overview. Example 1 is directed to (la) determine of the subcellular localization of core PCP components, (1b) testing of the role of all PCP components in mediating Wnt attraction and anterior turning of commissural axons, (1c) determination of the subcellular localization of core components of aPKC/A-B polarity signaling, and (1d) examination of the role of aPKC/A-B signaling components in Wnt attraction and A-P guidance of commissural axons.

### Rationle, Overall Hypothesis and Design.

Our results show that several components of PCP and A-B polarity signaling pathways are involved in A-P guidance of commissural axons, a process shown to be dependent on highly conserved functions of the Wnt family axon guidance molecules. These two cell polarity pathways, which are perpendicular to each other, are candidate pathways that may convey asymmetric signaling in the axonal growth cones. To determine whether these components truly operate in these polarity pathways, we studied the subcellular distribution of all core components and their function in mediating Wnt attraction and A-P guidance.

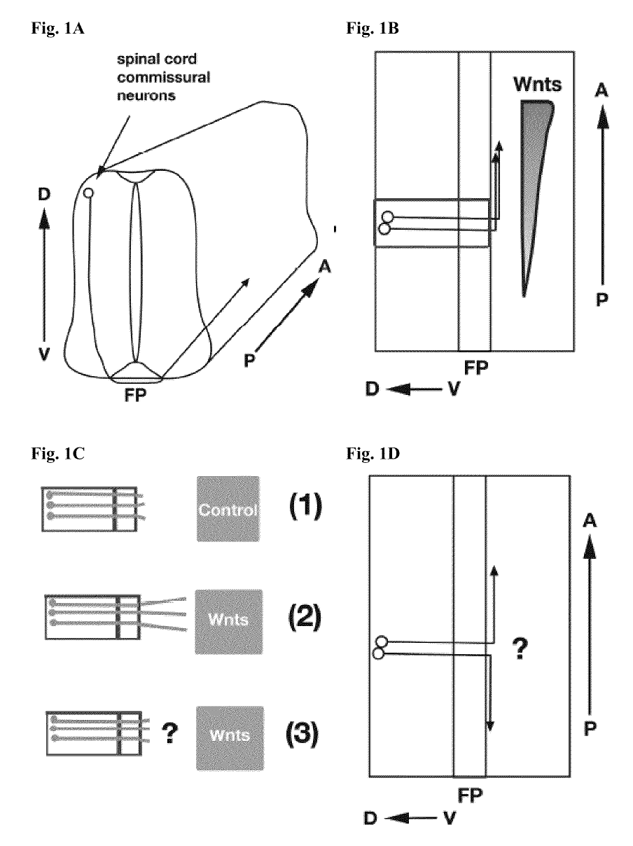

Confirmed antibodies are employed to detect endogenous proteins with or without Wnt gradients in dissociated commissural neurons from the spinal cord or cortical neurons. Antibodies are confirmed by Western Blotting and immunostaining from cells treated with RNAi knockdown or from knockout tissue/cells. GFP or mCherry fusion constructs are used to label signaling components for live imaging. The distribution of recombinant fusion proteins are verified by comparing the immunostaining patterning shown by confirmed antibodies. The function of signaling components in A-P guidance of commissural axons is tested in vivo using knockout animals (by lipophilic DiI injection) and ex vivo using “open-book” explants electroporated with RNAi or dominant-negative constructs. These methods have been established and used routinely in the laboratory to study the role of PKCζ and PI3K in A-P guidance of commissural axons (FIGS. 1A-1D)⁽⁵⁷⁾. The direct role in mediating response to Wnts is tested by “post-crossing” assays, a collagen gel assay developed for analyzing the response of commissural axons after they have crossed the midline and has been routinely used in the laboratory (FIG. 1C)^{(70,32,57)}. Explants dissected from knockout mice or from electroporated spinal cord tissues are employed for “post-crossing” assays to test whether the post-crossing commissural axons still respond to Wnt attraction when these components are either lost (in knockouts or RNAi knockdown) or blocked (in dominant-negative).

### Experimental Design

1a: Determination of the growth cone subcellular localization of core PCP components. We have confirmed specific antibodies to Frizzled3, Celsr3, Dishevelled1, Dishevelled2, Dishevelled3 and Vangl2. We perform immunostaining in pre-crossing commissural axon growth cones in dissociated culture, as well as in “post-crossing” commissural axons in collagen gel (FIG. 1C). We find that in the collagen gel, single growth cones navigate individually, and we have sufficient subcellular resolution and can perform live imaging of growth cones. The localization of these signaling components with or without Wnt gradients is compared. For dissociated culture on laminin-coated culture dish, we introduce Wnt gradients using a pipette and picospritzer (as in the “Mu-Ming Poo Assay”). In the collagen gel assay, we introduce Wnt gradients by placing cell aggregates secreting the Wnt proteins, a proven method we use routinely (FIGS. 1A-1D). In these localization studies, we can include actin and microtubule staining to help determine the location in reference to cytoskeleton.

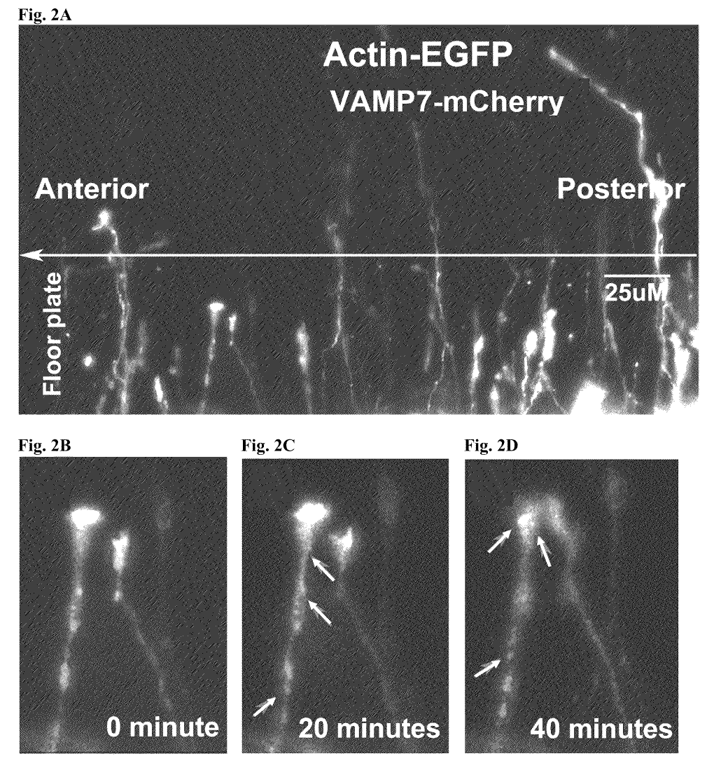

Immunostaining allows us to detect subcellular localization of signaling proteins in isolated growth cone. Without wishing to be bound by theory, it is believed that in the “open-book” explants, commissural axons are too closely located, and it is not possible to determine subcellular distribution by immuonstaining To visualize the localization of signaling components in the spinal cord, we express fluorescent fusion constructs by electroporation. We made GFP and mCherry fusion constructs of Frizzled3, Celsr3, Dishevelled2, Vangl2 and Prickle2 and plan to make fusions of the other components. The localizations of fluorescent fusion proteins all agree with the pattern of endogenous proteins shown by antibody immunostaining Using the fused fluorophore, we determine the subcellular localization of the signaling components during midline crossing and anterior turning within the spinal cord “open-book” explants (FIGS. 2A-2D).

Using fluorescent fusion constructs, we conduct live imaging experiments as shown in FIGS. 2A-2D to characterize the localization of the signaling components in response to Wnt gradients in dissociated culture, collagen gel assay and in “open-book” assay. The Wnt gradient in the “open-book” explants can be disrupted by addition of sFRP2 protein or placing Wnt-secreting cell aggregates to alter the gradient as shown previously⁽³²⁾. We test whether membrane vesicles, which contain the PCP signaling components, move towards or away from the Wnt gradient and whether the directed movement is altered when Wnt gradient is ablated or changed.

The vesicular and membrane compartment of PCP components are characterized by co-localization studies as exemplified by Frizzled3 localization. In addition, to test co-localization in heterologous cells like HEK293T cells, their co-localization is examined in growth cones of commissural neurons in dissociated cultures, in “post-crossing” commissural axon growth cones in collagen gel assays, and in “open-book” assays by co-electroporating the fusion constructs. Live imaging is performed as well to detect potential dynamic changes of localization when Wnt gradients are applied or during anterior turning in the “open-book” explants. Useful wildtype and dominant-negative vesicular markers/regulators include those tabulated in Table 2.

TABLE 2
Vesicular markers/regulator that have
been used as described herein.

| Name | Description |
|---|---|
| VAMP2-EGFP | VAMP2 |
| VAMP2-dsRed | VAMP2 |
| Synaptophysin-EGFP | Synaptophysin |
| VAMP2-Phluorin | VAMP2 |
| Synaptophysin-Phlu orin 1X (S1X) | Synaptophysin |
| Synaptophysin-Phluorin2X (S2X) | Synaptophysin |
| Synaptophysin-Phluorin4X (S4X) | Synaptophysin |
| GFP-Rab11 | Rab11 |
| GFP-Rab11 (S2SN) | Rab11 (S2SN) |
| Arf6-GFP | Arf6 |
| Arf6-HA | Arf6-HA |
| Arf6-HA(T27N) | Arf6-HA(T27N) |
| Arf6-HA(T1S7A) | Arf6-HA(T1S7A) |
| Rab8-EGFP | Rab8 |
| Rab4-EGFP | Rab4 |
| RabS-EGFP | RabS |
| Myc-Rab8 | Rab8 |

1b: Tests of the role of the PCP components in mediating Wnt attraction and anterior turning. We found that Frizzled3, Celsr3 and Vangl2 are required for A-P guidance of commissural axons because, in mice deficient with these genes, commissural axons project randomly along the A-P axis with approximate 50% growing anteriorly and the other 50% turning posteriorly. To obtain further evidence that these signaling components mediate Wnt attraction, we perform “post-crossing” assays using spinal cord explants from these knockout mice to test whether Wnt attraction is lost (FIG. 1C). To determine whether the other components are also required for AP guidance, we express RNAi or truncated constructs of Dvl1, Dvl2, Dvl3, Prickle and Diego in “open-book” assays first to determine whether A-P guidance is disrupted. We then use “post-crossing” to test whether Wnt attraction is affected when these other components are disrupted.

1c: Determination of the growth cone subcellular localization of core components of aPKC/A-B polarity signaling. Using similar approaches as described above, the subcellular localization of aPKC (PKCζ and PKCλ), Par3, Par6, Lgl1, FEZ1, DISC1, GSK3β and MARK2 is pinpointed. DISC1 (disrupted in schizophrenia 1), a binding partner of FEZ1 (a PKC substrate), is a gene mutated in a number of schizophrenic families⁽³³⁾. DISC1 is broadly expressed in the central nervous system and also involved in neurite outgrowth and therefore may be a partner of FEZ1 in mediating Wnt attraction downstream of PKCζ(³³). As FEZ1 is a kinesin-associated protein, it is possible that FEZ1/DISC complex may mediate kinesin-mediated transport of membrane-bound organelles such as nascent membrane towards the microtubule plus end to facilitate neurite outgrowth in response to Wnt-PKCζ activation.

The precise localization of all these components is determined using vesicular markers/regulators (as in Table 2) and microtubule markers/regulators, such as EB1, EB3, and APC. The localization before and after midline crossing, with and without Wnt gradients, is determined. To test our experimental system, we have obtained results of PKCζ and Lgl1 in cortical neurons. For example, our results show that Lgl1 is enriched on a subset of filapodia. We test whether Wnt causes changes in Lgl1 localization and whether Lgl1 localization is polarized in “open-book” explants. We have established three-dimensional growth cone imaging in “open-book” explants and have sufficient subcellular resolution (FIGS. 2A-2D).

1d: Examination of the role of aPKC/A-B signaling components in Wnt attraction and A-P guidance. Using similar approaches as described above, the role of components of aPKC/A-B polarity signaling pathway and substrates of aPKC in A-P axon guidance and Wnt-mediated axon attraction is tested. Knockout mice (e.g., Lgl1 straight knockout and Lgl1 conditional allele), RNAi construct and dominant-negative constructs are employed in pre- and post-crossing commissural axons.

### Results, Potential Problems and Alternative Approaches

We have set up high-resolutions confocal imaging techniques to obtain subcellular localization of all PCP and aPKC/A-B components in growth cones by a combination of antibody staining of fixed cells and GFP or mCherry fusion proteins for live imaging. To ensure the specificity, antibodies are confirmed by immunostaining and Western blotting of cells and tissues transfected with RNAi or from knockout animals. Fusion constructs are confirmed by Western blotting and immunostaining with the confirmed antibodies. If overepxression alters localization, the expression level can be reduced. So far, all overexpression patterns agree with antibody staining. Using our established system, their potential asymmetric localization can be readily observed with our imaging capability. For long-term live imaging, bleaching of signal under confocal microscope can be a problem if the laser power is too intense. We have been able to circumvent this by reducing laser power, shortening scanning time (by reducing the number of Z stacks and resolution without losing specific signals) or using spinning disc microscope. Overexpression of components may result in signals of too high intensity and interfere with the observation of dynamic changes of smaller portions of the labeled vesicles. To circumvent this problem, photoactivatable GFP and photo-convertible Dentra are employed to mark smaller portion of the labeled protein. We also use FRAP (fluorescence recovery after photobleaching) to detect the movement of vesicles.

Using an inverted scope, we deliver protein gradients by puffing proteins into culture medium close to the growth cone from a fine pipette and picospritzer. We found that our dissociated commissural neurons respond to Netrin-1 released from a pipette. However, pre-crossing commissural neurons do not respond to Wnts and may not respond to puffing of Wnt proteins. We found previously that overexpressing PI3Kγ can turn on Wnt-responsiveness in pre-crossing commissural axons⁽⁵⁷⁾. Therefore, we express PI3Kγ and image the changes of signaling components. Alternatively, we can use cortical neurons and retinal ganglion cell neurons, which do respond to Wnts. The direction of vesicular movement is determined relative to the Wnt gradients and quantified by counting the number of vesicles that pass through a filapodia or pass across the midplane of a growth cone per minute (from left to right or from bottom to top)^{(40,50)}.

The fact that Frizzled3 co-localizes with Arf6 suggests that Frizzled3 may be recycled from plasma membrane by endocytosis. It is interesting Frizzled3 does not overlap the Rabs that are known for recycling. It is possible that Arf6 is the GTPases for Frizzled3 trafficking or a different Rab is involved in moving Frizzled3 in the growth cone. We test other candidates. It is also possible that Frizzled3-Rab interaction depends on Wnt stimulation. We test whether addition of Wnts changes their localization. Although PCP components are highly conserved in structure and biochemical properties in a number of diverse morphogenetic processes, it is still possible that Celsr3 and Vang2 may function in a manner distinct from PCP in the growth cone. Therefore, it is necessary to evaluate the functions of all PCP components. If other PCP components are not involved, then Celsr3 and Vangl2 function in a non-PCP pathway. We then use Celsr3 and Vangl2 to search for their interacting proteins (i.e. using proteomics) and further our studies in cell biological mechanisms proposed in Aim 2a and 2c. In this case, we would enhance the knowledge about the function and mechanisms of Celsr3 and Vang2 outside the context of PCP signaling. Similarly, aPKC could function independent of Par3/Par6 complex and in a non-apical-basal polarity mechanism. We then identify novel aPKC substrates using proteomics and expand the knowledge on aPKC outside the context of apical-basal polarity signaling.

The Lgl1 knockout mice may have other earlier defects. We examine the patterning and cell fate carefully as we did with Frizzled3, Celsr3 and Looptail knockout mice, in which case no patterning and cell fate changes were observed. Should Lgl1 knockout have patterning defects, we use the conditional allele crossed to Nestin-Cre. In addition, RNAi approach can circumvent this problem because electroporation of RNAi constructs/or oligos in small number of cells will not disturb the overall development. A weaker RNAi knockdown can achieve partial (less-complete) eliminations of transcripts and can be introduced at a later time to avoid disruption of the earlier functions (i.e., electroporated after differentiation).

### Example 2

### Cellular Mechanisms of PCP and aPKC/A-B Polarity Signaling in Growth Cone Guidance

Overview. Example 2 is directed to the following: (2a) characterizing the function of PCP core components in membrane trafficking and the role of membrane trafficking in Wnt signaling and axon guidance; (2b): testing whether Celsr3 functions cell-autonomously, non-autonomously or both; (2c): characterizing the role of aPKC/A-B polarity signaling components in membrane trafficking and microtubule dynamics; and (2d): determining how PCP and aPKC/A-B signaling pathways are coordinated to guide axons and introduce asymmetry.

### Rationle, Overall Hypothesis and Design.

How directionality emerges in a growth cone exposed to a directional cue, and what cellular processes give rise to asymmetric signaling and then execute directional turning, have been largely unknown. Understanding how polarity signaling pathways regulate growth cone turning allows us to gain insights of the cell biology of growth cone guidance.

2a) Characterizing the function of PCP core components in membrane trafficking and the role of membrane trafficking in Wnt signaling and axon guidance. As axons elongate, new membrane is trafficked and inserted into the plasma membrane of the growth cone^{(5,12)}. In neurotrophin-induced turning, directed endo- and exocytosis in the growth cone have been shown to be required for attractive growth⁽⁵⁰⁾. An important question is whether directed membrane trafficking is the cause or the effect, i.e., the source of asymmetry/directionality or the consequence of directional decision. The cellular functions of some of the PCP components suggest that they are involved in or under the control of directed membrane trafficking. For example, Frizzled is found being trafficked in a polarized way along microtubule to the distal end of Drosophila epithelial cells, where it is concentrated⁽⁴⁷⁾. Therefore, in one line of investigation, we focus on the role of membrane trafficking in Wnt/PCP-mediated axon guidance.

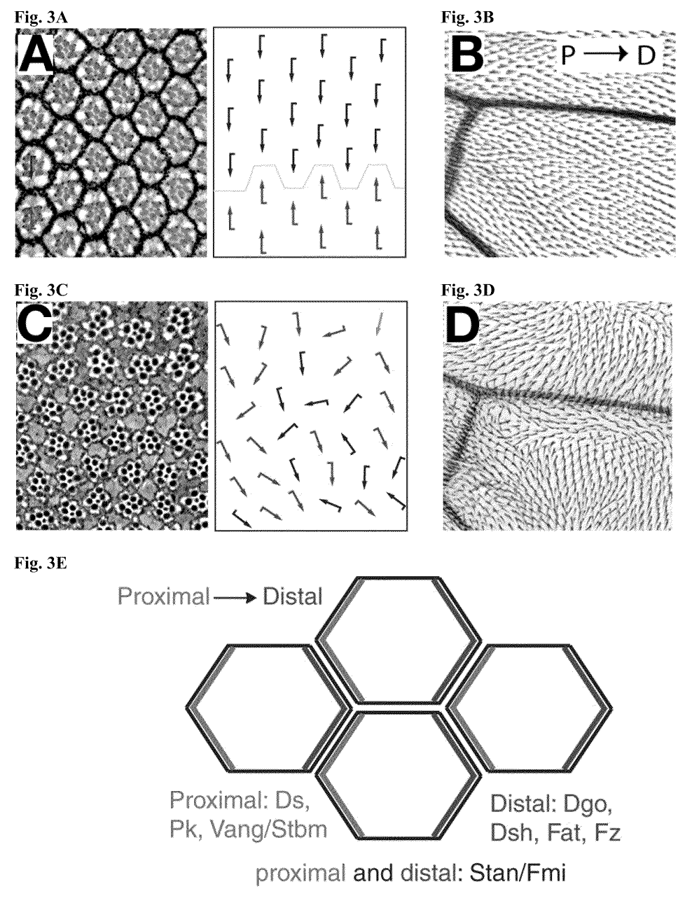

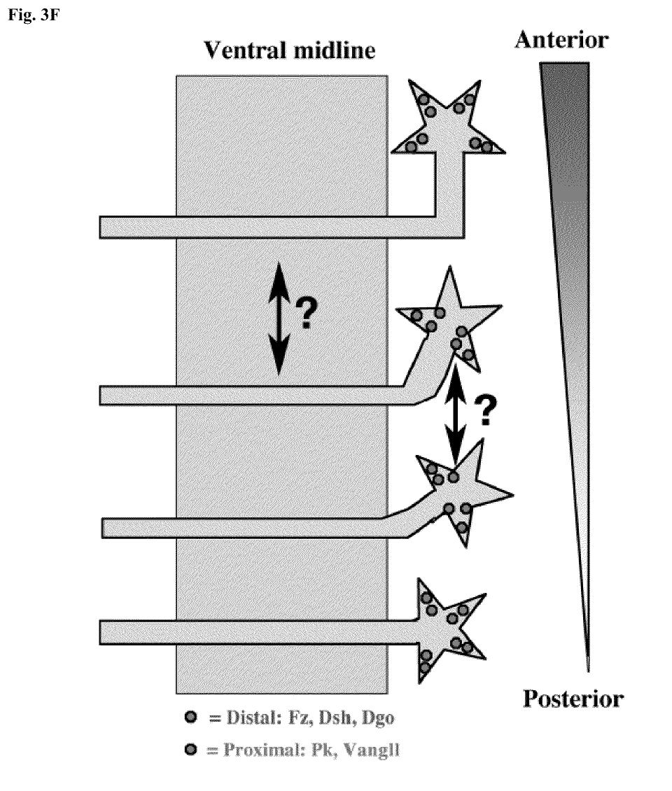

2b): Testing whether Celsr3 functions cell-autonomously, non-autonomously or both. An important feature of PCP signaling is the non-cell autonomous mechanism, which is thought necessary to ensure precision of polarized morphology. Without wishing to be bound by theory, it is believe that cell-cell interaction may be an amplification mechanism which sensitizes the detection of a directional cue. For example, a gradient may be presented in a very shallow gradient but a group of interacting cells span a larger area and may collectively sample a larger drop of gradient⁽⁶⁶⁾. Commissural axon guidance at the midline may involve axon-axon interaction and have similarity to a PCP process because many commissural neurons cross the midline and turn at the same time (FIGS. 3A-3F). Our live imaging results showed that it takes 8 hours to cross the midline and another 1-2 hours to turn anteriorly after crossing. There is ample time for the axons to interact among themselves. See e.g., question marks symbol in FIG. 3F). Another possible function of axon-axon interaction may be to ensure tiling of the same axons so that these axons can move in a coordinated way without crossing into each other's trajectories. Flamingo has both autonomous non-autonomous functions in regulating polarity of wing hair cells. Celsr3 has autonomous function in axon development in the forebrain⁽⁶⁵⁾. Flamingo also tiles dendrites and mediates axon-axon interactions^{(20,8)}. Therefore, we test whether axon interactions is necessary for coordinated anterior turning by testing whether Celsr3 and Vangl2 have non-autonomous function: whether the axons expressing normal Celsr3 and Vangl2 pathfind normally when Celsr3 and Vangl2 are mutated in their neighbors.

2c): Characterizing the role of aPKC/A-B polarity signaling components in membrane trafficking and microtubule dynamics. Several aPKC substrates are regulators of cellular processes that are highly relevant to growth cone guidance. See e.g., Table 3 following. Lgl1, FEZ1 and VAMP2 are known to be important for trafficking vesicular and membranous organelles. Lgl controls polarized exocytosis by binding to SNARE proteins in Drosophila neuroblasts and mammalian kidney cells; PKCζ phosphorylates FEZ1, a microtubule binding protein which regulates the trafficking of membranous organelles and vesicles; PKCζ phosphorylates and regulates VAMP2, a vSNARE protein involved in vesicular fusion^{(61,21,35,18)}. Like some of the PCP components discussed in above (2a), they may also play roles in regulating the asymmetric localization of signaling components or in regulating membrane movement. Other aPKC substrates, such as GSK3β and MARK2, are known regulator of microtubule stability^{(64,10)}. PKCζ phosphorylates and inhibits both GSK3β and MARK2, which normally destabilize microtubules. Therefore, PKCζ may regulate growth cone guidance by locally stabilize microtubule on one side of the growth cone or in a subset of filapodia because microtubules are known to be present in some filapodia, although most of the filapodia are actin-rich structures. In fact, we have observed enrichment of PKCζ in subsets of growth cone filapodia. Accordingly, similar approaches and experimental systems are employed to test the role vesicular/membrane trafficking and microtubule dynamics regulated by aPKC/A-B pathway as proposed above (2a).

TABLE 3
The six core PCP components in Drosophila wing epithelia

| Component | Frizzled | Flamingo | Dvl | Vang Gogh | Prickle | Diego |
|---|---|---|---|---|---|---|
| General | 7-pass | 7-pass | Intracellular | 4-pass | Lim | Ankyrin |
| information | transmembrane | transmembrane | protein; | transmembrane | domain | repeat |
| | protein | protein with | Binds to | protein | | |
| | | cadherin | Frizzled, | | | |
| | | domain | Vang | | | |
| Localization | Distal | Proximal and | Distal | Proximal | Proximal | Distal |
| | membrane | distal | membrane/ | membrane | membrane/ | membrane/ |
| | | membrane | cytoplasmic | | cytoplasmic | cytoplasmic |
| Function | Wnt receptor | Frizzled co- | Scaffold | | Exclude | Antagonize |
| | | receptor | protein | | Dvl | Prickle/Vang |
| Potential | Binding and | Cooperating | Organizing | Setting up | Setting up | Setting up |
| relevance to | detecting | with Frizzled; | signaling | asymmetry | asymmetry | asymmetry |
| growth cone | Wnt | axon-axon | complexes | | | |
| guidance | gradients | interaction | | | | |

2d): Determining how PCP and aPKC/A-B signaling pathways are coordinated to guide axons and introduce asymmetry. Without wishing to be bound by any theory, because both PCP and aPKC are specifically required for A-P guidance commissural axons after midline line crossing, and because these two pathways have been shown to potentially interact in the context of cell polarity signaling, it is believed that the coordinated activity of both pathways are essential for Wnt-mediated axon guidance. We have generated reagents to study the subcellular localization and to conduct live imaging to track the movement of the signaling components in grow cones. Thus, we are able to determine how these signaling pathway are integrated to give rise to directional choice.

### Experimental Design

2a) Characterizing the function of PCP core components in membrane trafficking and the role of membrane trafficking in Wnt signaling and axon guidance.

1) To address whether Wnt-mediated axon guidance requires polarized trafficking of PCP signaling components, we block directed vesicular/membrane trafficking using dominant-negative constructs, such as Arf6 dominant-negative, Rab dominant-negative or VAMP2 RNAi, to block trafficking (i.e., recycling endocytosis) and analyze the localization of Frizzled3, Celsr3, Dvls, Vangl2, Prickle and Diego. The localization experiments of the PCP components, is carried out, for example, in dissociated commissural neurons (with PI3Kγ expression to render them Wnt-responsive), dissociated cortical neurons (they are Wnt-responsive), commissural neurons in “post-crossing” assays (co-electroporated into the dorsal spinal cord with fluorescent fusion proteins and blockers or inhibitors of membrane trafficking before setting up for “post-crossing” assays) and in “open-book” explants (co-electroporated with fusion markers and inhibitors of trafficking) Localization of PCP components is determined by immunostaining of fixed cells or tissues or live imaging.

We also test whether Wnt-mediated attraction is blocked by these reagents using the “post-crossing” assay. To address whether vesicular trafficking is required for A-P guidance of commissural axons, we use, for example, “open-book” assay and block vesicular trafficking by electroporating dominant-negative or RNAi constructs into the spinal cord. We also block endocytosis in general by expressing dominant negative dynamin1 mutants and analyze the localization and function of PCP components.

2) To address whether Wnt-PCP signaling regulates vesicular trafficking to establish asymmetric or move membrane directionally to cause turning, we use Wnts to stimulate the signaling and observe whether the membrane trafficking system is mobilized, and if so which system is activated (as indicated by various markers, i.e., Rab GTPases). We also inhibit the function of the PCP components (by expressing RNAi and dominant-negative mutant constructs) and examine membrane trafficking We also analyze the trafficking in knockout mice of Frizzled3, Celsr3 and Looptail, etc., by expressing the membrane trafficking markers in the “open-book” or “post-crossing” explants electroporated with constructs expressing vesicular and membrane markers. We analyze the localization in both fixed cells or by live imaging. For some of the live imaging experiments, we focus on the direction of vesicular trafficking as our initial analyses. The direction of vesicular movement can be determined relative to the Wnt gradient and quantified by counting the number of vesicles that pass through a filapodia or pass across the midplane of a growth cone per minute (from left to right or from bottom to top)^{(40,50)}.

Microtubule dynamics can be co-imaged during these live imaging experiments by co-expressing α-tubulin-GFP. Imaging can be done in a number of systems, including organotypic “open-book” assays, dissociated cortical or spinal cord neurons, neuronal like N2A cells and heterologous HEK cells.

2b): Testing whether Celsr3 functions cell-autonomously, non-autonomously or both. We use Celsr3 conditional allele, as known in the art, to test whether Celsr3 is required cell autonomously and whether neighboring axons have tiling or A-P guidance defect⁽⁶⁵⁾. We electroporate β-actin/CMV::Cre-IRES-GFP constructs into the spinal cord from Celsr3 conditional knockout mice and culture the explants in “open-book” configuration. The axons with GFP expression do not have Celsr3 because of the activity of Cre driven by a CMV promote enhance by a β actin promoter. We then analyze the A-P guidance behavior of commissural axons where Celsr3 is mutated to determine whether Celsr3 is required cell autonomously. We then perform DiI injection into the area of electroporation and examine a large number of axons that have been DiI traced but was not electroporated to expressed Cre. These axons should have normal Celsr3 gene and we can test whether they project anteriorly properly. If they can still turn anteriorly, it means that Celsr3 is only required cell autonomously. If they are randomized along the A-P axis, it means that Celsr3 is required cell non-autonomously. The electroporation and “open-book” assay has been established and used routinely in the lab⁽⁵⁷⁾. To test the non-autonomous function of Vangl2, Vangl2 RNAi with a GFP marker (which is established in the lab) is electroporated into the “open-book” explants and the neighboring axons not expressing Vangl2 RNAi are analyzed by DiI injection.

2c): Characterizing the role of aPKC/A-B polarity signaling components in membrane trafficking and microtubule dynamics.

1) Membrane/vesicular trafficking. Using similar approaches detailed above, we test whether the function of Lgl1, FEZ1 and VAMP2 are required for Wnt attraction and A-P axon guidance by blocking the function of Lgl1 and FEZ1 and examining the localization of Frizzled3, PKCζ, Par3 and Par6. We also test whether Wnt signaling regulates the distribution and the function of Lgl1 and FEZ1 by either stimulating or inhibiting Wnt-aPKC signaling and examine the localization and function of Lgl1 and FEZ1.

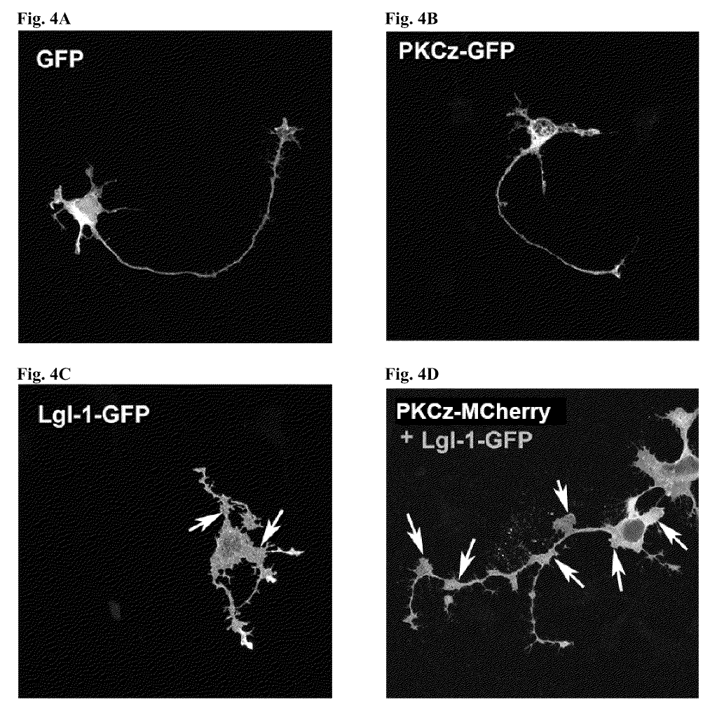

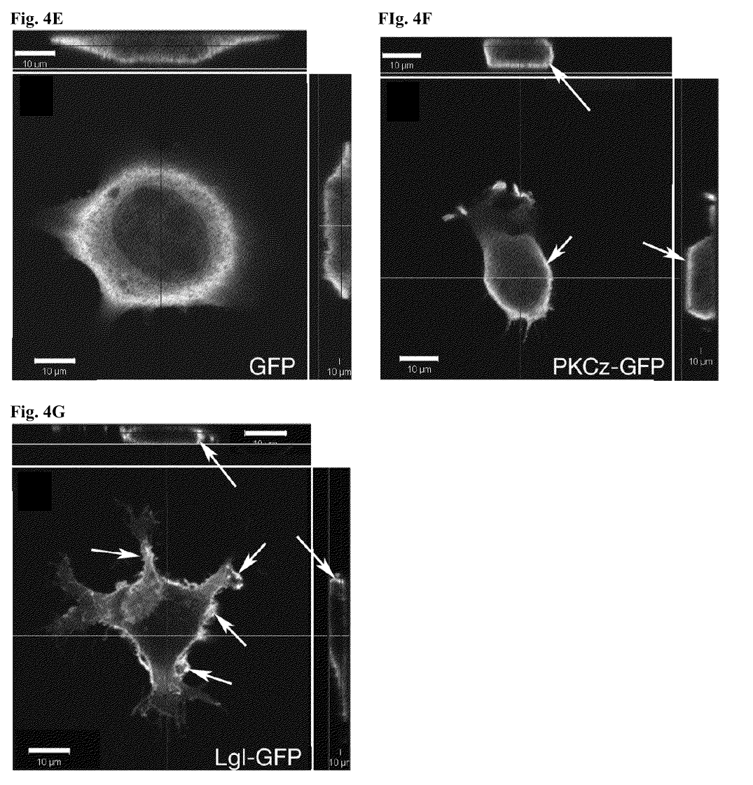

Our results show that Lgl1 changes membrane dynamics in cortical neurons and N2A cells and PKCζ appears to have opposite effects and can antagonize the membrane changes induced by Lgl1 (FIGS. 4A-4G). Our results suggest that they may have opposite effects on membrane flow, with Lgl1 promoting membrane movement towards the dendrite and PKCζ inhibiting that and promoting membrane flow towards the axon. Therefore, we hypothesize that they may have similar opposing effects on membrane flow across one growth cone during axon guidance. Therefore, it is informative to image the membrane changes induced by Lgl1 and whether or how Wnt signaling alters this flow.

We use Lgl1 RNAi, Lgl1 KO and conditional KO and image the membrane dynamics in fixed and live neurons and test how the membrane system of cells missing Lgl1 responds to Wnt gradient. Similar approaches can be taken to analyze the role of FEZ1 (and its partner DISC1) and VAMP2 in regulating membrane trafficking distribution and response to Wnt signals. As FEZ1 is a kinesin-associated protein, it is possible that FEZ1/DISC complex may mediate kinesin-mediated transport of membrane-bound organelles such as nascent membrane towards the microtubule plus end to facilitate neurite outgrowth in response to Wnt-PKCz activation. KIFs (kinesin superfamily proteins) are involved in transporting membrane organelles, protein complexes and RNAs along microtubules and required for axon growth. Recent studies show that they also transport PIP3, which is essential for signaling in neurite outgrowth and a key upstream regulator in the Wnt-PKCζ pathway⁽³⁶⁾. We examine the behaviors of KIFs in commissural axons by imaging various KIFs fused with fluorescent proteins and test whether Wnts can induce their directed movement along microtubules and whether blockade of FEZ1/DISC1 complex using the aforementioned methods causes defects in kinesin dynamics and vesicular trafficking We also express kinesin1 RNAi or dominant-negative construct to test whether A-P guidance or Wnt attraction are affected and perform live imaging of growth cones and their components in commissural axons using confocal microscopy and spinning disk confocal microscopy.

On the other hand, we test whether Wnt signaling regulates the distribution and function of the aPKC/A-B polarity components. We apply Wnts or Wnt gradients using similar methods described before or test the distribution of these components in “open-book” assays where Wnt gradients are disrupted by sFRPs or from spinal cords dissected from Frizzled3 knockout embryos. We also test the behavior of these polarity components in “open-book” explants expressing RNAi or dominant-negative constructs inhibiting Wnt signaling or in knockout mice with Wnt signaling defects.

2) Microtubule stability. We test whether GSK3β and MARK2 are required for stabilizing or destabilizing microtubules in growth cones and also whether microtubule stability is required for Wnt attraction. Microtubules can be imaged by immunostaining with α-Tubulin antibodies and α-Tubulin-GFP fusion constructs. We test whether GSK3β and MARK2 can affect the trafficking of signaling components, such as Frizzled3, Celsr3, Dvls, Vangl2, PKCζ, Par3 and Par6 by affecting microtubule stability. To destabilize microtubule, we add, for example, colchicine and nocodazole in the culture. To stabilize microtubule, we add, for example, taxol.

We test whether microtubule stability is affected when Wnt signaling is either activated or inhibited. To activate Wnt signaling, we expose growth cones with Wnt gradient. To inhibit Wnt signaling, we use knockout tissues or RNAi or dominant-negative constructs. We analyze the microtubule organization by staining for α-Tubulin and microtubule plus end binding proteins such as, APC, EB1, EB3 and CLIP170. We perform imaging of microtubules with fixed cells or by live imaging.

2d): Determining how PCP and aPKC/A-B signaling pathways are coordinated to guide axons and introduce asymmetry.

There are several possibilities of how these two signaling pathways are integrated. These models are not mutually exclusive. Some of the results from Aim 1 and the rest of Aim 2 reveal how they might interact and give rise to directed turning response to Wnt gradients. In this aim, we focus on consolidating those studies with the aim to understand the coordination.

Model 1: These two pathways may act in parallel by activating distinct turning machinery components which are ultimately coordinated. In this model, PCP may regulate actin (via RhoA) whereas aPKC/A-B polarity pathway regulates microtubule (via GSK3b and MARK2). Alternatively, PCP may regulate membrane trafficking whereas the aPKC/A-B polarity pathway (aPKC/Par) regulates microtubules.

We analyze actin cytoskeleton in neuronal growth cones in response to Wnts and ask whether actin cytoskeleton response undergo changes after PCP is ablated (in knockout mice, RNAi or dominant-negative). We have already proposed to analyze the microtubule organization/stability when aPKC/A-B polarity signaling is disrupted (Aim 2c). If model one is true (they regulate parallel processes), blocking one pathway should not affect the normal function of the other pathway. For example, MT change should still occur when PCP induced action change is blocked by RhoA, or Daam1 (specific mediator of PCP to RhoA not RhoA in general). PCKζ inhibition should not affect actin. In fact, our preliminary results showed that actin dynamics is still highly active when GSK3β is blocked in live imaging of growth cone in “open-book” explants, suggesting that aPKC/A-B regulates microtubule independent of actin. If these processes depend on each other, then Model 3 is correct.

We study membrane/vesicular trafficking when PCP is blocked (2a above) and microtubule stability when aPKC/A-B signaling is blocked (2c above). If model is true, when PCP is blocked, Wnt induced vesicular trafficking may be affected but the MT changes induced by aPKC should still occur. Likewise, when MT changes are blocked, then vesicular transport may still be fine if it does not depend on MT. If these processes depend on each other, then Model 3 is correct.

Model 2: These signaling pathways converge on a common regulator, which then triggers downstream guidance events. For example, JIP (JNK-interacting protein), a partner of JNK (a downstream PCP protein) collaborate with FEZ1, a PKCζ substrate cooperate to activate Kinesin 1, a microtubule motor for membrane organelles⁽⁴⁾. Using pharmacological inhibitors, we found that JNK is required for A-P guidance of commissural axons. We test whether JIP is required for A-P guidance and whether Kinesin1 is also required (Aim2c). A truncated JIP protein has been shown to be effective blocker of JIP function. We express the construct followed by IRES GFP (to mark the cells that are expressing this construct) and test A-P pathfinding and Wnt attraction.

Model 3: PCP and aPKC may control different aspects of the same process. PCP may regulate directed recycling (endo and exocytosis) to set up asymmetric receptor/or signaling components (via Arf6) and aPKC provides a final output by polarizing exocytosis (via Lgl1). If this model is true, blocking either one blocks A-P guidance and disrupts the potential asymmetric distribution of signaling components. The results from other Aim 2 help determine whether Model 3 is correct.

Model 4: One polarity pathway is upstream of the other and asymmetry is conveyed by only either one or both of the pathways. This is a formal possibility because these two polarity pathways do regulate each other. We test whether PKCζ, Lgl1, FEZ1 and DISC1 localization and function in the growth cone are affected in Celsr3 knockout embryos or in Looptail mice. For example, we examine the levels of PKCζ, whose stability is under the control of Dishevelled, a core component of PCP signaling. We also examine the levels, localization and function of all the PCP signaling components, when PKCζ is inhibited by RNAi or dominant-negative construct or by Par3 or Par6 inhibitors. We test the localization and function of the PCP components in Lgl1 knockout mice (conventional and floxed allele).

### Results, Potential Problems and Alternative Approaches

After identifying the vesicular trafficking markers and regulators, which co-localize with PCP signaling components (Aim1a), we manipulate the trafficking system to test their effects on Wnt signaling. Arf6 is likely one of such regulators because it co-localizes with Frizzled3 and probably other PCP components. We have obtained the dominant-negative Arf6 constructs from Jim Casanova from University of Virginia to block its function in growth cones. We identify additional regulators of the trafficking system and test their functions. In addition, by carefully imaging how vesicular markers or regulators move in a growth cone, we determine whether their patterns are altered when Wnt signaling is affected. These experiments allow us to determine the role of membrane dynamics in growth cone guidance. A potential problem is that endo- and exocytosis may be involved in setting up signal asymmetry as well as actually membrane movement during axon turning. Live imaging can solve this problem. We can observe whether blocking vesicular trafficking will affect the distribution of Wnt signaling components and whether blocking Wnt signaling will change vesicular trafficking. We have observed asymmetric localization of a number of components now, including Vangl2, Lgl1, aPKC and β-catenin. Moreover, our preliminary imaging showed that during growth cone turning, there is an earlier phase of polarization with one or two filapodia being stabilized followed by a second stage of rapid and direct membrane translocation towards the stabilized filapodia. Therefore, growth cone movement in the second phase may not rely on polarized endocytosis and exocytosis observed in the first phage. For most of the live imaging experiments, we focus, for example, on the direction of vesicular trafficking as our initial analyses. The direction of vesicular movement is determined, for example, relative to the Wnt gradient and quantified by counting the number of vesicles that pass through a filapodia or pass across the midplane of a growth cone per minute (from left to right or from bottom to top)^{(40,50)}). To further characterize endo- and exocytosis to determine whether endo- and exocytosis is involved in membrane movement, we plan to monitor endo- and exocytosis. Endocytosis is monitored, for example, by dyes, such as FM1-43, which are taken up by endocytic vesicles and become activated. Location of exocytosis can be monitored by pH-sensitive GFP fusion with vesicular proteins as in synaptophlorin and VAMPphlorin⁽¹⁹⁾. These reagents are available in the lab and allow us to monitor endo- and exocytosis in our proposed experiments.

Because Celsr3 is required cell autonomously in the forebrain axon guidance, we anticipate that it is also required cell autonomously in commissural axon guidance along the A-P axis. This does not exclude a cell non-autonomous function of Celsr3. Indeed, in PCP signaling in Drosophila wing epithelial cells, Flamingo, the fly homologue, has both autonomous and non-autonomous functions. Therefore, it is informative to determine whether Celsr3 has autonomous or non-autonomous function or both. The non-autonomous function may be important for either A-P guidance or for axon tiling, either case would be highly informative. There is evidence for direct Wnt involvement in axon guidance in vertebrates, fly and in C. elegans and planar cell polarity in C. elegans^{(59,37,24,39,22)}. It is possible that commissural axons do not need cell-cell communication to amplify the signal because individual growth cones have sufficient sensitivity to Wnt gradients. Therefore, non-autonomous function of Celsr3 may not be relevant to AP guidance but rather only to axon tiling. We did notice that the growth cones in Celsr3 knockout mice are much larger and the axon terminals appear to branch more in DiI labeled commissural axons right after midline crossing.

Polarity signaling is essential in multiple morphogenetic processes. The two polarity signaling pathways have intricate interactions and may belong to one highly coordinated polarizing system that many epithelial or epithelia-derived cells use. Our studies determine whether these two polarity-signaling pathways are parallel, convergent or upstream downstream to each other. By putting them in the context of growth cone cell biology, we help elucidate the mechanisms of these fundamental morphogenetic signaling pathways in nervous system development.

### Example 3

### Role of Wnt Signaling in Brain Circuit Wiring

Overview. Example 3 is directed to (3a) detailed phenotypic analyses of dopaminergic (mdDA) and serotonergic (5-HT) neuron wiring in mice deficient in Wnt-mediated axon guidance signaling, (3b) analyses of expression patterns of Wnts, Wnt-inhibitors and Wnt receptors in the brainstem, (3c) use of explant assays to test the effects of Wnts on mdDA and 5-HT axon growth and guidance, and (3d) analyses of overall brain development and behavioral studies in mice with specific defects in mdDA and 5-HT axon wiring using conditional alleles of Wnt signaling components.

### Rationle, Overall Hypothesis and Design.

Preliminary studies from the lab showed that both mdDA and 5-HT fibers displayed severe guidance defects along the A-P axis in Frizzled3 and Celr3 mutant mice, similar to spinal cord commissural axons, suggesting that Wnt signaling may also be important for the A-P guidance of axons in the brain. To determine whether the axon phenotype is due to abnormal patterning or cell fate determination, we perform analyses of monoaminergic neuron differentiation. We perform in situ and immunostaining of a number of markers in the midbrain and hindbrain areas to assess patterning and differentiation in the brainstem^{(58,52,48)}. In order to define the detailed phenotypes, we analyze the development of mdDA and 5-HT projections in different developmental stages in wildtype and mutant mice, including axon trajectory, targeting and synaptic distribution. To understand the role of Wnts in guiding monoaminergic axons, we examine the expression patterns of all members of Wnts, their inhibitors (sFRPs, secreted Frizzled-related proteins) and their receptors, the Frizzleds, Ryk and ROR2. If the Wnt inhibitors are expressed in a graded fashion in the brainstem and/or along the trajectory of these neurons, they may also contribute to the guidance because they may help create functional Wnt gradient. Therefore, we analyze the expression of the inhibitors.

The conventional Frizzled3, Celsr3 and Ryk knockout mice are perinatal lethal. They live long enough for analyses of axon pathfinding but not for behavioral studies. To study whether the axon miswiring caused by the abnormal Wnt signaling leads to behavioral defects, we use Floxed alleles and crossing them to specific Cre lines. We have already obtained the Celsr3 conditional allele and are in the process of making the Frizzled3 and Ryk conditional alleles. We perform standard behavioral analyses of conditional Frizzled3, Celsr3 or Ryk alleles to DAT-Cre (specifically expressed in embryonic DA neurons) and ePet1-Cre (expressed specifically in early embryonic 5-HT neurons)^{(66,63)}. It was reported that the serotonergic system may have a role in regulating brain circuit formation, neurogenesis, survival, migration and axon refinement⁽⁵³⁾. We analyze brain development overall by markers of cell fate and axon projections. In the longer term, these conditional knockout animals allow us to investigate the role of Wnt signaling in the development of other descending motor pathways, for example the rubrospinal tract, reticular spinal tract and tectal spinal tract axons as well as ascending sensory pathways, such as the dorsal column axons^{(29,16)}.

Experimental Design 3a: Detailed phenotypic analyses of dopaminergic (mdDA) and serotonergic (5-HT) neuron wiring in mice deficient in Wnt-mediated axon guidance signaling. We examine a panel of patterning and cell fate markers that are important in the midbrain and hindbrain development. We obtain or generate in situ hybridization probes and antibodies against FGF4, FGF8, Wnt1, Wnt5a, Shh, Engrailed1, Engrained2, DAT, cPet and test whether the pattern of expression are the same in wildtype and heterozygous and homozygous mutants of Frizzled3, Celrs3 and Ryk^{(58,52,48)}. We have all three knockout-mice lines in the lab. We also examine the Looptail mice, which we confirmed to be a null allele of Vangl2, to test whether monoaminergic axon guidance is affected.

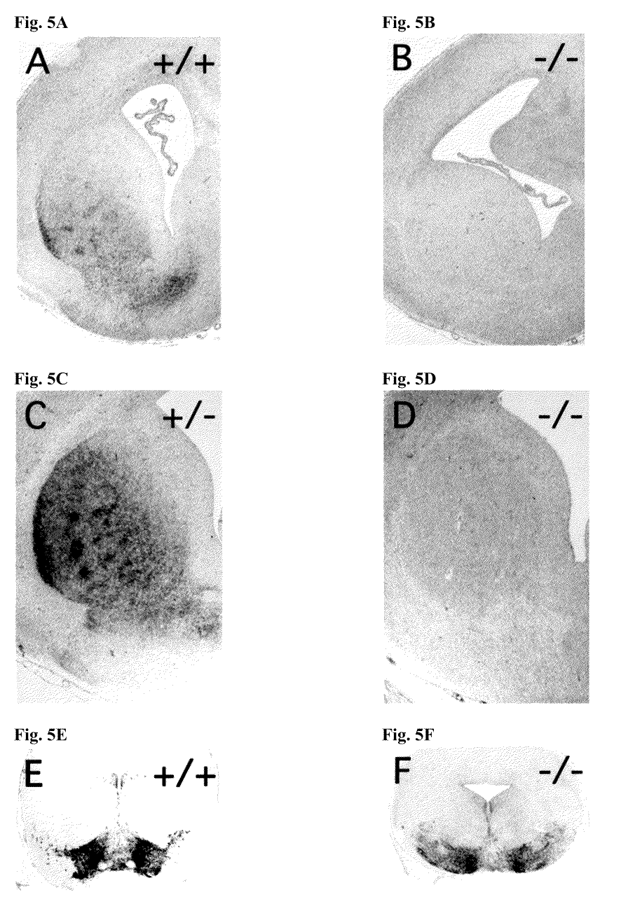

These mice survive until birth and therefore are suitable for analyses of circuit development for mdDA and 5-HT neurons. Dopaminergic axon development starts at E10.5 in mouse (E12 in rat) with the SNc mdDA neurons first project axons. And by E15.5 in mouse (E17 in rat), mdDA fibers start to innervate the striatum and extend further rostrally. By E17.5 in mouse (E19 in rat), both caudal and rostral parts of the striatum are heavily innervated by mdDA axons^{(48,51)}. It was reported that in Frizzled3 knock mice, TH staining in striatum was absent although the number of TH-positive dopaminergic neurons are normal at E18.5 (FIGS. 5A-5F)⁽⁵⁵⁾. This is consistent with our finding that in Frizzled3 knockout mice, TH axons are misguided and projected caudally instead of rostrally. We examine whether no TH axons project rostrally at all or some of them projected rostrally but never innervated the striatum. It was also reported that striatum contains a chemoattractant that attract TH-positive axons from SNc⁽⁵¹⁾. Our detailed analyses allow us to understand the formation of nigrostriatal pathway. We test whether the chemoattraction is mediated by Wnts. The development of 5HT axons is relatively less well known. The rostrally projecting 5HT neurons grow axons initially at E10.5 in mouse (E12 in rat). By E13.5, 5-HT neurons innervate broadly in the forebrain⁽¹⁾. Axons are stained, for example, with TH and 5-HT antibodies in whole mount staining and sections.

Some mdDA neurons and 5-HT neurons make synapses while others are extrasynaptic. Synaptic innervations in the forebrain targets, striatum, prefrontal cortex and limbic system are analyzed using VMAT2 and DAT to determine whether the number or pattern of TH and 5-HT innervations are altered in mutant mice^{(46,7)}.

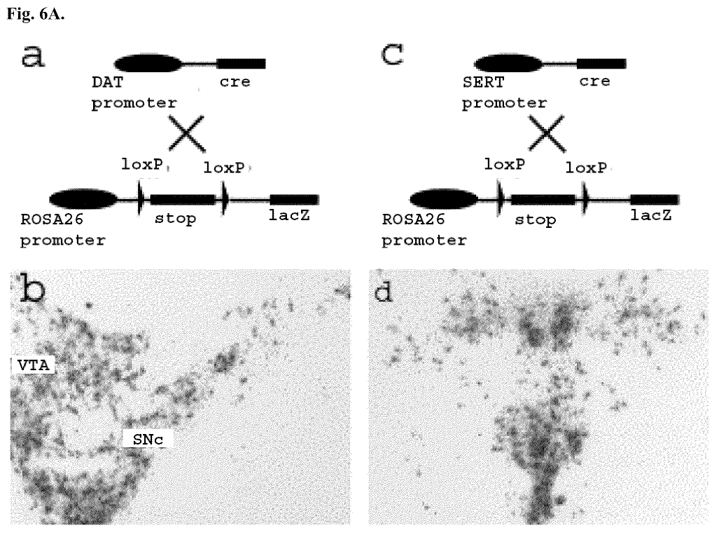

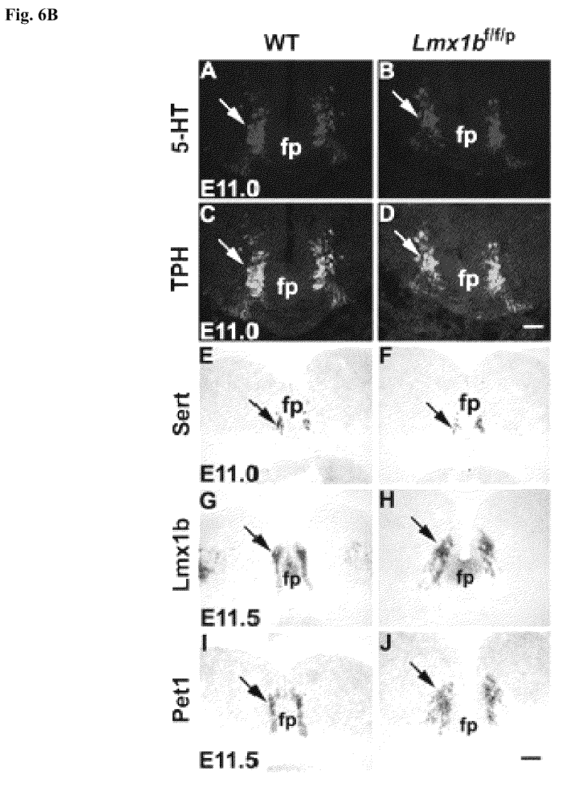

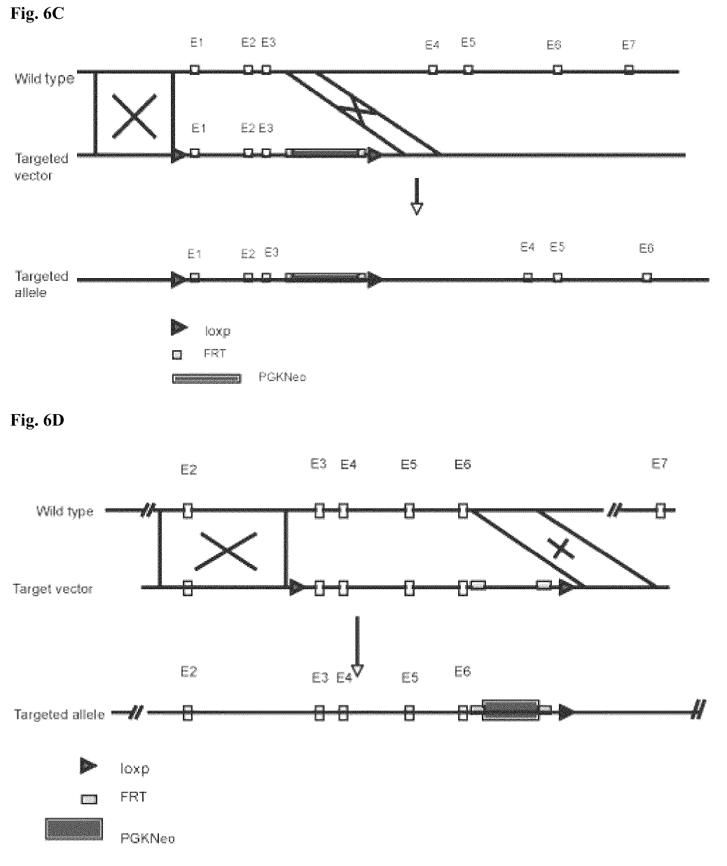

To create mice with specific monoaminergic axon defects, we use conditional alleles of Wnt receptor or signaling components and analyze their axon projection and innervations. They can be crossed to DAT-Cre and ePet1-Cre and perform similar axon wiring and synapse distribution studies (FIGS. 6A-6B). We have the Celr3 conditional allele⁽⁶⁵⁾. We are in the process of making Ryk and Frizzled3 conditional KOs. We clone LoxP sites to flank the same genomic regions that are targeted in the conventional knockout of Ryk and Frizzled3 (FIGS. 6C-6D). These constructs are electroporated and homologous recombination is selected, and blastocyst injection is performed to make conditional knockouts. We also analyze brain circuit development to test whether the absence of specific monoaminergic input affect brain development in these conditional knockout animals.

3b: Analyses of expression patterns of Wnts, Wnt-inhibitors and Wnt receptors in the brainstem. From published work, several Wnts are expressed in the brainstem region and in areas of forebrain and spinal cord where the TH and 5-HT neurons traverse or terminate⁽⁴⁸⁾. Currently, it is unknown whether any Wnts are expressed in a graded fashion overall. Wnt1 and Wnt5a are the best candidates because they are both expressed along the anterior-posterior trajectory of mdDA and 5-HT axons. We use in situ hybridization both whole mount and in sagittal sections or transverse sections (serial transverse sections along the A-P axis) to examine the expression patterns of all 19 members of the Wnt gene family (as we described in previous publications). We obtained several Wnt antibodies, which work in Western blots (Wnt3, Wnt4, Wnt5a), and use Western blotting to determine Wnt protein expression using protein extracts from tissues at different anterior-posterior positions. There are so far 6 sFRP genes have been reported. We have in situ hybridization probes for sFRP2 and clone the rest for thorough analyses of sFRP genes along the anterior-posterior axis in the brainstem.

Using in situ hybridization, we determine the expression of Frizzled3, Celsr3 and Ryk, which encode guidance receptors for Wnts. We test whether they are expressed in the mdDA and 5-HT neurons by double in situ hybridization with probes which mark mdDA and 5-HT neurons (FIGS. 6A-6B). We also use co-immunostaining with antibodies against Wnt receptors, Frizzled3, Celsr3 and Ryk, and markers for mdDA and 5-HT neurons, TH and 5-HT, respective. These analyses help determine which nuclei of mdDA (SNc, VTA or RRF) or 5-HT subnuclei neurons express which Wnt receptors. It appears that the most caudal part of the rostral 5-HT is affected more than the rostral 5-HT fibers. It is possible different receptors are expressed in different subnuclei. Identifying the guidance receptor in these neurons helps establish a direct role of Wnts in the guidance of functional monoaminergic circuits.

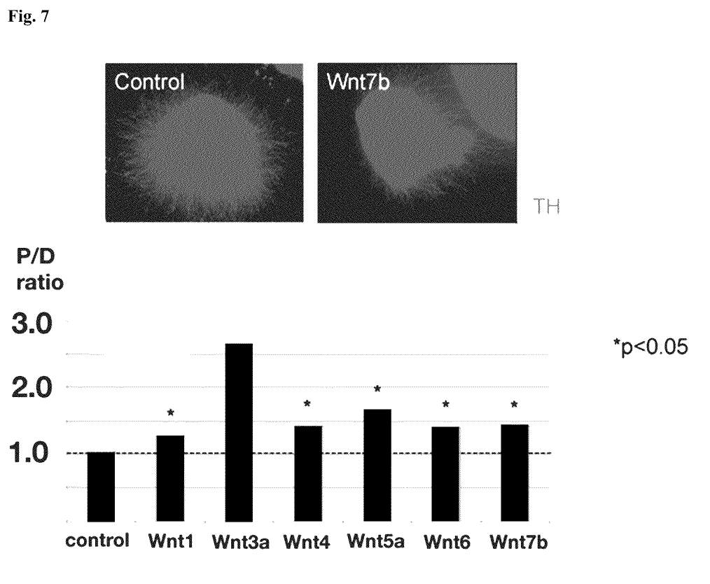

3c: Use of explant assays to test the effects of Wnts on mdDA and 5-HT axon growth and guidance. To directly address whether Wnts regulate the guidance of these monoaminergic axons, we use explant assays to test the function of the identified Wnts and determine whether they are attractive or repulsive. We found that several Wnts, particularly Wnt3a, attract DA neurons in explant assays (FIG. 7). We plan to continue test additional Wnts especially those which are expressed in the brainstem and the forebrain targets using the same assay.

Using the same explant assay, we then directly test which receptor is involved in attraction and which is for repulsion by using function blocking antibodies (against Ryk), sFPRs (against Frizzleds) or tissues from knockout mice missing specific Wnt receptors. Based on previous work, we hypothesize is that Frizzled3 and Celsr3 mediate attraction while Ryk mediates repulsion^{(32,30,44,27,57)}. The loss of TH neurons in the rostral target striatum in Frizzled3 knockout mice suggest that Frizzled3 is required for rostral growth⁽⁵⁵⁾. Our results show that Wnts are mostly attractive for TH axons, suggesting that Frizzled3 may mediate attraction anteriorly similar to the spinal cord commissural axons. The descending 5-HT axons are also severely misguided in Frizzled3 knockout mice, with very few descending axons and many axons were found oriented anteriorly improperly. Currently, the Wnt expression pattern around the midbrain hindbrain is unclear, particularly whether there is a gradient along the A-P axis. It is possible that Frizzled3 is a co-receptor required for Ryk mediated Wnt repulsion. Alternatively, Frizzled3 may mediate repulsion because, in C elegans, Frizzleds mediate repulsive guidance of Wnts^{(37,24,39)}. Therefore, it is important to delineate which receptor mediates attraction and which for repulsion. It should be noted that guidance cues other than the Wnts may play roles in shaping the trajectories of monoaminergic axons in the brainstem and in their forebrain or spinal cord target areas. However, it appears that at least the initial A-P decision in the brainstem is regulated by Wnt signaling.

3d: Analyses of overall brain development and behavioral studies in mice with specific defects in mdDA and 5-HT axon wiring using conditional alleles of Wnt signaling components. We perform systematic analyses of neurogenesis, survival, neuronal migration and axon dendrite patterning in brains with conditional knockouts of Wnt signaling components in mdDA and serotonergic neurons to determine whether these monoaminergic inputs from the brainstem play a role in regulating global brain development⁽⁵³⁾. We use a number of markers, such as NeuN, Tbr1, Brn2, and Ctip together with radial glial markers, such as BLP, RC2 and Nestin to assess overall neurogenesis, patterning and migration. We use L1, TAG1, neurofilament staining to assess overall wiring of axons. We perform Golgi staining to analyze the morphology of dendrites. For more specific circuits, we cross our mice lines into genetically labeled lines, such as Thy1-YFP lines, to test whether the wiring is correct. Characterizing the overall brain development in these conditional knockout mice provides important information for interpreting the results from behavioral studies, described below.

To assess dopaminergic function, the open field locomotor activity box is used to assess baseline locomotor activity as well as locomotor activity in response to dopaminergic drugs (e.g. dopamine transporter blockers). Reward based learning is assessed by both appetitive Pavlovian learning and appetitive instrumental learning. Reward based performance is assessed by motivation to obtain reward in a progressive ratio schedule. Motor coordination is assessed by the rotarod treadmill. We use, for example, 12 mice for each group for behavioral studies. Same mice are used for all the studies sequentially. Two-way ANOVA is used to analyze locomotor activity in response to dopaminergic drugs. One-way ANOVA is used for the rest.

To assess serotonergic function, we use the elevated plus maze to evaluate innate fear response, fear conditioning (both cued and contextual) to evaluate learned fear response, forced swimming test to evaluate “depression” like phenotype, and wheel running in constant darkness to evaluate circadian rhythm.

### Results, Potential Problems and Alternative Approaches

So far, no patterning and cell fate determination defects have been observed in either the spinal cord or the brain in Frizzled3 or Celsr3 knockout mice or Looptail mice. It is possible that PCP genes are not involved in cell fate determination in vertebrate nervous system development. Alternatively, other Frizzleds, Celsrs or Vangls may compensate their early cell fate functions. If there are patterning and fate defects in the brainstem, we resort to conditional allele and perform experiment as proposed to inactivate these genes after neuronal differentiation and then analyze axon wiring. The role of PCP signaling in cell fate determination or neurogenesis has not been observed in the vertebrate nervous system development. This may be due to the fact that most of the PCP signaling genes is redundant. If we observe cell fate changes in the brainstem in PCP gene mutants, we plan to study the role of Wnt-PCP signaling in neurogenesis and neuronal differentiation. We have in situ hybridization probes for all 19 Wnt family members and those for the receptors, the Frizzleds, Ryk and ROR2 from previous studies. The probes and immunostaining antibodies for mdDA and serotonergic neurons are also well defined and widely used. The detailed analyses of the receptor expression patterns may allow us to discern the differences in different subtypes of mdDA and 5-HT neurons, paving the way to analyze their specific projections and functions. The explants assays for mdDA neurons have already been established (FIG. 7). We set up similar assays for 5-HT neurons. In addition to collagen gel assays, we set up “open-book” assays using the brainstem tissues, a similar approach to the spinal cord “open-book” assay.

Locomotor activity is not a sensitive assay for dopamine loss. However, they do show drastically different responses to dopaminergic drugs (unpublished data from Xiaoxi Zhuang). Locomotor activity in response to dopaminergic drugs is assessed in the present study along with baseline locomotor activity. Reward based learning and motivation have been shown to be very sensitive to mild dopamine depletion or receptor blockade^{(41,56)}. In order to test whether SNc or VTA DA neurons are more affected in these knockout animals, we use, for example, behavioral studies sensitive to both nigra (locomotor activity response to dopaminergic drugs) and VTA (Reward based learning and motivation) dopamine deficiency. One concern is whether nigra dopamine deficiency leads to motor problems that could affect performance in learning and motivation studies. To address this potential problem, we have included appetitive Pavlovian learning that is minimally affected by motor dysfunctions to assess VTA dopamine deficiency⁽¹⁵⁾. The ascending serotonergic projections are quite diffused and functionally carried out by multiple receptor subtypes. At present time, there are no behavioral assays that target a specific pathway or receptor subtype. Whether the behavioral assays used here are valid behavioral models for anxiety and depression are still controversial in the field. Nevertheless, these are assays sensitive to serotonergic dysfunctions and they are assays of choice as a general assessment of serotonergic functions.

### REFERENCES

### Examples 1-3

1. Aitken, A. R., & I. Tork, 1988, J Comp Neurol 274:32-47.

2. Balasubramanian, N., et al., 2007, Nat Cell Biol 9:1381-91.

3. Beeler, J. A., B., et al., 2006, Physiol Behav 87:870-80.

4. Blasius, T. L., et al., 2007, J Cell Biol 176:11-7.

5. Bray, D. 1970, Proc Natl Acad Sci U S A 65:905-10.

6. Cagniard, B., et al., 2006, Neuropsychopharmacology 31:1362-70.

7. Can, D. B., et al., 1999, J Neurosci 19:11049-60.

8. Chen, P. L., & T. R. Clandinin, 2008, Neuron 58:26-33.

9. Chen, W. S., et al., 2008, Cell 133:1093-105.

10. Chen, Y. M., et al., 2006, PNAS 103:8534-8539.

11. Dickson, B. J., 2005, Nat Neurosci 8:1130-2.

12. Diefenbach, T. J., et al., 1999, J Neurosci 19:9436-44.

13. Djiane, A., et al., 2005, Cell 121:621-31.

14. Dollar, G. L., et al., 2005, Nature 437:1376-80.

15. Eyny, Y. S., & J. C. Horvitz, 2003, J Neurosci 23:1584-7.

16. FitzGerald, M. J. T., 1996. Neuroanatomy Basic and Clinical, 3rd ed. W. B. Sauders Company LTD.

17. Fradkin, L. G., et al., 2005, J Neurosci 25:10376-8.

18. Fritzius, T., et al., 2007, Febs J 274:1552-66.

19. Gandhi, S. P., & C. F. Stevens, 2003, Nature 423:607-13.

20. Gao, F. B., et al., 2000, Neuron 28:91-101.

21. Gindhart, J. G., et al., 2003, Mol Biol Cell 14:3356-65.

22. Goldstein, B., et al., 2006, Dev Cell 10:391-6.

23. Gordon, M. D., & R. Nusse, 2006, J Biol Chem 281:22429-33.

24. Hilliard, M. A., & C. I. Bargmann, 2006, Dev Cell 10:379-90.

25. Jordan, L. M., et al., 2008, Brain Res Rev 57:183-91.

26. Kamer, C., et al., 2006, Semin Cell Dev Biol 17:214-22.

27. Keeble, T. R., et al., 2006, J Neurosci 26:5840-8.

28. Knoblich, J. A., 2008, Cell 132:583-97.

29. Lemon, R. N., 2008, Annu Rev Neurosci 31:195-218.

30. Liu, Y., et al., 2005, Nat Neurosci 8:1151-9.

31. Logan, C. Y., & R. Nusse, 2004, Annu Rev Cell Dev Biol 20:781-810.

32. Lyuksyutova, A. I., et al., 2003, Science 302:1984-8.

33. Mackie, S., et al., 2007, Curr Opin Neurobiol 17:95-102.

34. Mohammad-Zadeh, L. F., et al., 2008, J Vet Pharmacol Ther 31:187-99.

35. Musch, A., et al., 2002, Mol Biol Cell 13:158-68.

36. Nakata, T., & N. Hirokawa, 2007, Sci STKE 2007: pe6.

37. Pan, C. L., et al., 2006, Dev Cell 10:367-77.

38. Pecina, S., et al., 2003, J Neurosci 23:9395-402.

39. Prasad, B. C., & S. G. Clark, 2006, Development 133:1757-66.

40. Sabo, S. L., & A. K. McAllister, 2003, Nat Neurosci 6:1264-9.

41. Salamone, J. D., et al., 2003, J Pharmacol Exp Ther 305:1-8.

42. Salinas, P. C., & Y. Zou, 2008, Annu Rev Neurosci 31:339-358.

43. Sato, M., et al., 2006, Nat Neurosci 9:67-75.

44. Schmitt, A. M., et al., 2006, Nature 439:31-7.

45. Schnorrer, F., & B. J. Dickson, 2004, Curr Biol 14:R19-21.

46. Sesack, S. R., et al., 1995, J Comp Neurol 363:264-80.

47. Shimada, Y., et al., 2006, Dev Cell 10:209-22.

48. Smidt, M. P., & J. P. Burbach, 2007, Nat Rev Neurosci 8:21-32.

49. Stenmark, H., & V. M. Olkkonen, 2001, Genome Biol 2:REVIEWS3007.

50. Tojima, T., et al., 2007, Nat Neurosci 10:58-66.

51. Van den Heuvel, D. M., & R. J. Pasterkamp, 2008, Prog Neurobiol 85:75-93.

52. Vitalis, T., et al., 2005, Exp Neurol 191 Suppl 1:S104-12.

53. Vitalis, T., & J. G. Parnavelas, 2003, Dev Neurosci 25:245-56.

54. Wang, Y., & J. Nathans, 2007, Development 134:647-58.

55. Wang, Y., et al., 2002, J Neurosci 22:8563-73.

56. Wise, R. A., 2004, Nat Rev Neurosci 5:483-94.

57. Wolf, A. M., et al., 2008, J Neurosci 28:3456-67.

58. Ye, W., et al., 1998, Cell 93:755-66.

59. Yoshikawa, S., et al., 2003, Nature 422:583-8.

60. Zallen, J. A., 2007, Cell 129:1051-63.

61. Zhang, X., et al., 2005, J Cell Biol 170:273-83.

62. Zhang, X., et al., 2007, Nat Cell Biol 9:743-754.

63. Zhao, Z. Q., et al., 2006, J Neurosci 26:12781-8.

64. Zhou, F. Q., et al., 2004, Neuron 42:897-912.

65. Zhou, L., et al., 2008, Science 320:946-9.

66. Zhuang, X., et al., 2005, J Neurosci Methods 143:27-32.

67. Zou, Y., 2006, Neuron 49:787-9.

68. Zou, Y., 2004, Trends Neurosci 27:528-32.

69. Zou, Y., & A. I. Lyuksyutova, 2007, Curr Opin Neurobiol 17:22-8.

70. Zou, Y., et al., 2000, Cell 102:363-75.

### Example 4

### Localization of PCP Components

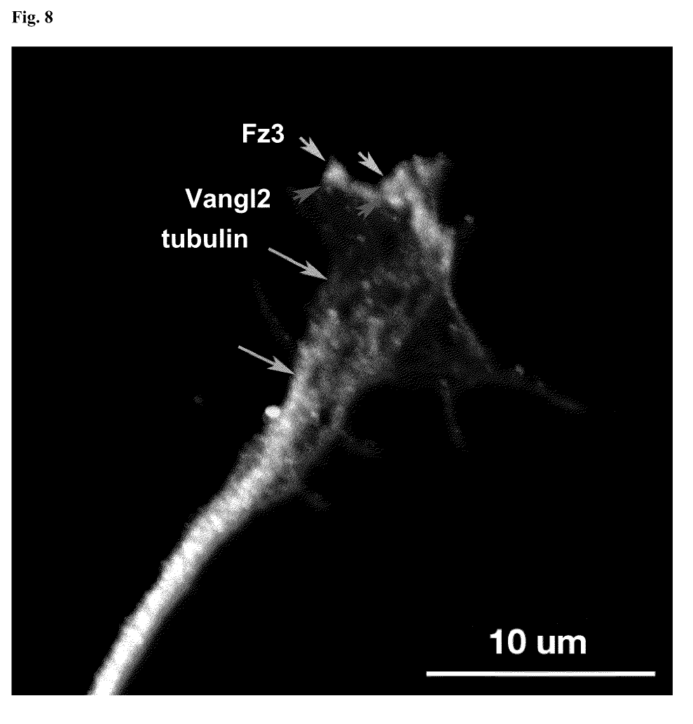

In order to understand how Wnt-planar cell polarity (PCP) signaling regulates directed growth of axons, we determine the subcellular localization of the Wnt/PCP components to determine whether any one of them is localized asymmetrically. In previous studies, we reported localization studies of Frizzled3, Celsr3, Vangl2 and Dishevelled2. We found that Vangl2 has a tendency to be localized asymmetrically. We now costained for Frizzled3 and Vangl2 and found that these two components tend to be localized close to each other with some overlap. See FIG. 8. Frizzled3 and Vangl2 appear to be either in the same large complexes or ensembles of vesicles or membrane domains, localized in specific regions of the commissural axon growth cone. These co-localizing “complexes” are present either on several neighboring filapodia or one area of lamellipodia, an indication of asymmetry.

### Example 5

### Localization of Component in the PKCζ Pathway

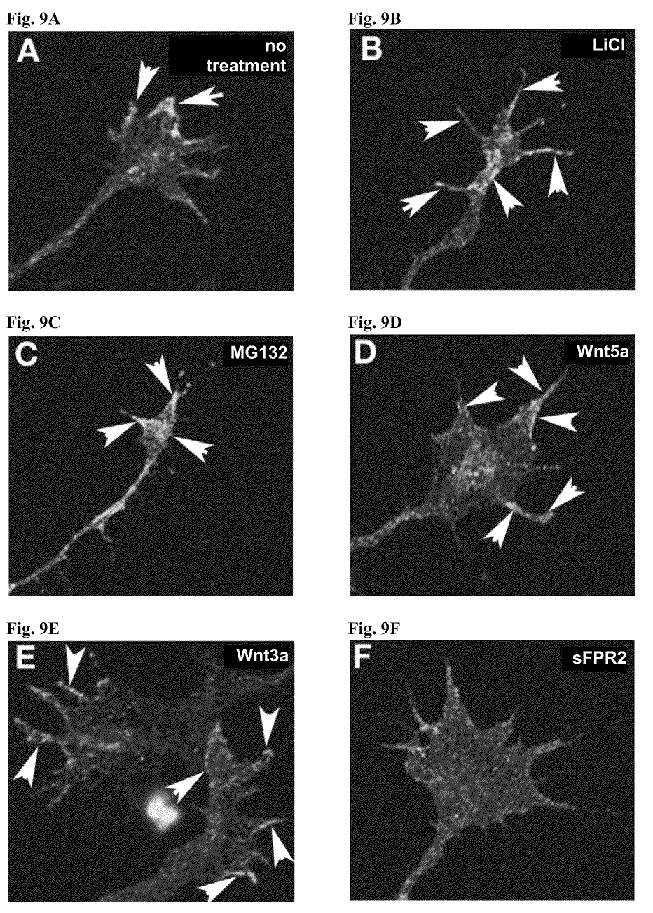

To understand how atypical PKC/Par pathway regulates axon guidance, we set out to determine the subcellular localization of aPKC, their substrates and downstream cellular effectors. We now found that β-catenin, a substrate of GSK3β, which is in turn a substrate of PKCζ is found localized asymmetrically in commissural axon growth cones (arrowheads in FIG. 9A). This asymmetry can be broken by LiCl (an inhibitor of GSK3β) (FIG. 9B) and MG132 (an inhibitor of proteosomes) (FIG. 9C), both of which elevated the level of β-catenin throughout the growth cone (arrowheads). Bath application of Wnt5a and Wnt3a also increased the levels of β-catenin throughout the growth cone, canceling the asymmetry (FIGS. 9D-9E). Bath application of a Wnt inhibitor, sFPR2, eliminated asymmetric distribution of β-catenin (FIG. 9F). Thus, our high-resolution confocal imaging detects a tendency of β-catenin being concentrated to one side of the growth cone (FIG. 9A). It is possible that there is low level of Wnt proteins in the culture (many neurons express Wnts). Recent work showed that neutrophils are naturally polarized without any extrinsic cues and this cellular chirality is mediated by atypical PKC/Par activity⁽⁴⁾. Therefore, it is also possible that intrinsic aPKC signaling can create chirality within the growth cone with out extrinsic signals. Such intrinsic polarity may then robustly regulate cellular morphology upon directional instructions from the environment such as a Wnt gradient. It is well known that GSK3β phosphorylates β-catenin, which then leads to proteosome-mediated degradation of β-catenin. And it is also known that GSK3β is phosphorylated and inhibited by PKCζ^{(1,2)}. Therefore, inhibiting GSK3β or the proteosome activity generally should increase β-catenin level throughout the growth cone, canceling the asymmetry. The general increase of β-catenin throughout the growth cone after bath application of Wnts and the decrease of β-catenin after sFRP2 addition are also consistent with the hypothesis that PKCζ-GSK3β regulates the levels of β-catenin in the growth cone. β-catenin has been shown to increase neurite outgrowth in a transcription-independent way, suggesting a local function of β-catenin in local growth cone, such as promoting cell adhesion (via cadherins) or capturing microtubule plus ends (APC is found enriched in the microtubule plus ends)⁽³⁾. We are now testing the hypothesis that Wnt proteins may locally activate PKCζ, which then inhibits GSK3β and locally stabilizes β-catenin, which is part of the asymmetric cellular mechanism leading to directional choice of a growth cone. We examine the localization of β-catenin in post-crossing commissural axon growth cones in the “post-crossing” assay and “open-book” assays.

### Example 6

### Improved TH Staining

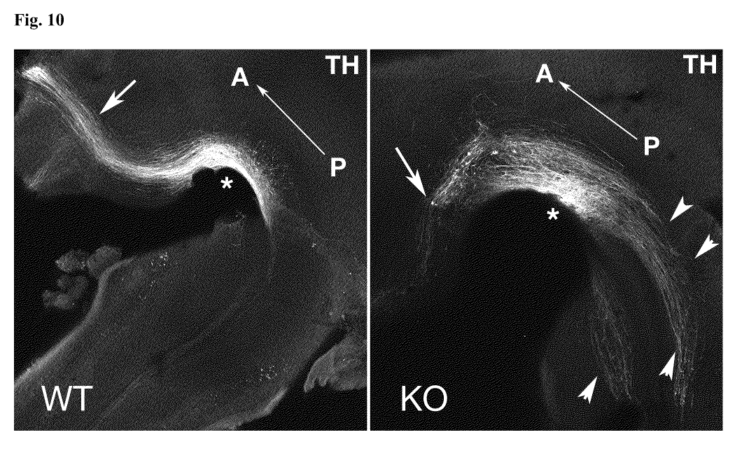

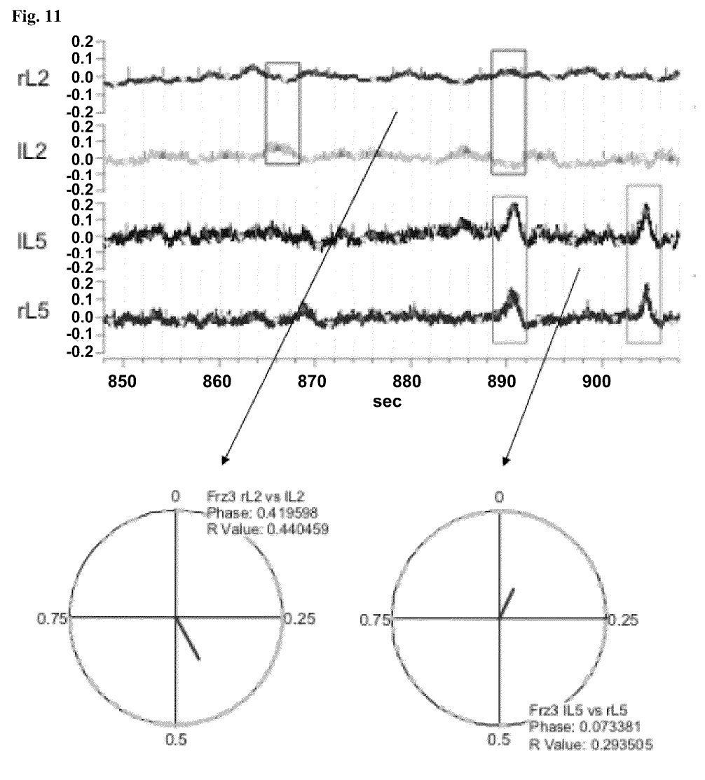

In preliminary studies, we used a mouse monoclonal antibody against Tyrosine Hydroxylase (TH) to label dopaminergic neurons in E12.5 wild type and Frizzled3 knockout embryos. We observed clear defects whereby many TH-positive fibers are now projecting posteriorly down towards the spinal cord. However, the background was high because the secondary antibody was against mouse IgG. Now we obtained a new rabbit polyclonal antibody against TH and the background is eliminated because the secondary antibody is against rabbit IgG (FIG. 10). A large number of TH axons are projecting posteriorly in homozygous mutants (white arrowheads in Frizzed3 knockout). This phenotype has 100% penetrence (homozygous mutants n=4). Asterisk shows the position of the cell bodies of DA neurons. We proposed to study the expression pattern of Wnts in the brainstem by a combination of in situ hybridization and Western Blotting. So far, we found that Wnt7b is expressed in an anterior-high to posterior-low gradient in the brainstem, suggesting that Wnt7b may be a major regulator of anterior-posterior guidance of both the TH and the 5-HT fibers. This is consistent with the finding that Wnt7b attracts the TH-positive neuron axons (FIG. 7). Because Frizzled3 is an attractive Wnt receptor, in the Frizzled3 mutant embryos shown above, TH axons project both anteriorly (white arrow) and posteriroly (white arrowhead) compared to wild type littermates, where all TH axons project anteriorly.

### Example 7

### Locomotor Behavioral Defects in Frizzled3 Knockout Animals

In disclosed in Example 3, we analyzed overall brain development and test behaviors in mice with defects in DA neurons and 5-HT neurons. We conduct behavioral analyses using conditional knockout mice of Wnt signaling genes. We also analyze the locomotor circuits and behaviors, which are known to be activated and modulated by serotonin, using electrophysiology recording to examine the locomotor output in spinal cord preparations from animals as early as E18.5. This allows us to test the feasibility of studying locomotor circuits because the Frizzled3 knockouts survive until birth. We found that the right-left alternation of L2 in the nulls was relatively normal, but the left right of L5 was synchronous. See FIG. 11.

### REFERENCES

### Examples 4-7

1. Etienne-Manneville, S. & A. Hall, 2003, Nature 421:753-6.

2. Etienne-Manneville, S. & A. Hall, 2003, Curr Opin Cell Biot 15:67-72.

3. Votin, V., et al., 2005, J Cell Sci 118:5699-708.

4. Xu, J., et al., 2007, Proc Natl Acad Sci U S A 104:9296-300.

### Example 8

### Further Approaches for Wnt Signaling

Approach and Overview. Neurodegeneration is the cause of many devastating nervous system disorders, including Alzheimer's Disease, amyotrophic lateral sclerosis (ALS) and Parkinson's Disease. Traumatic injury to the adult central nervous system or stroke also lead to neuronal cell death and loss of functions. Although much effort has been spent and several genes involved in inherited forms of neurodegeneration have been isolated, the cause of almost all neurodegenerative diseases remains unknown, let alone effective treatment or cure. Neurodegenerative research traditionally takes a “top-down” approach by studying patients and patient families and isolating genes that are mutated in patient families. These conventional approaches are clearly important and have given promising leads to understanding the pathogenesis. However, they are somewhat limited. Many patients do not have family history. The isolated genes are often under-characterized, making the progress of understanding diseases slow and costly.

There is disclosed herein a novel “bottom-up” approach. Our studies on axon growth and guidance lead to the discovery of a novel survival pathway of the central nervous system (CNS) neurons. We found that Wnt signaling stimulates the survival of CNS neurons via the activation of atypical protein kinase C (aPKC) polarity pathway as well as triggers an active degenerative pathway via a repulsive Wnt receptor, Ryk, which is involved in rapid killing of CNS neurons. Without wishing to be bound by any theory, it is believed that CNS degeneration diseases result from the inactivation of this common survival/death pathway and different forms of degenerative diseases are caused by disruption of different upstream pathways leading to the inactivation of this central survival pathway. This is a new concept challenging the conventional view that the neurodegenerative diseases are entirely different. Using this “bottom-up” approach, we may provide a complete new concept viewing neurodegeneration, which affects a large community around the world.

To this end, we have developed a complete set of in vitro and in vivo methods to assess the function of this common neuronal survival pathway in all major forms of neuronal degeneration. The steps included within the “bottom-up” approach include the following.

Step 1. To test whether the Wnt-aPKC pathway is a target of Aβ toxicity and in AD. A downstream target of aPKC is GSK3β, which is implicated in A-β toxicity and AD⁽¹⁾. Therefore, we hypothesize that aPKC may promote survival by keeping GSK3β inactivated and the inhibition of aPKC may cause GSK3β activation, leading to tau hyperphosphorylation and microtubule disassembly leading to axon fragmentation. 1a. We add Aβ oligomers and test whether the level of PKCζ, the localization or the activity of PKCζ is altered using biochemical assays and immunocytochemistry. 1b. It has been shown that Wnt3a can reduce Aβ toxicity². Based on our results, we suggest that Wnt3a may exert its protective function by stimulating the Wnt-aPKC survival pathway. We inhibit aPKC and test whether Wnt3a can still protect neurons against Aβ using the cell survival assay. 1c. Because our preliminary studies showed that Ryk function is required for neuronal death when the Wnt-aPKC pathway is inhibited, we test whether Ryk is also required for Aβ toxicity by blocking Ryk function using anti-Ryk antibodies^{(3,4)}. 1d. We examine the expression levels and activity of Wnt-aPKC in AD mouse models, such as Aβ(1-42) overexpression transgenic line⁽⁵⁾. And if Ryk antibodies inhibit Aβ toxicity, we test whether Ryk antibody injection can prevent neuronal death in AD mouse model.

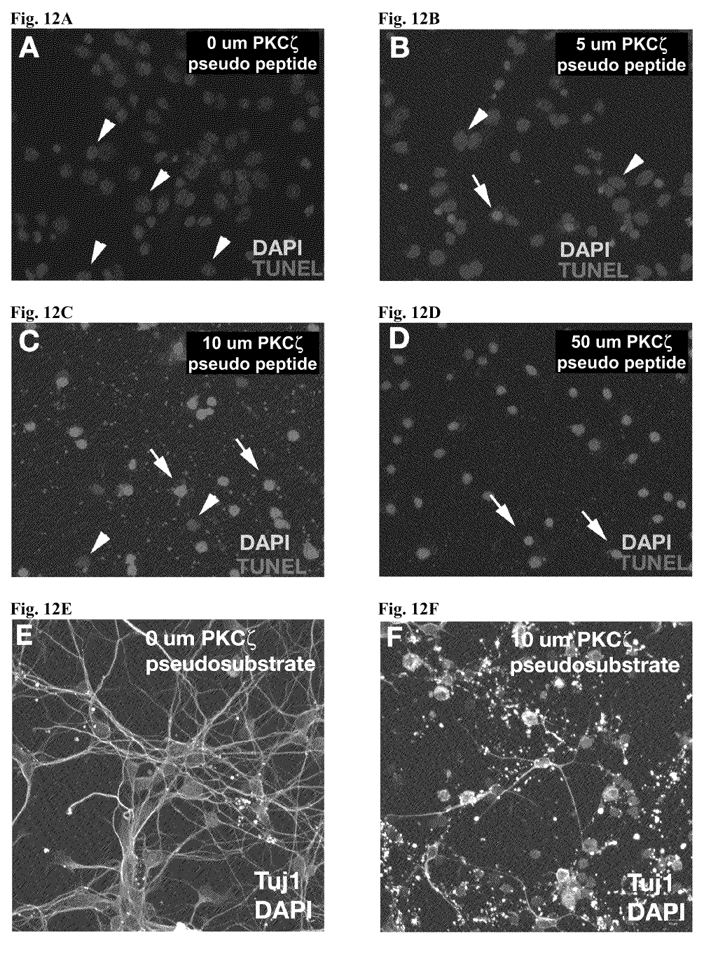

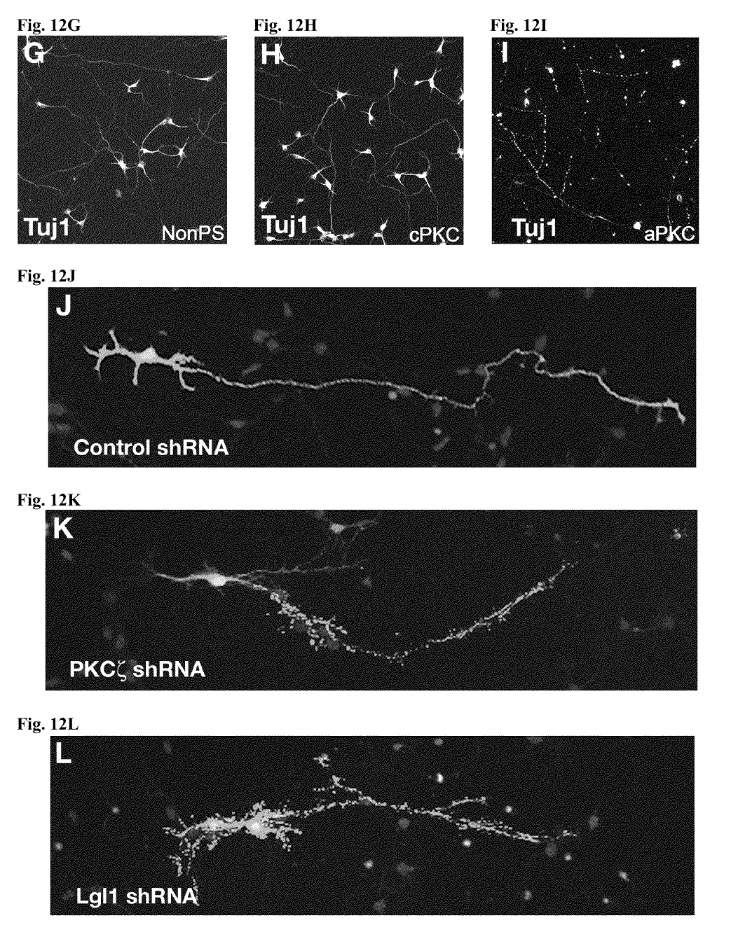

Step 2. To test whether the Wnt-aPKC pathway is affected in ALS. Studies using the SOD1 mouse model suggest that axon transport is a major early target of disease of familial ALS. In SOD1 (G93A) mice, fast-fatiguable (FF) motoneurons and the fast fatigue resistant (FR) motoneurons start to die at P48-50, showing classic features of axon degeneration, including rapid appearance of axon swelling, axon fragmentation and rapid removal of debris⁽⁶⁾. These processes are very similar to developmental death as has been observed. See FIGS. 12A-14D). 2a. We express SOD1 mutant construct (G93A) in dissociated ventral spinal cord cells (including motor neurons, interneurons and glia) and examine the level and activity of aPKC⁽⁷⁾. Recent results showed that SOD1 mutation (G93A) in the surrounding cells, not in the motor neurons themselves, causes motor neuron death. Therefore, we opt to culture a mixture of all ventral spinal cord cells. 2b. We express SOD1 (G93A) construct in ventral spinal cord using mouse in utero electroporation and chick in ovo electroporation and examine the levels and activity of aPKC. 2c. We apply Wnts and Ryk antibodies to test whether Wnts can counter cell death in ALS model⁽⁷⁾. 2d. We analyze the levels and activity of aPKC in SOD1 transgenic mouse model. If Ryk antibodies improve neuronal survival in culture, we inject Ryk antibodies in SOD1 mice to test whether axon degeneration is inhibited.

Step 3. To test whether the Wnt-aPKC pathway is affected in PD. Wnt signaling is crucial for the differentiation of dopaminergic neurons in development⁽⁸⁾. Our preliminary results showed that Wnt-Frizzled signaling is required for normal rostral projection of the DA neuron axons that selectively degenerate in PD patients, suggesting that the Wnt signaling system regulating axon growth operates in the DA neurons. We propose to test whether the levels and activity of aPKC are altered in Parkinsonian conditions. We plan to use in vitro and in vivo PD models to test the role of aPKC in DA neuron survival/death in PD pathogenesis. PI3K-Akt/PKB (protein serine/threonine kinase B), an upstream activator of aPKC, is required to protect neurons in rotenone-induced PD models in vivo and in vitro.⁽⁹⁾. Therefore, we test whether aPKC is a likely target of rotenone-induced PD pathogenesis. We also use N-methyl-4-phenyl-1,2,3,6-tetrahydropyridine (MPTP) to induced PD, a standard protocol in PD research⁽¹⁰⁾. 3a. Test the levels and activity of aPKC in PD models in culture, induced by rotenone, α-synuclein and MPTP. 3b. Examine the levels and activity of aPKC in mouse models of PD: MPTP treatment, rotenone and DJ-1 knockout and PINK1 knockout mice^{(11,12)}. 3c. Test whether Wnts or Ryk antibodies counter death induced in in vitro PD models. 3d. If Ryk antibodies, improve neuronal survival in vitro, we inject Ryk antibodies in PD mouse models and test whether DA neurons survive better in PD mice.

Step 4. To determine whether aPKC is required for neuronal survival in vivo using knockout mice and acute injury models. 4a. In Frizzled3 mutant mice, massive axon degeneration was observed in the neocortex shortly after the major axon tracks form⁽¹³⁾. Therefore, the Wnt-Fz-aPKC pathway may be involved in neuronal survival in vivo. However, it is not known why the neurons die. We propose to analyze the expression and activity of aPKC in Frizzled3 mutant mice and determine whether it is due to the inactivation of this survival pathway. 4b. Neurons undergo degenerative death upon traumatic injury. The mechanisms leading to their death are unknown but are potentially common to all degenerative death. We examine the expression of the PKC in crushed optic nerve⁽¹⁴⁾. In this injury paradigm, RGCs start do show death seven days after injury and fourteen days after, and 80% RGCs will die. Our preliminary results showed that the crushed nerve had reduced level of activated PKC three days after injury and at Day 8, much clear reduction was observed. We examine the level of activated PKC in other injury models, such as spinal cord injury⁽⁴⁾. 4c. If PKCζ level changes after injury, we then inject Ryk antibodies to test whether inhibiting Ryk function protects neurons.

Step 5. To develop monoclonal Ryk antibodies and carry out pilot compound screening for hits that block the degenerative death induced by inhibition of the Wnt-aPKC survival pathway. We develop therapeutic tools to promote neuronal survival based on the knowledge and assay system of this novel survival pathway. 5a. We made successful polyclonal function-blocking Ryk antibodies and produce monoclonal antibodies against the same Wnt binding domain in Ryk^{(3,4)}. So far, we have obtained two pure hybridoma clones producing antibodies against Ryk. These antibodies showed specific staining on cortical sections similar to our published polyclonal Ryk antibody^{(3,4)}. We have several more hybridoma with high titers and plan to isolate the pure clones using the same approach. We also the ability to produce more hybridoma clones. We test the function of monoclonal antibodies using the same cell death/survival assay as described. 5b. Using the survival same assay system, we take an unbiased approach to search for chemical compounds that may prevent axon degeneration⁽¹⁵⁾. Microtubule fragmentation can be readily quantified in a high-throughout format. The combinatorial chemical library are based on a novel concept of using molecular scaffolds of with “privileged structures”, which not only provide a great structural diversity but also increases the probability of obtaining functionally active molecules. We have the full access to the compound libraries and the high through put screening platform. Once we obtain compound hits, we confirm their effects in promoting neuronal survival and test whether greater effects can be achieved when both the compound and the Ryk antibodies are present.

### Background and Previous Studies

Compared to the peripheral nervous system, the mechanisms of how neuronal survival in the developing and in adult CNS are much less known. Wnts are secreted glycoproteins, which activate a number of diverse receptors, including the Frizzleds, Ryk and Ror2, and play a number of roles in development and function of the nervous system, including axon guidance^{(16,3,17,18,19,20)}. Our recent results showed that Wnts are also trophic factors and a Wnt-Frizzled-atypical PKC pathway is required for neuronal survival. Inhibiting this pathway by using a pseudosubstrate of PKC or knocking down PKC with siRNA can cause rapid axon degeneration initiating from microtubule fragmentation followed by nuclear shrinkage (pyknosis) and cell death in cultured cortical neurons. Wnt5a pretreatment can protect cortical neurons against death induced by PKCζ inhibition. Ryk is a Wnt-binding receptor that mediates repulsive response of axons to Wnts. Function-blocking Ryk antibodies also promoted neuronal survival against degenerative death induced by inhibition of PKCζ signaling (manuscript in preparation). Massive cortical axon degeneration was reported in Frizzled3 −/− mice a few days after initial axon outgrowth⁽¹³⁾.

1. PKCζ is required for survival of cortical neurons. E15.5 cortical neurons were dissociated and cultured for 2 days in the presence of various concentrations of a cell membrane penetrating myristoylated peptide derived in the pseudosubstrate domain of PKC (FIGS. 12A-12D). This peptide is a specific inhibitor of PKCζ. The pseudosubstrate peptide caused the death of cortical neurons and at 24 hours, massive axon degeneration can be observed by staining with neuronal-specific tubulin antibody, Tuj1 (FIGS. 12E-12F). We also found that only atypical PKC, such as PKCζ, is required, because addition of similar pseudosubstrate peptide of conventional PKC, such as PKCα does not cause neuronal death (FIGS. 12G-12I). We expressed shRNA constructs in cultured cortical neurons, using pSUPER vector with a GFP marker, and found that introduction of shRNA against PKC and its substrate lethal giant larvae (Lgl1), caused similar axon degeneration (FIGS. 12J-12L).

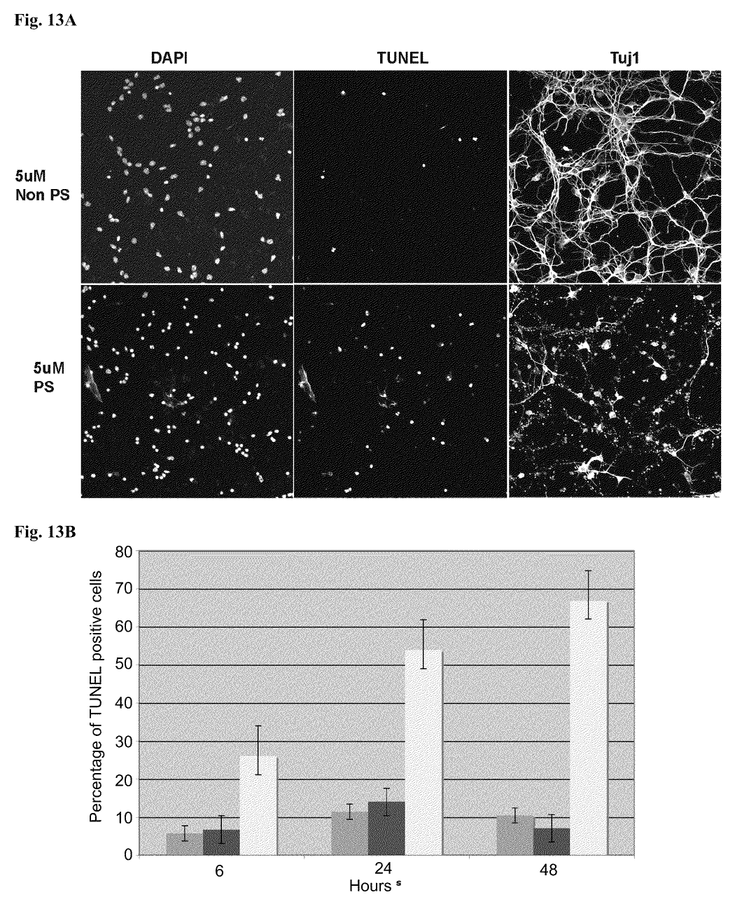

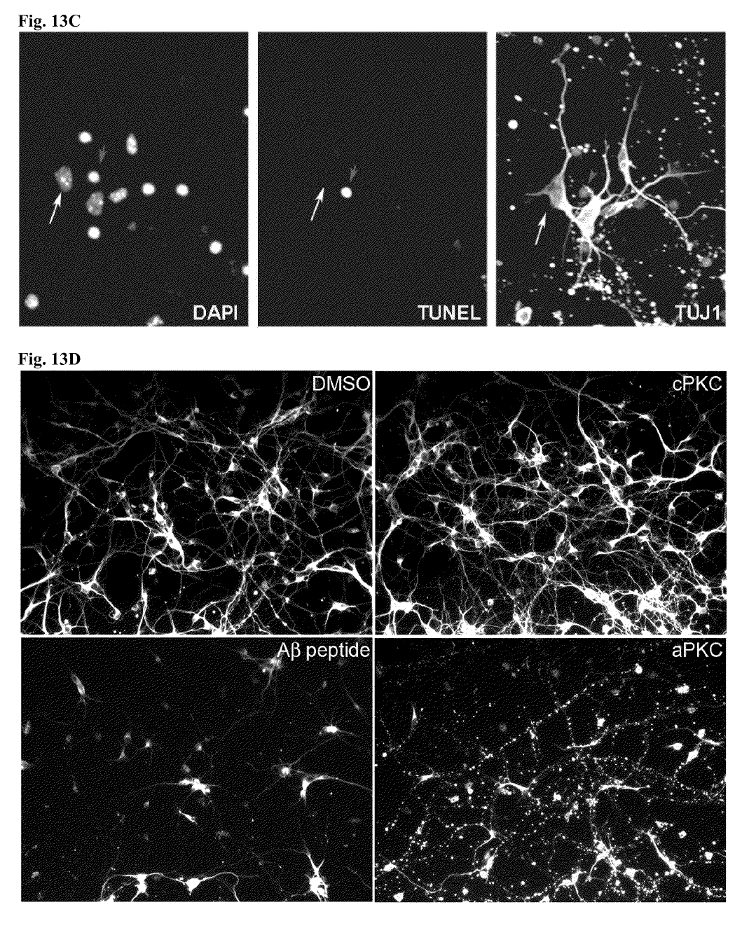

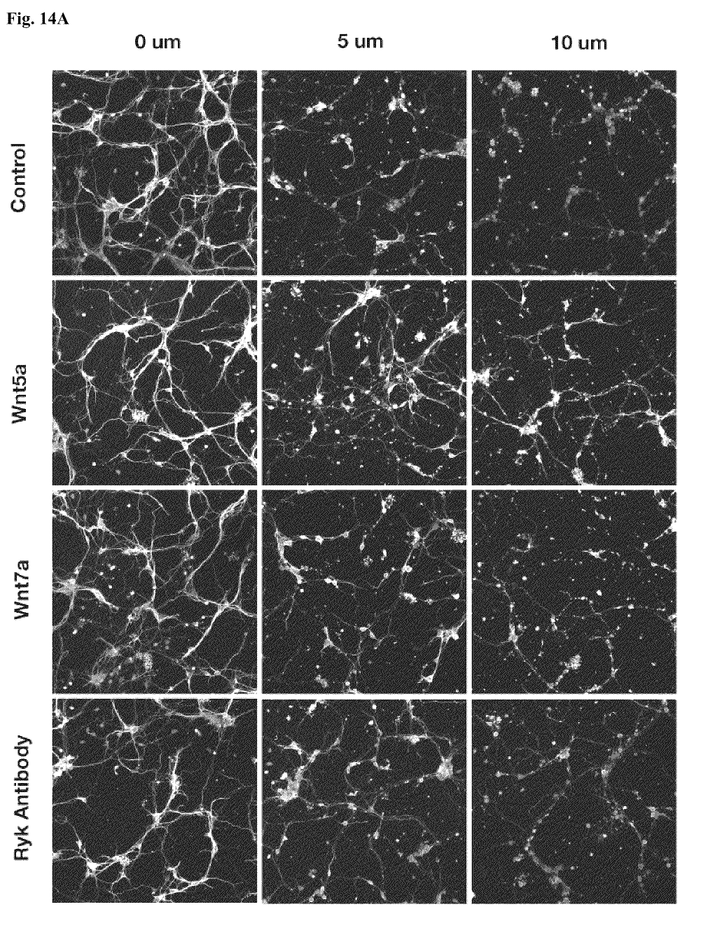

2. Axon degeneration procedes cell body death when PKC is inhibited. We examined the timing of axon degeneration and cell body death. We found that clear signs of microtubule disintegration/axon fragmentation can be observed after six hours of treatment. Many more neurons displayed axon fragmentation than TUNEL staining at 6 hours (FIGS. 13A, 13C), suggesting that axon degeneration occurs earlier than cell body death. The death induced by PKCζ inhibition is much faster than Aβ peptide induced neuronal death (FIG. 12D), probably because the PKCζ is an essential kinase and peptide inhibition causes instant death, whereas Aβ has to bind to cell surface receptor and trigger a signaling cascade to cause death, which requires a longer time.

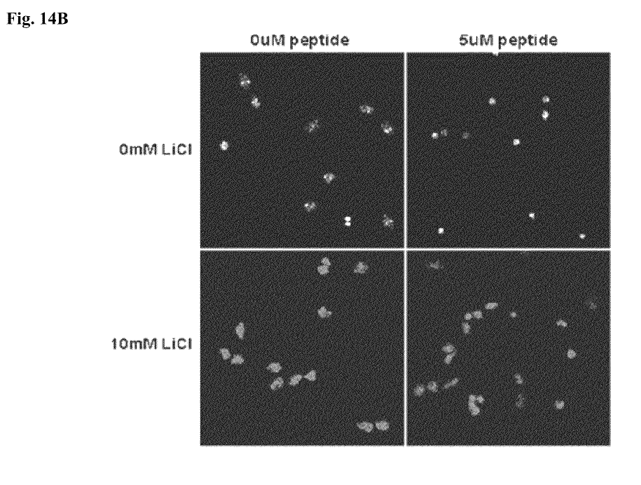

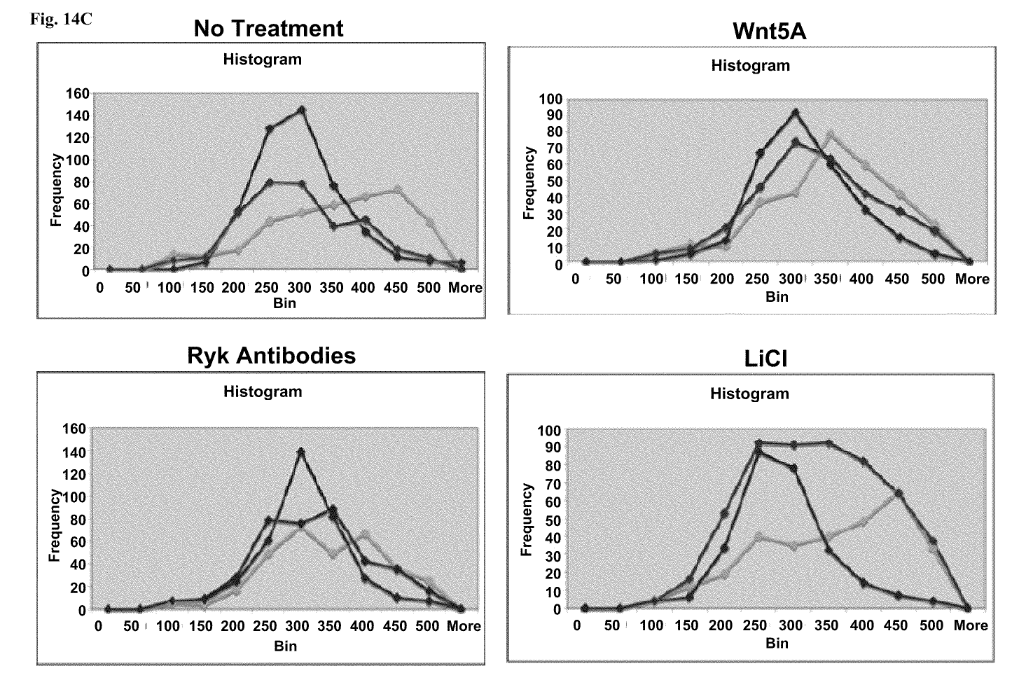

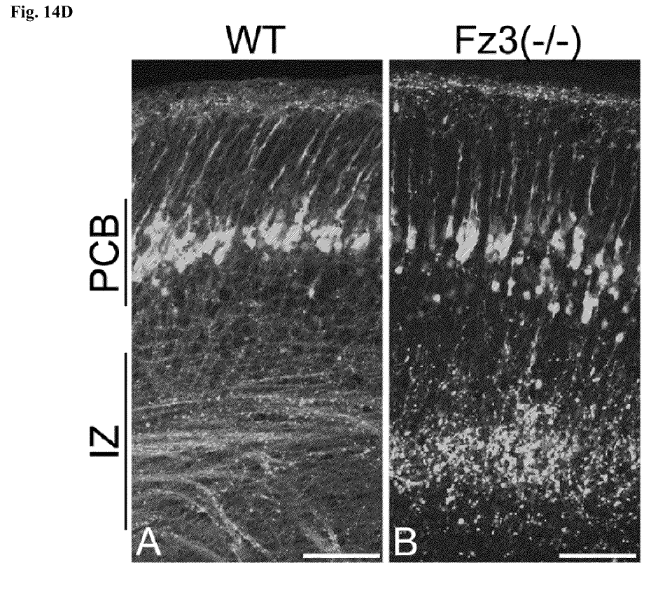

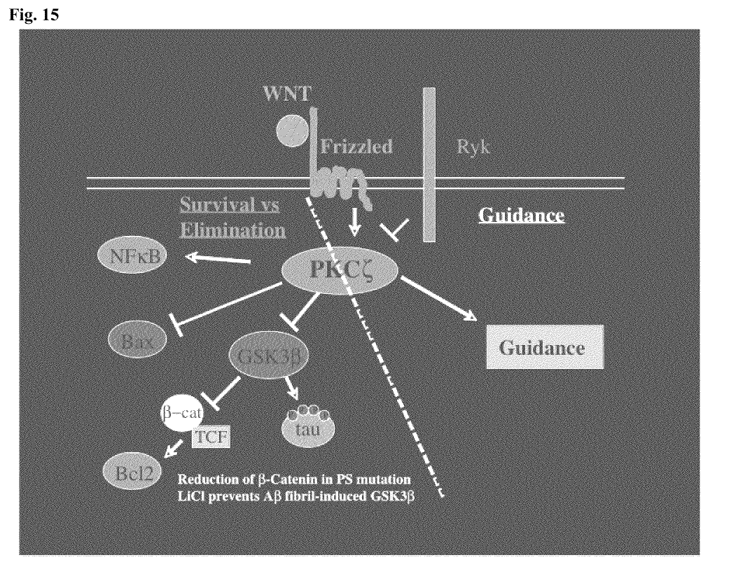

3. Wnts are trophic factors for cortical neurons. We found pretreatment with recombinant Wnt5a, Wnt7a, function-blocking antibodies against Ryk and LiCl, can block/reduce the cell death induced by PKC inhibition (FIGS. 14A-14C). After the addition of pseudosubstrate peptide, the amount of microtubule was clearly reduced and fragmentation of microtubules was detected using Tuj1 staining. At 5 um peptide, reduction of microtubule staining was severe in control conditions, whereas in the presence of Wnt5a, Wnt7a or Ryk antibodies, much less degeneration was seen (FIG. 14A). At 10 um peptide, more severe axon degeneration was seen in control conditions and even in the presence of Wnt5a, Wnt7a and Ryk antibodies. However, more axons still remain when Wnts and Ryk antibodies were added judging by Tuj1 staining. Nuclear shrinkage (pyknosis) is a hallmark of neuronal cell death. Using DAPI staining, we can visualize the cell nuclei (FIG. 14B). We developed a macro using Image J to quantify the neuronal death induced by PKCζ inhibition and found that in the presence of Wnts and LiCl (inhibitor of GSK3β), pyknosis was partially blocked whereas Ryk antibody almost blocked all pyknosis (FIG. 14C). Published work showed that Frizzled3 is required for survival of cortical neurons in vivo. Initial cortical axons and dendrite form and grow but axons start to disintegrate at E18.5 (FIG. 14D, from Wang et al 2006),⁽¹³⁾ (FIGS. 12F, 12K). Based on our preliminary results, we propose that Wnts are not only axon guidance molecules but also regulate neuronal survival and axon degeneration acting via PKC (FIG. 15).

### Methods and Experimental Design.

1. Assays for PKCζ levels and activity in cultured cortical neurons treated with Aβ oligomers (for AD), DA neurons treated with rotenone or MPTP (for PD), spinal cord motoneurons transfected with the SOD1 mutant (G93A) construct (for ALS) or cortical dissociated from the Frizzled3 mutant mice. PKCζ may be regulated by the amount of total PKCζ protein, the amount of activated PKCζ (phosphorylated) protein or the localization of the protein to plasma membrane and/or microtubules. Reduction of total PKCζ protein, activated PKCζ protein and delocalization of PKCζ from plasma membrane or microtubule can be detected by Western Blot of the total and membrane fractions of the culture neurons and immunocytochemistry of dissociated cortical cells treated with various reagents. Using high-resolution imaging with confocal microscopy, we observe that PKCζ is found localized on the tips of the plus end microtubules, consistent with its potential function of microtubule stabilization and, therefore, any changes of this localization are easily detected using this method. Treatment with Aβ oligomers, rotenone and MPTP is carried out according standard protocols. SOD1 mutant construct are transfected to dissociated ventral spinal cord motoneurons. Cortical neurons are dissociated from the Frizzled3 mutant mice at E16.5 and cultured for two days before assays as described in FIGS. 12A-14D, because axon degeneration occurs at E18.5 in Frizzled3 mutant mice.

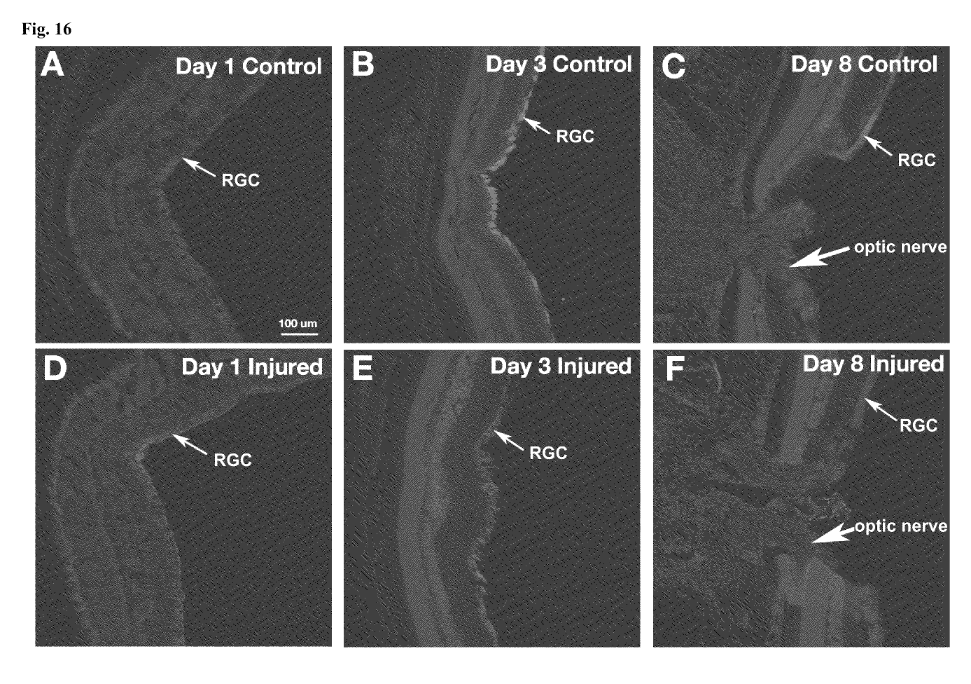

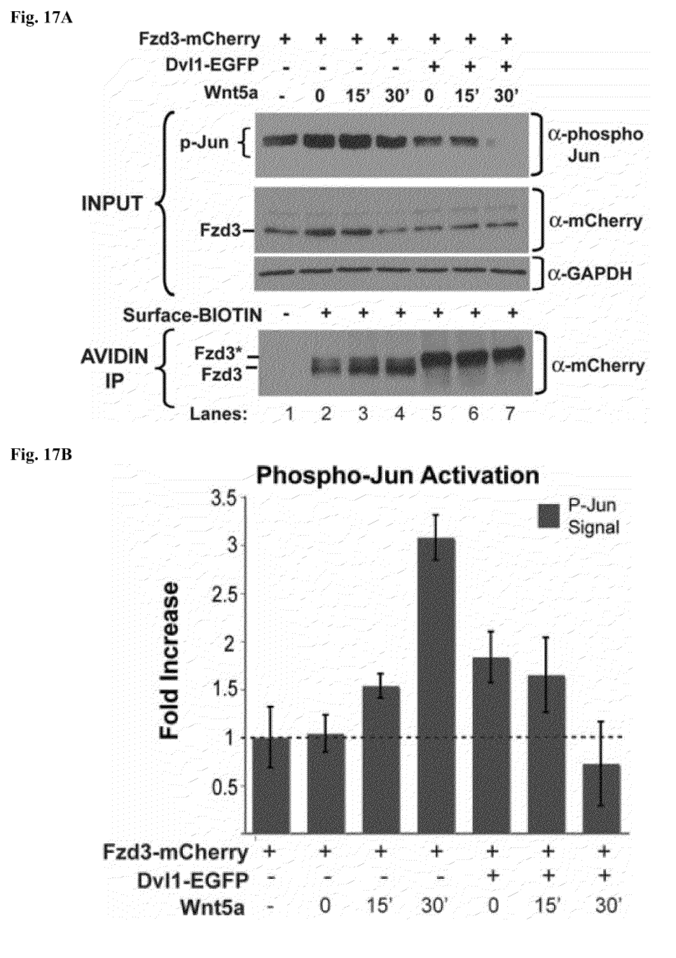

2. Immunostaining of PKCζ in mouse models of neurodegenerative diseases (AD, ALS and PD), Frizzled3 mutant mice and adult CNS injury models (optic nerve and cortical spinal tract). We use antibody against activated PKCζ (phosphorylated) to detect the level of PKC activity in normal mice of mice from various conditions, because CNS neurons undergo cell death, which, we propose, may be a result of inactivation of the PKCζ signaling in these CNS neurons. AD and ALS models are available from Jackson laboratory. Seven days after optic nerve crush, retinal ganglion cells (RGCa) start to show death and at fourteen days, 80% of the RGCs are dead. Our preliminary results showed that phosphorylated PKCζ level clearly dropped in RGCs after optic nerve crush three day after injury and eight days after injury (FIG. 16, Panels B and E), the levels of activated PKCζ is much reduced (FIG. 16, Panels C and F).

3. Survival and cell death assays for screening of monoclonal antibodies and small compounds. We use the same cell death/survival assay as described in FIGS. 12A-14D and use microtubule staining to analyze the integrity of microtubules, DAPI stain to measure the size of cell nuclei and TUNEL staining to assay cell death. Microtubule disassembly is quantified by staining intensity of Tuj1 and Tau antibodies. The size of cell nuclei is binned into difference sizes and the distribution of the nuclei size is used as quantification of pyknosis (FIG. 14C).

4. Screening of monoclonal antibodies with ELISA, Western Blotting and immunohistochemistry. We have been generating monoclonal hybridoma cell lines with Covance, Inc. Supernatants of mixed lines were first tested with ELISA using the Ryk ectodomain as the ligand. In the initial screen of 49 pools, we obtained 4 pools with the higher titers. We then used Western Blotting to confirm that the antibodies recognize the antigen. The cell lines were then subjected to further purification into individual clones. To date, 2 of the 4 pools of the highest titers have been purified (#107 and #121). We have also obtained the Ryk knock mouse line so that we can use the Ryk knockout mice to confirm the specificity of the Ryk monoclonal antibodies.⁽²²⁾ We then test the monoclonal clones, which can block the cell death induced by PKCζ inhibition using the cell death assay. Once function-blocking hybridoma are identified and cloned, we extract the mRNA and use RT-PCR to clone the immunoglobulin gene from that line and obtain the sequence of the variable region of the mouse monoclonal antibodies for therapeutic development.

5. Small compounds screening. These experiments are carried out using a large chemical library and is highly automated screen platform. Cell death is assayed 24 hours after inhibition of PKCζ by quantifying the level of Tuj1 staining. In degenerated axons, Tuj1 staining is significantly reduced (FIGS. 12E and 12F). The readout is robust. Once a compound hit is found, we verify the hit and test dose response and determine the optimum condition. We then test whether the combined monoclonal antibody and the hit compound produce a stronger protective effect against death induced by PKC inhibition. We then also test whether the monoclonal antibodies or the hit compound can also protect cortical neurons treated with Aβ oligomers (for AD), DA neurons treated with rotenone or MPTP (for PD), spinal cord motoneurons transfected with the SOD1 mutant (G93A) construct (for ALS) or cortical dissociated from the Frizzled3 mutant mice.

6. Injection of Ryk antibodies and compound hits. In a previous study, we injected Ryk antibodies into injured spinal cord to block repulsive function of the injury-induced Wnts to cortical spinal tract axons (CSTs). We found that Ryk antibody injection blocked retraction of injured CST axons and promoted sprouting of new collateral branches.⁽⁴⁾ We use similar approach to inject Ryk antibodies to test whether Ryk antibodies also promote neuronal survival in neurodegenerative conditions. We deliver the antibodies into the cortex, the hippocampus and the spinal cord in AD, PD and ALS and injury models to test whether Ryk antibodies improve survival of neurons. Compound hits are tested in similar ways in vivo.

### Example 9

### Vangl2 Antagonism

Summary. Planar cell polarity (PCP) signaling regulates tissue morphogenesis in a variety of cell types including axon wiring. Although core PCP components have been identified and characterized through genetic analyses, their biochemical and cell biological functions remain poorly known, greatly hindering the understanding of how a common set of proteins can mediate distinct developmental processes in different cell types. We show here Vangl2 can antagonize Dishevelledl-mediated feedback inhibition of Wnt/PCP signaling. This novel feedback inhibition by Dishevelledl is achieved by increasing hyperphosphorylation of Frizzled3 and preventing Frizzled3 internalization, which is required for PCP signaling. Vangl2 reduces Frizzled3 phosphorylation and promotes Frizzled3 internalization and thus prolongs PCP signaling. We found that Wnt/PCP signaling is required for A-P guidance of commissural axons and Fzd3 and Dvl1 co-localize each other on the plasma membrane in their growth cones. Vangl2 antagonizes Dvl1's function in localizing Frizzled3 on growth cone plasma membrane and promotes Frizzled3 internalization and is required for anterior-posterior guidance in vivo. This cell-autonomous mechanism likely operates in other cell types during PCP signaling.

Introduction. Planar cell polarity (PCP) refers to cell and tissue polarity along the planar axis of epithelia or mesenchymal cell sheets, perpendicular to the apical-basal axis (Wang and Nathans, 2007); (Zallen, 2007); (Goodrich, 2008); (Simons and Mlodzik, 2008). PCP signaling pathway is highly conserved and regulates the polarized cellular and tissue morphology exhibited in a number of processes, including orientation of epithelial prehair in the Drosophila wing, directed cell movement during vertebrate gastrulation, the polarized organization of mammalian stereocilia of cochlear hair cells, axon guidance and neuronal migration. Furthermore, in C elegans Wnts are instructive signals for PCP and control spindle orientation during neuroblast division (Goldstein et al., 2006). This impressive list of tissue morphogenesis functions of PCP signaling raises the question of how this one signaling system can elicit such a wide array of cellular outcomes.

The PCP signaling pathway involves two sets of regulators, the Frizzled/Flamingo core group and the Fat/Dachsous PCP system (Simons and Mlodzik, 2008). The Frizzled/Flamingo group of conserved core components include the seven transmembrane domain protein Frizzled (Fzd), the atypical cadherin with seven-pass transmembrane domains Flamingo/starry night (Fmi/Stan or Celsrs in vertebrates), the four-pass transmembrane protein Van Gogh or Strabismus (Vang/Stbm or Vgl), the Fzd-binding intracellular protein Dishevelled (Dsh, Dvl), the ankyrin repeat protein Diego (Dgo) and the Fzd-binding Lim domain protein Prickle (Prk1, Pk). Furthermore, it is known that PCP signaling leads to activation of c-Jun N-terminal kinase and c-Jun by phosphorylation (Boutros et al., 1998) (Yamanaka et al., 2002). Until now, very little is known about the biochemical functions of the PCP signaling components and their cell biological mechanisms of action with the exception that some components appear to directly bind to each other and in some cases their proper subcellular locations are correlated to intact PCP signaling. For example, Fzd and Dvl colocalize to the distal membrane of the epithelial cells of the Drosophila wing epithelial cells and Vang, Pk and Dgo are localized to the proximal membrane. How their subcellular localization is regulated biochemically and what signaling effects these cellular localizations trigger or reflect are completely unknown.

Wnt/PCP signaling involves both cell-autonomous and non-cell-autonomous mechanisms. Fzds, Dvls and Vgls have clear cell-autonomous functions. Prkls and Celsrs (homologues of Flamingo) have important non-cell-autonomous functions. However, cell-autonomous function and non-cell-autonomous function are two aspects of intimately related processes and in fact some components have both functions, such as Vgl2 and Celsr. Here, we focus on the interaction of the core PCP components, which are known to have cell-autonomous functions, and test their function and mechanisms of action in growth cone response to Wnt proteins.

We found here that upon Wnt addition, Fzd3 mediates PCP signaling but with time Dvl1, an intracellular signaling component, inhibits PCP signaling in a negative feedback fashion. Vgl2 itself, however, does not mediate PCP signaling in response to Wnt but can prolong Fzd3-mediated PCP signaling and antagonize the feedback inhibition caused by Dvl1. To further explore this mechanism, we found that Dvl1 increases Fzd3 phosphorylation and cell surface level (measured by surface biotinylation/immunoprecipitation and immunocytochemistry) and Vgl2 does the opposite, inhibiting Fzd3 phosphorylation and reducing its cell surface level. It has been shown that Dvl binds to AP-2 and clathrin-mediate endocytosis of Fzd is required for PCP signaling (Kishida et al., 2007; Sato et al.; Yu et al., 2007), although how Frizzled internalization is regulated, particularly by PCP signaling, is unknown. We show that Wnt5a does cause the reduction of cell surface levels of Fzd3 upon the addition of Wnt5a. Therefore, we propose a novel model that a negative Fzd-Dvl feedback signaling (by Fzd3 hyperphosphorylation) inhibits Fzd internalization and Vgl2 promotes PCP signaling by inhibiting Fzd3 phosphorylation and allowing Fzd3 internalization.

To further test this model, we examined the role of PCP signaling in commissural axon guidance. We found that indeed Wnt5a activates JNK signaling in commissural neurons and Fzd3 mediates Wnt stimulated axon elongation. Fzd3 and Dvl1, localizing primarily in intracellular vesicles by themselves, colocalize each other to the growth cone plasma membrane when co-expressed. Vgl2, which is primarily localized on growth cone plasma membrane, can also target Dvl1 to plasma membrane. Vgl2 prevents Fzd3 from being localized to the plasma membrane, suggesting that Vgl2 when activated may promote Fzd3 endocytosis in commissural axon growth cones, which is required for PCP signaling. Finally, Vgl2 is required for proper A-P guidance of commissural axons in vivo similar to previously observed function of Fzd3. Therefore, Vgl2 antagonizes Dvl1's inhibitory function by having opposite effects on Fzd3 membrane targeting in growth cone guidance.

### Results

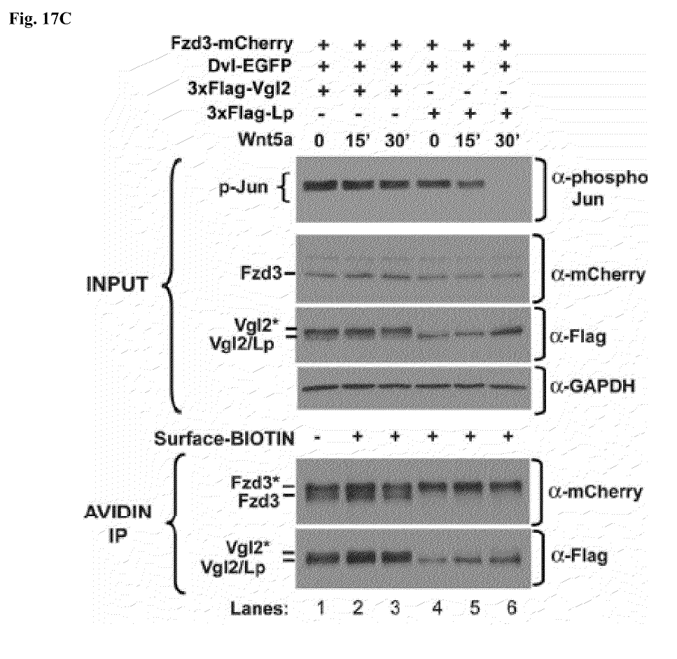

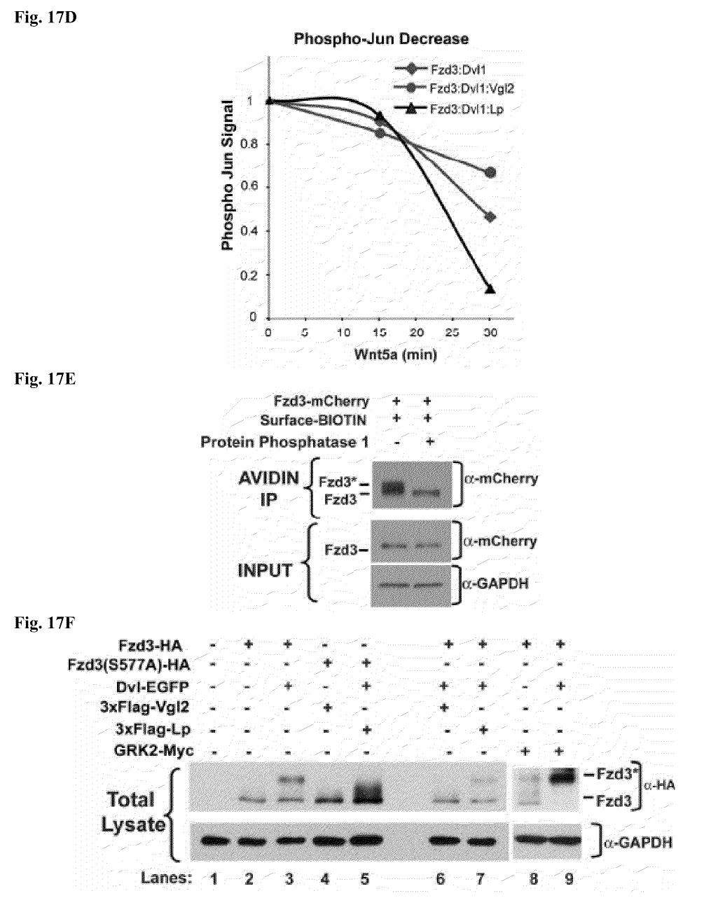

Vgl2 antagonizes Dvl1's feedback inhibition of Fzd3 mediated Wnt/PCP Signaling. To elucidate how core PCP components function cell autonomously, we first explored how Dvl1, Vgl2 and Fzd3 interact with each other. It is known that Dvl is a central cytoplamsic molecule in the activation of Wnt-PCP signaling. In non-canonical signaling, Fzd is thought recruit Dvl to the plasma membrane and Dvl then subsequently activates the downstream signaling, such as Jun N terminal kinases (JNK), Rac1 and RhoA. When Wnt5a was added to HEK293T cells expressing Fzd3-mCherry and, we found that p-Jun was indeed increased (Yao et al., 2004; Yu et al., 2007) (FIG. 17A lanes 2-4, quantified in FIG. 17B). However, upon co-expression of Fzd3 and Dvl1, we found a surprising Wnt5a-dependent down regulation of p-Jun level (FIG. 17A, lanes 5-7, quantified in FIG. 17B), uncovering a novel feedback inhibition mechanism in PCP signaling. With 30 minutes of Wnt5a addition p-Jun signal is essentially gone in Fzd3 and Dvl1 co expressing cells, (FIG. 17A lane 7).

Vgl is thought to have opposite function of Dvl in PCP signaling. In addition, Vgl and Dvl are found on the membrane on opposite sides of many epithelial cells, where PCP signaling is essential for establishing tissue polarity (Simons and Mlodzik, 2008). We asked if Vgl2 could affect this Dvl1 mediated feedback on PCP signaling. We found when Vgl2 is present with Fzd3 and Dvl1, the p-Jun signaling was prolonged and was not diminished after 30 minutes of Wnt stimulation (FIG. 17C lanes 3). Triple transfection of 3×Flag-Vg12, Fzd3 and Dvl1 and followed by Wnt5a stimulation resulted in a decrease in the decay of PCP signaling measured by p-Jun levels over time (FIG. 17C lane 1-3).

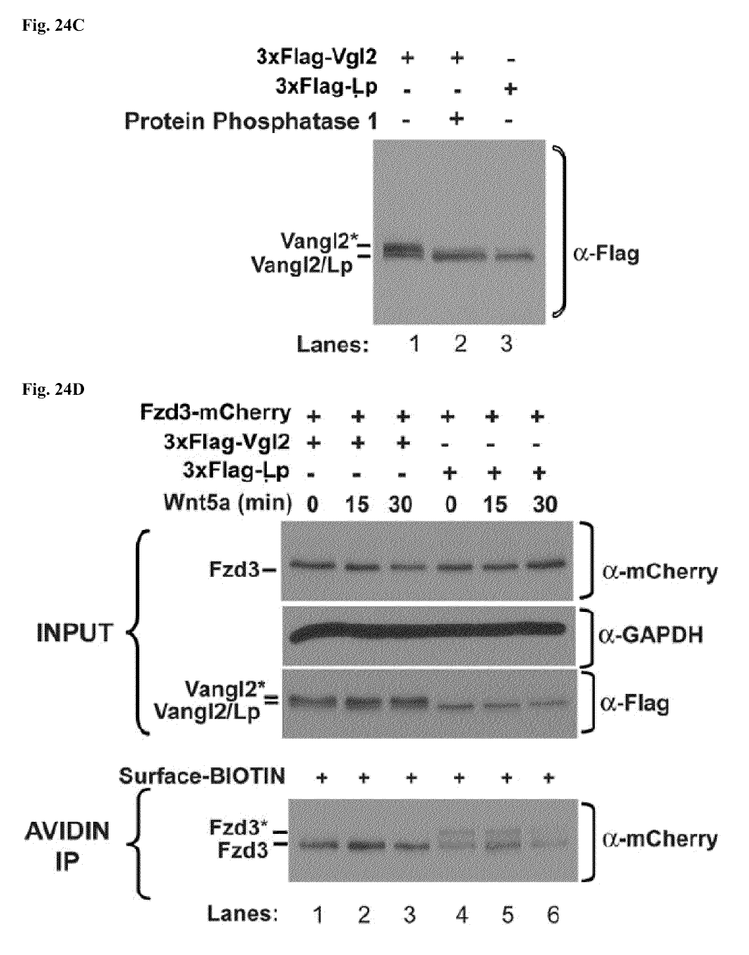

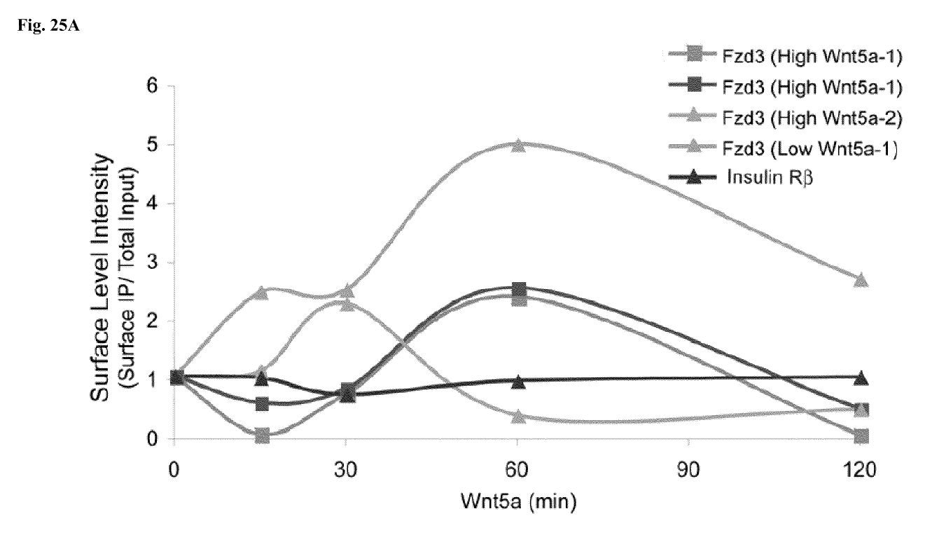

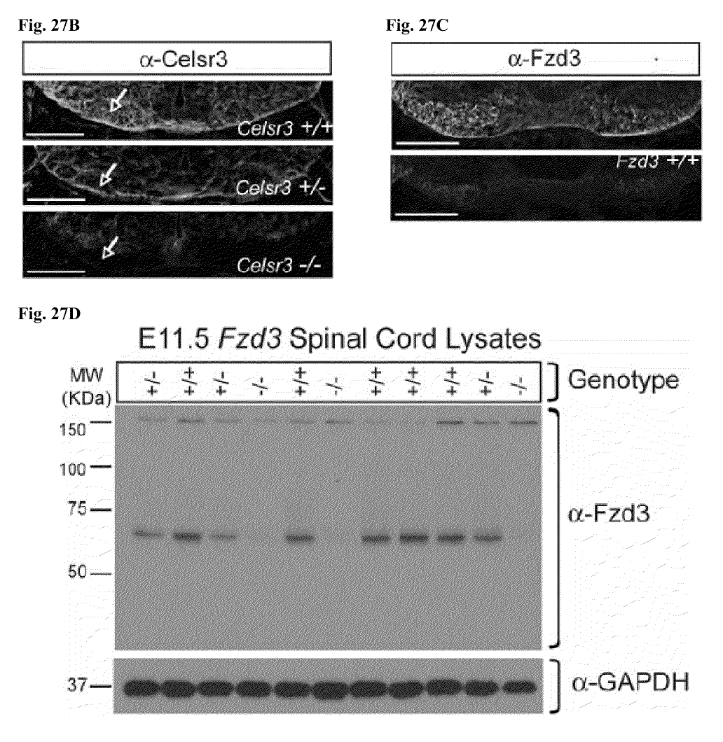

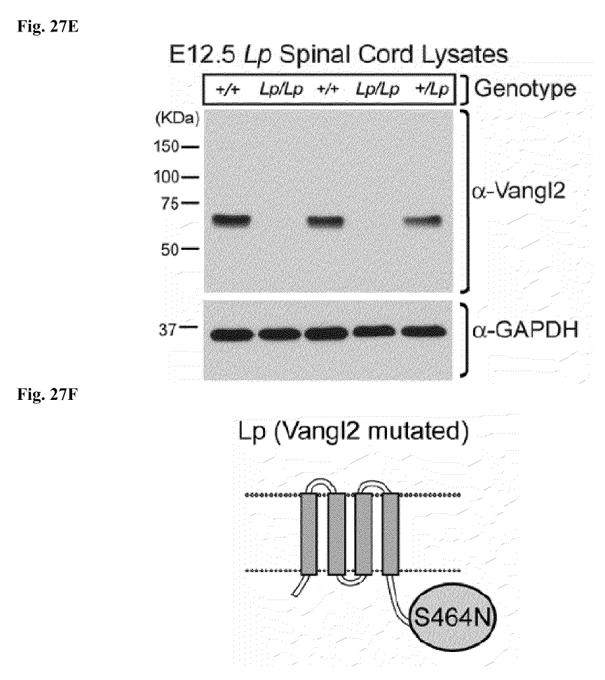

A well-characterized Vgl2 mutant is the looptail with an S464N amino acid substitution, which renders the protein ineffective in Dvl binding (Torban et al., 2004b) (FIG. 27F). Triple transfection of 3×Flag-Lp, Fzd3 and Dvl1 followed by Wnt5a stimulation resulted in a decrease of p-Jun signal as with Dvl1 and Fzd3 co-transfection (FIG. 17C lanes 4-6). When quantified, the presence of Lp protein appears to accelerate the loss of pJun signal, compared to the wildtype Vgl2 protein (FIG. 17D triangle symbol line). Therefore, Vgl2 is able to antagonize Dvl1, where as mutant Vgl2, Lp, fails to. Furthermore, S464 in Vgl2 is a likely phosphorylation site, because Vgl2 was phosphorylated whereas the Lp protein shows no such modification (FIG. 17C, anti-Flag IB, and FIG. 24C).

Dvl and Vgl have antagonistic functions on Fzd3 phosphorylation. To understand how Dvl1 and Vangl2 may antagonize each other, we set out to analyze the protein level of Fzd3 receptor at the cell surface, because Fzd endocytosis has been shown to be required for PCP signaling (Yu et al., 2007). We transfected Fzd3-mCherry in HEK293T cells then surface biotinylated and immunoprecipitated (IP) the surface molecules with strepavidin-conjugated beads to obtain membrane-localized proteins. These surface fractions were then analyzed on immunoblots (IB), and by anti-mCherry IB, and two different bands for Fzd3-mCherry were observed at the cell membrane (FIG. 17A, Avidin IP, lanes 2-4). We tested whether these two Fzd3 bands represent phosphorylation states of the Frizzled3 protein, one being phosphorylated/hyperphosphorylated Fzd3 (p-Fzd3) and the other non-phosphorylated Fzd3. When the membrane fractions were subjected to protein phosphatase I treatment the upper Fzd3-mCherry band disappeared suggesting the band shift indeed represents phosphorylation or hyperphosphorylation of the Fzd3 protein (FIG. 17E). Phosphorylation of Fzds has been shown previously in Xenopus oocytes

and the Drosophila eye (Djiane et al., 2005) (Yanfeng et al., 2006); furthermore, in Xenopus embryos phosphorylation of XFzd3 is XDsh dependent. We found when Fzd3-mCherry was co-transfected with Dvl1-EGFP, the total Fzd3 phosphorylation was indeed increased as previously reported and interestingly only the p-Fzd3 band was present at the membrane when Dvl1 was over-expressed (FIG. 17A, Avidin IP, lanes 5-7). To verify that only the surface-biotinylated proteins were precipitated, we performed a no-biotin membrane label control and found that Fzd3-mCherry was absent, suggesting the IP was specific to surface biotin-labeled proteins (FIG. 17A, Avidin IP, lane 1). The membrane IP faction showed an absence of the cytosolic protein GAPDH though the proteins were present in the total cell lysate input (FIG. 17A, anti-GAPDH IB).

We next asked whether Vgl2 has any effect on the Dvl1-induced phosphorylation of Fzd3 at the cell membrane. We triple transfected Fzd3-mCherry, Dvl1-EGFP and 3×Flag-Vgl2 and found that co-expression with Vgl2 resulted in a re-appearance of the non-phosphorylated Fzd3 band at the membrane (FIG. 17C, Avidin IP and anti-mCherry IB, lanes 1-3). Therefore Vangl2 either prevented Dvl1-dependent phosphorylation of Fzd3 at the membrane or promoted the dephosphorylation of membrane bound Fzd3 that initially resulted from Dvl1 over-expression. In either case, the presence of Vgl2 promotes a reduction of p-Fzd3 at the membrane. Interestingly, when Vgl2 was transfected with Fzd3 in the absence of Dv11, only the non-phospho-Fzd3 was present at the membrane (FIG. 24D, lanes 1-3), further supporting that notion that Vgl2 promotes the non-phosphorylated form of Fzd3 at the cell surface. When we triple transfected the mutant Vgl2, 3×Flag-Lp, with Fzd3-mCherry and Dvl1-EGFP, we did not observe the non-phosphorylated band of Fzd3-mCherry, suggesting that the mutant Vgl2 protein, Lp, cannot antagonize the phosphorylation of Fzd3 induced by Dvl over-expression (FIG. 17C lanes 4-6). In addition, 3×-Flag-Lp and Fzd3-mCherry transfection in the absence of Dvl1 resulted in the same Fzd3 banding patterns as Fzd3-mCherry alone, suggesting that Lp cannot change the phosphorylation state of Fzd3 at the membrane (FIG. 24D, lanes 4-6).

To verify that Dvl1 induces Fzd3 hyperphosphorylation, we mutated a potential conserved phosphorylation site in the Fzd3 cytoplasmic domain, a serine at amino acid 577 to alanine (S577A) and found that Dvl1 induced Fzd3 phosphorylation is indeed affected by this mutation as previously observed in Xenopus oocytes (Yanfeng et al., 2006). When Fzd3-HA is cotransfected with Dvl1-EGFP, we observed a Fzd3 phospho shift in our total cell lysates (FIG. 17F, anti-HA IB, lane 3). However, when our mutant Fzd3, Fzd3(S577A) was co expressed with Dvl1-EGFP, the phospho shift that is indicative of hyperphosphorylation disappeared (FIG. 17F, anti-HA, lane 5). Interestingly, the hyperphosphorylation of Fzd3 was promoted by G-protein receptor kinase 2, GRK2 (FIG. 17F, lane 9). GRK2 is responsible for the phosphorylation of Smoothened (Chen et al., 2004) (Philipp et al., 2008), a Frizzled-like receptor, and here we demonstrate for the first time that GRK2 is the kinase that promoted the Dvl1-dependent phosphorylation of the Fzd3 receptor.

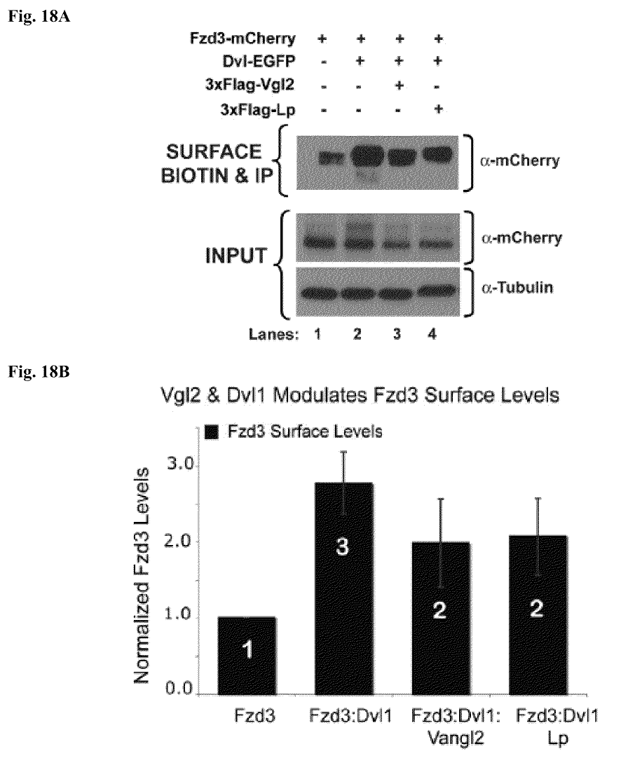

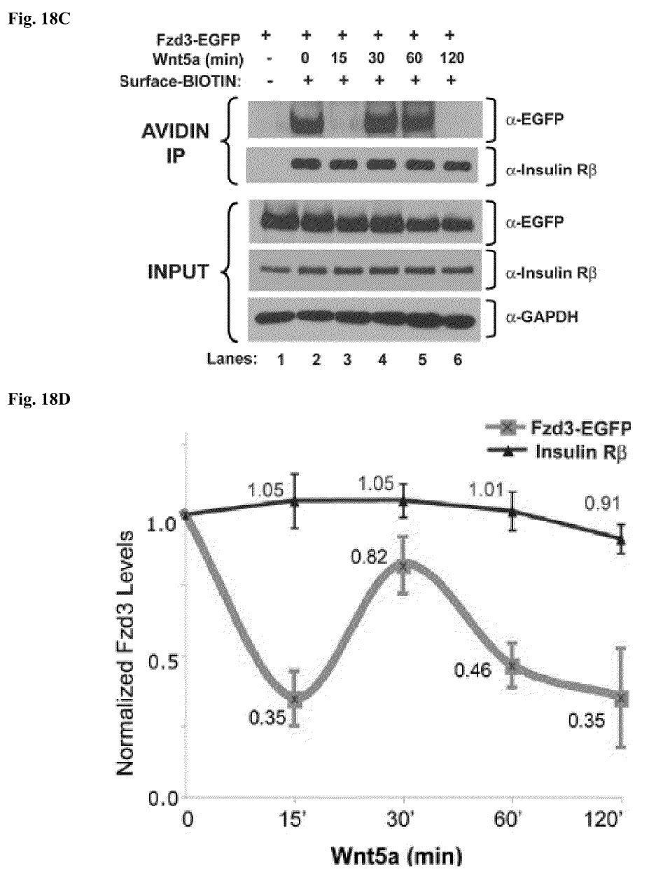

Dvl and Vgl have antagonistic functions on Fzd3 membrane localization. Our results also showed that Dvl1 and Vangl2 affect the total levels of Fzd3 at the membrane. In the presence of Dvl, the surface IP showed approximately 3 fold more Fzd3 at the membrane then Fzd3-mCherry transfected alone (FIG. 18A lanes 1-2, FIG. 18B). However, when Vgl2 was present, Fzd3 surface levels were reduced to a 2-fold increase, suggesting that the Vgl2 promotes Fzd3 internalization.

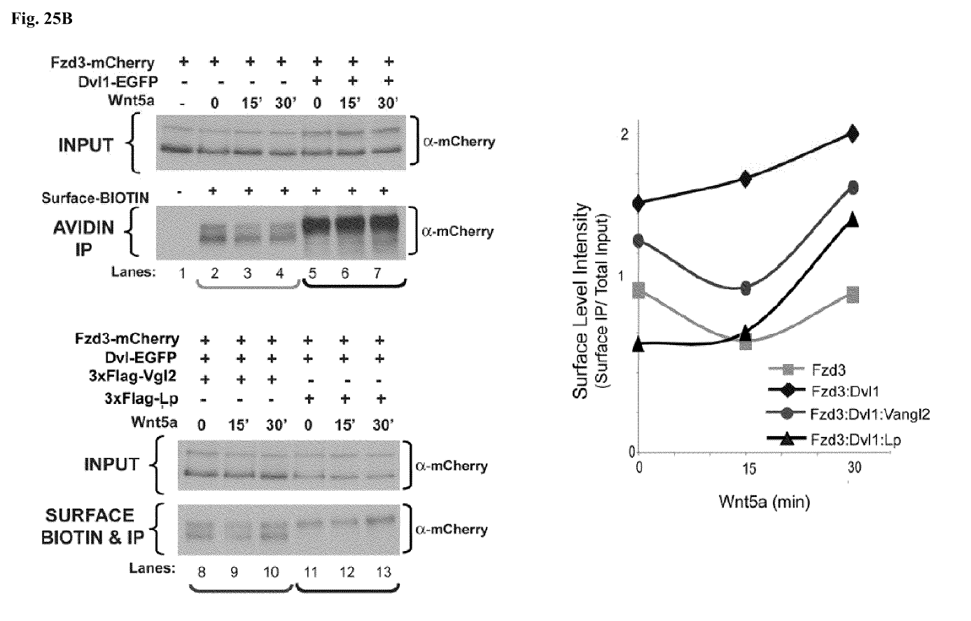

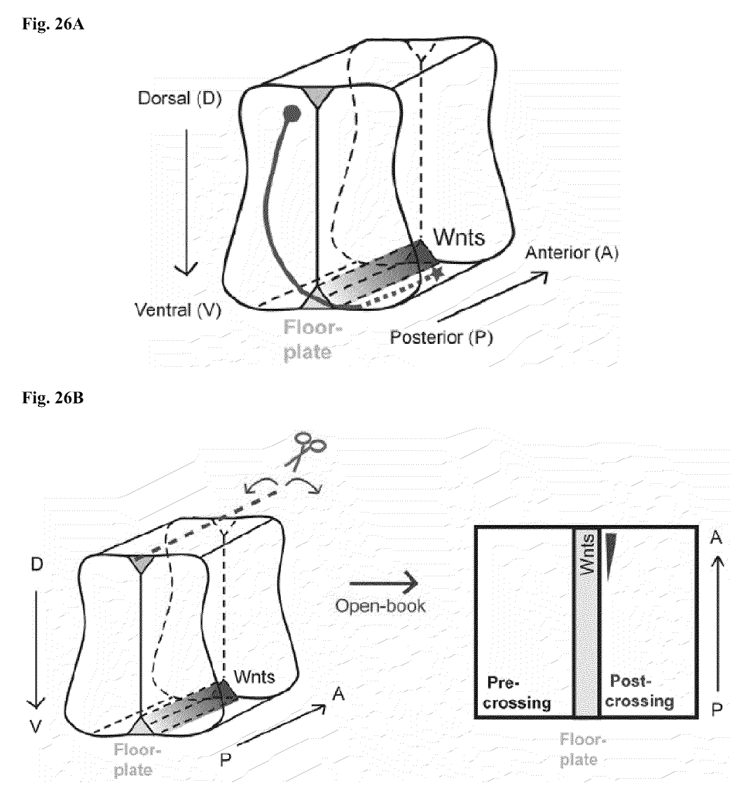

It has been proposed that Frizzled internalization is necessary for PCP signaling and phenotypes (Gagliardi et al., 2008; Yu et al., 2007). However, it has never been directly tested whether the cell surface level of Fzd, the Wnt-PCP receptor, changes upon Wnt addition. By using our surface biotinylation and immunoprecipitation with avidin, we show here, 15 minutes after Wnt addition, cell surface level of Fzd3-EGFP is diminished (FIG. 18C, Avidin IP, lane 3 anti-EGFP IB). Moreover, we found that 15 additional minutes after Wnt addition, Fzd3-EGFP levels increased again on the cell surface (FIG. 18C, Avidin IP, lane 4 anti-EGFP IB) and after two hours we found Fzd3 levels reduces once again from the membrane fraction (FIG. 18C, Avidin IP, lane 6 anti-EGFP IB). This Fzd3 receptor oscillation from and to the membrane appeared to be a Wnt-Fzd specific effect because a non-Wnt binding receptor, such as the Insulin receptor stayed the same throughout the Wnt stimulation (FIG. 18C, Avidin IP, anti-Insulin Rβ IB). The Wnt-dependent Fzd3 oscillation is quantified in FIG. 18D (cross symboled line). However, we found that the time of Fzd3-EGFP internalization is dependent on the Wnt source and the concentration of Wnts (FIG. 25A), but all treatments with Wnts resulted in the oscillation of Fzd3 on the plasma membrane. In addition, PCP components appeared to effect this Wnt dependent oscillation of Fzd3 to and from the membrane. When Dvl1 is over expressed with Fzd3, we found within 30 minutes Fzd3 is unable to be internalized (FIG. 25B, lanes 5-7 and line). However, upon the introduction of Vangl2 into the transfection mix, Fzd3 oscillation is resumed (FIG. 25B, lanes 8-10 and circle symboled line). This data suggest that Dvl1 and Vangl2 have opposing functions on Wnt-dependent Fzd3 internalization as well as phosphorylation

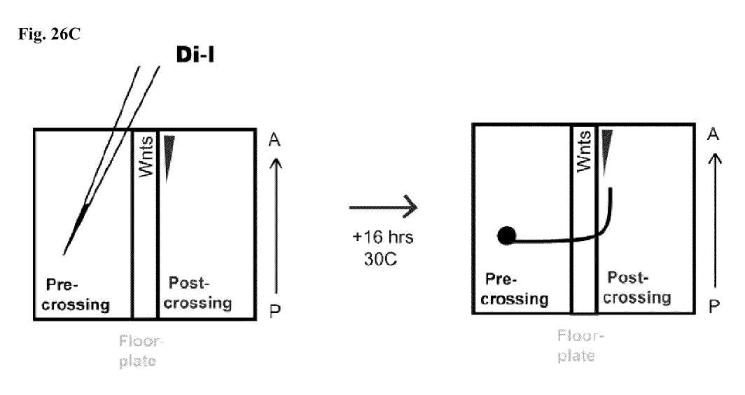

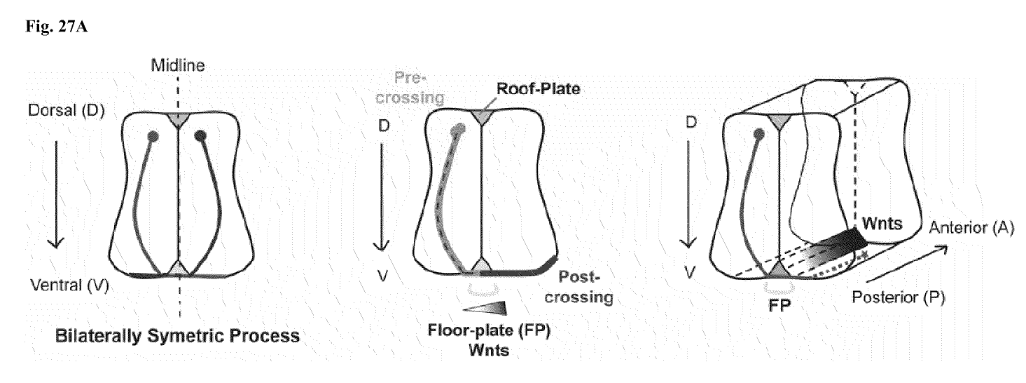

PCP components are expressed on commissural axons. During development commissural axons make a series of known changes in trajectory en route to the brain, commissural axons are first guided along the dorso-ventral (D-V) plane of spinal cord then they turn anteriorly toward the brain following a Wnt protein gradient secreted from the floor plate of the spinal cord (FIG. 26A). Commissural axons become Wnt responsive when emerging from midline of spinal cord and subsequent turning and growth along the anterior posterior (A-P) axis is a Wnt-Fzd dependent process (Lyuksyutova et al., 2003) (Wolf et al., 2008).

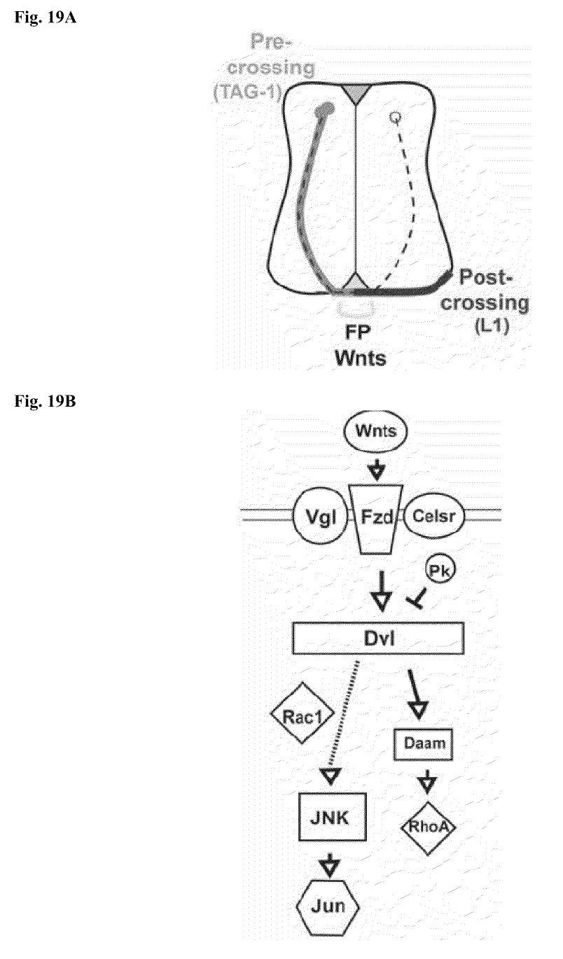

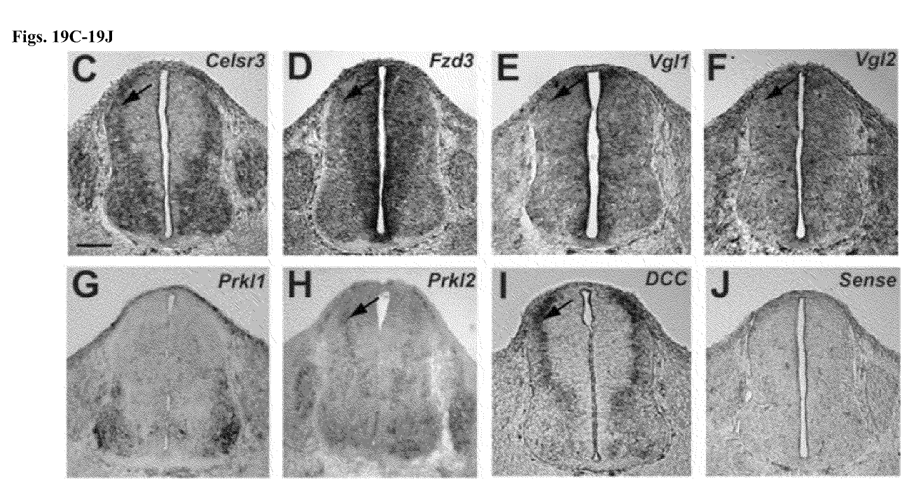

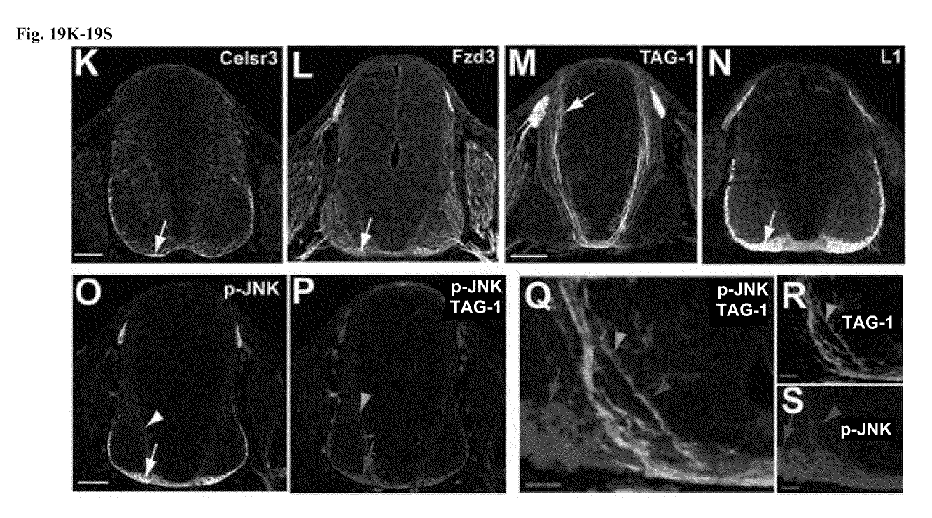

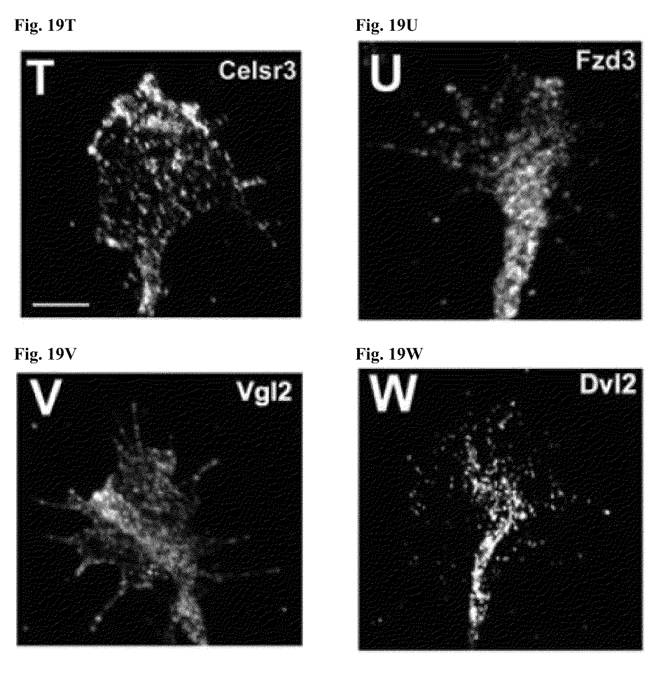

To test whether the Wnt-PCP pathway is involved in anterior-posterior guidance of commissural axons, we first analyzed the expression patterns of the core PCP components in the developing spinal cord using in situ hybridization (The PCP pathway with its components are listed in the schematic in FIG. 19B). We found that all core PCP components (FIGS. 19C-19H) are expressed in commissural neurons at mouse E11.5, a time when many axons are turning anteriorly. Celsr1 and Celsr2 were expressed in the ventricular zone whereas Celsr3 transcripts were found selectively in the post-mitotic mantle zone of the spinal cord (FIG. 19C) and were particularly abundant in the areas encompassing commissural neuron cell bodies and regions expressing the Netrin-1 receptor DCC (FIG. 19I), a marker for commissural neurons (Keino-Masu et al., 1996). As previously reported, Fzd3 mRNA was broadly expressed in the spinal cord, including the mantle zone where commissural neuron cell bodies reside (FIG. 19D) (Lyuksyutova et al., 2003). Both Vgl1 and Vgl2 were also expressed broadly in the spinal cord (FIGS. 19E-19F). Prkl2 expression was observed in the dorsal commissural neurons as well as in the ventral spinal cord (FIG. 19H), whereas Prkl1 was expressed primarily in the ventro-lateral regions of the spinal cord. The Dsh genes were widely expressed in the central nervous system, as previously reported (Tissir and Goffinet, 2006).

To characterize the expression patterns of the core PCP proteins in the spinal cord, we performed immunohistochemistry on mouse E11.5 spinal sections. Commissural axons have a pre-crossing and a post-crossing segment (segments in FIG. 19A, respectively) and a short crossing segment through the floor-plate (FP). Since, commissural axon guidance is a bi-laterally symmetric process (FIG. 27A), the pre- and post-crossing segments occur on both sides of the spinal cord. TAG-1 is expressed on the pre-crossing and crossing segments in the spinal cord (FIG. 19M) (Dodd et al., 1988) and L1 delineates post-crossing axons or growth cones (FIG. 19N) (Zou et al., 2000). Celsr3 is broadly expressed in the spinal cord but enriched in the post-crossing segment (FIG. 19K), along with Fzd3 protein (FIG. 19L). We tested the specificity of these post crossing enrichments by immunohistochemistry on E11.5 spinal sections wildtype and knockout embryos and the post-crossing staining of the antibodies is diminished in Celsr3 and Fzd3 homozygous mutants (FIG. 27B and FIG. 27C, respectively). Vgl2 protein has previously been shown to also be enriched in the post crossing axons of the spinal cord (Torban et al., 2007).

JNK (FIG. 19B) is a downstream signaling component of PCP and PCP signaling activation is often measured by increased phosphorylation of JNK and/or Jun (Boutros et al., 1998). We found that the phosphorylated-JNK is present on commissural axons, as shown by co-immunoreactivity with TAG-1 (FIGS. 19P-19S, short arrow heads), and is enriched in the post-crossing segment of the E11.5 spinal cord (FIG. 19O and FIG. 195, long arrows) in a similar manner to Vgl2, Celsr3 and Fzd3 protein expression. In addition, to test whether PCP signaling components are present in axonal growth cones (the motile sensing center for the neuron), we analyzed their distribution in dissociated commissural neuron cultures. We cultured dissociated commissural-specific neurons using a previously established method (Augsburger et al., 1999). We found that Celsr3, Fzd3, Vgl2 and Dvl are all present in dorsal commissural neurons and their growth cones after 24 hours of culture (FIGS. 19T-19W, respectively). Taken together, PCP components are expressed in the developing spinal cord in the right spatio-temporal pattern necessary to regulate anterior-posterior guidance of commissural axons.

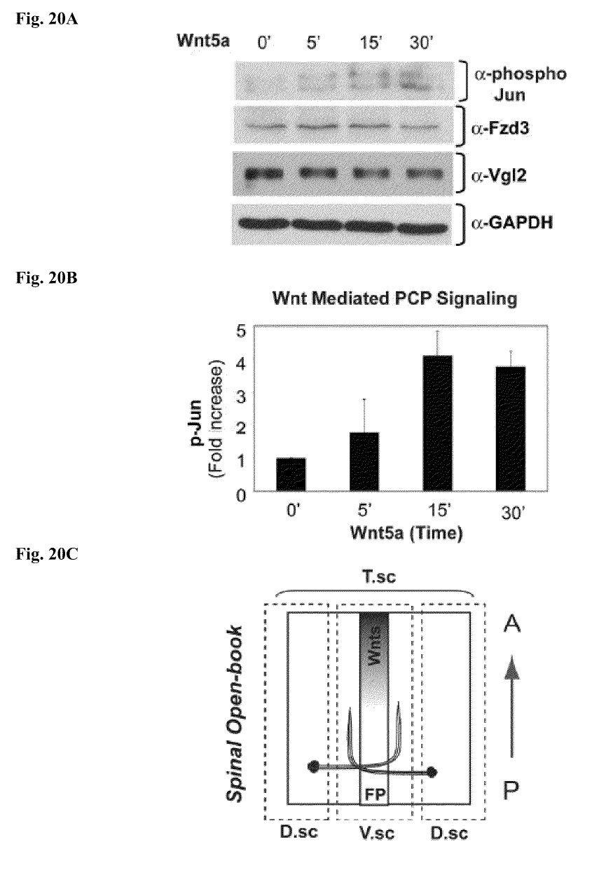

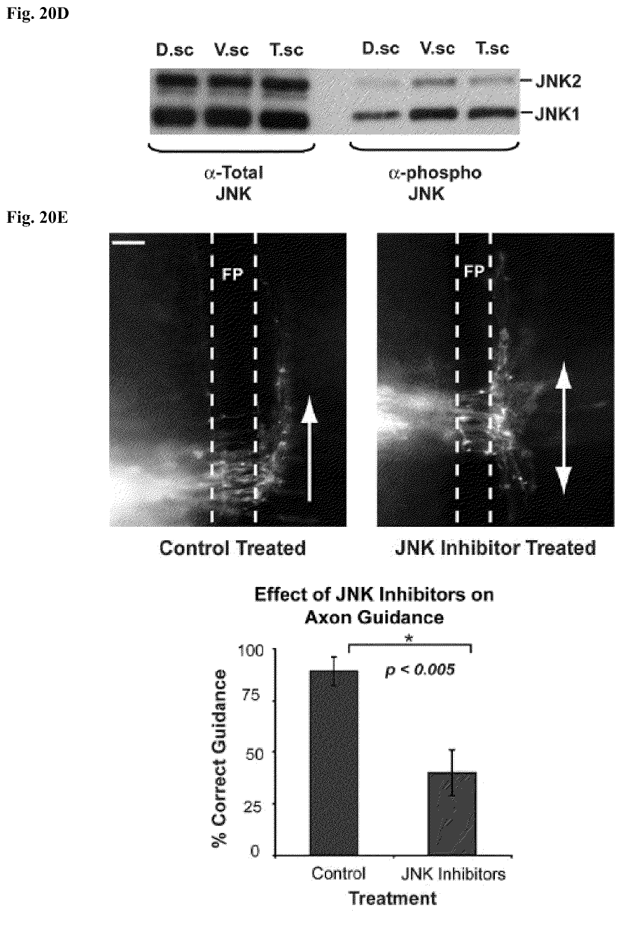

JNK is activated by Wnt5a in commissural axons and required for their A-P guidance. Several Wnt genes are expressed in an anterior-posterior gradient in or around the floor plate of the spinal cord. However, the mechanisms of how Wnt signaling mediates axon attraction and growth have yet to be elucidated. We tested whether JNK signaling is activated in commissural neurons via Wnt5a stimulation by measuring the levels of phospho Jun (FIG. 20A) and also examined whether JNK is required for A-P guidance of commissural axons. Commissural neurons were cultured for 36 hrs and subject to bath application of Wnt5a. After 15 and 30 minutes treatment, we found that endogenous phospho-Jun levels were increased (FIG. 20A), with a 4-fold increase within 15 minutes (FIG. 20B). Furthermore, endogenous Fzd3 and Vgl2 proteins can be detected in rat E13 commissural neuron lysates after 36 hours in culture (anti-Fzd3 and anti-Vgl2 IB, FIG. 20A). The specificity of Fzd3 and Vgl2 antibodies for western blot analysis was validated using knock and mutant mouse line (FIG. 27D and FIG. 27E).

Next, to determine whether inhibition of JNK activity is important to axon guidance in vivo, we carried out the spinal “open-book” assay and DiI injection (FIGS. 26B-26C). Phosphorylation Jun and JNK are a classic readout of JNK activation (Boutros et al., 1998; Jaeschke et al., 2006), and we found that phospho-JNK appeared highly enriched in post-crossing regions of the commissural axons in vivo (FIG. 19O). Due to the high levels of redundancy and their importance for many processes in early development, analyzing knockout mice of JNK gene families to test whether JNK activity is required for anterior-posterior (A-P) guidance of commissural axons was not feasible. Therefore, we applied JNK inhibitors JNKI-1 and SP600125 in the spinal “open-book” explant assay at a time when commissural axons are making their anterior turning decisions, rat E13 (FIG. 20E). These inhibitors block all three specific JNKs (Jun N-terminal Kinases 1-3) that are found in vertebrates.

First, we extracted lysates from the E13 rat spinal cord to characterize the levels of activated JNK in the ventral spinal cord (FIGS. 20C-20D). Levels of both activated JNK1 and JNK2 were indeed much higher in the ventral spinal cord (V.sc) as compared to the dorsal spinal cord (D.sc), (phospho-JNK IB, FIG. 20D), though no difference in total JNK was observed (Total JNK IB, FIG. 20D). More importantly, inhibiting JNK activity with the aforementioned inhibitors lead to anterior-posterior (A-P) randomization and misguidance of commissural axons. We analyzed the post-crossing trajectory of commissural axons in “open-book” explants treated with JNK inhibitors by DiI injection at rat E13 following one day culture. Control commissural axons turn and grow anteriorly toward the brain after crossing the floor-plate (FP) (FIG. 26A and FIG. 26C). And approximately 91% (±SEM 5.57%) of DiI injected control axons showed correct guidance as compared to 39% (±SEM 6.39%) of JNK-inhibited axons (FIG. 20E). About ⅔ of the JNK inhibitor treated axons showed randomize direction. Therefore, a downstream effector of PCP signaling, JNK, is required for the A-P guidance of commissural axons.

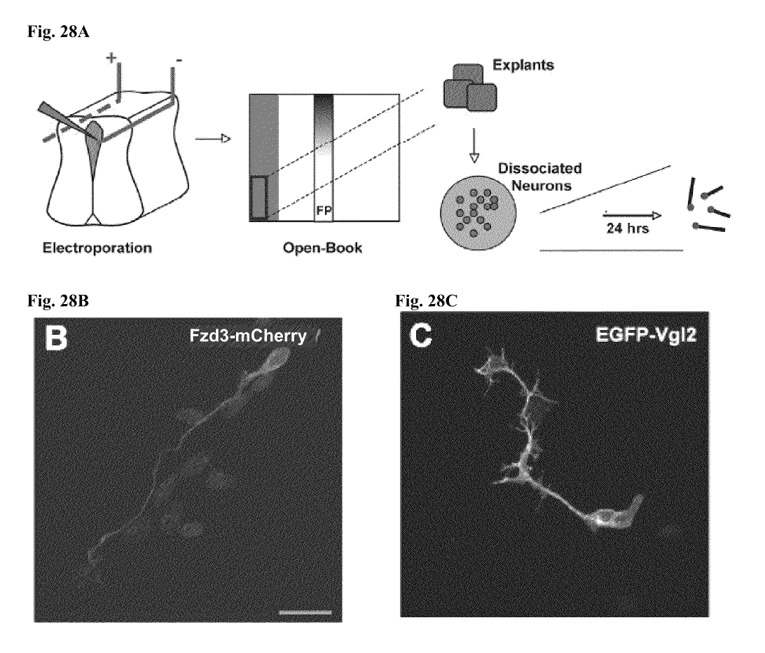

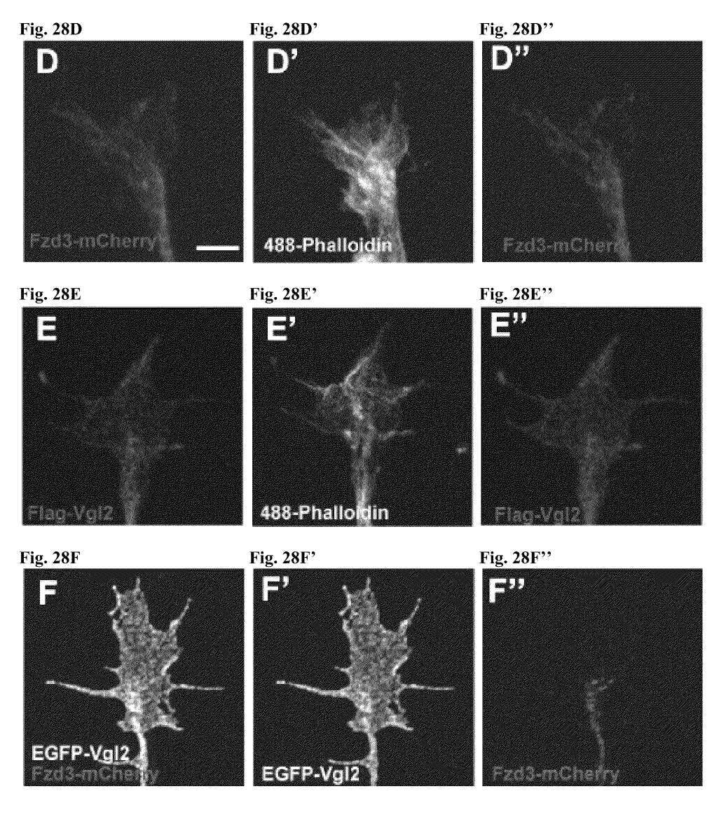

Fzd3 mediates Wnt-stimulated commissural axon elongation. To directly test the function of PCP components in Wnt-mediated outgrowth of commissural axons, we electroporated DNA constructs to express PCP components in dissociated commissural neurons. The spinal cord of E11.5 mouse or E13 rat contains a ventricle into which plasmid DNA can be introduced (FIG. 28A). After injecting DNA into the central canal and electroporation, the spinal cord was dissected into an “open-book” configuration and then the dorsal spinal cord margin including the progenitor domains was dissociated as previously described ((Augsburger et al., 1999), FIG. 28A). More than 90% of the electroporated and dissociated neurons were TAG-1 immuno-reactive, confirming that they are commissural neurons (Wolf et al., 2008).

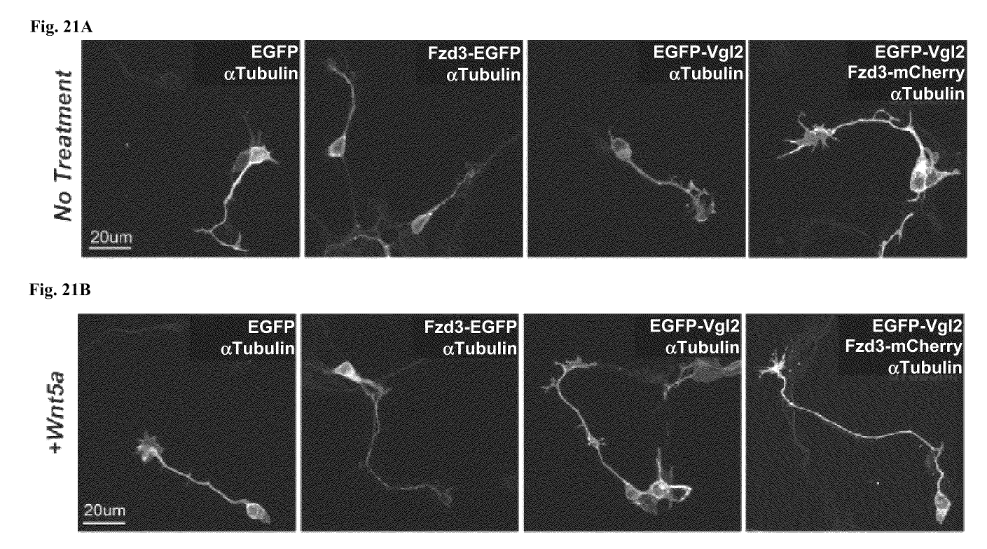

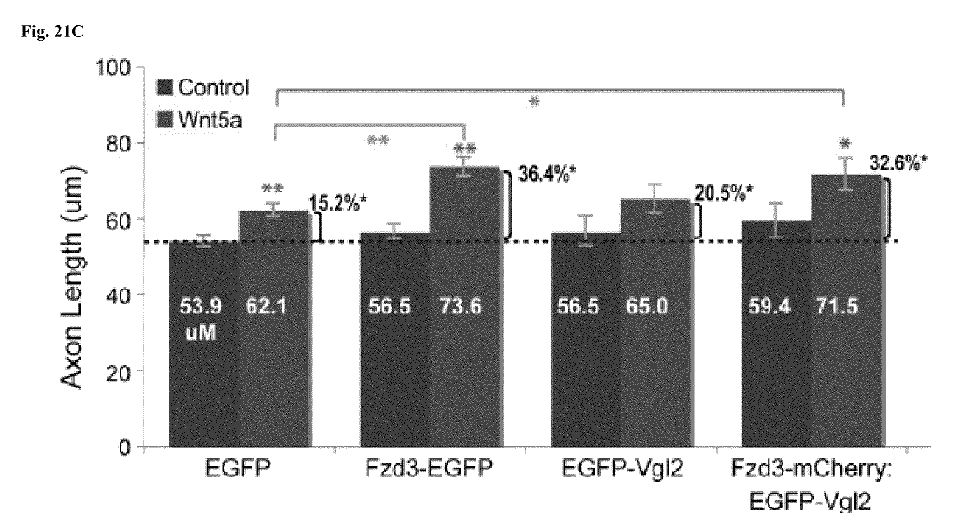

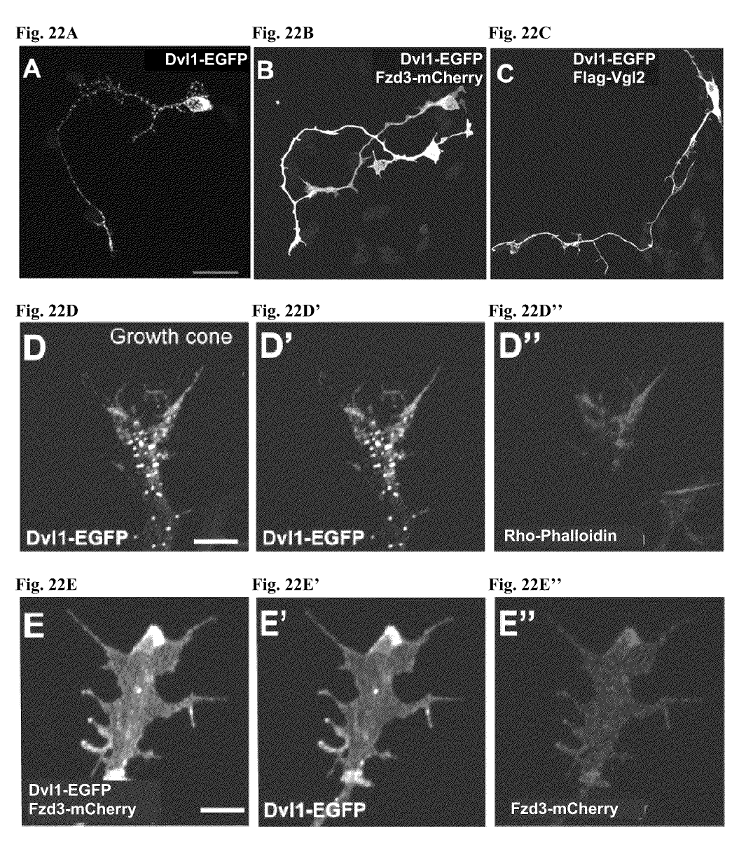

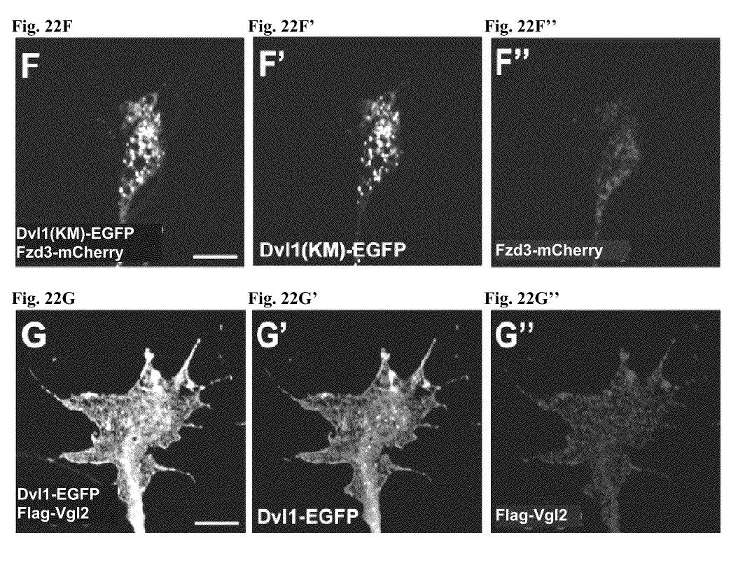

Fzd3 and Vgl2 over-expression in pre-crossing neurons did not affect the growth of commissural axons compared to controls (FIG. 21A and FIG. 21C), and an average axon length of approximately 55-60 um was observed after 24 hrs or culture (FIG. 21C). Similarly, co-expression of both Fzd3 and Vgl2 showed no effect on axon length (FIG. 21A last panel). However, when we cultured the commissural neurons on Wnt5a-coated coverslips, we found that the electroporated over-expression of the Wnt-receptor, Fzd3-EGFP, enhanced axon length by ˜36% within 24 hours (FIG. 21B second panel, FIG. 21C). Neurons co-expressing Fzd3 and Vgl2 also showed a similar increase in average axon length in the presence of Wnt5a (FIG. 21B last panel, FIG. 21C). Control neurons expressing EGFP exhibited a smaller, but still significant increase of ˜15% in axon growth in response to Wnt5a (FIG. 21B, first panel and FIG. 21C). This may be due the presence of low endogenous levels of PCP components in these axons following 24 hours of culture (FIGS. 20A-20E). EGFP-Vgl2 expression alone, in the absence of Fzd3, did not cause a statistically significant increase by Wnts compared to no EGFP-Vgl2 expression within 24 hrs. Therefore, our culture results show additional evidence that Fzd3 mediates Wnt response in commissural axons.

Both Fzd3 and Vgl2 can localize Dvl1 to plasma membrane in commissural axon growth cones. We then analyzed the subcellular localization of PCP components in commissural axon growth cones. It is known that both Fzd3 and Vgl2 can both bind to Dvls (Wong et al., 2003); (Torban et al., 2004a). Because Fzd3 is a cell surface Wnt-binding receptor it may recruit or stabilize Dvl at the growth cone plasma membrane. To test whether Fzd3 affects Dvl1 localization we co-expressed both Fzd3-mCherry and Dvl1-EGFP in dissociated commissural neurons (FIG. 28A). Dvl1-EGFP expression alone in commissural neurons resulted in a punctate vesicular pattern along the neurite and in the growth cone (FIG. 22A, FIGS. 22D-22D″). However, upon co-expression with Fzd3-mCherry, the punctate aggregates completely dispersed and translocated to the membrane of the commissural axon (FIG. 22B and FIG. 22E). This finding is similar to previous studies in other cellular contexts where Dvl was found recruited to the membrane by Fzds in the drosophila epithelia (Axelrod et al., 1998). More importantly membrane localization of Dvl has been shown to be critical and specific to activation of PCP signaling (Axelrod et al., 1998) (Park et al., 2005), and our data shows that Fzd3 co-expression with Dvl1 results in the membrane localization of Dvl in our commissural neuron growth cone (FIG. 22E′), which suggest a protein localization consistent with the activation of PCP signaling.

Interestingly, co-expression also resulted in Fzd3-mCherry stabilizing at the plasma membrane. Fzd3-mCherry normally localizes both to the plasma membrane and cytoplasmic compartments in the commissural growth cone (FIG. 28B, FIGS. 28D-28D″), similar to the endogenous Fzd3 protein expression in the commissural growth cone. However, Fzd3-mCherry when co-expressed with Dvl1-EGFP, solely localized to the plasma membrane (FIG. 22E and FIG. 22E″). This is consistent with our finding that Dvl1 increases Fzd3 levels on the cell surface (FIG. 17A, lanes 5-7 and FIG. 18A, lane 2). Furthermore, mutant Dvl1 (with DEP domain mutation) is unable to target Fzd3 and itself on the plasma membrane (FIG. 22F″) and promote Fzd3 phosphorylation at the membrane (FIG. 24B, lane 6). This Lysine 438 to a Methionine substitution in Dvl1 (FIG. 24A) has been shown to be essential for PCP signaling and Dvl membrane association (Boutros et al., 1998) (Moriguchi et al., 1999) (Park et al., 2005) (Simons et al., 2009). To test whether the translocation of Fzd3 is indeed dependent on PCP signaling, we expressed the Dvl1(KM)-EGFP with Fzd3-mCherry and asked whether Fzd3 and Dvl1 mutant could be targeted to plasma membrane. Our results showed that Fzd3-mCherry localization in commissural neurons does not change when co-expressed with Dvl1(KM)-EGFP (FIG. 22F″) nor can Fzd3 recruit the DEP domain mutant Dvl1 to plasma membrane (FIG. 22F′). Therefore, our data is consistent with a model that predicts endogenous Fzd3 and Dvl1 may interact in axonal growth cones in a PCP-like fashion, perhaps by stabilizing each other at the plasma membrane and promoting growth cone membrane protrusion towards the source of Wnts

We found that Vgl2 can also target Dvl1 to the plasma membrane in the commissural growth cone (FIG. 22C and FIG. 22G). Vgl1 and Vgl2 have been shown to bind Dvls (Park and Moon, 2002; Suriben et al., 2009; Torban et al., 2004b), we therefore asked if one cell biological consequence would be the translocation of Dvl1 to the plasma membrane. Vgl2 over expression by itself resulted in localization primarily to the plasma membrane in commissural neurons (FIG. 28C, FIGS. 28E-28E″), although Vgl2 localization appeared unaltered when co-expressed with Dvl1 (FIG. 22G″), Dvl1 translocated to the membrane (FIG. 22G′), where it appears to co-localize with Vgl2 in certain membrane compartments (FIG. 22C and FIG. 22G). Therefore, Vgl2 promoted Dvl1 localization to the membrane. Interestingly when Vangl2 and Fzd3 are over expressed, Fzd3 becomes more cytoplasmic and less membraneous in the commissural growth cone (FIGS. 28F-28F″). And because Vangl2 appears to associate with Dvl1 at the plasma membrane, we hypothesize that Vangl2 and Fzd3 may complete for binding to Dvl1, therefore, Vgl2 may antagonize Dvl's promotion of Fzd3 membrane localization by removing Dvl1 from Fzd3.

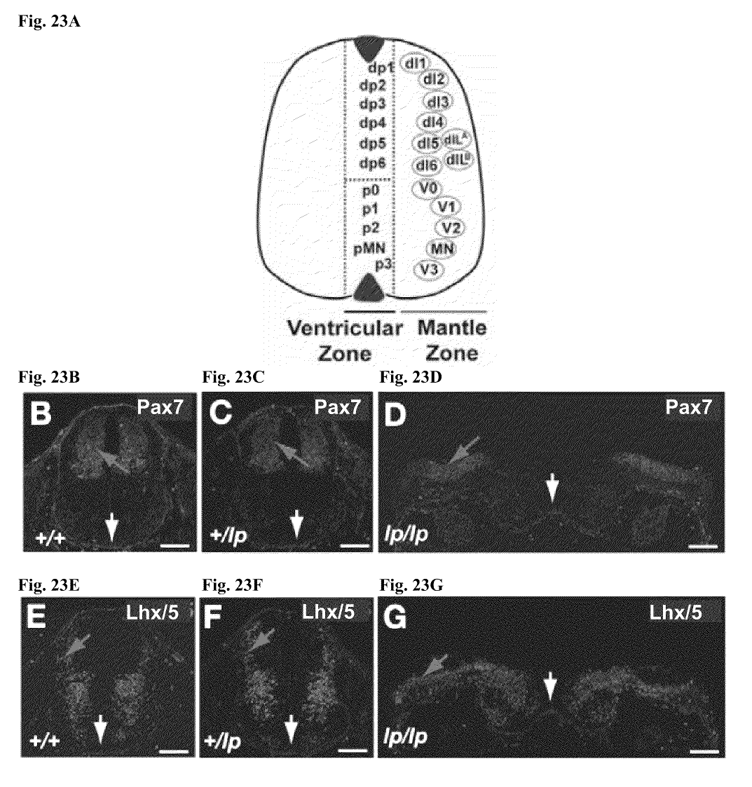

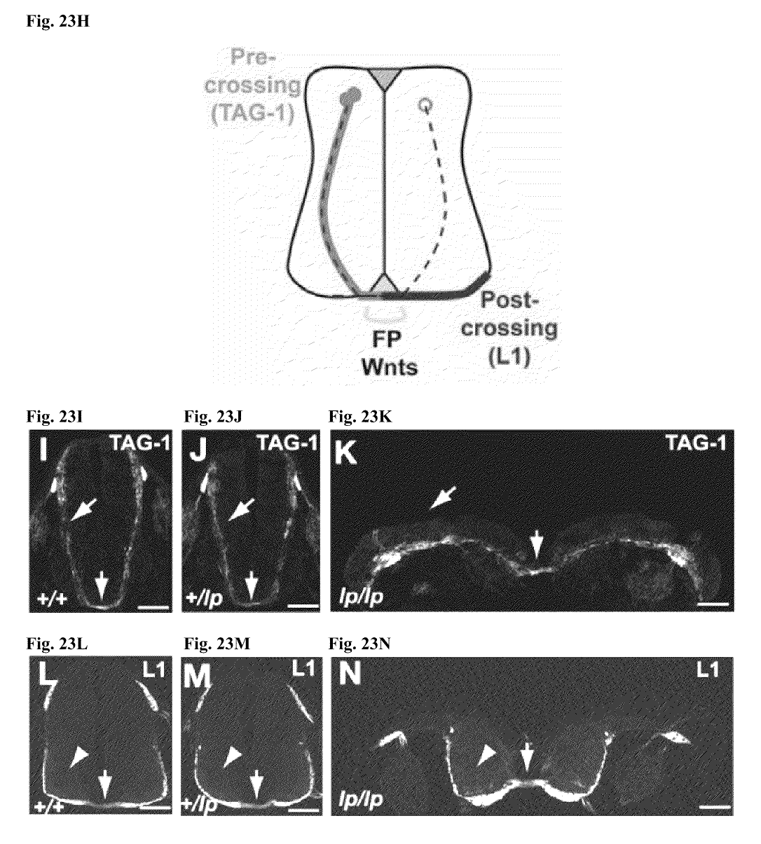

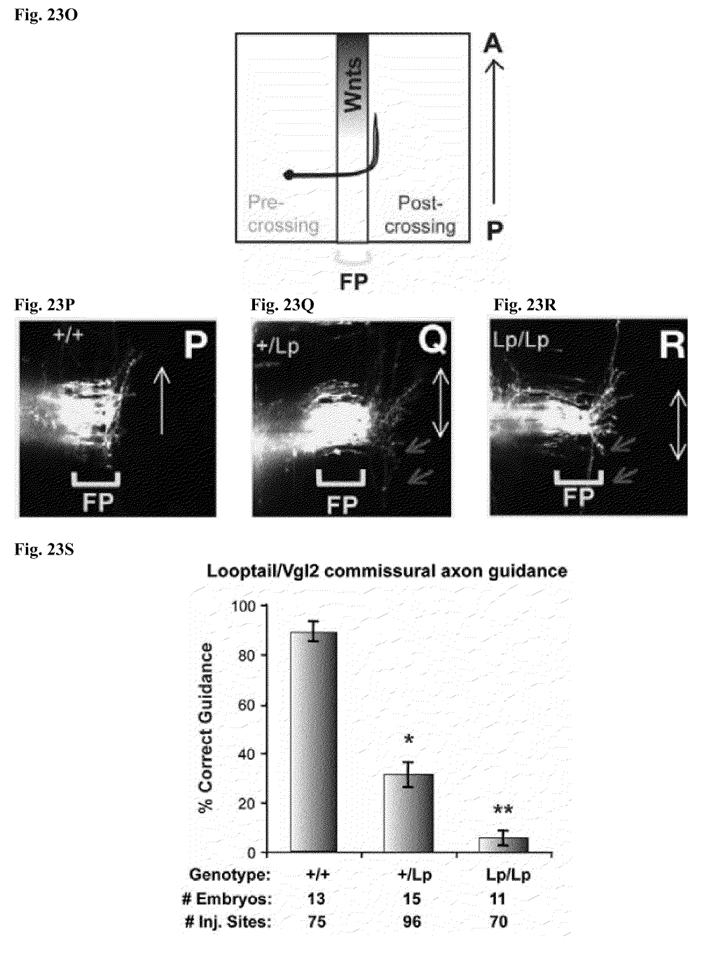

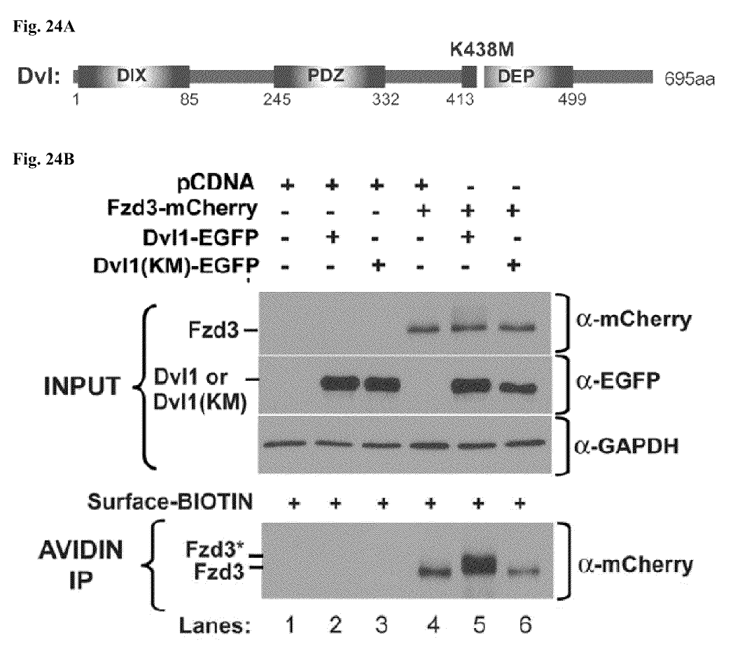

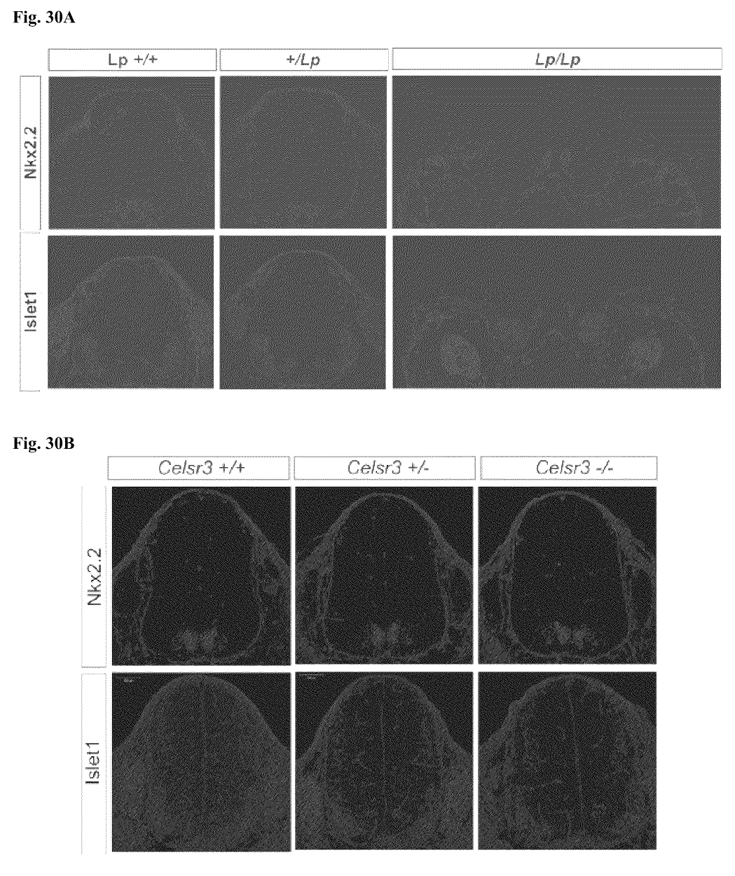

The Looptail embryos display anterior-posterior guidance defects in commissural axon guidance. Previous work showed that Fzd3 is required for proper A-P guidance of post-crossing commissural axons (Lyuksyutova et al., 2003). Fzd3 is a known component in multiple Wnt signaling pathways, including canonica/β-catenin, Wnt-Ca2+, Wnt-PKC, Wnt-P13Kinase and PCP signaling. To address whether PCP signaling is required for A-P guidance of commissural axons in vivo, we analyzed other mouse mutants with a specific deficiency in PCP signaling. A well-known PCP mutant mouse, the loop-tail mouse, has a point mutation of the Vangl2 gene (S464N) that renders the protein ineffective and unstable. To confirm the loss of protein expression, we immunoblotted for Vangl2 protein in spinal lysates from wildtype, heterozygous and Lp/Lp embryos and found that protein levels were absent in the homozygotes (FIG. 27E). However, because the loop-tail mouse displays an open neural tube (caused by secondary convergent extension defects), and because the roof plate is an essential source of morphogens, we examined the cell patterning and fate markers in Lp mutants. A schematic of the dorsal progenitor domains and post-mitotic neurons in the developing spinal cord is shown in FIG. 23A. Pax7, which defines the spinal dorsal progenitor domains dp3, dp4, dp5, dp6 and part of the ventral progenitor domain p0, appeared normal in the +/+, Lp/+, and Lp/Lp embryos (FIGS. 23B-23D). Nkx2.2 immunoreactivity for pMN and p3 domains showed no defects in embryos of all three genotypes (FIG. 30A), and the post-mitotic dorsal interneuron (dI) markers Lhx1/5 for dI2, dI3, dI4 were unaffected by the open neural tube. Finally, the immunoreactivity for Iselet1 showing the dI3 and some motor neuron populations appeared normal (FIG. 30A).

We next examined the trajectory of commissural axons in transverse sections using TAG-1 and L1 staining. The diagram of dorso-ventral (D-V) trajectory of commissural axons is shown in FIG. 23H. TAG-1 immunoreactivity showed that commissural axons projected with no gross defects from the dorsal spinal cord to the ventral midline in the +/+, Lp/+, even in the open neural tube Lp/Lp embryos (FIGS. 23I-23K). Furthermore, after crossing the axons grew within the ventral and lateral funiculis normally, as shown by L1 staining (FIGS. 23L-23N). These results demonstrate that commissural neurons in the developing spinal cord of the Lp mouse project without defect in the dorso-ventral direction, but show severe anterior-posterior guidance defects.

We then analyzed the post-crossing trajectory of commissural axons in the Lp mouse by open-book DiI injection at E11.5, the time when commissural axons are beginning their anterior turn (FIG. 23O). Our analysis and quantification of DiI injections in the spinal open-book prep revealed that while all commissural axons crossed the midline, the post-crossing axons in the Lp/Lp embryos became randomized (FIG. 23R). The heterozygous and homozygous embryos both displayed clear anterior-posterior axon guidance defects (FIG. 23Q, FIG. 23R), as compared to the wildtype littermates, which primarily turned anteriorly at E11.5 (FIG. 23P, quantified in FIG. 23S). Of the 70 DiI injection sites in 11 Lp/Lp embryos, 94.6% (±SEM 2.92) of the labeled axons showed an aberrant trajectory. About half of these axons projected anteriorly (up) and the other half posteriorly (down), suggesting that growth along the longitudinal axis was intact, but up or down directionality was lost. In the 15 heterozygous littermates analyzed, 68.2% (±SEM 5.14) of axons from the 96 injection sites showed aberrance, and only ⅓ of the axons turned in the anterior direction (FIG. 23S).

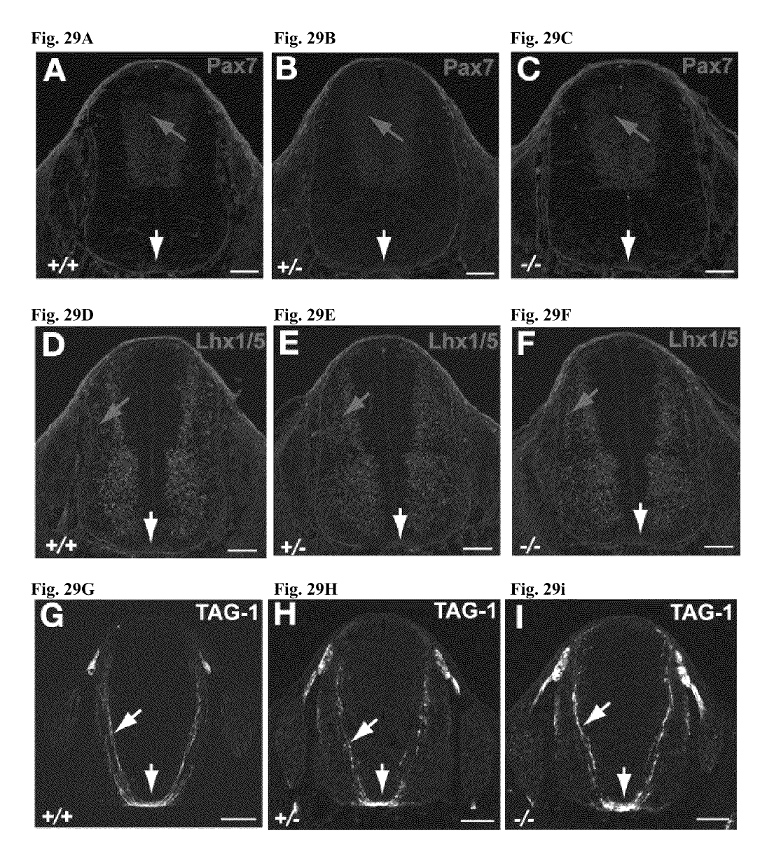

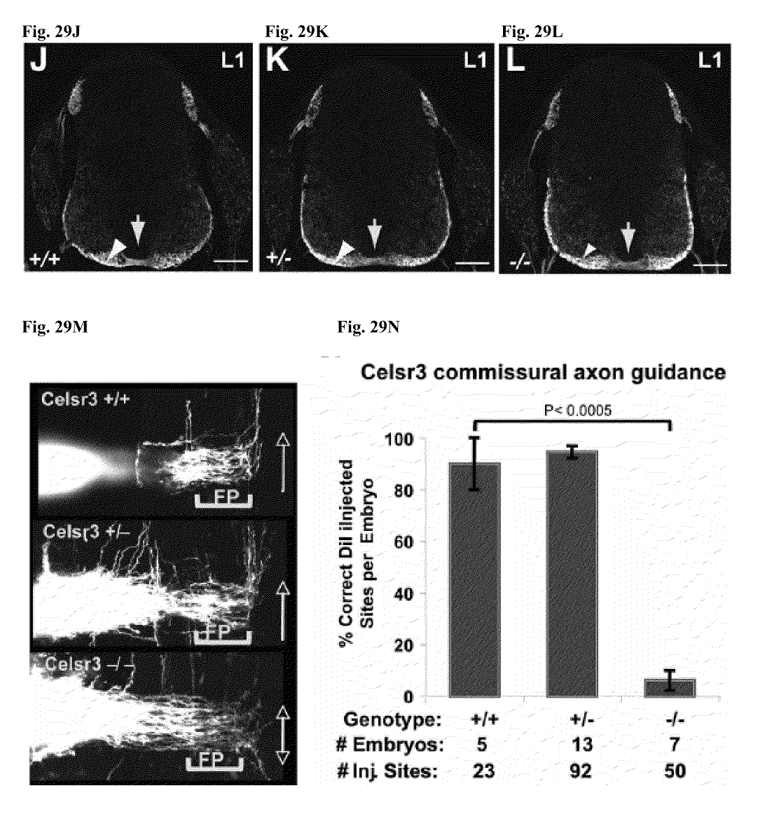

To further demonstrate that the PCP signaling pathway is responsible for proper anterior-posterior guidance of commissural axons, we assessed whether the third transmembrane component of the PCP signaling pathway, Celsr3, was required. Similar to the analysis for the loop-tail mouse, we first verified that the Celsr3 null embryos did not have defects in cell specification or dorso-ventral axon guidance. There were no observable differences in the Pax7 and Nkx2.2-positive spinal progenitor pools in the mutants, and expression of the post-mitotic markers Lhx1/5 and Islet1 were comparable to the Celsr3 wild type (FIGS. 29A-29F, FIG. 30B). TAG-1 and L1 immunoreactivity suggested normal dorso-ventral commissural axon trajectory, and no differences were observed in the homozygous Celsr3 null embryos as compared to the wildtype or heterozygotes (FIGS. 29G-29L). However, Celsr3 null embryos at E11.5 showed severe defects in anterior-posterior commissural axon guidance, while the wildtype and heterozygous littermates remained normal. Examination of open-book DiI injections in Celsr3 +/+ and Celsr3+/− embryos at E11.5 showed that axons turned in the correct anterior orientation, 90.0% (±SEM 10.0%) and 94.6% (±SEM 2.34%) of injection sites, respectively (FIG. 29M top and middle panels respectively, FIG. 29N). However, axons in the Celsr3−/− embryos that were randomized such that only 6.00% (±SEM 3.88%) of DiI injection sites were classified as normal. In 50 injection sites and 7 embryos, 94% of axons displayed defects in anterior (up)-posterior (down) direction (FIG. 29M lower panel and FIG. 29N).

Taken together, we have shown a requirement for Vangl2 and Celsr3 in the anterior-posterior (A-P) guidance of commissural axons. The post-crossing A-P randomization phenotypes were similar to those of the Fzd3 null mice, which indicating that all three membranous components of the PCP pathway are required for proper Wnt mediated commissural axon guidance.

### Discussion

Our study reveals a novel cell-autonomous mechanism of how Vgl and Dvl exert antagonistic biochemical and cell biological functions in the same cell during the establishment of cell polarity. Dvl1 promotes Fzd3 phosphorylation and increases Fzd3 cell surface level and Vangl2 reduces Fzd3 phosphorylation and reduces Fzd3 cell surface level probably by increasing Fzd3 endocytosis, which is a requirement for PCP signaling. Furthermore, this antagonistic interaction appears to operate in growth cones during axon guidance and is likely the underlying mechanism of how PCP signaling regulates axon growth, turning, as well as general mechanism for PCP.

Cell autonomous interactions of Vgl2 and Dvl1 in PCP signalingPCP signaling is a potent cell signaling system, which regulates polarized tissue morphogenesis and directed cell movement, including growth cone guidance. In recent years, progress has been made on non-cell autonomous mechanisms, which involve cell-cell interactions. For example, Flamingo has been shown to bind to Van Gogh in a neighboring cell via the extracellular domain as well as Fzds, helping to establish an asymmetry (Chen et al., 2008) (Devenport and Fuchs, 2008). The cell-autonomous mechanisms, such as how Vgl and Dvl perform antagonistic functions to promote PCP signaling remains elusive. Cell autonomous mechanisms are clearly important regardless of whether they are upstream or downstream of non-cell autonomous events because asymmetric signaling activities within the cell are an essential part of the cell polarity signaling. Growth cone is a highly motile structure, distinct from the non-mobile epithelial cells, where adherence junction mediates cell-cell interactions. Therefore, the cell autonomous mechanisms are likely more robust and important. This novel negative feedback loop mediated by Dvl1 may be a general mechanism in PCP signaling. Vgl on the other hand may promote PCP by locally antagonizing the function of Dvl and by promoting Fzd internalization. This provides a rational to why Vgl and Fzd are usually not colocalized and are often found in opposite sides of cells. In fact, in the Drosophila wing epithelial cells, Fzd is absent on the proximal side of the cell where Van Gogh is. This also implies that the promixal side of the cell may have some type of active PCP signal as well as the distal side may have a different type of PCP signal where Dvl is localized. Furthermore, if Flm recruits Van Gogh to the proximal membrane from the neighboring cell, Fzd is excluded from the proximal membrane because of Van Gogh's function of promoting endocytosis. This may potentially explain how cell-cell interaction is translated into intrinsic asymmetry, which helps to further increase the fidelity of planar cell polarity signaling. This finding also provide clues to how polarity signaling components may interact with each other in motile growth cones to impart signaling asymmetry.

Fzd3 phosphorylation and endocytosis. Although Fzd has been shown phosphorylated, how Fzd phosphorylation affected PCP signaling has not been elucidated (Djiane et al., 2005) (Yanfeng et al., 2006). We show here for the first time that hyperphosphorylated Fzd3 is enriched in plasma membrane and hypo-or non-phosphorylated Fzd3 is internalized. In addition, we identified a critical amino acid Ser 577, which is necessary for hyperphosphorylation and showed for the first time that GRK2 promotes Fzd3 hyperphosphorylation. Fzd3 can be phosphorylated on at least 6-7 amino acids and our results also demonstrated that Fzd3 is indeed phosphorylated on multiple sites. Different phosphorylation sites may potentially result in distinct aspect of Fzd3 regulation, including internalization, subcellular targeting, activation/inactivation, interaction with other signaling components and stability/degradation. However, our results suggest that at least the plasma membrane bound form of Fzd3 tend to be hyperphosphorylated and dephosphorylation of Fzd3 correlates with endocytosis and may represent active form of Fzd3. Therefore, in the Drosophila wing epithelial cells, the distal membrane, where Fzd is present on the cell surface may be the area where PCP signaling in inactivated (Simons and Mlodzik, 2008). By this prediction, Fzd protein on the distal membrane maybe phosphorylated or hyperphosphorylated. One such inactivating kinase could be atypical PKC, which phosphorylates Ser 500 of Fzd1 (Djiane et al., 2005). Furthermore, Fzd phosphorylation may affect how it interacts with other components. For example, the aPKC site is the Dvl interacting domain. Therefore, the phosphorylation state may affect Fzd's ability to bind to Dvl and thus regulate PCP signaling. For example, binding to Dvl could be a requirement for signaling and/or endocytosis.

Vgl2 may mediate another input of Wnt signaling in PCP. Our study also highlights the role of Vgl. Because Vgl2 can compete with Dvl1 Sin Fzd3 phosphorylation and its plasma membrane localization, Vgl appears to lie upstream of Fzd-Dvl module. Therefore, an automatic question is how is Vgl regulated. Via non-autonomous mechanisms, Vang has been shown recruited by Flm through direct binding between their extracellular domains of neighboring cells. In addition, other Wnt receptors, such as Ror2, can also potentially regulate Vgl2 (Minami et al.). Therefore, Wnt may exert its function through both Fzd and Ror2 in the same cell and these two routes of Wnt signaling entry may coordinate the signaling events spatially or temporally. Because both Vgl and Fzd bind to Dvl, it is appealing to propose that Vgl, once activated, may simply compete with Fzd for Dvl and once Dvl is taken away from Fzd, Fzd can no longer stay on plasma membrane and is quickly subject to endocytosis.

Cell polarity signaling pathways may convey asymmetric signaling in growth cone guidance. Although many families of axon guidance molecules have been identified, how they signal to provide directional control of axon growth is still unknown. This study directly shows that the core PCP components are not only present in navigating growth cones but also function in a PCP-like signaling mechanism. This provides a powerful handle to study signaling and cell biological mechanisms of axon guidance and cell migration. In both axon guidance and cell migration, the dynamic nature of these processes precludes the formation of stable adhesions, therefore, the type of cell-cell interaction observed in a stationary sheet of epithelial tissue is likely minor or non-existent. Therefore, navigating growth cones and migrating cells must be endowed with more sensitive cell autonomous mechanisms to detect and amplify the difference between the two sides of the growth cone and/or cell body. Cell polarity molecules are therefore particularly appealing candidates for these key regulators. In addition, cell polarity regulators are often directly linked to effectors of cytoskeletal or membrane trafficking regulators. Characterizing their functions in axon guidance accelerates the understanding of growth cone cell biology. It should be noted that another important cell polarity signaling pathway, the apical-basal polarity pathway, which intimately interact with the PCP pathway in epithelial cells also provides additional guidance function. Atypical PKC (aPKC) is also required for A-P guidance of commissural axons (Wolf et al., 2008). aPKC has been shown to be a regulator of PCP signaling in the Drosophila eye (Djiane et al., 2005). Studies address the interactions between PCP and apical-basal polarity signaling systems.

### Materials & Methods

Surface biotinylation and immunopreciptiation. 30 hours after transfection with the indicated constructs into HEK293T cells (FuGene, Roche), followed by 8-hour incubation in Optimem and subsequent bath application of recombinant Wnt5a (R&D Systems), surface proteins were subsequently labeled on ice with 1 mg/ml of Sulfo-NHS-LC-Biotin reagent (Pierce). The cell lysates were collect over a period of 30 minutes on ice in the following lysis buffer: 20 mM Tris pH7.6, 150 mM NaCl, 1% Triton, 0.1% SDS, 2.5 mM EDTA, 2.5 mM EGTA, protease Inhibitors (Roche). For the membrane immunoprecipitation, 600 ug of protein extracts were incubated overnight and retrieved by NeutrAvidin-coated Agarose beads (Pierce), the bound fraction was boiled and loaded on SDS-PAGE gel and analyzed by western blot analysis. To visualize the input amount 20 ug of total lysate was loaded, and for the Avidin IPs the total amount was loaded for western blot analysis. The following primary antibodies were used for the immunoblots: α-Frizzled3 (Wang et al., 2006), α-Vangl2 (Santa Cruz), α-mCherry/DsRed (Clontech), α-Jun (Cell Signaling), α-phospho-Jun (Cell Signaling), α-JNK (Cell Signaling), α-phospho-JNK (Cell Signaling), α-Flag (Sigma), α-GAPDH (Chemicon), α-Insulin Rβ (Santa Cruz).

Commissural neuron culture, treatment and lysis. Rat E13 embryos were dissected and commissural neuron culture was prepared as previously described (Augsburger et al., 1999). 1 million commissural neurons were seeded per 6 well/treatment on plates coated with PDL/laminin and grown in commissural neuron media (Neurobasal media supplemented with 2% B27, 40 mM Glucose, 1× Pen/Strep (Gibco) and 1× Glutamax (Gibco)) for 24 hrs. They were subsequently treated with Optimen for 8 hours, followed by Wnt5a addition for the indicated time points (200 ng/ml). The cells were then lysed on ice and the lysates were analyzed on immunoblots.

Plasmid DNA constructs. Frizzled3 and Vangl2 was amplified from mouse E11.5 cDNA library and subcloned into modified mCherry and EGFP expression vectors (pZou-mCherry and pZou-EGFP vectors). 3×Flag-Vangl2 and 3×Flag-Lp were kindly given by D. Davenport and E. Fuches. Dvl1 and Dvl1(KM) constructs were kindly given by A. Wynshaw-Boris.

In situ hybridization. Mouse E11.5 embryos were fixed overnight at 4C in 4% DEPC treated PFA, frozen and sectioned. The in situ hybridization of the spinal sections were completed as previously described (Lyuksyutova et al., 2003), using digoxigenin-labeled riboprobes (Roche). All specific probes were obtained by RT-PCR from E11.5 mouse mRNA and subcloned into TOPO II vector (Invitrogen).

Immunohistochemistry. E11.5 mouse embryos of all wildtype, heterozygous, knock out and mutant embryos were fixed in 4% PFA for 2 hours on ice, frozen in OCT and sectioned at 14 uM slices for immunostaining Immunostaining of spinal cord sections were performed as described previously (Lyuksyutova et al., 2003). The following primary antibodies were used: α-TAG-1 (Developmental Hybridoma), α-L1 (Developmental Hybridoma), α-Frizzled3 (Wang et al, J. Neurosci, 2006), α-phospho-JNK (Cell Signaling), α-Vangl2 (Santa Cruz), Dvl2 (Santa Cruz), Pax7 (Developmental Hybridoma), Lhx1/5 (Developmental Hybridoma), α-tubulin (Sigma), DAPI (Sigma), Phalloidin-488 (Invitrogen), α-EGFP (Invitrogen), α-HA (Covance). Confocal images were taken with Zeiss LSM510.

Electroporated commissural culture. Electroporations of rat E13 spinal cords were completed as previously described (Wolf et al., 2008). Neurons were dissociated from these spinal preps and grown as previously described (Augsburger et al., 1999). After 24 hours of growth in commissural neuron media they were fixed with 2% PFA at 37C for 15 min and immunostained as described (Wolf et al., 2008). Confocal images were taken with Zeiss LSM510. The axons lengths were quantified with Zeiss LSM510 software, verified with ImageJ quantification, and the data were analyzed with an unpaired two-tailed t test for each electroporation condition.

Mouse lines and breeding. Looptail mutant mice (LPT/Le stock, Jackson Laboratories) were provided by A. Wynshaw-Boris (UCSF, San Francisco, Md.). Celsr3 mutant mice were described previously (Tissir et al., 2005). Fzd3 mutant mice generated by J. Nathans (J.H.U. School of Medicine, Baltimore, Md.; (Wang et al., 2002); (Lyuksyutova et al., 2003).

Open-book preparation and DiI axon labeling. Rat E13 spinal-open books were prepared as previously described (Wolf et al., 2008) and cultured for 5-6 hours ex-vivo then for an additional 18 thrs with either control or JNK inhibitors (25 uM JNKI-1 and 50 uM SP600125) to obtain axons that are making their anterior-posterior turning decision before fixation with 4% PFA. Mouse E11.5 spinal-open books were prepared and fixed immediately. Next, to visualize anterior posterior projection of commissural axons DiI labeling was used in the spinal open book preparation. Mouse open-book assay and DiI injections were completed as previously described (Zou et al., 2000); (Lyuksyutova et al., 2003).

### REFERENCES

### For Example 9

Augsburger, A., et al., 1999, Neuron 24, 127-141.

Axelrod, J. D., et al., 1998, Genes Dev 12, 2610-2622.

Boutros, M., et al., 1998, Cell 94, 109-118.

Chen, W., et al., 2004, Science 306, 2257-2260.

Chen, W. S., et al., 2008, Cell 133, 1093-1105.

Devenport, D. & Fuchs, E., 2008, Nat Cell Biol 10, 1257-1268.

Djiane, A., et al., 2005,Cell 121, 621-631.

Dodd, J., et al., 1988,Neuron 1, 105-116.

Gagliardi, M., et al., 2008,Traffic 9, 1-9.

Gao, C. & Chen, Y. G., Cell Signal 22, 717-727.

Goldstein, B., et al., 2006,Dev Cell 10, 391-396.

Goodrich, L. V., 2008, Neuron 60, 9-16.

Jaeschke, A., et al., 2006, Molecular cell 23, 899-911.

Keino-Masu, K., et al., 1996, Cell 87, 175-185.

Kishida, S., et al., 2007, Genes Cells 12, 49-61.

Lyuksyutova, A. I., et al., 2003, Science 302, 1984-1988.

Minami, Y., et al., Dev Dyn 239, 1-15.

Moriguchi, T., et al., 1999, J Biol Chem 274, 30957-30962.

Park, M., & Moon, R. T., 2002, Nature cell biology 4, 20-25.

Park, T. J., et al., 2005, Curr Biol 15, 1039-1044.

Philipp, M., et al., 2008, Mol Biol Cell 19, 5478-5489.

Sato, A., et al., EMBO J 29, 41-54.

Simons, M., et al., 2009, Nat Cell Biol 11, 286-294.

Simons, M., & Mlodzik, M., 2008, Annu Rev Genet 42, 517-540.

Suriben, R., et al., 2009, Nat Genet 41, 977-985.

Tissir, F., et al., 2005, Nat Neurosci 8, 451-457.

Tissir, F. & Goffinet, A. M., 2006, Eur J Neurosci 23, 597-607.

Torban, E., et al., Trends Genet 20, 570-577.

Torban, E., et al., 2004b, J Biol Chem 279, 52703-52713.

Torban, E., et al., 2007, Gene Expr Patterns 7, 346-354.

Wang, Y., & Nathans, J., 2007, Development 134, 647-658.

Wang, Y., et al., 2002, J Neurosci 22, 8563-8573.

Wang, Y., et al., 2006, J Neurosci 26, 355-364.

Wolf, A. M., et al., 2008, J Neurosci 28, 3456-3467.

Wong, H. C., et al., 2003, Molecular cell 12, 1251-1260.

Yamanaka, H., et al., 2002, EMBO Rep 3, 69-75.

Yanfeng, W. A., et al., 2006, J Biol Chem 281, 11603-11609.

Yao, R., et al., 2004, Oncogene 23, 6023-6030.

Yu, A., et al., 2007, Dev Cell 12, 129-141.

Zallen, J. A., 2007, Cell 129, 1051-1063.

Zou, Y., et al., 2000, Cell 102, 363-375.

### Example 10

### Sonic Hedgehog Induced Response of Commissural Axons to Semaphorin Repulsion During Midline Crossing

Pathfinding axons change responses to guidance cues at intermediate targets. During midline crossing, spinal cord commissural axons acquire responsiveness to class 3 Semaphorins and Slits, which regulate their floor plate exit and restrict their post-crossing trajectory into a longitudinal pathway. We found that Sonic Hedgehog (Shh) can activate the repulsive response of pre-crossing axons to Semaphorins. Blocking Shh function with a function blocking Shh antibody, 5E1, in “open-book” explants or by expressing a dominant-negative form of Patched-1, Patched1^{Δloop2}, or a Smoothened (Smo) shRNA construct in commissural neurons resulted in severe guidance defects, including stalling and knotting inside the floor plate, recrossing, and randomized anterior-posterior projection/overshooting after crossing, reminiscent of Neuropilin-2 mutant embryos. Altering Protein Kinase A (PKA) activity in pre-crossing axons diminished Shh induced Semaphorin repulsion and caused profound midline stalling and overshooting/wandering of post-crossing axons. Therefore, a morphogen, Shh, cat act as a switch of axon guidance responses.

Introduction. Commissural axons of the developing spinal cord are guided to the ventral midline by a collaboration of chemoattractants (Netrin-1 and Sonic Hedgehog (Shh)) and chemorepellents (Bone Morphogenetic Proteins (BMPs)) secreted by midline floor plate and roof plate cells, respectively. Once these axons reach the floor plate they switch off their responsiveness to chemoattractants from the floor plate and become responsive to chemorepulsive cues also expressed by the floor plate cells and the surrounding ventral gray matter, including members of the Class 3 Semaphorins (Sema3B and Sema3F) and the Slit family proteins^{(1,2,3,4,5,6)}. Neuropilin-2 mutant embryos showed severe guidance defects including stalling in the midline, overshooting to the contralateral side of the spinal cord and randomly projecting along the anterior-posterior axis⁽⁴⁾. These phenotypes are consistent with the proposed function of the floor plate-secreted chemorepellents to ensure proper midline exit and channel post-crossing commissural axons to turn into their correct (longitudinal) trajectory after exiting the midline. Nonetheless, the molecular mechanisms underlying changes in responsiveness to guidance cues, particularly the extrinsic signals that trigger these changes are still not largely known. Here, we studied how commissural axons gain responsiveness to Semaphorin repulsion during midline crossing and found that Shh, a morphogen highly enriched in the floor plate, may act as an “on” switch for Semaphorin repulsion in commissural axons. The Shh receptors Patched-1 (Ptc-1) and Smoothened (Smo) are both necessary for this switch and for proper midline guidance of commissural axons. Shh likely sensitizes growth cone responsiveness to Class 3 Semaphorins by down regulating the activity of the cAMP/Protein kinase A (PKA) pathway. Therefore, we propose a novel role of morphogen Shh in regulating the responses of other guidance cues.

### Results

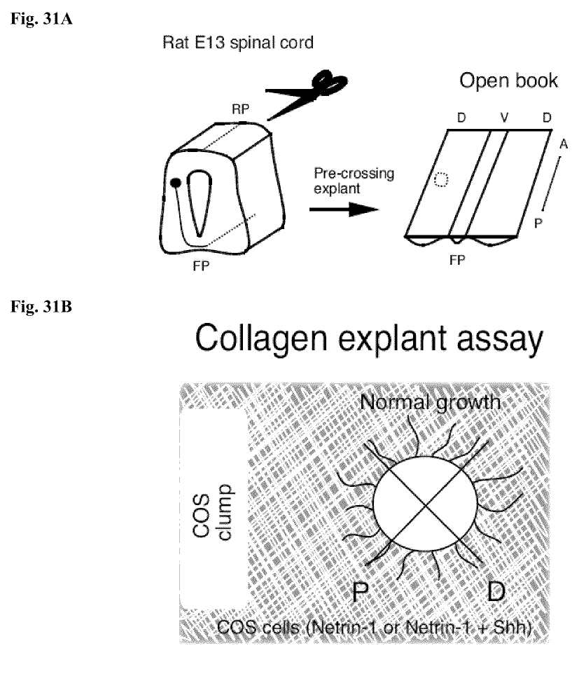

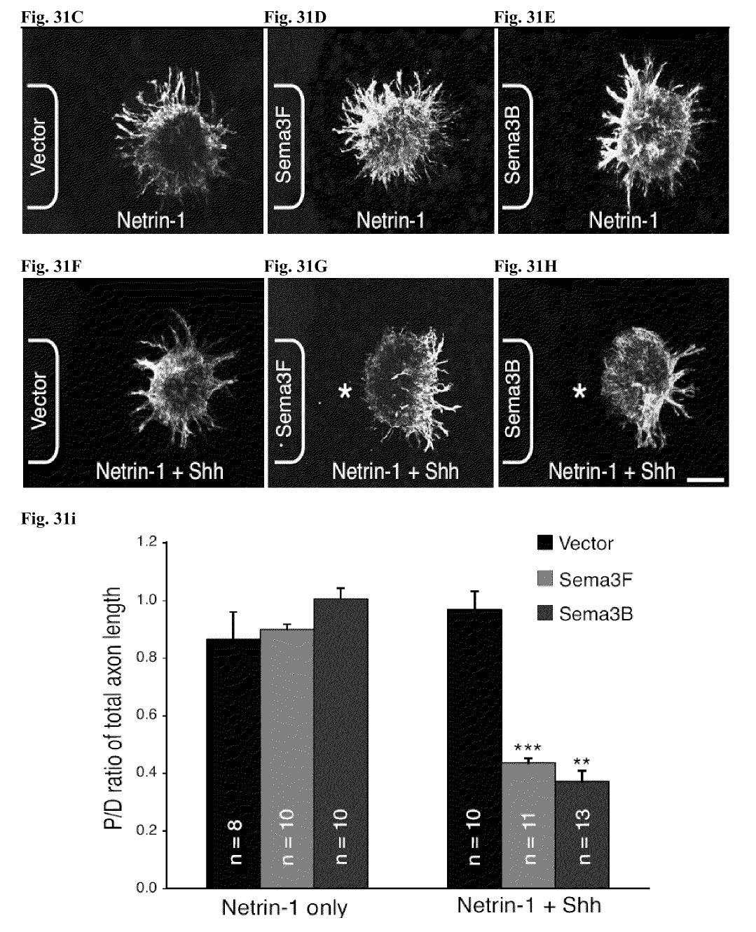

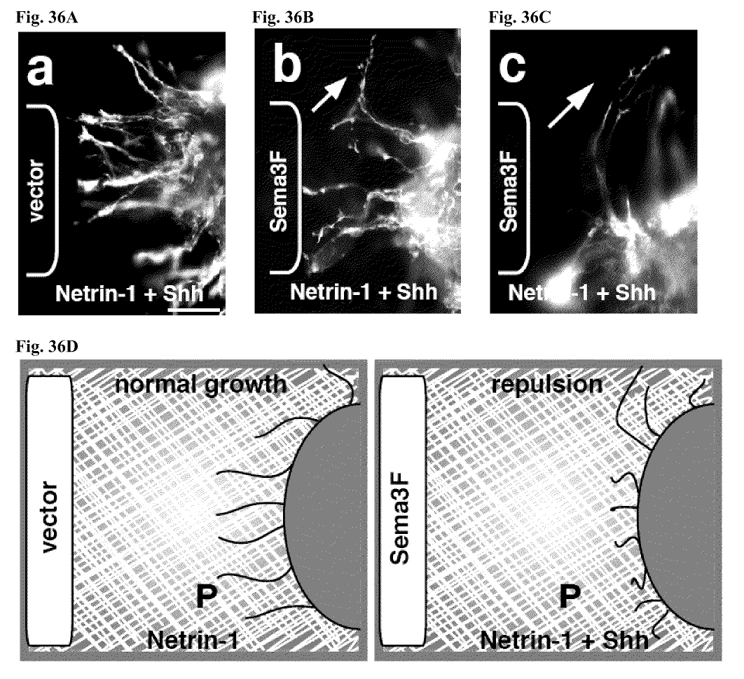

Shh activates Semaphorin repulsion in commissural axons. We investigated the mechanisms by which Semaphorin repulsion in commissural axons is switched on during spinal cord midline pathfinding. Commissural neuron cell bodies are located in the dorsal spinal cord and initially extend axons to the ventral midline, the floor plate, in the process of several days (from late E8.9/early E9.5 in mouse and late E10 and E11 in rats till at least E12.5 in mouse and E14 in rat). The commissural axons in the rostral (anterior) spinal cord differentiate and their axons develop earlier than their caudal (posterior) counterparts. At each axis level, the more dorsal populations tend to differentiate and extend axons later than the ventral population in the dorsal spinal cord during this stage of development. At E13 in rats, many rostral commissural axons have reached and crossed the midline while the more caudal populations are still growing towards the midline or are just starting to cross the floor plate. To obtain pre-crossing commissural axons, we use the dorsal-most margin of the caudal spinal cord (hindlimb level) from E13 rat embryos. These are the younger axons, which have not reached/contacted the floor plate. We used a modified pre-crossing explant assay in collagen gel so that we could test whether any of the diffusible signaling molecules expressed in the floor plate can activate Semaphorin repulsion in pre-crossing commissural axons, which have not yet encountered the floor plate (FIGS. 31A-31B). In this assay, the bottom collagen layer contains dispersed COS-7 cells expressing Netrin-1 alone or Netrin-1 plus a secreted molecule from the floor plate (FIG. 31B, gray scratched pattern). In the top collagen gel, explants of dorsal commissural neurons from E13 rat embryos (equivalent to E11.5 mouse embryos) were embedded next to COS-7 cell aggregates (clumps) expressing Sema3B or Sema3F. We have used similar the approach to introduce secreted Frizzled-related proteins (sFRPs) in “open-book” spinal cord explant cultures to test their effect on the anterior turning of the commissural axons⁷. In our assay, explants cultured in the presence of Netrin-1 alone grew radially around their circumference and did not respond to Sema3B- or Sema3F-expressing COS-7 cell aggregates⁴ (FIGS. 31D-31E) similar to the vector only cell aggregate control (FIG. 31C). This is consistent with previous finding that Sema3B and Sema3F do not repel pre-crossing commissural axons. Interestingly, to our surprise, pre-crossing commissural axons grown on top of collagen gel containing Netrin-1 and Shh expressing cells were repelled by Sema3B- and Sema3F-secreting cell clumps (FIGS. 31G-31H) compared to the radial growth seen with the vector control in the presence of Netrin and Shh (FIG. 31F). To quantify these effects, the total length of axons extending towards (proximal) or away (distal) from cell aggregates (FIG. 31B) was calculated in each experiment as the ratio of axons growing proximally (P) divided by axons growing distally (D) (FIG. 31i). This induced Semaphorin activity is a true repulsion because axons were observed bending away from the cell clumps at higher magnification (arrows in FIGS. 36B-36C). Our results indicated that the repulsive activities of Sema3B and Sema3F could be switched on by Shh using this in vitro assay. Because Shh is highly abundant in the floor plate, surrounding tissue, and the notochord of the developing spinal cord, Shh is an excellent candidate for a midline switch signal.

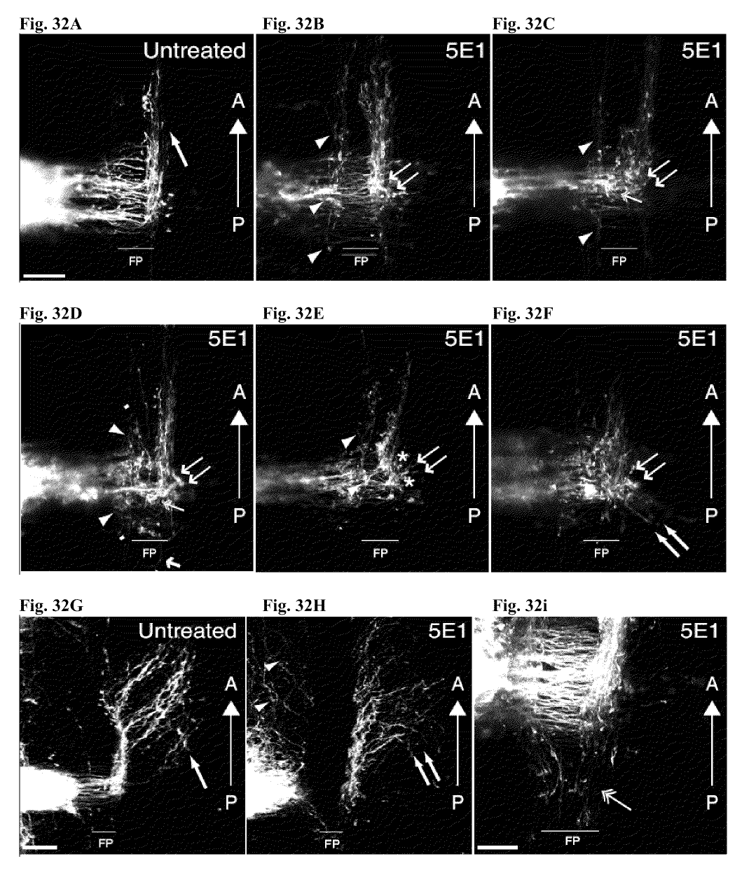

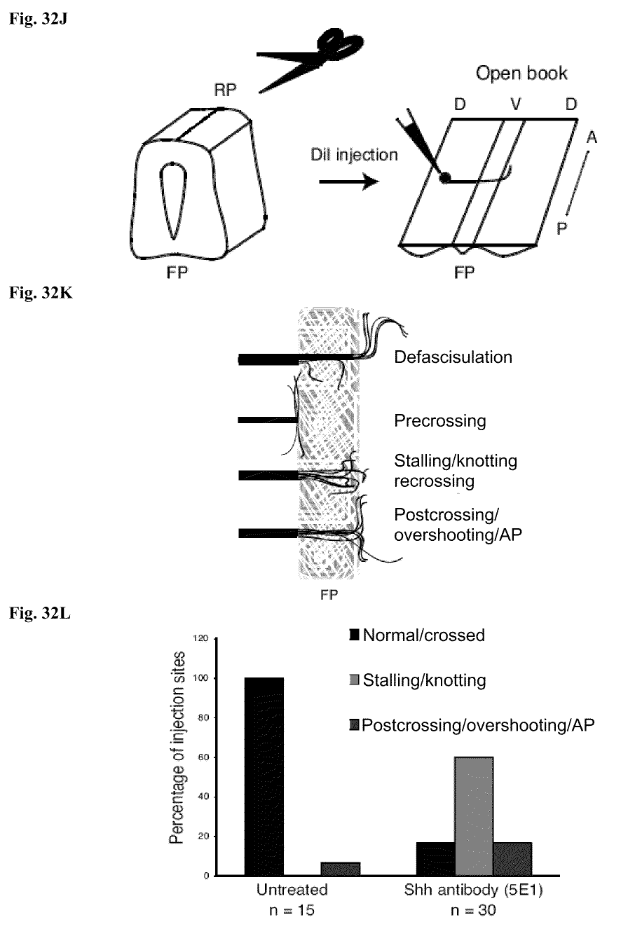

Blocking Shh activity causes midline guidance defects. To determine the role of Shh in regulating midline pathfinding, we analyzed the trajectory of commissural axons in rat E13 “open-book” spinal cord preparations treated with the function-blocking antibody for Shh, 5E1. The projections of these commissural axons were visualized by injecting the lipophilic dye, DiI, to the dorsal margin of the spinal cord (FIG. 32J). In “open-book” explants, commissural axons can recapitulate the in vivo pathfinding and project from the dorsal spinal cord margin across the floor plate and then turn sharply anteriorly on the contralateral side.^{(7,8)}. At E13, the spinal cord patterning has been completed and many commissural axon have extended close to the midline, allowing us to specifically analyze the role of Shh on commissural axon midline pathfinding.⁽⁵⁾ Our 5E1 treatment results showed that some commissural axons were blocked from extending towards the floor plate and projected along the anterior-posterior direction close to the ipsilateral border of the floor plate (FIGS. 32B-32E, arrowheads) when compared to untreated controls (FIG. 32A), consistent with the role of Shh as chemoattractant for pre-crossing commissusral axons⁵. In addition, the majority of commissural axons stalled and knotted within the midline (FIGS. 32C-32D, two-headed arrows) as well as at the contralateral edge of the floor plate (FIGS. 32B-32F, short double arrows), suggesting that the axons are no longer expelled out of the midline properly. The 5E1 antibody treatment also caused wandering and overshooting of post-crossing axons on the contralateral side of the spinal cord (FIG. 32F, long double arrows) with a few post-crossing axons recrossed the floor plate (FIG. 32D, single arrow). Normally, after midline crossing, commissural axons make a sharp anterior turn and either project into the ventral funiculus alongside the floor plate or fan out in a highly organized manner to grow laterally into the lateral funiculus⁽⁷⁾ (FIG. 32G). In contrast, commissural axons in “open-book” explants treated with 5E 1 deviated and defasciculated from that organized trajectory after midline crossing (FIG. 32H, double arrows) with some axons misprojecting posteriorly (FIG. 32i, two-headed arrow) as shown in these longer cultured explants, suggesting that they did not respond to the repulsive signals that channel them into an organized ventral/lateral funiculus. The spinal cord midline is a three-dimensional structure. After crossing the midline, commissural axons initially project right next to the floor plate within the same plane of the floor plate before they fan out gradually to join the ventral funiculus, which is out of the plane of the floor plate. FIG. 32H is on focal plane featuring how the post-crossing axons wander away from their original trajectory and therefore the floor plate and the crossing axons are not visible. In these antibody treatment experiments, commissural axons of slightly different stages are crossing the midline and turning anteriorly. When antibody treatments started to take effect, for the commissural axons, which are still in the floor plate, they may stall in the midline, but for the commissural axons that have finished midline crossing, antibody treatments may lead to their post-crossing wandering/random phenotype. Therefore, we see a range of phenotypes representing the interruption of pathfinding at different stages. These phenotypes are similar to those seen in mice deficient in Neuropilin-2, which encodes a key receptor component for Class 3 Semaphorins, Sema3B and Sema3F⁴. We quantified the percentage of injection sites, which showed these various classes of phenotypes, not the percentage of axons. When we label commissural axons using DiI injection, we often identify a cohort of axons. Within these cohorts, there were occasions where multiple guidance defects were observed. Some of the injection sites were therefore scored for more than one category of phenotypes. Therefore, the total percentage can be higher than 100%. Similar methods were used in previous studies^{(4,7,8)}.

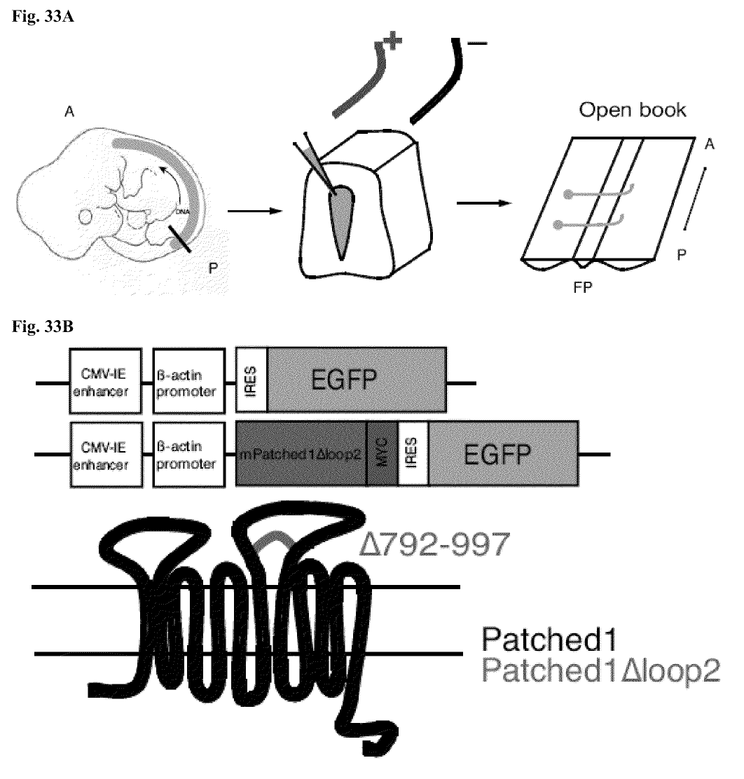

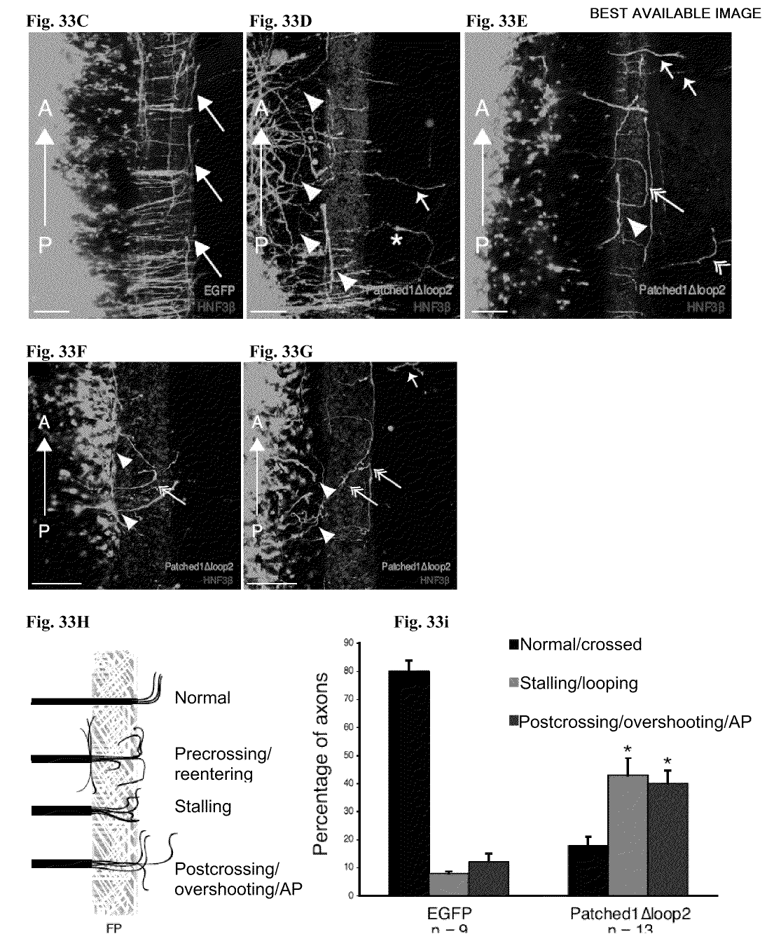

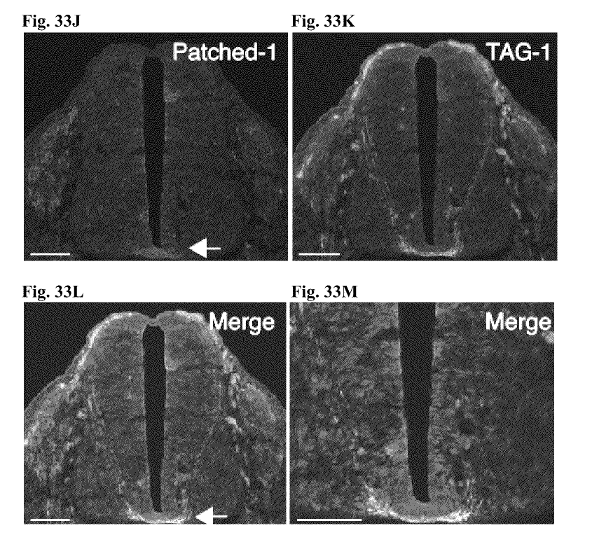

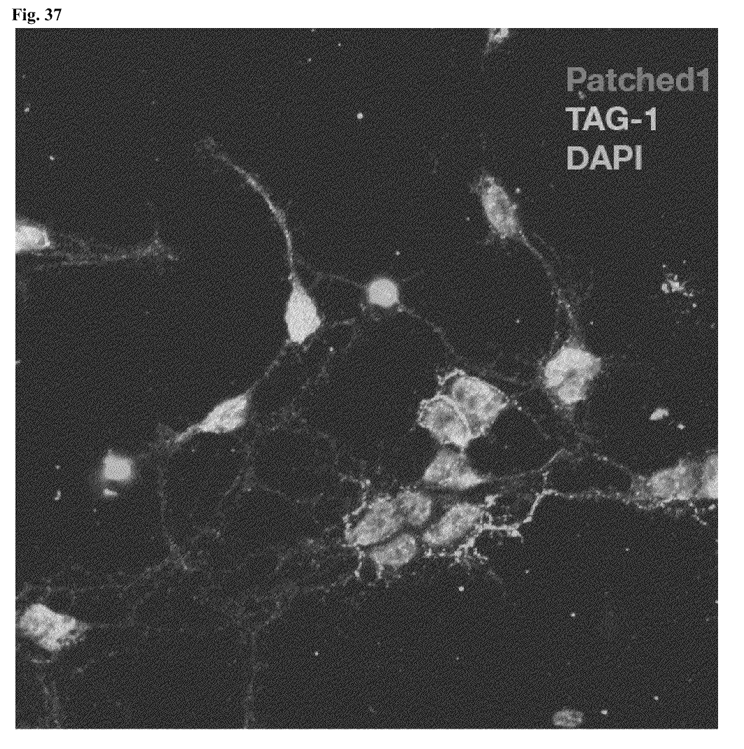

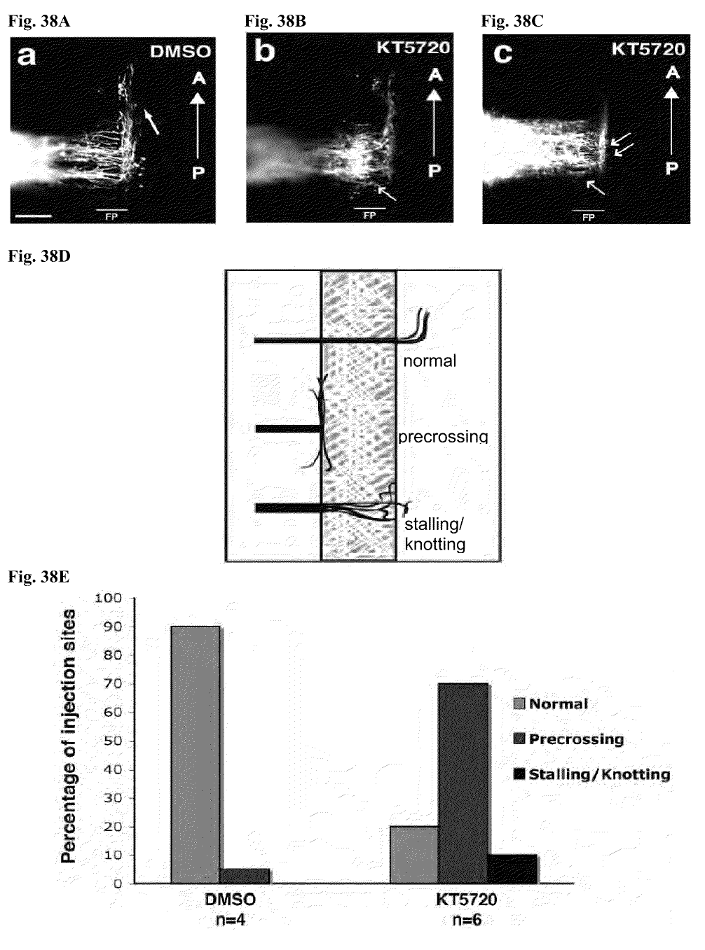

Perturbing Shh-Ptc-1 and Smo causes midline defects. To examine how Shh may regulate response of commisural axons to repulsive guidance cues and their midline pathfinding, we first tested the function of the Shh receptor, Patched-1 (Ptc-1), by expressing a dominant-negative construct in commissural axons using ex utero electroporation in “open-book” spinal cord cultures⁽⁸⁾ (FIG. 33A). Here, we generated a deleted form of Ptc-1, Patched1^{Δloop2}, as previously described⁽⁹⁾, which lacks the Shh-binding domain in its second extracellular loop, rendering it constitutively active in its ability to inhibit Smoothened (Smo). Patched1^{Δloop2} was cloned into the IRES-pClG2 vector driven by a β-actin promoter to allow expression in commissural neurons and their visualization of neurons expressing the mutant Patched construct under the green fluorescence protein (FIG. 33B). Rat E13 electroporated spinal cords were cultured in collagen gel for three days, during which time pCIG2-IRES-GFP expressing commissural axons reached and crossed the ventral midline and turned anteriorly in control “open-book” electroporated explants (FIG. 33C). In contrast, expression of Patched^{Δloop2} resulted in severe midline pathfinding defects (FIGS. 33D-33G). It should be noted that the number of commissural axons reaching and entering the midline were significantly reduced, consistent with the finding that Shh is a chemoattractant for pre-crossing axons⁽⁵⁾ (FIG. 33D, arrowheads) and many axons were stuck right at the floor plate entrance (FIGS. 33E-33G, arrowheads). We then focused on the axons entering the midline to examine the function of Shh in regulating subsequent commissural axon trajectory. In support of a role in regulating responses to chemorepellents at the midline by Shh, the majority of commissural axons expressing Patched1^{Δloop2} entering and crossing the midline were clearly abnormal, with many axons looping back and recrossing the midline, a phenomenon similar to the Drosophila midline defect in roundabout mutants⁽¹⁰⁾ (FIGS. 33E-33G, double headed arrows). A significant number of axons failed to make a sharp anterior turn after midline crossing, a misguidance defect also described in the rostral turn of chicken embryos after in ovo Shh RNAi electroporation⁽¹¹⁾. Instead, these axons projected contralaterally and made gradual turns randomly along the A-P axis (FIG. 33d, FIG. 33E, FIG. 33G, arrows). These results suggested a failure to respond to a repulsive cue from the contralateral direction, which helps commissural axons form the sharp anterior turn once they exit the floor plate. These phenotypes are again reminiscent of the Neuropilin-2 mutants.⁽⁴⁾ To determine whether Ptc-1 was expressed in commissural axons during midline pathfinding, we performed immunohistochemistry of E11.5 mouse embryos and found that Ptc-1 was highly expressed in the commissural axon segment at the ventral midline and immediately after crossing (FIGS. 33J-33M). We also found that in dissociated dorsal spinal cord cultures, the Ptc-1 protein is present in all TAG-1-positive neurons (red and cyan co-localization along axons in FIG. 38). The immunostaining pattern is also consistent with in situ data in chick embryos (Ptc-1 transcript was found in dorsal root ganglion at equivalent stages).^{(12,13)} It should be noted that Ptc-1 is strongly induced by Shh and the expression level of Shh is highest at the ventral spinal cord.^{(14,5)} Together these data confirmed that Ptc-1 was expressed in the right place and at the right time when commissural axons needed to acquire responsiveness to repulsive cues.

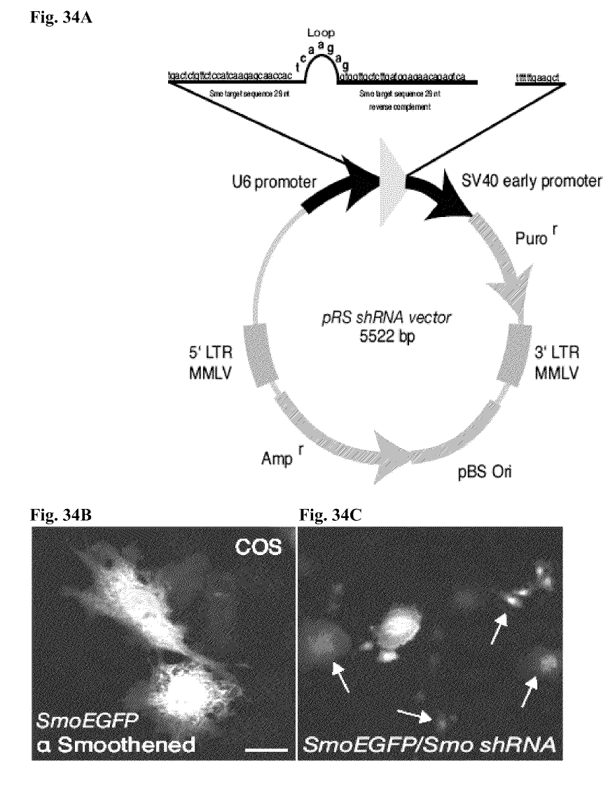

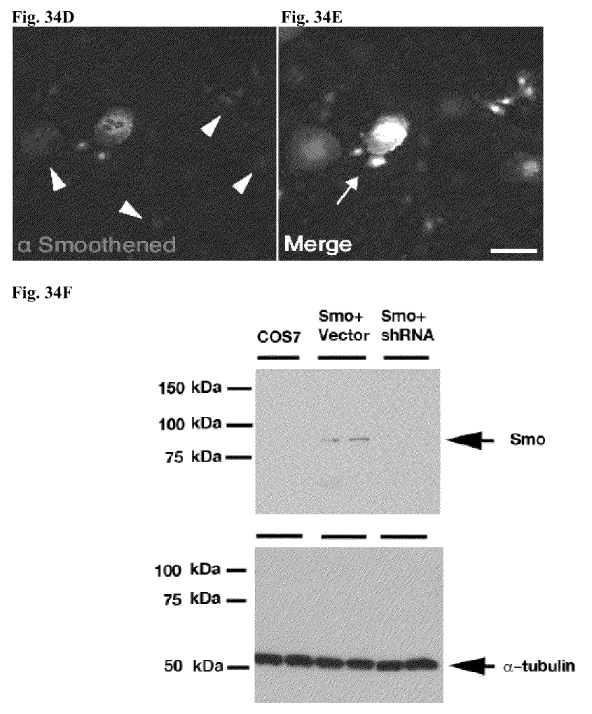

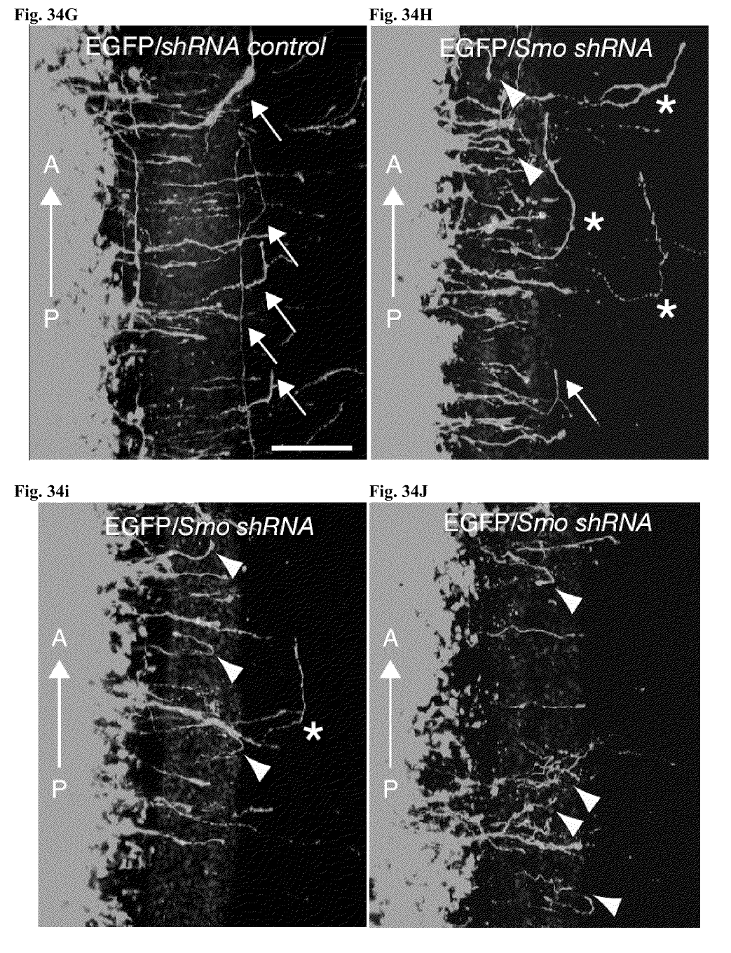

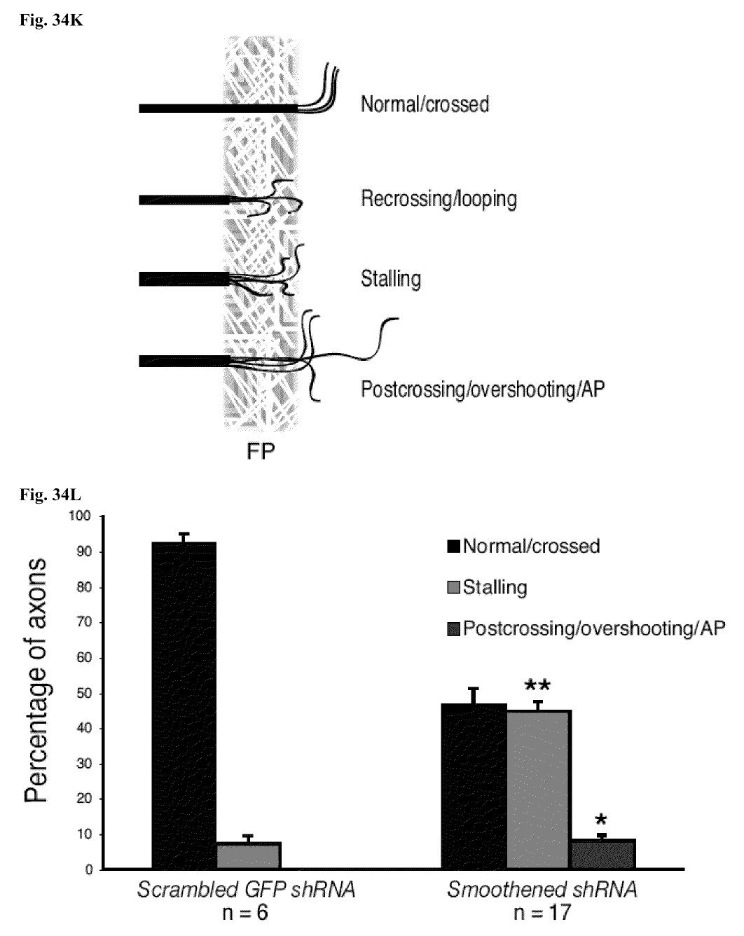

To test the role of Smo during midline pathfinding, we obtained shRNA constructs (Origene) targeting Smo (FIG. 34A) and expressed them in commissural neurons by electroporation of E13 rat embryos as described in FIG. 33A. Smo is broadly expressed in the spinal cord and we also found that endogenous Smo protein is present in the growth cones of developing commissural axons. Here, we show that the Smo shRNA construct can effectively down regulate Smo protein levels assessed by immunocytochemistry (FIGS. 34B-34E, arrowheads) and by Western blotting (FIG. 34F). In our studies, we found that knocking down Smo caused severe midline guidance defects. Similar to the dominant-negative form Patched, Patched1^{Δloop2}, many axons turned back into the midline as soon as they reached the center of the floor plate or the contralateral border of the floor plate (FIG. 34G-34J, arrowheads). In addition, most of the axons, which have crossed the midline, wandered after midline crossing and gradually turned anteriorly after crossing instead of making a sharp anterior turn right after midline crossing (FIGS. 34H-34i, asterisks). We tested a different Smo shRNA construct, which also down regulates Smo protein, and found very similar midline guidance defects. However, when we tried to rescue the phenotype with a wildtype Smo expression construct, we found that expressing Smo in the dorsal spinal cord progenitor cells using electroporation causes severe disruption of the neural epithelium (with prominent rosettes in the “open-book” explants) and these neurons failed to extend axons. Therefore, we were unable to perform rescue experiments after Smo shRNA electroporation. Taken together, we propose that the Ptc-1/Smo signaling pathway is required for correct midline pathfinding and likely mediates the “on” switch of Semaphorin repulsion.

Shh may induce Sema repulsion by reducing cAMP/PKA signaling. Cyclic nucleotides play important roles in signaling in response to guidance cues and axonal responses of attraction and repulsion depend on the ratios of cAMP to cGMP⁽¹⁵⁾ Overall, higher cAMP and lower cGMP associate with attraction and lower cAMP favors repulsion. PKA activity has been shown coupled with Semaphorin-PlexinA-mediated repulsion in vivo⁽¹⁶⁾ and we hypothesized that growth cone cAMP levels may be down regulated by floor plate signals, such as Shh, causing axons to become sensitive to Semaphorin chemorepulsive activity. To test this, we first examined whether appropriate cAMP/PKA activity was necessary for correct midline guidance. In “open-book” explant cultures we treated commissural axons with the activator for adenylyl cyclase (and consequently cAMP), Forskolin and the PKA inhibitor KT5720 and analyzed commissural axon pathfidning by DiI tracing as shown in FIG. 32J.

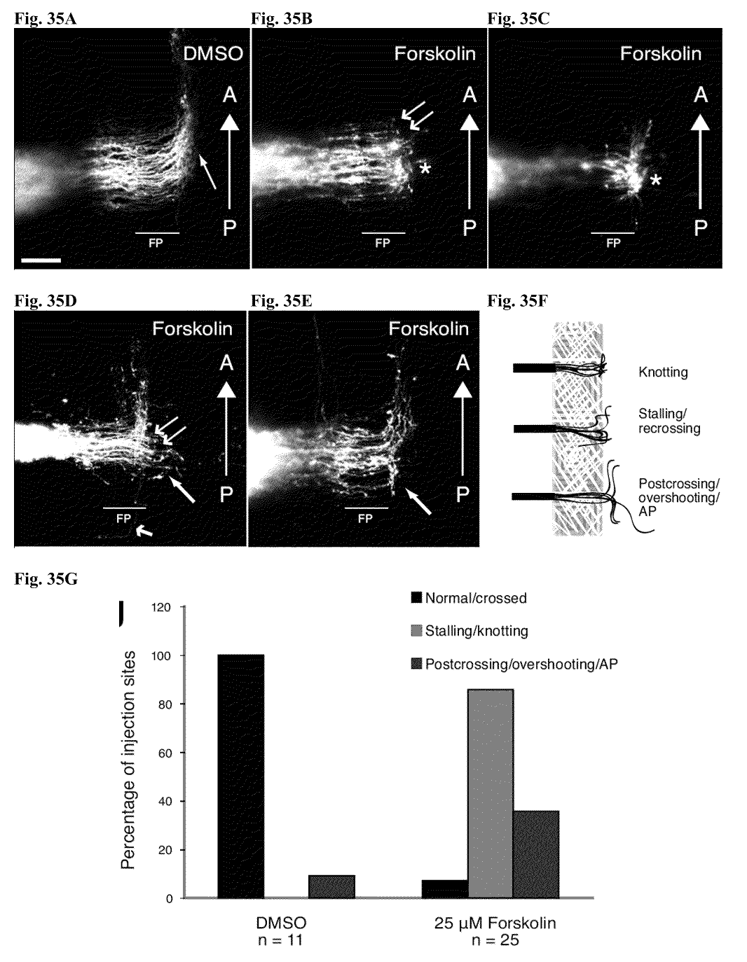

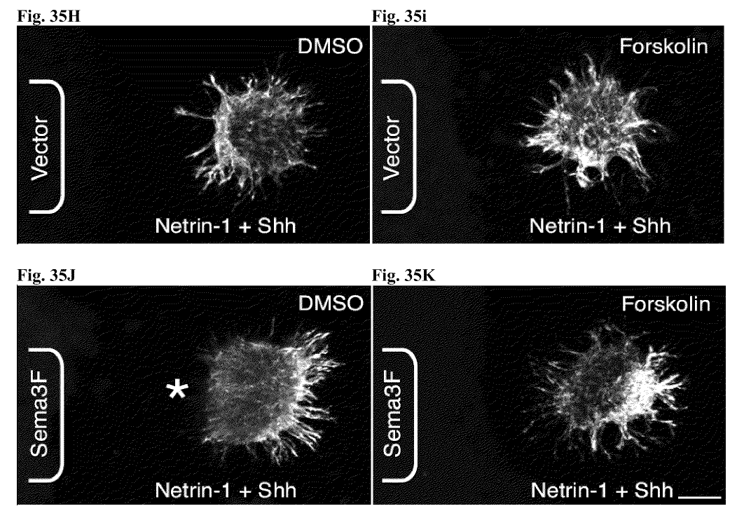

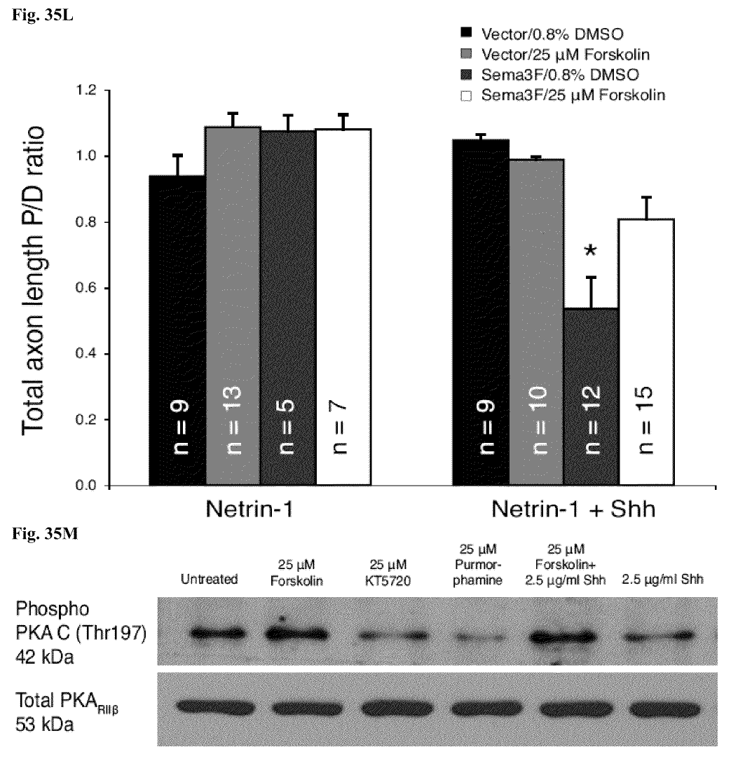

In Forskolin treated “open-book” explants, many axons stalled (FIG. 35B, FIG. 35D, double arrows) and formed knots at the contralateral edge of the floor plate (FIGS. 35b-35C, asterisks), whereas others displayed wandering behaviors after midline crossing (FIG. 35D) and yet others overshot (FIG. 35D, long single arrow) or turned widely and randomly along the A-P axis (FIG. 35E, long single arrow). In some injections, a few of these wandering axons looped back and reentered the floor plate (FIG. 35D, short single arrow). In these experiments, commissural axons of slightly different stages are crossing the midline and turning anteriorly. When drug treatments started to take effect, for the commissural axons that are still in the floor plate they may stall in the midline, but for the axons that have finished midline crossing, drug treatments may lead to their post-crossing wandering/random phenotype. Therefore, we see a range of phenotypes representing the interruption of pathfinding at different stages. These defects are again very similar to those seen in Neuropilin2 −/− embryos⁽⁴⁾ and in 5E1-treated “open-book” explants (FIG. 32), suggesting that Forskolin addition may interfere with the normal switching of Semaphorin responsiveness needed for proper midline pathfinding. In addition, we found that down regulating PKA with the PKA inhibitor, KT5720, prevented a vast number of axons from entering the floor plate (upward arrows in FIGS. 37B-37C). This could be due to the inhibition of an attractive mediated by PKA at the floor plate so that these axons can no longer enter the floor plate. Alternatively, inhibiting PKA may cause sensitization of commissural axons to chemorepellents, such as Semaphorins, in these “open-book” assays, similar to how Shh activates Semaphorin responsiveness.

To further test the hypothesis that reducing PKA activity is required for inducing responsiveness to class 3 Semaphorins by Shh, we performed the precrossing collagen explant assay (FIGS. 31A-31B) and found that Shh-induced Semaphorin repulsion in pre-crossing explants (FIG. 35J) can indeed be diminished in the presence of Forskolin (FIG. 35K). To confirm that PKA activity can be reduced in commissural neurons exposed to Shh, we treated dissociated spinal cord commissural neurons with Shh and analyzed the level of activated PKA. 24 hr after plating, commissural neurons were serum starved overnight and then treated with 2.5 μg/ml (150 nM) of Shh-N recombinant protein (R&D) for one hour prior to lysate collection. Forskolin (25 μM), and the PKA inhibitor, KT5720 (25 μM) were added to the commissural neuron culture as controls for 15 minutes before lysis. We then tested these lysates for levels of activated PKA with an antibody that specifically recognizes an activated form of the catalytic domain of PKA (phosphorylated at Thr197). Western blot analysis showed a significant reduction of activated PKA with Shh-N recombinant protein treatments compared to the lysate controls (FIG. 35M) suggesting that Shh may cause a significant reduction of PKA activity in the growth cones of commissural axons once they enter the floor plate thus allowing repulsion to take place. Together, these data suggest that Shh may regulate growth cone sensitivity to Semaphorins, at least in part, by regulating cAMP levels.

### Discussion

The midline is a major organizer for patterning of axonal connections throughout the nervous system and an important intermediate target. Axons often change trajectory after midline crossing due to the dynamic regulation of growth cone responsiveness to guidance cues. The extrinsic and intrinsic mechanisms of this switch of responsiveness are still not well known. Our results suggest that Shh may be an extrinsic midline switch for turning on Semaphorin repulsion of commissural axons. This study suggests that morphogens, such as Shh, may directly modulate growth cone signaling of other guidance cues and orchestrate growth cone remodeling at intermediate targets, further extending the role of morphogens in axon development.

Shh binds to Ptc-1 and signals through Smo by suppressing the inhibition of Ptc-1 on Smo.⁽⁹⁾ In this study, we found that both Ptc-1 and Smo are both required for proper midline pathfinding of commissural axons. The guidance defects caused by blocking Shh signaling, either by constitutively active Ptc-1 or Smo shRNA knockdown, are consistent with the role of Shh in inducing Semaphorin repulsive response, which is essential for correct midline pathfinding.⁽⁴⁾

Our current study is the first example of how a morphogen can act as a switch for other axon guidance molecules at an intermediate target. Because morphogens are commonly enriched at organizing centers during early nervous system pattern, such as the spinal cord ventral midline, and these organizing centers are often places where axons make abrupt trajectory changes, it is possible that other morphogens may also act as switches at many intermediate targets. Along the same line, Shh may also regulate responses of other guidance cues at the midline besides Semaphorin signaling. This finding does not exclude other potential functions of Shh.

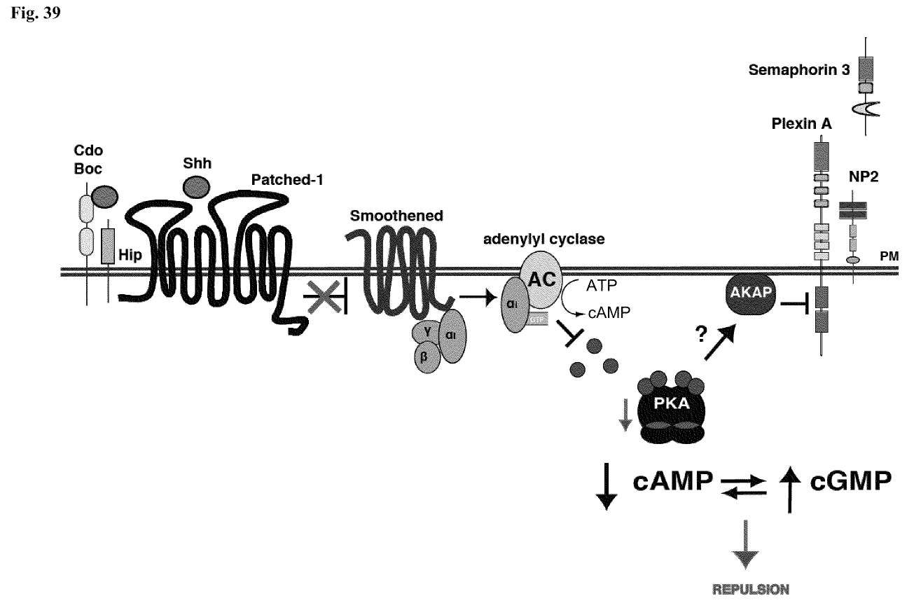

The signaling mechanisms mediating class 3 Semaphorin repulsion are still not completely understood beyond their receptor complexes, particularly in vertebrates.⁽¹⁷⁾ The role of some of the downstream components specific to Semaphorin signaling in vertebrates are not known and let alone the availability of the appropriate reagents to intervene their functions. Cyclic nucleotides are well known for their ability to convert the actions of guidance cues from attraction to repulsion.^{(15,18,19)} For example, the attraction of Xenopus spinal cord neurons can be converted to repulsion by inhibiting cAMP or PKA⁽¹⁵⁾ or by modulating cAMP/cGMP levels⁽¹⁹⁾. In Drosophila, the Semaphorin receptor Plexin can be regulated by cyclic nucleotides. An A-kinase anchoring protein (AKAP), Nervy, was shown to link PKA to Plexin, which counteracted Semaphorin-directed repulsion.⁽¹⁶⁾ Moreover, recent evidence showed that Smo can act a as a true GPCR, signaling via Gαᵢ.⁽²⁰⁾ Shh has also been shown to be a negative regulator of growth cone movement by decreasing cAMP levels in mouse retinal ganglion cell axons.⁽²¹⁾ We therefore tested the hypothesis that growth cone cAMP levels may be regulated by floor plate signals causing commissural axons to become sensitive to the Semaphorin chemorepellent activities. Our results show that Shh does reduce PKA activity in commissural neurons and elevating cAMP levels blocked the Shh induced Semaphorin repulsion, causing midline pathfinding defects consistent with the notion that Semaphorin response failed to be activated. Taken together, we propose that Shh activates the Smo-Gαᵢ pathway and decreases the level of cAMP, which, in turn, relieves the inhibition on PlexinA signaling imposed by the AKAPs (FIG. 39). This mechanism is tested further as more information and reagents related to the vertebrate AKAPs are available. Although reduction of cAMP/PKA activity is required for switching on Semaphorin repulsion by Shh, we found that lowering/inhibiting cAMP/PKA activity was not sufficient to turn on the response of Semaphorin repulsion in pre-crossing commissural axons (FIG. 37). This suggests that Shh likely activates additional mechanisms to complete this switch.

Midline pathfinding is a complex process and involves several attractive and repulsive cues, although the repulsive mechanisms have been much better studied. Another well-characterized midline guidance system is the Slit-Robo system, which is required for commissural axons at both the Drosophila and the vertebrate midline.⁽¹⁰⁾ In addition to the Slit-Robo system, the Semaphorin-Neuropilin system also plays important roles in vertebrates.⁽⁴⁾ Indeed, when the Slit-Robo system is disrupted genetically, many axons are still channeled into the corridor between the floor plate and the ventral spinal cord (“squeezed out of the gray matter”).^{(6,22)} The exact functions of the Slit-Robo and Sema-Neuropilin systems need to be further characterized. First, it is possible that different populations of commissural axons use different repulsive mechanisms during this process, some using the Slit-Robo system and the others Sema-Neuropilin system. Second, it is possible that both repulsive systems act in concern to ensure the maximal precision and efficiency. A third possibility is that Semaphorins and Slits may act in sequence to mediate midline expulsion. A fourth possibility is that Slits may be involved in regulating midline entrance where as Semaphorins are more important for midline expulsion, preventing midline re-crossing and overshooting in rodent midline.^{(23,6)} Studies address how these two repulsive systems are integrated to orchestrate the crossing and turning of commissural axons and how Shh may play a role in this intricate mechanism.

Here, we show that a diffusible morphogen Shh can activate Semaphorin repulsion. An intriguing question is why commissural axons are not repelled by Semaphorins before crossing even when they are close to the floor plate where Shh is abundant and why they are still able to cross the midline. We hypothesize that certain components of Semaphorin signaling are only produced when commissural axons have reached or entered the midline. This is achieved, for example, by a fine temporal program or triggered by a local floor plate signal, involving de novo transcription, local protein synthesis and/or modulation signal transduction.

### Methods

Animals. Embryos from staged Sprague Dawley pregnant rats (Charles River) were collected at embryonic day 13 (E13). CD-1 mice (The Jackson Laboratory) were staged in house and embryos were collected at day post coitum (dpc) 11.5. Animals were handled according to The University of California, San Diego Institutional Animal Care and Use Committee.

Expression constructs. Netrin-1 and Semaphorin3F cDNA expression constructs were as described⁽⁴⁾. Shh≢CT (N-Shh) was a gift from Marc Tessier-Lavigne . The Semaphorin3B expression construct was a generous gift from V. Castellani (Universite Claude Bernard) and was subcloned into a pcDNA™ 3.1 vector (Invitrogen) for COS-7 cell expression. Patched1 (GenBank accession number U46155) was amplified from an 11.5 dpc whole embryo mouse cDNA library using the forward primer 5′-ATG GCC TCG GCT GGT AAC GCC GCC GGG GCC-3′ (SEQ ID NO:39) and the reverse primer 5′-GTT GGA GCT GCT CCC CCA CGG CCT CTC CTC -3′ (SEQ ID NO:40). Deletion of the second extracellular loop, which blocks the ability to sequester Shh and transduces Shh signals was obtained by polymerase chain reaction deletion of amino acids 768-1026 as previously described⁽¹³⁾ and blunt-end ligated with a modified PmeI linker site (GTTTcAACc). The coding sequence at the join is GLDLTDIVP/VST/LVCAVFLLN (SEQ ID NO:41), where VST codes for the modified PmeI linker site. A Myc-tag epitope was added to the C-terminus to generate Ptc1^{Δloop2} Myc. The resulting Ptc1^{Δloop2} Myc construct was inserted in frame between the AscI and XmaI sites of a pCIG2-IRES-GFP vector (from Franck Polleux) driven under the control of a CMV enhancer and chicken β-actin promoter (modified to include a unique AscI insertion site). Smoothened (GenBank accession number BC048091) was amplified from an 11.5 dpc whole embryo mouse cDNA library using the forward primer 5′-ATG GCC GCT GGC CGC CCC GTG CGT GGG CCC-3′ (SEQ ID NO:42) and reverse 5′-GAA GTC TGA GTC TGC ATC CAA GAT CTC AGC-3′ (SEQ ID NO:43). A Myc-tag epitope was added to the C-terminus to generate Smo-Myc. The resulting construct was inserted in frame at the AscI insertion site of the pCIG2-IRES-GFP vector as described above. A synthesized 29 nucleotide short hairpin RNA (shRNA) construct against Smoothened (Smo) was purchased in a pRS plasmid under U6 promoter coding for Smo sequence 5′-TGACTCTGTTCTCCATCAAGAGCAACCAC-3′ (SEQ ID NO:44) (Origene).

Immunohistochemistry. Rat E13 spinal cord commissural explants were fixed in the collagen matrix at 37° C. with 4% PFA, at blocked in 5% PDT (1×PBS, 5% normal donkey serum, 1% BSA, 0.1% Triton X-100) and processed for TAG-1 immunostaining (1:50; 4D7, Developmental Studies Hybridoma Bank) followed by secondary staining with Cy3 donkey anti-mouse IgM antibody (1:300; 715-165-140, Jackson ImmunoResearch). Rat E13 electroporated open book spinal cultures were fixed in the collagen matrix at 37° C. with 4% PFA and incubated overnight at 4° C., then harvested from the collagen and blocked with 5% PDT as previously described₈. This was followed by overnight incubation at 4° C. with anti-GFP antibody (1:10000; A11122, Invitrogen) and the floor plate marker antibody, HNF-3β (1:50; 4C7, Developmental Studies Hybridoma Bank). The next day, open book explants were washed in 1% PDT and incubated overnight with Alexa Fluor® 488 donkey anti-rabbit IgG (1:1500; A21206, Invitrogen) and Cy3 donkey anti-mouse IgG (1:300; 715-166-150, Jackson ImmunoResearch) respectively. Lastly, these explants were mounted in Fluoromount G (Fisher) between two glass coverslips for microscopic analysis. E11.5 mouse embryos were harvested from CD-1 pregnant mice and fixed at 4° C. in 4% PFA with rotation. Fixed embryos were washed with 1×PBS and equilibrated overnight with 30% Sucrose followed by OCT embedding. OCT blocks were frozen in a CO₂/ethanol bath and kept at 80° C. for cryostat sectioning. Cryostat sections (20 μm) were processed on Superfrost Plus slides (Fisher), air dried, and blocked in 5% PDT for 1 hour followed by 4° C. overnight incubation with the primary antibodies TAG-1 (1:50) and Patched-1 (1:100; ab53715, Abcam). Slides were washed the next day three times with 1% PDT and incubated for lhr at room temperature (RT), with secondary antibodies, Cy2 donkey anti-mouse IgM (1:300, 715-226-020, Jackson ImmunoResearch) and Cy3 donkey anti-rabbit IgG (1:300) respectively. The slides were subsequently washed in 1% PDT before mounted in Fluoromount G (Fisher) for microscopic analysis. Dissociated commissural neuron cultures from rat E13 embryos were fixed at 37° C. with 4% PFA for 20 minutes as describeds. After fixation, the commissural cultures were blocked for 1 hr in 1% PDT and stained for TAG-1, DAPI, or Smoothened (1:100; ab60016, Abcam) overnight. This was followed the next day with secondary antibody incubation of Cy3 donkey anti-mouse IgM or Cy3 donkey anti-rabbit IgG respectively for 2 hrs before mounting the coverslips for microscopy. COS-7 cells were plated on glass coverslips for 24 hours before co-transfected with shRNA-Smo and pCIG2-Smo-IRES-GFP constructs at a 1:3 ratio respectively using FuGENE 6 (Roche). Cells were then cultured for an additional 24 hours before fixation and processed for GFP and Smoothened immunostaining as described above.

Precrossing commissural explant assay. The pre-crossing assay to study repulsion was carried out as described.^{(3,4)} Rat E13 spinal cords were isolated by microdissection and dorsal explants from the caudal most region of the spinal cord were collected. Explants (˜200 μM width) were embedded in a rat-tail collagen gel matrix containing a resuspension of COS-7 cells transiently transfected with FuGENE 6 to express Netrin-1 or both Netrin-1 and Shh-N constructs placed 200-400 μm from the explants. In addition, explants were cocultured with COS-7 cell aggregates expressing either pcDNA™ 3.1 (control), Sema3F, or Sema3B constructs. Pre-crossing explant cultures were incubated at 37° C. with 5% CO₂ for 16 hours in spinal cord dorsal medium before fixation. The culture medium was changed into drug containing medium 2 hours after explants were embedded when treated with Forskolin (25 μM; F3917, Sigma) or vehicle control (0.8% DMSO, Sigma) and cultured a total of 14 hrs before fixation. For quantification the total length of axon bundle was measured in the proximal (P) and distal (D) quadrants as depicted in FIG. 31B. The P/D ratio is a measure of attractive or repulsive activity, with a ratio of >1 indicating attraction and <1 indicating repulsion.

Open book spinal cord explants treatments and DiI injections. Open book preparations of rat E13 spinal cords were isolated as previously described.^{(7,8)l At} 2-4 hrs of culture, open book explants were treated with a vehicle control (DMSO), a Shh blocking antibody (5E1; 100 ng/ml, DSHB), Forskolin (25 μM; F3917, Sigma), or a PKA inhibitor (25 μM KT5720; EI-199, Biomol) and incubated 37° C. with 5% CO₂ for an additional 12-14 hrs. After culture, open books were fixed at 37° C. with 4% PFA, washed in RT 1×PBS and injected with the lipophilic dye DiI (D282; 1 mg/ml, Molecular Probes) via iontophoresis (˜7 volts) into the dorsal region were commissural neuron cell bodies lay. The DiI was allowed to diffuse for 1 day at 37° C. to enable visualization of commissural axon trajectory before mounting in Fluoromount-G between two coverslips for confocal microscopy analysis. Injection sites were scored based on their overall behavior at the ventral midline and presented as a percentage of all injected sites from at least three experiments.

Rat spinal cord ex utero electroporation. Ptc1^{Δloop2} Myc-IRES-GFP or pCIG2-IRES-GFP (1 μg/μl) constructs were electroporated ex utero in Rat E13 embryos with three 100 ms pulses of 25V at 1 s intervals using a BTX#ECM 830 electroporator as described.⁽⁸⁾ Spinal cords were microdissected immediately after electroporation and cultured as open book explants in a collagen matrix at 37° C., 5% CO₂ for 3 days before processing for fixation (culture medium was replenished daily). shRNA constructs against Smo (Origene) were co-electroporated into rat E13 spinal cords with the EGFP-N2 vector (Clontech) modified to include the chick β-actin promoter₈ to allow GFP visualization of electroporated axons using the same electroporation parameters as above at a 1:3 GFP vector to shRNA ratio. For quantification, the total number of axons crossing or abnormally crossing the midline were counted and presented as the percentage of the total number of electropotated axons from at least three independent experiments.

Dissociated commissural neuronal culture. Spinal cords were isolated from rat E13 embryos and commissural neurons were dissociated as previously described.⁽⁸⁾ In brief, embryos were treated with Dispase I (0.25 mg/ml; D4818, Sigma) for 5 minutes prior isolation of spinal cords, followed by collection of spinal cord dorsal domains by microdissection and incubation with Trypsin (1 mg/ml; T47-99, Sigma) for 10-12 min in a 37° C. water bath. The tissue was then triturated with flamed glass pipettes and a single cell suspension was plated on poly-D-lysine (PDL; 20 μg/ml) and laminin (10 μg/ml) coated coverslips or 12 well plates (Corning). More than 90% cells in these cultures express TAG-1, a marker for spinal cord commissural neurons (FIG. 35B). 10⁵ dissociated cells were plated on PDL/laminin coated coverslips and cultured for 24 hrs before processed for immunohistochemistry as described above. For Western blot analysis, 10⁶ dissociated cells were plated on PDL/laminin coated 12-well tissue culture plates and cultured initially for 24 hrs in dorsal explant media (replenished daily) then changed to low serum medium overnight prior to treatments. Commissural dissociated cells were subsequently treated with 25 μM Forskolin, 25 μM KT5720 (PKA inhibitor) for 15 min, or 2.5 μg/ml recombinant mouse Sonic Hedgehog, amino-terminal peptide (125 nM Shh-N; 461-SH, R&D) for 1 hr and lysed in a buffer of 20 mM Hepes, 1% Triton X-100, 150 mM NaCl, 2 mM CaCl₂, 2 mM MgCl₂, 50 m/ml BSA, 10% glycerol, 1 mM EDTA, 1 mM EGTA containing a cocktail mix of protease and phosphatase inhibitors (Sigma). Samples were centrifuged and the supernatants without insoluble proteins were collected for protein concentration determination (Bio-Rad Protein Assay; 500-0006). Cell lysates were separated by SDSPAGE gel electrophoresis in 1×SDS loading buffer after DNA disruption by sonication, transferred onto a nitrocellulose membrane, blocked with 5% w/vBSA in TBST and probed for relative amounts of phosphorylated levels of PKA C (-α, β, γ; MW 42 KDa) at Thr197 (1:1000; 4781, Cell Signaling), total PKA levels of regulatory subunit PKA_{RIIβ}, MW 53 KDa (1:1000; 610625, BD Biosciences), phosphorylated levels of CREB at Ser133, MW 43 KDa (1:1000; 9198, Cell Signaling), and total CREB (48H2), MW43 KDa (1:1000; 9197, Cell Signaling). The blots were developed using the ECL™ (enhanced chemiluminescence) protein detection system (Armersham) for horseradish peroxidase conjugated secondary antibodies; anti-rabbit for phospho-PKA C, phospho-CREB, total CREB and anti- mouse for PKA_{RIIβ}.

Microscopy. 1024*1024 fluorescent photographs were taken using a Zeiss LSM510 confocal laserscanning microscope with identical acquisition parameters for each experiment. Electroporated spinal cord images were at least 12 Z-slices of a 100 μm thick Z stack.

Statistical analysis. Statistical analysis of experiments were performed using unpaired Student's two tail t-test of data analyzed from at least 3 independent experiments using ImageJ, Excel and SigmaPlot. Results are expressed as the mean average ±SEM. P≦0.05 was considered significant and was denoted by *, P≦0.05, **, P≦0.01, ***, P≦0.0025.

### REFERENCES cl For Example 10

1. Serafini, T., et al., 1994, Cell 78, 409-424.

2. Kennedy, T. E., et al., 1994, Cell 78, 425-435.

3. Serafini, T., et al., 1996, Cell 87, 1001-1014.

4. Zou, Y., et al., 2000, Cell 102, 363-375.

5. Charron, F., et al., 2004, Cell 113, 11-23.

6. Long, H., et al., 2004, Neuron 42, 213-223.

7. Lyuksyutova, A. I., et al. 2003, Science 302, 1984-1988.

8. Wolf, A. M., et al., 2008, J Neurosci 28, 3456-3467.

9. Taipale, J., et al., 2002, Nature 418, 892-897.

10. Dickson, B. J. & Gilestro, G. F., 2006, Annu Rev Cell Dev Biol 22, 651-675.

11. Bourikas, D., et al., 2005, Nat Neurosci 8, 297-304.

12. Marigo, V., et al., 1996, Development 122, 1225-1233.

13. Guan, W., et al., 2008, Dev Biol 314, 317-328.

14. Marigo, V., et al., 1996, Nature 384, 176-179.

15. Song, H., et al., 1998, Science 281, 1515-1518.

16. Terman, J. R. & Kolodkin, A. L., 2004, Science 303, 1204-1207.

17. Pasterkamp, R. J. & Giger, R. J., 2009, Curr Opin Neurobiol.

18. Hopker, V. H., et al., 1999, Nature 401, 69-73.

19. Nishiyama, M., et al. 2003, Nature 423, 990-995.

20. Ogden, S. K., et al., 2008, Nature 456, 967-970.

21. Trousse, F., et al., 2001, Development 128, 3927-3936.

22. Chen, Z., et al., 2008, Neuron 58, 325-332.

23. Sabatier, C., et al., 2004, Cell 117, 157-169.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A method for inhibiting degeneration of a neuron, the method comprising contacting the neuron with a Wnt compound or a Fzd3 dephosphorylating agent thereby inhibiting degeneration of the neuron.

2. The method of claim 1, wherein the Wnt compound is a Wnt peptide, a small molecule Wnt mimetic, or a Wnt agonist.

3. The method of claim 2, wherein the Wnt peptide is a polypeptide comprising an amino acid sequence having at least 90% identity to SEQ ID NO:1, 2, 3, 4, 5, 6, 7, 8, 9, 10, 11, 12, 13, 14, 15, 16, 17, 18, or 19.

4. The method of claim 1, wherein the Fzd3 dephosphorylating agent is a Vgl2 peptide or a Vgl2 mimetic.

5. The method of claim 1, wherein the Fzd3 dephosphorylating agent is a Dvl1 antagonist.

6. The method of claim 5, wherein the Dvl1 antagonist is a siRNA targeting Dvl1.

7. The method of claim 1, wherein the degeneration of an axon of said neuron is inhibited or wherein degeneration of a cell body of said neuron is inhibited.

8. The method of claim 7, wherein the axon is a spinal cord commissural axon.

9. The method of claim 7, wherein the axon is an upper motor neuron axon.

10. The method of claim 7, wherein the axon is a central nervous system axon.

11. The method of claim 1, wherein the neuron is a damaged spinal cord neuron.

12. The method of claim 1, wherein the neuron is a sensory neuron.

13. The method of claim 1, wherein the neuron is a motor neuron.

14. The method of claim 1, wherein the neuron is a cerebellar granule neuron, a dorsal root ganglion neuron, a cortical neuron, a sympathetic neuron, or a hippocampal neuron.

15. The method of claim 1, wherein the neuron forms part of a nerve graft or a nerve transplant.

16. The method of claim 1, wherein the neuron is ex vivo or in vitro.

17. The method of claim 15, wherein the nerve graft or the nerve transplant forms part of an organism.

18. The method of claim 17, wherein the organism is a human.

19. The method of claim 17, wherein the organism is a mammal.

20. A method of treating a neurodegenerative disease in a subject having or being at risk of developing the neurodegenerative disease by administering to the subject a Wnt compound or a Fzd3 dephosphorylating agent.

21. The method of claim 20, wherein the neurodegenerative disease is amyotrophic lateral sclerosis, Alzheimer's disease or Parkinson's disease.

22. The method of claim 20, wherein the Wnt compound is a Wnt peptide, a small molecule Wnt mimetic, or a Wnt agonist.

23. The method of claim 20, wherein wherein the Wnt peptide is a polypeptide comprising an amino acid sequence having at least 90% identity to SEQ ID NO: 1, 2, 3, 4, 5, 6, 7, 8, 9, 10, 11, 12, 13, 14, 15, 16, 17, 18, or 19.

24. The method of claim 20, wherein the Fzd3 dephosphorylating agent is a Vgl2 peptide or a Vgl2 mimetic.

25. The method of claim 1, wherein the Fzd3 dephosphorylating agent is a Dvl1 antagonist.

26. The method of claim 5, wherein the Dvl1 antagonist is a siRNA targeting Dvl1.

27. A method of identifying an agent for use in inhibiting degeneration of a neuron, the method comprising:
(a) contacting a neuron with a candidate agent;
(b) determining a level of degeneration of the neuron, wherein a lower level of degeneration of the neuron relative to a control, indicates the candidate agent inhibits degeneration of the neuron.

28. A method of identifying an agent for use in inhibiting degeneration of a neuron, the method comprising:
(a) contacting a cell with a candidate agent;
(b) determining a level of Fzd3 phosphorylation or a level of Fzd3 internalization;
wherein a reduced level of Fzd3 phosphorylation or an increased level of Fzd3 internalization relative to a control, indicates the candidate agent inhibits degeneration of the neuron

29. A method for promoting degeneration of a neuron, the method comprising contacting the neuron with a Fzd3 phosphorylating agent thereby promoting degeneration of the neuron.

30. The method of claim 1, wherein the Fzd3 phosphorylating agent is a Dvl1 peptide or a Dvl1 mimetic.

31. The method of claim 1, wherein the Fzd3 phosphorylating agent is a Vgl2 antagonist.

32. The method of claim 31, wherein the Vgl2 antagonist is a siRNA targeting Vgl2.

33. A method for modulating neuron cell guidance of a neuron comprising contacting the neuron with a SHH compound thereby modulating neuron cell guidance.

34. The method of claim 33, wherein the SHH compound is a SHH peptide.

35. The method of claim 33, wherein the SHH compound is a small molecule SHH mimetic.

36. The method of claim 34, wherein the SHH peptide is a polypeptide comprising an amino acid sequence having at least 90% identity to SEQ ID NO:51.

37. The method of claim 33, wherein the neuron is a spinal cord commissural axon.

38. The method of claim 33, wherein the neuron is an upper motor neuron axon.

39. The method of claim 33, wherein the neuron is a central nervous system axon.

40. The method of claim 33, wherein the neuron is a damaged spinal cord neuron.

41. The method of claim 33, wherein the neuron is a sensory neuron.

42. The method of claim 33, wherein the neuron is a motor neuron.

43. The method of claim 33, wherein the neuron cell guidance facilitates regeneration of the neuron.


SEQUENCE LISTING (52 sequences)

SEQ ID NO: 1 - protein, 370 aa, Homo sapiens
MGLWALLPGWVSATLLLALAALPAALAANSSGRWWGIVNVASSTNLLTDSKSLQLVLEPSLQLLSRKQRRLIRQNPGILHSVSGGLQSAVRECKWQFRNRRWNCPTAPGPHLFGKIVNRGCRETAFIFAITSAGVTHSVARSCSEGSIESCTCDYRRRGPGGPDWHWGGCSDNIDFGRLFGREFVDSGEKGRDLRFLMNLHNNEAGRTTVFSEMRQECKCHGMSGSCTVRTCWMRLPTLRAVGDVLRDRFDGASRVLYGNRGSNRASRAELLRLEPEDPAHKPPSPHDLVYFEKSPNFCTYSGRLGTAGTAGRACNSSSPALDGCELLCCGRGHRTRTQRVTERCNCTFHWCCHVSCRNCTHTRVLHECL

SEQ ID NO: 2 - protein, 360 aa, Homo sapiens
MNAPLGGIWLWLPLLLTWLTPEVNSSWWYMRATGGSSRVMCDNVPGLVSSQRQLCHRHPDVMRAISQGVAEWTAECQHQFRQHRWNCNTLDRDHSLFGRVLLRSSRESAFVYAISSAGVVFAITRACSQGEVKSCSCDPKKMGSAKDSKGIFDWGGCSDNIDYGIKFARAFVDAKERKGKDARALMNLHNNRAGRKAVKRFLKQECKCHGVSGSCTLRTCWLAMADFRKTGDYLWRKYNGAIQVVMNQDGTGFTVANERFKKPTKNDLVYFENSPDYCIRDREAGSLGTAGRVCNLTSRGMDSCEVMCCGRGYDTSHVTRMTKCGCKFHWCCAVRCQDCLEALDVHTCKAPKNADWTTAT

SEQ ID NO: 3 - protein, 391 aa, Homo sapiens
MLRPGGAEEAAQLPLRRASAPVPVPSPAAPDGSRASARLGLACLLLLLLLTLPARVDTSWWYIGALGARVICDNIPGLVSRQRQLCQRYPDIMRSVGEGAREWIRECQHQFRHHRWNCTTLDRDHTVFGRVMLRSSREAAFVYAISSAGVVHAITRACSQGELSVCSCDPYTRGRHHDQRGDFDWGGCSDNIHYGVRFAKAFVDAKEKRLKDARALMNLHNNRCGRTAVRRFLKLECKCHGVSGSCTLRTCWRALSDFRRTGDYLRRRYDGAVQVMATQDGANFTAARQGYRRATRTDLVYFDNSPDYCVLDKAAGSLGTAGRVCSKTSKGTDGCEIMCCGRGYDTTRVTRVTQCECKFHWCCAVRCKECRNTVDVHTCKAPKKAEWLDQT

SEQ ID NO: 4 - protein, 355 aa, Homo sapiens
MEPHLLGLLLGLLLGGTRVLAGYPIWWSLALGQQYTSLGSQPLLCGSIPGLVPKQLRFCRNYIEIMPSVAEGVKLGIQECQHQFRGRRWNCTTIDDSLAIFGPVLDKATRESAFVHAIASAGVAFAVTRSCAEGTSTICGCDSHHKGPPGEGWKWGGCSEDADFGVLVSREFADARENRPDARSAMNKHNNEAGRTTILDHMHLKCKCHGLSGSCEVKTCWWAQPDFRAIGDFLKDKYDSASEMVVEKHRESRGWVETLRAKYSLFKPPTERDLVYYENSPNFCEPNPETGSFGTRDRTCNVTSHGIDGCDLLCCGRGHNTRTEKRKEKCHCIFHWCCYVSCQECIRIYDVHTCK

SEQ ID NO: 5 - protein, 352 aa, Homo sapiens
MAPLGYFLLLCSLKQALGSYPIWWSLAVGPQYSSLGSQPILCASIPGLVPKQLRFCRNYVEIMPSVAEGIKIGIQECQHQFRGRRWNCTTVHDSLAIFGPVLDKATRESAFVHAIASAGVAFAVTRSCAEGTAAICGCSSRHQGSPGKGWKWGGCSEDIEFGGMVSREFADARENRPDARSAMNRHNNEAGRQAIASHMHLKCKCHGLSGSCEVKTCWWSQPDFRAIGDFLKDKYDSASEMVVEKHRESRGWVETLRPRYTYFKVPTERDLVYYEASPNFCEPNPETGSFGTRDRTCNVSSHGIDGCDLLCCGRGHNARAERRREKCRCVFHWCCYVSCQECTRVYDVHTCK

SEQ ID NO: 6 - protein, 351 aa, Homo sapiens
MSPRSCLRSLRLLVFAVFSAAASNWLYLAKLSSVGSISEEETCEKLKGLIQRQVQMCKRNLEVMDSVRRGAQLAIEECQYQFRNRRWNCSTLDSLPVFGKVVTQGTREAAFVYAISSAGVAFAVTRACSSGELEKCGCDRTVHGVSPQGFQWSGCSDNIAYGVAFSQSFVDVRERSKGASSSRALMNLHNNEAGRKAILTHMRVECKCHGVSGSCEVKTCWRAVPPFRQVGHALKEKFDGATEVEPRRVGSSRALVPRNAQFKPHTDEDLVYLEPSPDFCEQDMRSGVLGTRGRTCNKTSKAIDGCELLCCGRGFHTAQVELAERCSCKFHWCCFVKCRQCQRLVELHTCR

SEQ ID NO: 7 - protein, 380 aa, Homo sapiens
MKKSIGILSPGVALGMAGSAMSSKFFLVALAIFFSFAQVVIEANSWWSLGMNNPVQMSEVYIIGAQPLCSQLAGLSQGQKKLCHLYQDHMQYIGEGAKTGIKECQYQFRHRRWNCSTVDNTSVFGRVMQIGSRETAFTYAVSAAGVVNAMSRACREGELSTCGCSRAARPKDLPRDWLWGGCGDNIDYGYRFAKEFVDARERERIHAKGSYESARILMNLHNNEAGRRTVYNLADVACKCHGVSGSCSLKTCWLQLADFRKVGDALKEKYDSAAAMRLNSRGKLVQVNSRFNSPTTQDLVYIDPSPDYCVRNESTGSLGTQGRLCNKTSEGMDGCELMCCGRGYDQFKTVQTERCHCKFHWCCYVKCKKCTEIVDQFVCK

SEQ ID NO: 8 - protein, 359 aa, Homo sapiens
MPSLLLLFTAALLSSWAQLLTDANSWWSLALNPVQRPEMFIIGAQPVCSQLPGLSPGQRKLCQLYQEHMAYIGEGAKTGIKECQHQFRQRRWNCSTADNASVFGRVMQIGSRETAFTHAVSAAGVVNAISRACREGELSTCGCSRTARPKDLPRDWLWGGCGDNVEYGYRFAKEFVDAREREKNFAKGSEEQGRVLMNLQNNEAGRRAVYKMADVACKCHGVSGSCSLKTCWLQLAEFRKVGDRLKEKYDSAAAMRVTRKGRLELVNSRFTQPTPEDLVYVDPSPDYCLRNESTGSLGTQGRLCNKTSEGMDGCELMCCGRGYNQFKSVQVERCHCKFHWCCFVRCKKCTEIVDQYICK

SEQ ID NO: 9 - protein, 365 aa, Homo sapiens
MLPPLPSRLGLLLLLLLCPAHVGGLWWAVGSPLVMDPTSICRKARRLAGRQAELCQAEPEVVAELARGARLGVRECQFQFRFRRWNCSSHSKAFGRILQQDIRETAFVFAITAAGASHAVTQACSMGELLQCGCQAPRGRAPPRPSGLPGTPGPPGPAGSPEGSAAWEWGGCGDDVDFGDEKSRLFMDARHKRGRGDIRALVQLHNNEAGRLAVRSHTRTECKCHGLSGSCALRTCWQKLPPFREVGARLLERFHGASRVMGTNDGKALLPAVRTLKPPGRADLLYAADSPDFCAPNRRTGSPGTRGRACNSSAPDLSGCDLLCCGRGHRQESVQLEENCLCRFHWCCVVQCHRCRVRKELSLCL

SEQ ID NO: 10 - protein, 349 aa, Homo sapiens
MNRKARRCLGHLFLSLGMVYLRIGGFSSVVALGASIICNKIPGLAPRQRAICQSRPDAIIVIGEGSQMGLDECQFQFRNGRWNCSALGERTVFGKELKVGSREAAFTYAIIAAGVAHAITAACTQGNLSDCGCDKEKQGQYHRDEGWKWGGCSADIRYGIGFAKVFVDAREIKQNARTLMNLHNNEAGRKILEENMKLECKCHGVSGSCTTKTCWTTLPQFRELGYVLKDKYNEAVHVEPVRASRNKRPTFLKIKKPLSYRKPMDTDLVYIEKSPNYCEEDPVTGSVGTQGRACNKTAPQASGCDLMCCGRGYNTHQYARVWQCNCKFHWCCYVKCNTCSERTEMYTCK

SEQ ID NO: 11 - protein, 349 aa, Homo sapiens
MHRNFRKWIFYVFLCFGVLYVKLGALSSVVALGANIICNKIPGLAPRQRAICQSRPDAIIVIGEGAQMGINECQYQFRFGRWNCSALGEKTVFGQELRVGSREAAFTYAITAAGVAHAVTAACSQGNLSNCGCDREKQGYYNQAEGWKWGGCSADVRYGIDFSRRFVDAREIKKNARRLMNLHNNEAGRKVLEDRMQLECKCHGVSGSCTTKTCWTTLPKFREVGHLLKEKYNAAVQVEVVRASRLRQPTFLRIKQLRSYQKPMETDLVYIEKSPNYCEEDAATGSVGTQGRLCNRTSPGADGCDTMCCGRGYNTHQYTKVWQCNCKFHWCCFVKCNTCSERTEVFTCK

SEQ ID NO: 12 - protein, 351 aa, Homo sapiens
MGNLFMLWAALGICCAAFSASAWSVNNFLITGPKAYLTYTTSVALGAQSGIEECKFQFAWERWNCPENALQLSTHNRLRSATRETSFIHAISSAGVMYIITKNCSMGDFENCGCDGSNNGKTGGHGWIWGGCSDNVEFGERISKLFVDSLEKGKDARALMNLHNNRAGRLAVRATMKRTCKCHGISGSCSIQTCWLQLAEFREMGDYLKAKYDQALKIEMDKRQLRAGNSAEGHWVPAEAFLPSAEAELIFLEESPDYCTCNSSLGIYGTEGRECLQNSHNTSRWERRSCGRLCTECGLQVEERKTEVISSCNCKFQWCCTVKCDQCRHVVSKYYCARSPGSAQSLGKGSA

SEQ ID NO: 13 - protein, 351 aa, Homo sapiens
MFLSKPSVYICLFTCVLQLSHSWSVNNFLMTGPKAYLIYSSSVAAGAQSGIEECKYQFAWDRWNCPERALQLSSHGGLRSANRETAFVHAISSAGVMYTLTRNCSLGDFDNCGCDDSRNGQLGGQGWLWGGCSDNVGFGEAISKQFVDALETGQDARAAMNLHNNEAGRKAVKGTMKRTCKCHGVSGSCTTQTCWLQLPEFREVGAHLKEKYHAALKVDLLQGAGNSAAGRGAIADTFRSISTRELVHLEDSPDYCLENKTLGLLGTEGRECLRRGRALGRWERRSCRRLCGDCGLAVEERRAETVSSCNCKFHWCCAVRCEQCRRRVTKYFCSRAERPRGGAAHKPGRKP

SEQ ID NO: 14 - protein, 365 aa, Homo sapiens
MLDGSPLARWLAAAFGLTLLLAALRPSAAYFGLTGSEPLTILPLTLEPEAAAQAHYKACDRLKLERKQRRMCRRDPGVAETLVEAVSMSALECQFQFRFERWNCTLEGRYRASLLKRGFKETAFLYAISSAGLTHALAKACSAGRMERCTCDEAPDLENREAWQWGGCGDNLKYSSKFVKEFLGRRSSKDLRARVDFHNNLVGVKVIKAGVETTCKCHGVSGSCTVRTCWRQLAPFHEVGKHLKHKYETALKVGSTTNEAAGEAGAISPPRGRASGAGGSDPLPRTPELVHLDDSPSFCLAGRFSPGTAGRRCHREKNCESICCGRGHNTQSRVVTRPCQCQVRWCCYVECRQCTQREEVYTCKG

SEQ ID NO: 15 - protein, 357 aa, Homo sapiens
MRPPPALALAGLCLLALPAAAASYFGLTGREVLTPFPGLGTAAAPAQGGAHLKQCDLLKLSRRQKQLCRREPGLAETLRDAAHLGLLECQFQFRHERWNCSLEGRMGLLKRGFKETAFLYAVSSAALTHTLARACSAGRMERCTCDDSPGLESRQAWQWGVCGDNLKYSTKFLSNFLGSKRGNKDLRARADAHNTHVGIKAVKSGLRTTCKCHGVSGSCAVRTCWKQLSPFRETGQVLKLRYDSAVKVSSATNEALGRLELWAPARQGSLTKGLAPRSGDLVYMEDSPSFCRPSKYSPGTAGRVCSREASCSSLCCGRGYDTQSRLVAFSCHCQVQWCCYVECQQCVQEELVYTCKH

SEQ ID NO: 16 - protein, 417 aa, Homo sapiens
MGSAHPRPWLRLRPQPQPRPALWVLLFFLLLLAAAMPRSAPNDILDLRLPPEPVLNANTVCLTLPGLSRRQMEVCVRHPDVAASAIQGIQIAIHECQHQFRDQRWNCSSLETRNKIPYESPIFSRGFRESAFAYAIAAAGVVHAVSNACALGKLKACGCDASRRGDEEAFRRKLHRLQLDALQRGKGLSHGVPEHPALPTASPGLQDSWEWGGCSPDMGFGERFSKDFLDSREPHRDIHARMRLHNNRVGRQAVMENMRRKCKCHGTSGSCQLKTCWQVTPEFRTVGALLRSRFHRATLIRPHNRNGGQLEPGPAGAPSPAPGAPGPRRRASPADLVYFEKSPDFCEREPRLDSAGTVGRLCNKSSAGSDGCGSMCCGRGHNILRQTRSERCHCRFHWCCFVVCEECRITEWVSVCK

SEQ ID NO: 17 - protein, 389 aa, Homo sapiens
MLEEPRPRPPPSGLAGLLFLALCSRALSNEILGLKLPGEPPLTANTVCLTLSGLSKRQLGLCLRNPDVTASALQGLHIAVHECQHQLRDQRWNCSALEGGGRLPHHSAILKRGFRESAFSFSMLAAGVMHAVATACSLGKLVSCGCGWKGSGEQDRLRAKLLQLQALSRGKSFPHSLPSPGPGSSPSPGPQDTWEWGGCNHDMDFGEKFSRDFLDSREAPRDIQARMRIHNNRVGRQVVTENLKRKCKCHGTSGSCQFKTCWRAAPEFRAVGAALRERLGRAIFIDTHNRNSGAFQPRLRPRRLSGELVYFEKSPDFCERDPTMGSPGTRGRACNKTSRLLDGCGSLCCGRGHNVLRQTRVERCHCRFHWCCYVLCDECKVTEWVNVCK

SEQ ID NO: 18 - protein, 354 aa, Homo sapiens
MRARPQVCEALLFALALQTGVCYGIKWLALSKTPSALALNQTQHCKQLEGLVSAQVQLCRSNLELMHTVVHAAREVMKACRRAFADMRWNCSSIELAPNYLLDLERGTRESAFVYALSAAAISHAIARACTSGDLPGCSCGPVPGEPPGPGNRWGGCADNLSYGLLMGAKFSDAPMKVKKTGSQANKLMRLHNSEVGRQALRASLEMKCKCHGVSGSCSIRTCWKGLQELQDVAADLKTRYLSATKVVHRPMGTRKHLVPKDLDIRPVKDSELVYLQSSPDFCMKNEKVGSHGTQDRQCNKTSNGSDSCDLMCCGRGYNPYTDRVVERCHCKYHWCCYVTCRRCERTVERYVCK

SEQ ID NO: 19 - protein, 365 aa, Homo sapiens
MDRAALLGLARLCALWAALLVLFPYGAQGNWMWLGIASFGVPEKLGCANLPLNSRQKELCKRKPYLLPSIREGARLGIQECGSQFRHERWNCMITAAATTAPMGASPLFGYELSSGTKETAFIYAVMAAGLVHSVTRSCSAGNMTECSCDTTLQNGGSASEGWHWGGCSDDVQYGMWFSRKFLDFPIGNTTGKENKVLLAMNLHNNEAGRQAVAKLMSVDCRCHGVSGSCAVKTCWKTMSSFEKIGHLLKDKYENSIQISDKTKRKMRRREKDQRKIPIHKDDLLYVNKSPNYCVEDKKLGIPGTQGRECNRTSEGADGCNLLCCGRGYNTHVVRHVERCECKFIWCCYVRCRRCESMTDVHTCK

SEQ ID NO: 20 - protein, 370 aa, Mus musculus
MGLWALLPSWVSTTLLLALTALPAALAANSSGRWWGIVNIASSTNLLTDSKSLQLVLEPSLQLLSRKQRRLIRQNPGILHSVSGGLQSAVRECKWQFRNRRWNCPTAPGPHLFGKIVNRGCRETAFIFAITSAGVTHSVARSCSEGSIESCTCDYRRRGPGGPDWHWGGCSDNIDFGRLFGREFVDSGEKGRDLRFLMNLHNNEAGRTTVFSEMRQECKCHGMSGSCTVRTCWMRLPTLRAVGDVLRDRFDGASRVLYGNRGSNRASRAELLRLEPEDPAHKPPSPHDLVYFEKSPNFCTYSGRLGTAGTAGRACNSSSPALDGCELLCCGRGHRTRTQRVTERCNCTFHWCCHVSCRNCTHTRVLHECL

SEQ ID NO: 21 - protein, 360 aa, Mus musculus
MNVPLGGIWLWLPLLLTWLTPEVSSSWWYMRATGGSSRVMCDNVPGLVSRQRQLCHRHPDVMRAIGLGVAEWTAECQHQFRQHRWNCNTLDRDHSLFGRVLLRSSRESAFVYAISSAGVVFAITRACSQGELKSCSCDPKKKGSAKDSKGTFDWGGCSDNIDYGIKFARAFVDAKERKGKDARALMNLHNNRAGRKAVKRFLKQECKCHGVSGSCTLRTCWLAMADFRKTGDYLWRKYNGAIQVVMNQDGTGFTVANKRFKKPTKNDLVYFENSPDYCIRDREAGSLGTAGRVCNLTSRGMDSCEVMCCGRGYDTSHVTRMTKCECKFHWCCAVRCQDCLEALDVHTCKAPKSADWATPT

SEQ ID NO: 22 - protein, 389 aa, Mus musculus
MLKLQGEDEAAQLAPRRARVPVPRPTAPDVSPSSARLGLACLLLLLLLTLPARVDTSWWYIGALGARVICDNIPGLVSRQRQLCQRYPDIMRSVGEGAREWIRECQHQFRHHRWNCTTLDRDHTVFGRAMLRSSREAAFVYAISSAGVVHAITRACSQGELSVCSCDPYTRGRHHDQRGDFDWGGCSDNIHYGVRFAKAFVDAKEKRLKDARALMNLHNNRCGRTAVRRFLKLECKCHGVSGSCTLRTCWRALSDFRRTGDYLRRRYDGAVQVTATQDGANFTAARQGYRHATRTDLVYFDNSPDYCVLDKAAGSLGTAGRVCSKTSKGTDGCEIMCCGRGYDTTRVTRVTQCECKFHWCCAVRCKECRNTVDVHTCKAPKKAEWLDQT

SEQ ID NO: 23 - protein, 355 aa, Mus musculus
MEPHLLGLLLGLLLSGTRVLAGYPIWWSLALGQQYTSLASQPLLCGSIPGLVPKQLRFCRNYIEIMPSVAEGVKLGIQECQHQFRGRRWNCTTIDDSLAIFGPVLDKATRESAFVHAIASAGVAFAVTRSCAEGTSTICGCDSHHKGPPGEGWKWGGCSEDADFGVLVSREFADARENRPDARSAMNKHNNEAGRTTILDHMHLKCKCHGLSGSCEVKTCWWAQPDFRAIGDFLKDKYDSASEMVVEKHRESRGWVETLRAKYALFKPPTERDLVYYENSPNFCEPNPETGSFGTRDRTCNVTSHGIDGCDLLCCGRGHNTRTEKRKEKCHCVFHWCCYVSCQECIRIYDVHTCK

SEQ ID NO: 24 - protein, 352 aa, Mus musculus
MAPLGYLLVLCSLKQALGSYPIWWSLAVGPQYSSLSTQPILCASIPGLVPKQLRFCRNYVEIMPSVAEGVKAGIQECQHQFRGRRWNCTTVSNSLAIFGPVLDKATRESAFVHAIASAGVAFAVTRSCAEGSAAICGCSSRLQGSPGEGWKWGGCSEDIEFGGMVSREFADARENRPDARSAMNRHNNEAGRQAIASHMHLKCKCHGLSGSCEVKTCWWSQPDFRTIGDFLKDKYDSASEMVVEKHRESRGWVETLRPRYTYFKVPTERDLVYYEASPNFCEPNPETGSFGTRDRTCNVSSHGIDGCDLLCCGRGHNARTERRREKCHCVFHWCCYVSCQECTRVYDVHTCK

SEQ ID NO: 25 - protein, 351 aa, Mus musculus
MSPRSCLRSLRLLVFAVFSAAASNWLYLAKLSSVGSISEEETCEKLKGLIQRQVQMCKRNLEVMDSVRRGAQLAIEECQYQFRNRRWNCSTLDSLPVFGKVVTQGTREAAFVYAISSAGVAFAVTRACSSGELEKCGCDRTVHGVSPQGFQWSGCSDNIAYGVAFSQSFVDVRERSKGASSSRALMNLHNNEAGRKAILTHMRVECKCHGVSGSCEVKTCWRAVPPFRQVGHALKEKFDGATEVEPRRVGSSRALVPRNAQFKPHTDEDLVYLEPSPDFCEQDIRSGVLGTRGRTCNKTSKAIDGCELLCCGRGFHTAQVELAERCGCRFHWCCFVKCRQCQRLVEMHTCR

SEQ ID NO: 26 - protein, 380 aa, Mus musculus
MKKPIGILSPGVALGTAGGAMSSKFFLMALATFFSFAQVVIEANSWWSLGMNNPVQMSEVYIIGAQPLCSQLAGLSQGQKKLCHLYQDHMQYIGEGAKTGIKECQYQFRHRRWNCSTVDNTSVFGRVMQIGSRETAFTYAVSAAGVVNAMSRACREGELSTCGCSRAARPKDLPRDWLWGGCGDNIDYGYRFAKEFVDARERERIHAKGSYESARILMNLHNNEAGRRTVYNLADVACKCHGVSGSCSLKTCWLQLADFRKVGDALKEKYDSAAAMRLNSRGKLVQVNSRFNSPTTQDLVYIDPSPDYCVRNESTGSLGTQGRLCNKTSEGMDGCELMCCGRGYDQFKTVQTERCHCKFHWCCYVKCKKCTEIVDQFVCK

SEQ ID NO: 27 - protein, 372 aa, Mus musculus
MLVPGHWDGLRPAMPSLLLVVVAALLSSWAQLLTDANSWWSLALNPVQRPEMFIIGAQPVCSQLPGLSPGQRKLCQLYQEHMSYIGEGAKTGIRECQHQFRQRRWNCSTVDNTSVFGRVMQIGSRETAFTYAVSAAGVVNAISRACREGELSTCGCSRAARPKDLPRDWLWGGCGDNVEYGYRFAKEFVDAREREKNFAKGSEEQGRALMNLQNNEAGRRAVYKMADVACKCHGVSGSCSLKTCWLQLAEFRKVGDRLKEKYDSAAAMRITRQGKLELANSRFNQPTPEDLVYVDPSPDYCLRNETTGSLGTQGRLCNKTSEGMDGCELMCCGRGYDRFKSVQVERCHCRFHWCCFVRCKKCTEVVDQYVCK

SEQ ID NO: 28 - protein, 364 aa, Mus musculus
MLPPVPSRLGLLLLLLCPAHVDGLWWAVGSPLVMDPTSICRKARRLAGRQAELCQAEPEVVAELARGARLGVRECQFQFRFRRWNCSSHSKAFGRVLQQDIRETAFVFAITAAGASHAVTQACSMGELLQCGCQAPRGRAPPRPSGLLGTPGPPGPTGSPDASAAWEWGGCGDDVDFGDEKSRLFMDAQHKRGRGDIRALVQLHNNEAGRLAVRSHTRTECKCHGLSGSCALRTCWQKLPPFREVGARLLERFHGASRVMGTNDGKALLPAVRTLKPPGRADLLYAADSPDFCAPNRRTGSPGTRGRACNSSAPDLSGCDLLCCGRGHRQESVQLEENCLCRFHWCCVVQCHRCRVRKELSLCL

SEQ ID NO: 29 - protein, 349 aa, Mus musculus
MTRKARRCLGHLFLSLGIVYLRIGGFSSVVALGASIICNKIPGLAPRQRAICQSRPDAIIVIGEGSQMGLDECQFQFRNGRWNCSALGERTVFGKELKVGSREAAFTYAIIAAGVAHAITAACTQGNLSDCGCDKEKQGQYHRDEGWKWGGCSADIRYGIGFAKVFVDAREIKQNARTLMNLHNNEAGRKILEENMKLECKCHGVSGSCTTKTCWTTLPQFRELGYVLKDKYNEAVHVEPVRASRNKRPTFLKIKKPLSYRKPMDTDLVYIEKSPNYCEEDPVTGSVGTQGRACNKTAPQASGCDLMCCGRGYNTHQYARVWQCNCKFHWCCYVKCNTCSERTEMYTCK

SEQ ID NO: 30 - protein, 349 aa, Mus musculus
MHRNFRKWIFYVFLCFGVLYVKLGALSSVVALGANIICNKIPGLAPRQRAICQSRPDAIIVIGEGAQMGIDECQHQFRFGRWNCSALGEKTVFGQELRVGSREAAFTYAITAAGVAHAVTAACSQGNLSNCGCDREKQGYYNQAEGWKWGGCSADVRYGIDFSRRFVDAREIKKNARRLMNLHNNEAGRKVLEDRMKLECKCHGVSGSCTTKTCWTTLPKFREVGHLLKEKYNAAVQVEVVRASRLRQPTFLRIKQLRSYQKPMETDLVYIEKSPNYCEEDAATGSVGTQGRLCNRTSPGADGCDTMCCGRGYNTHQYTKVWQCNCKFHWCCFVKCNTCSERTEVFTCK

SEQ ID NO: 31 - protein, 354 aa, Mus musculus
MGHLLMLWVAAGMCYPALGASAWSVNNFLITGPKAYLTYTASVALGAQIGIEECKFQFAWERWNCPEHAFQFSTHNRLRAATRETSFIHAIRSAAIMYAVTKNCSMGDLENCGCDESQNGKTGGHGWIWGGCSDNVEFGEKISRLFVDSLEKGKDARALVNLHNNRAGRLAVRASTKRTCKCHGISGSCSIQTCWLQLADFRQMGNYLKAKYDRALKIEMDKRQLRAGNRAEGRWALTEAFLPSTEAELIFLEGSPDYCNRNASLSIQGTEGRECLQNARSASRREQRSCGRLCTECGLQVEERRAEAVSSCDCNFQWCCTVKCGQCRRVVSRYYCTRPVGSARPRGRGKDSAW

SEQ ID NO: 32 - protein, 368 aa, Mus musculus
MLPISQCLSRSPCLSPKTMFLMKPVCVLLVTCVLHRSHAWSVNNFLMTGPKAYLVYSSSVAAGAQSGIEECKYQFAWDRWNCPERALQLSSHGGLRSANRETAFVHAISSAGVMYTLTRNCSLGDFDNCGCDDSRNGQLGGQGWLWGGCSDNVGFGEAISKQFVDALETGQDARAAMNLHNNEAGRKAVKGTMKRTCKCHGVSGSCTTQTCWLQLPEFREVGAHLKEKYHAALKVDLLQGAGNSAAGRGAIADTFRSISTRELVHLEDSPDYCLENKTLGLLGTEGRECLRRGRALGRWERRSCRRLCGDCGLAVEERRAETVSSCNCKFHWCCAVRCEQCRRRVTKYFCSRAERPPRGAAHKPGKNS

SEQ ID NO: 33 - protein, 365 aa, Mus musculus
MLDGSLLARWLAAAFGLTLLLAALRPSAAYFGLTGSEPLTILPLTLETEAAAQAHYKACDRLKLERKQRRMCRRDPGVAETLVEAVSMSALECQYQFRFERWNCTLEGRYRASLLKRGFKETAFLYAISSAGLTHALAKACSAGRMERCTCDEAPDLENREAWQWGGCGDNLKYSSKFVKEFLGRRSSKDLRARVDFHNNLVGVKVIKAGVETTCKCHGVSGSCTVRTCWRQLAPFHEVGKHLKHKYETSLKVGSTTNEATGEAGAISPPRGRASGSGGGDPLPRTPELVHLDDSPSFCLAGRFSPGTAGRRCHREKNCESICCGRGHNTQSRVVTRPCQCQVRWCCYVECRQCTQREEVYTCKG

SEQ ID NO: 34 - protein, 359 aa, Mus musculus
MRPAPALALAALCLLVLPAAAAAAAYFGLTGREVLTPFPGLGTAAAPAQAGAHLKQCDLLKLSRRQKQLCRREPGLAETLRDAAHLGLLECQFQFRQERWNCSLEGRTGLLQRGFKETAFLYAVSAAALTHALARACSAGRMERCTCDDSPGLESRQAWQWGVCGDNLKYSTKFLSNFLGPKRGSKDLRARADAHNTHVGIKAVKSGLRTTCKCHGVSGSCAVRTCWKQLSPFRETGQVLKLRYDTAVKVSSATNEALGRLELWAPAKPGGPAKGLAPRPGDLVYMEDSPSFCRPSKYSPGTAGRVCSRDSSCSSLCCGRGYDTQSRMVVFSCHCQVQWCCYVECQQCAQQELVYTCKR

SEQ ID NO: 35 - protein, 417 aa, Mus musculus [sequence omitted]

SEQ ID NO: 36 - protein, 389 aa, Mus musculus
MLEEPRSRPPPLGLAGLLFLALFSRALSNEILGLKLPGEPPLTANTVCLTLSGLSKRQLGLCLRSPDVTASALQGLHIAVHECQHQLRDQRWNCSALEGGGRLPHHSAILKRGFRESAFSFSMLAAGVMHAVATACSLGKLVSCGCGWKGSGEQDRLRAKLLQLQALSRGKTFPISQPSPVPGSVPSPGPQDTWEWGGCNHDMDFGEKFSRDFLDSREAPRDIQARMRIHNNRVGRQVVTENLKRKCKCHGTSGSCQFKTCWRAAPEFRAIGAALRERLSRAIFIDTHNRNSGAFQPRLRPRRLSGELVYFEKSPDFCERDPTLGSPGTRGRACNKTSRLLDGCGSLCCGRGHNVLRQTRVERCHCRFHWCCYVLCDECKVTEWVNVCK

SEQ ID NO: 37 - protein, 354 aa, Mus musculus
MRARPQVCEALLFALALHTGVCYGIKWLALSKTPAALALNQTQHCKQLEGLVSAQVQLCRSNLELMRTIVHAARGAMKACRRAFADMRWNCSSIELAPNYLLDLERGTRESAFVYALSAATISHTIARACTSGDLPGCSCGPVPGEPPGPGNRWGGCADNLSYGLLMGAKFSDAPMKVKKTGSQANKLMRLHNSEVGRQALRASLETKCKCHGVSGSCSIRTCWKGLQELQDVAADLKTRYLSATKVVHRPMGTRKHLVPKDLDIRPVKDSELVYLQSSPDFCMKNEKVGSHGTQDRQCNKTSNGSDSCDLMCCGRGYNPYTDRVVERCHCKYHWCCYVTCRRCERTVERYVCK

SEQ ID NO: 38 - protein, 364 aa, Mus musculus
MDRAALLALPSLCALWAAVLSLLPCGTQGNWMWLGIASFGVPEKLGCADLPLNSRQKELCKRKPYLLPSIREGARLGIQECRSQFRHERWNCMVATTTSTQLATAPLFGYELSSGTKETAFIYAIMAAGLVHSVTRSCSAGNMTECSCDTTLQNGGSPSEGWHWGGCSDDVQYGMWFSRKFLDLPIRNTTGKESRVLLAMNLHNNEAGRQAVAKLMSVDCRCHGVSGSCAVKTCWKTMSSFEKIGHFLKDKYENSIQISDKTKRKMRRREKDQRQTPILKDDLLYVHKSPNYCVENKKLGIPGTQGRECNRTSGGADGCNLLCCGRGYNTHVVRHVERCECKFIWCCYVRCRRCESMTDVHTCK

SEQ ID NO: 39 - DNA, 30 bp, Artificial Sequence
  Synthetic DNA construct of forward primer for Patched1
atggcctcggctggtaacgccgccggggcc

SEQ ID NO: 40 - DNA, 30 bp, Artificial Sequence
  Synthetic DNA construct for reverse primer for Patched1
gttggagctgctcccccacggcctctcctc

SEQ ID NO: 41 - protein, 21 aa, Artificial Sequence
  Synthetic peptide sequence including PmeI linker site
GLDLTDIVPVSTLVCAVFLLN

SEQ ID NO: 42 - DNA, 30 bp, Artificial Sequence
  Synthetic DNA construct of forward primer for Smoothened
atggccgctggccgccccgtgcgtgggccc

SEQ ID NO: 43 - DNA, 30 bp, Artificial Sequence
  Synthetic DNA construct of reverse primer for Smoothened
gaagtctgagtctgcatccaagatctcagc

SEQ ID NO: 44 - DNA, 29 bp, Artificial Sequence
  Synthetic DNA of sequence of Smo target for shRNA
tgactctgttctccatcaagagcaaccac

SEQ ID NO: 45 - DNA, 12 bp, Artificial Sequence
  Synthetic DNA sequence of pRS shRNA vector at 5′ end of SV40 early promoter
ttttttgaagct

SEQ ID NO: 46 - protein, 521 aa, Homo sapiens
MDTESQYSGYSYKSGHSRSSRKHRDRRDRHRSKSRDGGRGDKSVTIQAPGEPLLDNESTRGDERDDNWGETTTVVTGTSEHSISHDDLTRIAKDMEDSVPLDCSRHLGVAAGATLALLSFLTPLAFLLLPPLLWREELEPCGTACEGLFISVAFKLLILLLGSWALFFRRPKASLPRVFVLRALLMVLVFLLVVSYWLFYGVRILDARERSYQGVVQFAVSLVDALLFVHYLAVVLLELRQLQPQFTLKVVRSTDGASRFYNVGHLSIQRVAVWILEKYYHDFPVYNPALLNLPKSVLAKKVSGFKVYSLGEENSTNNSTGQSRAVIAAAARRRDNSHNEYYYEEAEHERRVRKRRARLVVAVEEAFTHIKRLQEEEQKNPREVMDPREAAQAIFASMARAMQKYLRTTKQQPYHTMESILQHLEFCITHDMTPKAFLERYLAAGPTIQYHKERWLAKQWTLVSEEPVTNGLKDGIVFLLKRQDFSLVVSTKKVPFFKLSEEFVDPKSHKFVMRLQSETSV

SEQ ID NO: 47 - protein, 284 aa, Homo sapiens
ELRQLQPQFTLKVVRSTDGASRFYNVGHLSIQRVAVWILEKYYHDFPVYNPALLNLPKSVLAKKVSGFKVYSLGEENSTNNSTGQSRAVIAAAARRRDNSHNEYYYEEAEHERRVRKRRARLVVAVEEAFTHIKRLQEEEQKNPREVMDPREAAQAIFASMARAMQKYLRTTKQQPYHTMESILQHLEFCITHDMTPKAFLERYLAAGPTIQYHKERWLAKQWTLVSEEPVTNGLKDGIVFLLKRQDFSLVVSTKKVPFFKLSEEFVDPKSHKFVMRLQSETSV

SEQ ID NO: 48 - protein, 521 aa, Mus musculus
MDTESQYSGYSYKSGHSRSSRKHRDRRDRHRSKSRDGSRGDKSVTIQAPGEPLLDNESTRGDERDDNWGETTTVVTGTSEHSISHDDLTRIAKDMEDSVPLDCSRHLGVAAGAILALLSFLTPLAFLLLPPLLWREELEPCGTACEGLFISVAFKLLILLLGSWALFFRRPKASLPRVFVLRALLMVLVFLLVISYWLFYGVRILDARERSYQGVVQFAVSLVDALLFVHYLAVVLLELRQLQPQFTLKVVRSTDGASRFYNVGHLSIQRVAVWILEKYYHDFPVYNPALLNLPKSVLAKKVSGFKVYSLGEENSTNNSTGQSRAVIAAAARRRDNSHNEYYYEEAEHERRVRKRRARLVVAVEEAFTHIKRLQEEEQKNPREVMDPREAAQAIFASMARAMQKYLRTTKQQPYHTMESILQHLEFCITHDMTPKAFLERYLAAGPTIQYHKERWLAKQWTLVSEEPVTNGLKDGIVFLLKRQDFSLVVSTKKVPFFKLSEEFVDPKSHKFVMRLQSETSV

SEQ ID NO: 49 - protein, 670 aa, Homo sapiens
MAETKIIYHMDEEETPYLVKLPVAPERVTLADFKNVLSNRPVHAYKFFFKSMDQDFGVVKEEIFDDNAKLPCFNGRVVSWLVLAEGAHSDAGSQGTDSHTDLPPPLERTGGIGDSRPPSFHPNVASSRDGMDNETGTESMVSHRRERARRRNREEAARTNGHPRGDRRRDVGLPPDSASTALSSELESSSFVDSDEDGSTSRLSSSTEQSTSSRLIRKHKRRRRKQRLRQADRASSFSSITDSTMSLNIVTVTLNMERHHFLGISIVGQSNDRGDGGIYIGSIMKGGAVAADGRIEPGDMLLQVNDVNFENMSNDDAVRVLREIVSQTGPISLTVAKCWDPTPRSYFTVPRADPVRPIDPAAWLSHTAALTGALPRYELEEAPLTVKSDMSAVVRVMQLPDSGLEIRDRMWLKITIANAVIGADVVDWLYTHVEGFKERREARKYASSLLKHGFLRHTVNKITFSEQCYYVFGDLCSNLATLNLNSGSSGTSDQDTLAPLPHPAAPWPLGQGYPYQYPGPPPCFPPAYQDPGFSYGSGSTGSQQSEGSKSSGSTRSSRRAPGREKERRAAGAGGSGSESDHTAPSGVGSSWRERPAGQLSRGSSPRSQASATAPGLPPPHPTTKAYTVVGGPPGGPPVRELAAVPPELTGSRQSFQKAMGNPCEFFVDIM

SEQ ID NO: 50 - protein, 695 aa, Mus musculus
MAETKIIYHMDEEETPYLVKLPVAPERVTLADFKNVLSNRPVHAYKFFFKSMDQDFGVVKEEIFDDNAKLPCFNGRVVSWLVLAEGAHSDAGSQGTDSHTDLPPPLERTGGIGDSRPPSFHPNVASSRDGMDNETGTESMVSHRRERARRRNRDEAARTNGHPRGDRRRDLGLPPDSASTVLSSELESSSFIDSDEEDNTSRLSSSTEQSTSSRLVRKHKCRRRKQRLRQTDRASSFSSITDSTMSLNIITVTLNMERHHFLGISIVGQSNDRGDGGIYIGSIMKGGAVAADGRIEPGDMLLQVNDVNFENMSNDDAVRVLREIVSQTGPISLTVAKCWDPTPRSYFTIPRADPVRPIDPAAWLSHTAALTGALPRYGTSPCSSAITRTSSSSLTSSVPGAPQLEEAPLTVKSDMSAIVRVMQLPDSGLEIRDRMWLKITIANAVIGADVVDWLYTHVEGFKERREARKYASSMLKHGFLRHTVNKITFSEQCYYVFGDLCSNLASLNLNSGSSGASDQDTLAPLPHPSVPWPLGQGYPYQYPGPPPCFPPAYQDPGFSCGSGSAGSQQSEGSKSSGSTRSSHRTPGREERRATGAGGSGSESDHTVPSGSGSTGWWERPVSQLSRGSSPRSQASAVAPGLPPLHPLTKAYAVVGGPPGGPPVRELAAVPPELTGSRQSFQKAMGNPCEFFVDIM

SEQ ID NO: 51 - protein, 462 aa, Homo sapiens
MLLLARCLLLVLVSSLLVCSGLACGPGRGFGKRRHPKKLTPLAYKQFIPNVAEKTLGASGRYEGKISRNSERFKELTPNYNPDIIFKDEENTGADRLMTQRCKDKLNALAISVMNQWPGVKLRVTEGWDEDGHHSEESLHYEGRAVDITTSDRDRSKYGMLARLAVEAGFDWVYYESKAHIHCSVKAENSVAAKSGGCFPGSATVHLEQGGTKLVKDLSPGDRVLAADDQGRLLYSDFLTFLDRDDGAKKVFYVIETREPRERLLLTAAHLLFVAPHNDSATGEPEASSGSGPPSGGALGPRALFASRVRPGQRVYVVAERDGDRRLLPAAVHSVTLSEEAAGAYAPLTAQGTILINRVLASCYAVIEEHSWAHRAFAPFRLAHALLAALAPARTDRGGDSGGGDRGGGGGRVALTAPGAADAPGAGATAGIHWYSQLLYQIGTWLLDSEALHPLGMAVKSS

SEQ ID NO: 52 - protein, 437 aa, Mus musculus
MLLLLARCFLVILASSLLVCPGLACGPGRGFGKRRHPKKLTPLAYKQFIPNVAEKTLGASGRYEGKITRNSERFKELTPNYNPDIIFKDEENTGADRLMTQRCKDKLNALAISVMNQWPGVKLRVTEGWDEDGHHSEESLHYEGRAVDITTSDRDRSKYGMLARLAVEAGFDWVYYESKAHIHCSVKAENSVAAKSGGCFPGSATVHLEQGGTKLVKDLRPGDRVLAADDQGRLLYSDFLTFLDRDEGAKKVFYVIETLEPRERLLLTAAHLLFVAPHNDSGPTPGPSALFASRVRPGQRVYVVAERGGDRRLLPAAVHSVTLREEEAGAYAPLTAHGTILINRVLASCYAVIEEHSWAHRAFAPFRLAHALLAALAPARTDGGGGGSIPAAQSATEARGAEPTAGIHWYSQLLYHIGTWLLDSETMHPLGMAVKSS